(19) United States
(12) Patent Application Publication
(10) Pub. No.: US 2011/0305749 A1
(43) Pub. Date: Dec. 15, 2011

(54) HIV-1 Envelope Polypeptides for HIV Vaccine

(76) Inventors: Mogens Ryttergaard Duch, Risskov (DK); Finn Skou Pedersen, Arhus V (DK); Shervin Bahrami, Arhus C (DK); Palle Villesen Fredsted, Marslet (DK); Carsten Wiuf, Arhus N (DK); Lars Jørgen Østergaard, Lystrup (DK); Martin Tolstrup, Risskov (DK)

(21) Appl. No.: 13/060,564

(22) PCT Filed: Aug. 28, 2009
(86) PCT No.: PCT/DK2009/050221

§ 371 Date: Sep. 1, 2011

(30) Foreign Application Priority Data
Aug. 28, 2008 (DK) PA 2008 01184
Mar. 23, 2009 (DK) PA 2009 00398

(51) Int. Cl. A61K 39/21; A61K 9/127; C12N 5/071; C12N 1/10; C07K 16/10; A61K 39/395; C07K 14/16; A61P 37/04; A61P 31/18; C12N 7/00; C12N 1/20; B82Y 5/00

(52) U.S. Cl. 424/450; 424/188.1; 435/235.1; 435/325; 435/258.1; 435/252.1; 424/85.2; 530/387.9; 424/139.1; 530/324; 530/350; 977/773; 977/906

(57) ABSTRACT

Immunogenic HIV-1 envelope polypeptides are provided, wherein specific amino acid residues are mutated to repress immunosuppression in GP41, thereby boosting the immune response against HIV-1. Specifically, mutation of those specific residues does not affect the fusogenic properties of the viral particle and/or the overall protein structure of the viral envelope protein. The invention further provides peptides and antigens based on the immunogenic HIV-1 envelopes as well as nucleic acid sequences and vectors encoding those. Moreover, biological entities such as viral particles are provided, as well as use of the provided components for preparation of a vaccine against HIV-1/AIDS.

### FIELD OF INVENTION

The present invention relates to HIV-1 envelope polypeptides, which may be used in a vaccine for HIV. The invention also encompasses biological entities comprising such polypeptides or nucleic acids encoding those, in particular retroviral particles. Moreover the invention relates to vaccine compositions, which comprise a polypeptide of the present invention and an adjuvant as well as kits comprising said vaccine compositions.

### BACKGROUND OF INVENTION

Human immunodeficiency virus (HIV) is according to WHO one of the most serious health crisis the world faces today. AIDS has killed more than 25 million people since 1981. In the most severely affected countries, the average life expectancy is now declining to 49 years of age-13 years less than in the absence of AIDS. According to UNAIDS an estimated number of 39.5 million people were living with HIV virus in 2006 and 4.3 million were infected in 2006. In many regions new infections are heavily concentrated in the younger generations (15-24 years of age). Access to treatment and care has greatly increased in recent years. Determining real time trends to HIV incidence and in particular the impact of prevention programmes ideally requires long studies of a large number of people. Given the practical difficulties of conducting such studies focus has been placed on young women and their infants. Children living with HIV typically acquire infection through a mother-to-child-transmission (MTCT), which occur during pregnancy, delivery or during breastfeeding. Renewed efforts are urgently required to increase access to comprehensive and integrated programmes to prevent HIV infection in infants and young children, which will indicate a route to HIV-free generations.

There are two known types of HIV; HIV-1 and HIV-2 that infect humans. They belong to a group of retroviruses called Lentiviruses and a virus similar to HIV has been found in African monkeys. Retroviruses transfer their genes from a producer cell to a target cell as a genomic RNA transcript, which is reverse-transcribed after infection and integrated into the DNA genome of the target cell.

The first person with a documented HIV-infection died in 1959. In the early 1980s doctors in the US become aware, that more and more patients suffered from abnormal infections and showed signs of immune failure. The syndrome was named Acquired Immune Deficiency Syndrome (AIDS) and it was soon after discovered that HIV was the causative agent for the observed destruction of the immune system. Initially patients were offered a treatment based solely on pain relief and almost all inevitably died. In mid 1990s there were two important breakthroughs in treatment. Firstly, a new group of antiretroviral agents were discovered and secondly it became possible to measure the amount of HIV virus in blood. These two advances made it possible to treat patients with a combination of different agents and doctors were able to check, whether the treatment actually worked. The result was that the immune system of infected patients gradually became normal and patients lived longer. Today infected people in Western countries are having the same level of quality of life as those not infected and they are able to have children, although the economical and psychological consequences of having HIV are huge. The situation is, however, even more severe in developing countries, where more than 95% of those people infected with HIV/AIDS are living. Worldwide more than 25 million people have died from AIDS in the last 25 years.

Approximately 95% of the people who get infected today live in the developing countries, where expensive antiviral drugs are not available. Therefore, there is an urgent need for an effective vaccine—the only effective solution to the uncontrolled HIV pandemic. During the last few years research has brought up new knowledge on the fundamental biology of HIV-virus which is leading to new antiviral drugs and strategies for vaccine design. In spite of these substantial advances, an effective vaccine does not yet exist. Only attenuated (that is live but weakened) HIV-strains has been able to provide immunity in primate studies even though they will never reach a required safety profile suitable for mass vaccination.

The replication process for HIV-1 has an error rate of about one per 10,000 base pairs. Since the entire viral genome is just under 10,000 base pairs, it is estimated that on average one error is introduced into the HIV-1 genome at each viral replication cycle. This high mutation rate contributes to extensive variability of the viruses inside any one person and an even wider variability across populations.

This variability has resulted in three HIV-1 variants being described, and the subspecies of virus called “clades.” The distinctions are based on the structure of the envelope proteins, which are especially variable. The M (for major) variant is by far the most prevalent world wide. Within the M variant are clades A, B, C, D, E, F, G, H, I, J and K, with clades A through E representing the vast majority of infections globally. Clades A, C and D are dominant in Africa, while clade B is the most prevalent in Europe, North and South America and Southeast Asia. Clades E and C are dominant in Asia. These clades differ by as much as 35%. Another variant is clade O, which is observed in Cameroun isolates of HIV-1. The greatest variation in structure is seen in the envelope proteins gpl20 and gp41.

There are two important results from the very high mutation rate of HIV-1 that have profound consequences for the epidemic. First, the high mutation rate is one of the mechanisms that allow the virus to escape from control by drug therapies. These new viruses represent resistant strains. The high mutation rate also allows the virus to escape the patient's immune system by altering the structures that are recognized by immune components. An added consequence of this extensive variability is that the virus can also escape from control by vaccines, and vaccines based on envelope proteins will likely be non-effective.

### SUMMARY OF INVENTION

The present invention provides a new approach to designing immunogenic HIV-1 envelope polypeptides derived from HIV-1 envelope protein, GP41 and/or GP120. Specific amino acid residues are mutated to repress immunosuppression in GP41, thereby boosting the immune response against HIV-1. Mutation of those specific residues however, does not affect the fusogenic properties of the viral particle and/or the overall protein structure of the viral envelope protein. The polypeptides of the present invention can be inserted in a suitable construct and expressed in an organism to produce a vaccine or an immunogenic response against HIV.

In one aspect, the present invention relates to an HIV-1 envelope polypeptide comprising an amino acid sequence selected from the group of amino acid sequences consisting of: X(1-22)-C(23)-X(24-28)-C(29)-X(30-50), or any part thereof, or functional homolog or any amino acid sequence with at least 90% identity to said amino acid sequence or part thereof. Thus, the claimed envelope polypeptide comprise a 50 amino acid sequence, wherein X(1-22) indicates an unbranched sequence of 22 amino acid residues, selected from any known amino acid, while C(23) denotes that amino acid residue number 23 is cysteine; followed by 5 residues of any amino acid, X(24-28), a cysteine in position 29 (C(29)), and another unbranched sequence of 21 amino acid residues selected from any amino acid (X(30-50)).

The 50 amino acid sequence is derived from any known HIV-1 envelope subtype, such as those identified by SEQ ID NO: 74-119. Thus, the specific amino acid in position X of the amino acid sequence above may be selected from an amino acid in the corresponding position of any of those and other HV-1 subtypes. In a preferred embodiment, the present invention relates to an HIV-1 envelope as defined in the aspect above, wherein the amino acid residues in the amino acid sequence of the HIV-1 envelope polypeptide are selected from the groups of residues consisting of:

### X(1): L, S, R, P, F, A, V, M, and I; and

### X(2): Q, R, K, H, L, M, and P; and

### X(3): A, T, V, H, S, R, Q, G, M, and E; and

### X(4): R, K, G, E, T, S, C, M, and H; and

### X(5): V, I, L, D, A, S, F, M, and G; and

### X(6): L, Q, V, M, P, W, T, and I; and

### X(7): A, S, T, V, L, G, F, D, M, and E; and

### X(8): V, L, I, M, A, W, K, G, and E; and

### X(9): E, K, G, D, A, V, M, and F; and

### X(10): X; and

### X(11): Y, L, F, H, C, I, T, M, and N; and

### X(12): L, I, V, M, Q, P, T, Y, and A; and

### X(13): K, R, Q, G, S, E, H, W, T, V, M, N, Z, Y, A, P, and C; and

### X(14): D, N, G, E, Y, V, S, H, A, M, and I; and

### X(15): Q, R, H, K, P, L, M, and N; and

### X(16): Q, K, R, T, H, E, S, P, M, and L; and

### X(17): L, F, I, R, V, P, S, M, and H; and

### X(18): L, M, P, I, H, and S; and

### X(19): X; and

### X(20): I, L, M, V, S, F, T, D, A, R, P, and J; and

### X(21): W, R, G, F, L, M, and T; and

### X(22): G, D, A, R, M, and C; and

### X(24): X; and

### X(25): G, R, E, N, A, M, and D; and

### X(26): K, R, N, E, Q, T, S, I, M, and G; and

### X(27): L, H, I, T, V, F, R, Q, S, P, A, J, M, and Y; and

### X(28): I, V, T, L, R, F, and M; and

### X(30): T, P, Y, A, N, S, I, V, R, L, M and H; and

### X(31): T, S, P, N, M and I; and

### X(32): A, N, T, S, D, R, F, Q, P, I, E, V, M, L, K, H, C, and B; and

### X(33): V, A, L, M, G, R, and C; and

### X(34): X; and

### X(35): W, R, G, L, M, and P; and

### X(36): N, S, D, B, K, E, R, Q, M, and G; and

### X(37): S, T, A, N, D, V, I, E, Y, K, L, R, G, P, M, F, W, H, Q, B, and C; and

### X(38): S, T, N, I, G, R, L, C, A, W, M and E; and

### X(39): W, G, A, R, E, C, Y, V, S, M, and H; and

### X(40): X; and

### X(41): N, G, K, S, D, E, T, R, H, P, A, B, V, Q, Y, M, and I; and

### X(42): K, R, N, D, S, T, G, E, I, V, Y, Q, P, H, A, W, M, and C; and

### X(43): S, T, N, K, I, R, D, E, P, L, A, W, G, M, H, Y, F, V, and C; and

### X(44): L, Y, Q, F, E, H, S, V, K, M, T, I, W, N, D, R, P, A, and G; and

### X(45): D, E, N, S, T, K, G, L, A, Q, H, I, Y, B, R, V, P, M, F, W, Z, and C; and

### X(46): E, D, Q, Y, K, N, T, S, A, W, H, M, R, I, G, L, V, Z, F, B, and P; and

### X(47): I, D, E, M, G, T, Q, S, W, L, N, Y, K, V, R, F, A, P, and H; and

### X(48): W, I, T, N, D, E, L, G, S, Y, R, V, K, H, A, Q, M, and F; and

### X(49): D, N, E, G, W, Q, K, H, L, B, S, I, Y, T, A, R, M, Z, and V; and

### X(50): N, D, T, K, S, H, L, G, E, W, I, Q, M, R, B, Y, P, and A;

where the amino acids are designated by their conventional single letter code.

According to the present invention, the amino acids in position 10, 19, 24, 34, and 40 affect the immunogenic properties of the HIV-1 envelope polypeptide. Thus, the amino acid in those positions alone or in combination affect the immunogenicity of the envelope polypeptide. In a preferred embodiment, the amino acid residue in position X(10) is selected from the group consisting of: R, S, T, K, G, A, N, Q and I. In another preferred embodiment, the amino acid residue in position X(19) is selected from the group consisting of: G, N, S, R, E, T, D, V, C and A. In another preferred embodiment, the amino acid residue in position X(24) is selected from the group consisting of: S, K, T, R, A, P, Y, F, G, Q, I and H. In another preferred embodiment, the amino acid residue in position X(34) is selected from the group consisting of: P, K, R, S, A, L, Q, E, H, T, I, V, and F. In another preferred embodiment, the amino acid residue in position X(40) is selected from the group consisting of: S, N, G, T, R, I, V, K, W, A, P, Y, D, Q, H, E, and C. In one preferred embodiment, the amino acid residue in position X(10) is threonine (T). In another preferred embodiment, the amino acid residue in position X(19) is asparagine (N). In another preferred embodiment, the amino acid residue in position X(24) is lysine (K). In another preferred embodiment, the amino acid residue in position X(34) is lysine (K). In yet another preferred embodiment, the amino acid residue in position X(40) is serine (S).

Specific embodiments comprise HIV-1 envelope polypeptides comprising an amino acid sequence selected from the group consisting

SEQ ID NO: 4:
LRARLLALETFIQNQQLLNLWGCKGNLICYTSVKWNDTWKGNSDTSLEN
IWDN
SEQ ID NO: 5:
LQARILAVETYLKDQQLLNIWGCKGKLICTTAVKWNASWSNKSLEQIWNH
SEQ ID NO: 6:
LQARILAVETYLKDQQLLGIWGCSGKLICTTAVPWNASWSNKSLEQIWNH
SEQ ID NO: 7:
LQARILAVERYLKDQQLLNIWGCSGKLICTTAVPWNASWSNKSLEQIWNH
SEQ ID NO: 8:
LQARILAVERYLKDQQLLGIWGCKGKLICTTAVPWNASWSNKSLEQIWNH
SEQ ID NO: 9:
LQARILAVERYLKDQQLLGIWGCSGKLICTTAVKWNASWSNKSLEQIWNH
SEQ ID NO: 10:
LQARILAVETYLKDQQLLNIWGCSGKLICTTAVPWNASWSNKSLEQIWNH
SEQ ID NO: 11:
LQARILAVETYLKDQQLLGIWGCKGKLICTTAVPWNASWSNKSLEQIWNH
SEQ ID NO: 12:
LQARILAVETYLKDQQLLGIWGCSGKLICTTAVKWNASWSNKSLEQIWNH
SEQ ID NO: 13:
LQARILAVETYLKDQQLLNIWGCKGKLICTTAVPWNASWSNKSLEQIWNH
SEQ ID NO: 14:
LQARILAVETYLKDQQLLNIWGCSGKLICTTAVKWNASWSNKSLEQIWNH
SEQ ID NO: 15:
LQARILAVETYLKDQQLLGIWGCKGKLICTTAVKWNASWSNKSLEQIWNH
SEQ ID NO: 16:
LQARILAVERYLKDQQLLNIWGCKGKLICTTAVPWNASWSNKSLEQIWNH
SEQ ID NO: 17:
LQARILAVERYLKDQQLLNIWGCSGKLICTTAVKWNASWSNKSLEQIWNH
SEQ ID NO: 18:
LQARILAVERYLKDQQLLNIWGCKGKLICTTAVKWNASWSNKSLEQIWNH
SEQ ID NO: 19:
LQARILAVERYLKDQQLLGIWGCKGKLICTTAVKWNASWSNKSLEQIWNH
SEQ ID NO: 20:
LQARIMAVERYLKDQQLLGIWGCSGKLICTTAVPWNASWSNKSLEQIWNH
SEQ ID NO: 21:
LQARILAMERYLKDQQLLGIWGCSGKLICTTAVPWNASWSNKSLEQIWNH
SEQ ID NO: 22:
LQARILAVERYMKDQQLLGIWGCSGKLICTTAVPWNASWSNKSLEQIWNH
SEQ ID NO: 23:
LQARILAVERYLKDQQLMGIWGCSGKLICTTAVPWNASWSNKSLEQIWNH
SEQ ID NO: 24:
LQARILAVERYLKDQQLLGMWGCSGKLICTTAVPWNASWSNKSLEQIWNH
SEQ ID NO: 25:
LQARILAVERYLKDQQLLGIWGCSGKLICTTAMPWNASWSNKSLEQIWNH
SEQ ID NO: 26:
LQARILAVERYLKDQQLLGIWGCSGKLICTTAVPWNASWSNKSLEQMWNH
SEQ ID NO: 27:
LRARLLALERYLKDQQLLGIWGCSGKLICTTAVPWNASWSNKSLEQIWNH
SEQ ID NO: 28:
LRARLLALETFIQNQQLLNLWGCSGKLICTTAVPWNASWSNKSLEQIWNH
SEQ ID NO: 29:
RQTEVLAIERYLKDQQLLGIWGCSGKLICTTAVPWNASWSNKSLEQIWNH
SEQ ID NO: 30:
LRTRVLAIERYLKDQQLLGIWGCSGKLICTTAVPWNASWSNKSLEQIWNH
SEQ ID NO: 31:
LRTRVQAIERYLKDQQLLGIWGCSGKLICTTAVPWNASWSNKSLEQIWNH
SEQ ID NO: 32:
LRTKVQTLETLIRNRKFMNLWGCSGKLICTTAVPWNASWSNKSLEQIWNH
SEQ ID NO: 33:
LRTRVLALETLIQNQQLLGIWGCSGKLICTTAVPWNASWSNKSLEQIWNH
SEQ ID NO: 34:
LQTRIQAMETYIRDQQFMGIWGCSGKLICTTAVPWNASWSNKSLEQIWNH
SEQ ID NO: 35:
LQTRIQAVETFIRDQQFMGIWGCSGKLICTTAVPWNASWSNKSLEQIWNH
SEQ ID NO: 36:
SQARIQAVETFIRDQQFMGIWGCSGKLICTTAVPWNASWSNKSLEQIWNH
SEQ ID NO: 37:
LQTRIQAVETFIRDQQLLGMWGCSGKLICTTAVPWNASWSNKSLEQIWNH
SEQ ID NO: 38:
LQARILAMERYMKDQQLMGMWGCSGKLICTTAVPWNASWSNKSLEQIWNH
SEQ ID NO: 39:
LRARILAMERYMKDQQLMGMWGCSGKLICTTAVPWNASWSNKSLEQIWNH
SEQ ID NO: 40:
LRARILAMERYLKDQQLLGIWGCSGKLICTTAVPWNASWSNKSLEQIWNH
SEQ ID NO: 41:
LRARILAMETYLKDQQLLGIWGCSGKLICTTAVPWNASWSNKSLEQIWNH
SEQ ID NO: 42:
LRARILAMETYMKDQQLMGMWGCSGKLICTTAVPWNASWSNKSLEQIWNH
SEQ ID NO: 43:
LRTRILAMETYLKDQQLLGIWGCSGKLICTTAVPWNASWSNKSLEQIWNH
SEQ ID NO: 44:
LRTRVLALETLIQNQQLLNIWGCSGKLICTTAVPWNASWSNKSLEQIWNH
SEQ ID NO: 45:
LQTRIQAVETFIRDQQLLNMWGCSGKLICTTAVPWNASWSNKSLEQIWNH
SEQ ID NO: 46:
LQARILAVERYLKDQQLLGIWGCKGNLICTTAVPWNASWSNKSLEQIWNH
SEQ ID NO: 47:
LQARILAVERYLKDQQLLGIWGCFGKLICTTAVPWNASWSNKSLEQIWNH
SEQ ID NO: 48:
LQARILAVERYLKDQQLLRIWGCSGKLICTTAVPWNASWSNKSLEQIWNH
SEQ ID NO: 49:
LRARLLALETFIQNQQLLRLWGCKGNLICYTSVKWNDTWKGNSDTSLENI
WDN
SEQ ID NO: 50:
LRARLLALETFIQNQQLLRLWGCFGNLICYTSVKWNDTWKGNSDTSLENI
WDN
SEQ ID NO: 51:
LRARLLALETFIQNQQLLNLWGCFGNLICYTSVKWNDTWKGNSDTSLENI
WDN
SEQ ID NO: 52:
LQTRIQAVETFIRDQQLLGMWGCKGNLICTTAVPWNASWSNKSLEQIWNH
SEQ ID NO: 53:
LQTRIQAVETFIRDQQFMGIWGCKGNLICTTAVPWNASWSNKSLEQIWNH
SEQ ID NO: 54:
LQTRIQAVETFIRDQQLLNMWGCKGNLICTTAVPWNASWSNKSLEQIWNH
SEQ ID NO: 55:
LQTRIQAVETFIRDQQFMNIWGCKGNLICTTAVPWNASWSNKSLEQIWNH
SEQ ID NO: 56:
LQTRIQAVETFIRDQQFMRIWGCFGNLICTTAVPWNASWSNKSLEQIWNH
SEQ ID NO: 57:
LRTRVLALETLIQNQQLLRIWGCFGKLICTTAVPWNASWSNKSLEQIWNH
SEQ ID NO: 58:
LQTRIQAMETYIRDQQFMRIWGCFGKLICTTAVPWNASWSNKSLEQIWNH
SEQ ID NO: 59:
LQTRIQAVETFIRDQQFMRIWGCFGKLICTTAVPWNASWSNKSLEQIWNH
SEQ ID NO: 60:
SQARIQAVETFIRDQQFMRIWGCFGKLICTTAVPWNASWSNKSLEQIWNH
SEQ ID NO: 61:
LQTRIQAVETFIRDQQLLRMWGCFGKLICTTAVPWNASWSNKSLEQIWNH
SEQ ID NO: 62:
LQARILAMERYMKDQQLMRMWGCFGKLICTTAVPWNASWSNKSLEQIWNH
SEQ ID NO: 63:
LRARILAMERYMKDQQLMRMWGCFGKLICTTAVPWNASWSNKSLEQIWNH
SEQ ID NO: 64:
LQARILAVERYLKDQQLLRIWGCFGKLICTTAVPWNASWSNKSLEQIWNH
SEQ ID NO: 65:
LQARILAVETLIQNQQRLNLWGCKGKLICYTSVKWNTSWSNKSLEQIWNH
SEQ ID NO: 66:
LQARILAVETYLKDQQLLNIWGCKGKLICTTAVKWNASWSNKSLEQIWNH
SEQ ID NO: 67:
LQARILAVETYLKDQQLLGIWGCSGKLICTTAVPWNASWSNKSLEQIWNH,
and/or
SEQ ID NO: 68:
LQARILAVKTYLKDQQLLNIWGCKGKLICTTAVKWNASWSNKSLEQIWNH

In another aspect, the present invention relates to an antigen comprising at least one peptide with an amino acid sequence as defined herein, or a functional homolog thereof having at least 70% identity to said peptide or an immunological active fragment comprising a consecutive sequence of at least 10 amino acids selected from a region of said peptide.

Another aspect of the present invention relates to a nucleic acid sequence encoding at least one HIV-1 envelope polypeptide with an amino acid sequence as defined above or a fragment thereof and/or a nucleic acid sequence encoding at least one antigen according to the present invention.

Yet another aspect relates to an isolated eukaryotic expression vector comprising at least one nucleic acid sequence as defined above or a fragment thereof. Another aspect of the present invention relates to a biological entity comprising at least one HIV-1 envelope as defined in the present invention, and/or at least one antigen as defined in the present invention, and/or at least one nucleic acid as defined in the present invention, and/or at least one vector as defined in the present invention.

Yet another aspect of the present invention relates to a vaccine composition comprising
a) an HIV-1 envelope polypeptide as defined herein, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined herein, and/or
c) a nucleic acid as defined herein, and/or
d) a vector as defined herein, and/or
e) a biological entity as defined herein, and
f) an adjuvant
   for use as a medicament.

A further aspect relates to a kit-of-parts comprising the vaccine composition as defined above, and a second active ingredient.

Another aspect relates to a method of treating, preventing or ameliorating a clinical condition, said method comprising administering to an individual suffering from said clinical condition an effective amount of an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention.

Yet another aspect of the present invention relates to the use of an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention for the manufacture of a medicament for the treatment, amelioration or prevention of a clinical condition.

A further aspect of the present invention relates to an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention for treating, ameliorating or preventing a clinical condition.

In another aspect the present invention relates to a pharmaceutical composition comprising an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention for treating, ameliorating or preventing a clinical condition.

A further aspect of the present invention relates to a method of reducing the risk of an individual encountering a clinical condition, said method comprising administration of an HIV-1 envelope polypeptide or part thereof as defined in the present invention, an antigen as defined in the present invention, a nucleic acid as defined in the present invention, a vector as defined in the present invention, a biological entity as defined in the present invention, a vaccine composition as defined in the present invention, or a kit-of-parts as defined in the present invention to said individual in an amount sufficient to generate a protective immune response.

In yet another aspect, the present invention relates a method of producing the vaccine composition of the present invention, comprising combining
a) an HIV-1 envelope polypeptide as defined in the present invention, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined in the present invention, and/or
c) a nucleic acid as defined in the present invention, and/or
d) a vector as defined in any of the present invention, and/or
e) a biological entity as defined in the present invention, and
f) an adjuvant.

In yet another aspect, the present invention relates to an antibody, antigen binding fragment or recombinant protein thereof, which is specific for an HIV-1 envelope polypeptide or part thereof as defined in the present invention, and/or a nucleic acid as defined in the present invention, and/or an antigen as defined in the present invention, and/or a biological entity as defined in the present invention.

In another aspect, the present invention relates to an antibody obtainable by immunizing a host with an HIV-1 envelope polypeptide or part thereof as defined herein, and/or a nucleic acid as defined herein, and/or an antigen as defined herein, and/or a biological entity as defined herein, a vaccine composition as defined herein, and/or a kit-of-parts as defined herein.

Furthermore, an aspect of the present invention relates to a method of producing an antibody as defined herein, said method comprising the steps of
a) administering an HIV-1 envelope polypeptide or part thereof as defined herein, an antigen as defined herein, a nucleic acid as defined herein, and/or a vector as defined herein, a biological entity as defined herein, a vaccine composition as defined herein, and/or a kit-of-parts as defined herein to an animal, and
b) obtaining said antibody from said animal

A further aspect of the present invention relates to a method of monitoring immunization, said method comprising the steps of
a) providing a blood sample from an individual
b) providing an HIV-1 envelope polypeptide or part thereof as defined herein, an antigen as defined herein, a nucleic acid as defined herein, and/or a vector as defined herein, a biological entity as defined herein, a vaccine composition as defined herein, and/or a kit-of-parts as defined herein, and
c) determining whether said blood sample comprises antibodies or T-cells comprising T-cell receptors specifically binding the protein or peptide
d) thereby determining whether an immune response to said protein or peptide has been raised in said individual.

### DESCRIPTION OF DRAWINGS

FIG. 1. Genomic structure of HIV-1 showing the location of env and gp41

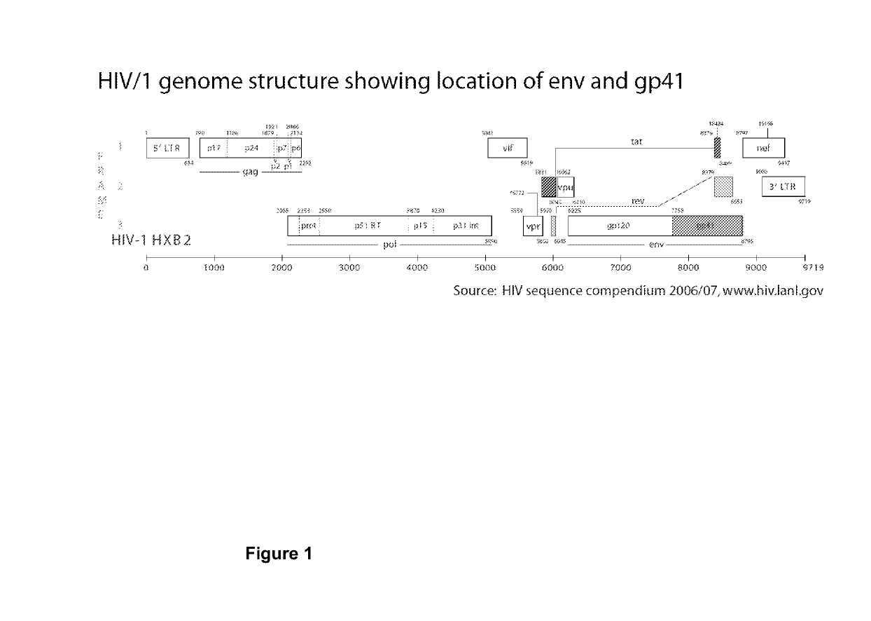

FIG. 2. HIV-1 gp41 alignment.

FIG. 3. HIV-1 gp41 alignment.

FIG. 4. Syncytia with wt HIV (both the visible and fluorescence micrograf)

FIG. 5. Syncytia made by mutant R10T (both the visible and fluorescence micrograf)

FIG. 6. Syncytia made by mutant Db mut (both the visible and fluorescence micrograf)

FIG. 7. Syncytia made by mutant O 10-40 (both the visible and fluorescence micrograf)

FIG. 8. Syncytia made by Pent mut (both the visible and fluorescence micrograf)

FIG. 9. Syncytia made by Pent +E9K (both the visible and fluorescence micrograf)

FIG. 10: Proliferation inhibition of the four peptides at 50 uM concentration

FIG. 11: The proliferation of lymphocytes in response to ConA (5 ug/mL) with a dose-dependence on immunosuppressive peptides.

FIG. 12: Lymphocyte proliferation. The fluorescence level is proportional to lymphocyte proliferation. The figure illustrates PBMC lymphocyte proliferation in cells treated as indicated. The column indicated “jurkat” are non-transduced jurkat cells. The other columns represent lymphocyte proliferation in the presence of jurkat cells transduced with a murine leukemia virus vector expressing either an eGFP marker gene alone (eGFP) or the eGFP marker gene in addition to a HIV envelope variant: HIV (WT), M/O, or HIV G19R.

FIG. 13: Normalized stimulation index. The graph shows a lymphocyte stimulation index, calculated as the proliferation in question/proliferation in UT. The column labels are the same as in FIG. 12.

FIG. 14: Cytokine secretion assay. Immunemodulatory properties of different peptides. IFN-γ (grey bar) and TNF-α (black bar). Cytokine secretion is evaluated by OD₄₅₀ level for PBMC untreated (UT), and in the presence of HIV WT, HIV G19R, or HIV M/O peptide, as indicated. HIV WT significantly reduces both IFN-γ and TNF-α secretion upon Con A stimulation of PBMC's. The mutant G19R downregulates TNF-α but not IFN-γ.

### DETAILED DESCRIPTION OF THE INVENTION

It is a major objective of the present invention to provide HIV-1 polypeptides and nucleic acids encoding said polypeptides for production of a vaccine against HIV-1.

The present invention provides immunogenic HIV-1 envelope polypeptides, antigens comprising said HIV-1 envelope polypeptides or part thereof, and nucleic acids encoding said polypeptides or part thereof, as well as eukaryotic expression vectors comprising at least one said nucleic acid or part thereof. Also provided are biological entities such as eukaryotic cells, prokaryotic cells and/or viral particles, in particular retroviral particles, which may be infectious or non-infectious, said biological entities comprising at least one HIV-1 envelope polypeptides and/or nucleic acid according to the present invention. The invention also provides vaccine composition comprising at least one HIV-1 envelope polypeptide, antigen, nucleic acid, vector, and/or biological entity of the present invention, and an adjuvant. A kit-of-parts is also provided comprising said vaccine composition a second active ingredient, such as an immunostimulating composition, for example one or more interleukins and/or an antibiotic, such as amoxicillin, penicillin, acyclovir and/or vidarabine. Specifically, the invention also provides for the use of the polypeptides, antigens, nucleic acids, vectors and biological entities for the manufacture of a medicament, and for methods of treating, preventing or ameliorating a clinical condition, said method comprising administering at least one HIV-1 envelope polypeptide, antigen, nucleic acid, vector, and/or biological entity of the present invention to a person in need thereof. The invention also provides methods for producing a vaccine and/or an antibody by administering a polypeptide, antigen, nucleic acid, vector and/or biological entity of the invention.

The components of the present invention can be used to induce an immune response against HIV, as well as to enhance the immune response in an immunized mammal relative to HIV antigen alone. Advantageously, the vaccine composition and/or components thereof of the invention can also induce HIV specific CD4 T helper cells and CD8+ T cells yielding potent Th1 immune responses against a broad spectrum of HIV epitopes, providing a strong HIV-specific cytotoxic T lymphocyte response. Thus, the vaccine compositions and components thereof of the invention are useful for preventing HIV infection and/or slowing progression to AIDS in infected individuals. The compositions, components and methods can be used to elicit potent Th1 cellular and humoral immune responses specific for conserved HIV epitopes, elicit HIV-specific CD4 T helper cells, HIV-specific cytotoxic T lymphocyte activity, stimulate production of chemokines and cyotokines such as beta-chemokines, interferon-gamma, interleukin 2 (IL2), interleukin 7 (IL7), interleukin 15 (IL15), alpha-defensin, and the like, and increase memory cells. The vaccine compositions and components thereof can be administered via various routes of administration, and can be used to prevent maternal transmission of HIV, for vaccination of newborns, children and high-risk individuals, and for vaccination of infected individuals. The components of the present invention can also be used in combination with other HIV therapies, including antiretroviral therapy (ART) with various combinations of nuclease and protease inhibitors and agents to block viral entry, such as T20.

A main aspect of the present invention is to provide retroviral particles, which express HIV-1 envelope polypeptide, or part thereof, and display the envelope on the surface of a retroviral particle, such as a lentiviral particle. The retroviral particle may be fusogenic or non-fusogenic. A non-fusogenic particle will promote an immunological response against HIV envelope on the particle surface. The fusogenic particle is infectious and mediate fusion with target cells.

### Terms and Definitions

To facilitate understanding of the invention, a number of terms are defined below.

The term “a retroviral vector” comprises a retroviral vector capable of being transcribed into RNA, which can be packaged into a retroviral particle, reverse transcribed into double stranded DNA and inserted into the host genome by the retroviral enzymatic machinery.

The term “heterologous” is used hereinafter for any combination of nucleic acid sequences that is not normally found intimately associated in nature.

The terms “bicistronic” and “polycistronic” as used herein, relates to a transcript encoding a transcript, which comprise two or more open reading frames, respectively. In one embodiment the replication deficient vector of the present invention comprises one open reading frame, two open reading frames, three, four, five or six open reading frames.

The term “polynucleotide” or “nucleic acid sequence” refers to a polymeric form of nucleotides at least 2 bases in length. By “isolated nucleic acid sequence” is meant a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA or RNA which is incorporated into a viral vector. The nucleotides of the invention can be ribonucleotides, deoxyribonucleotides, or modified forms of either nucleotide. The term includes single and double stranded forms of DNA.

The term “polynucleotide(s)” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can also refer to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term “polynucleotide” includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

The term “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

Thus, the term “amino acid” comprises any synthetic or naturally occurring amino carboxylic acid, including any amino acid occurring in peptides and polypeptides including proteins and enzymes synthesized in vivo thus including modifications of the amino acids. The term amino acid is herein used synonymously with the term “amino acid residue” which is meant to encompass amino acids as stated which have been reacted with at least one other species, such as 2, for example 3, such as more than 3 other species. The generic term amino acid comprises both natural and non-natural amino acids any of which may be in the “D” or “L” isomeric form.

| One-letter | Three-letter | |
|---|---|---|
| symbol | symbol | Amino acid |
| A | Ala | alanine |
| B | Asx | aspartic acid or asparagine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| U* | Sec | selenocysteine |
| V | Val | valine |
| W | Trp | tryptophan |
| X | Xaa | unknown or other amino acid, |
| | | i.e. X can be any of the |
| | | conventional amino acids. |
| Y | Tyr | tyrosine |
| Z | Glx | glutamic acid or glutamine (or substances such |
| | | as 4-carboxyglutamic acid and |
| | | 5-oxoproline that yield glutamic |
| | | acid on acid hydrolysis of peptides) |

A “detectable label” refers to a reporter molecule or enzyme that is capable of generating a measurable signal and is covalently or noncovalently joined to a polynucleotide or polypeptide.

A “fragment” is a unique portion of the polynucleotide encoding the HIV-1 envelope polypeptide of the present invention which is identical in sequence to but shorter in length than the parent sequence. Similarly the term ‘fragment’ refers to an HIV-1 envelope polypeptide of the present invention a fragment may comprise up to the entire length of the defined sequence, minus one nucleotide or amino acid residues. For example, a fragment may comprise from 5 to 2500 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250, 500 or at least 700 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

The term “Homology” refers to sequence similarity or, interchangeably, sequence identity, between two or more polynucleotide sequences or two or more polypeptide sequences.

Methods of alignment of sequences for comparison are well-known in the art. Various programs and alignment algorithms are described and present a detailed consideration of sequence alignment methods and homology calculations, such as VECTOR NTI. The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences will be.

The NCBI Basic Local Alignment Search Tool (BLAST) is available from several sources, including the National Center for Biotechnology Information (NBCI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

Homologs of the disclosed polypeptides are typically characterised by possession of at least 94% sequence identity counted over the full length alignment with the disclosed amino acid sequence using the NCBI Basic Blast 2.0, gapped blastp with databases such as the nr or swissprot database. Alternatively, one may manually align the sequences and count the number of identical amino acids. This number divided by the total number of amino acids in your sequence multiplied by 100 results in the percent identity.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), other nucleic acid analog, or to any DNA-like or RNA-like material.

The term “operably linked” refers to the situation in which a first nucleic acid sequence, amino acid sequence or ligand is placed in a functional relationship with a second nucleic acid sequence, amino acid sequence or ligand. For instance, a promoter is operably linked to a coding sequence if the promoter affects the transcription or expression of the coding sequence. Operably linked DNA sequences or protein or ligands may be in close proximity or contiguous and, where necessary to join two protein coding regions, in the same reading frame.

The term “internal ribosome entry site” (IRES) defines a sequence motif which promotes attachment of ribosomes to that motif on internal mRNA sequences. Furthermore, all factors needed to efficiently start translation at the AUG-start-codon following said IRES attach to this sequence motive. Consequently, an mRNA containing a sequence motive of a translation control element, e.g. IRES, results in two translational products, one initiating from the 5′ end of the mRNA and the other by an internal translation mechanism mediated by IRES. Accordingly, the insertion of a translational control element, such as IRES, operably linked to an ORF into a retroviral genome allows the translation of this additional ORF from a viral RNA transcript. Such RNA transcripts with the capacity to allow translation of two or more ORF are designated bi- or polycistronic RNA transcripts, respectively.

The term “treatment”, as used anywhere herein comprises any type of therapy, which aims at terminating, preventing, ameliorating and/or reducing the susceptibility to a clinical condition as described herein. In a preferred embodiment, the term treatment relates to prophylactic treatment, i.e. a therapy to reduce the susceptibility of a clinical condition, a disorder or condition as defined herein.

Thus, “treatment,” “treating,” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or immune deficiency.

The terms “prevent,” “preventing,” and “prevention”, as used herein, refer to a decrease in the occurrence of pathological cells in an animal. The prevention may be complete, e.g., the total absence of pathological cells in a subject. The prevention may also be partial, such that for example the occurrence of pathological cells in a subject is less than that which would have occurred without the present invention. Prevention also refers to reduced susceptibility to a clinical condition.

A “replication-deficient retroviral vector” according to the present invention, is a vector, which does not comprise all essential genes for viral propagation. The vector may comprise one or more nucleic acid sequences encoding retroviral components, such as an envelope polypeptide. However, the replication-deficient retroviral vector is for example devoid of nucleic acids encoding one or more of the retroviral components gag, pol, and/or rev, which are normally required for retroviral lifecycle, and Rev/Rex for lentiviral lifecycel. Generation of retroviral particles derived from a replication-deficient retroviral vector, thus, requires that the remaining components are provided in trans, for example encoded by a nucleic acid sequence comprised in a producer cell.

Moreover, a “replication-competent retroviral vector” according to the present invention, is a vector, which comprises all essential genes for viral propagation, i.e. gag, pol and ENV.

A “pharmaceutically acceptable carrier,” “pharmaceutically acceptable diluent,” or “pharmaceutically acceptable excipient”, or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed and is compatible with other ingredients of the formulation. For example, the carrier for a formulation containing polypeptides would not normally include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants of the invention include, but are not limited to Freunds's, Montanide ISA Adjuvants &Isqb; Seppic, Paris, France&rsqb; Ribi's Adjuvants (Ribi ImmunoChem Research, Inc., Hamilton, Mont.), I Hunter's TiterMax (CytRx Corp., Norcross, Ga.), Aluminum Salt Adjuvants (Alhydrogel—Superfos of Denmark/Accurate Chemical and Scientific Co., Westbury, N.Y.), Nitrocellulose-Adsorbed Protein, Encapsulated Antigens, and Gerbu Adjuvant (Gerbu Biotechnik GmbH, Gaiberg, Germany/C-C Biotech, Poway, Calif.). Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

“Pharmaceutically acceptable salts” include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, and histidine.

Compositions for oral administration can form solutions, suspensions, tablets, pills, capsules, sustained release formulations, oral rinses, or powders.

The term “unit dosage form,” as used herein, refers to physically discrete units suitable as unitary dosages for human and animal subjects, each unit containing a predetermined quantity of compounds of the present invention calculated in an “effective amount,” that is, a dosage sufficient to produce the desired result or effect in association with a pharmaceutically acceptable carrier. The specifications for the novel unit dosage forms of the present invention depend on the particular compound employed, the host, and the effect to be achieved, as well as the pharmacodynamics associated with each compound in the host.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “fusion” according to the present invention comprise cell-cell fusion as well as virus-cell fusion. Cell-cell Fusion or Syncytia formation is a process by which the plasma membranes of two cells merge to form a single continuous double lipid membrane. This process does not happen spontaneously and is often mediate by the surface proteins of enveloped viruses such as the envelope proteins of retroviruses. Virus cell fusion is process by which an enveloped virus mediates merging of its lipid membrane with that of a target cell through interaction of the viral coat protein with a cellular receptor. The result of viral cell fusion process is entry of the viral core into the cytoplasm of a target cell, which is necessary for productive infection.

As used herein, “AIDS” refers to the symptomatic phase of HIV infection, and includes both Acquired Immune Deficiency Syndrome (commonly known as AIDS) and “ARC,” or AIDS Related Complex. The immunological and clinical manifestations of AIDS are well known in the art and include, for example, opportunistic infections and cancers resulting from immune deficiency.

The term “inhibiting AIDS” as used herein, refers to a beneficial prophylactic or therapeutic effect of a composition or component in relation to HIV infection or AIDS symptoms. Such beneficial effects include, for example, preventing or delaying initial infection of an individual exposed to HIV; reducing viral burden in an individual infected with HIV; prolonging the asymptomatic phase of HIV infection; maintaining low viral loads in HIV infected patients whose virus levels have been lowered via anti-retroviral therapy (ART); increasing levels of CD4 T cells or lessening the decrease in CD4 T cells, both HIV-1 specific and non-specific, in drug naive patients and in patients treated with ART, increasing overall health or quality of life in an individual with AIDS; and prolonging life expectancy of an individual with AIDS. A clinician can compare the effect of immunization with the patient's condition prior to treatment, or with the expected condition of an untreated patient, to determine whether the treatment is effective in inhibiting AIDS.

As used herein, the term “enhances,” with respect to an immune response is intended to mean that the immunogenic agents and/or components elicits a greater immune response than does a composition containing HIV antigen alone. An enhanced immune response can be, for example, increased production of chemokines and/or cytokines that promote memory cells, an increase in memory cells, an increase in IgG2b production, in increase in cytotoxic T lymphocyte activity, an increase in beta-chemokine or IL15 production, and the like. As an example of an enhanced immune response, the immunogenic agents of the invention can increase production of gamma-interferon by both CD4 cells (helper function) and CD8 cells (cytotoxic T lymphocytes; CTLs).

The term “beta-chemokine” refers to a member of a class of small, chemoattractive polypeptides that includes RANTES, macrophage inflammatory protein-1.beta. (MIP-1.beta.) and macrophage inflammatory protein-1.alpha. (MIP-1.alpha.). The physical and functional properties of beta-chemokines are well known in the art. In the case of enhanced beta-chemokine production, the beta-chemokine production can be “HIV-specific, beta-chemokine production,” which refers to production of a beta-chemokine in response to stimulation of T cells with an HIV antigen. Alternatively, or additionally, the beta-chemokine production that is enhanced can be “non-specific beta-chemokine production,” which refers to production of a beta-chemokine in the absence of stimulation of T cells with an HIV antigen.

As used herein, the term “biological entity” relates to any matter of biological origin, such as a prokaryotic cell, a eukaryotic cell, a virus, a liposome, a nanoparticle and/or a retroviral cell, and/or any components and/or compartments thereof, such as for example a liposome.

The term “component of the present invention” as used herein is meant to incorporate any aspect an embodiment as of the present invention as defined herein. Thus, a component of the present invention includes an HIV-1 envelope polypeptide as defined herein, for example as identified by any of IGP1-7, SEQ ID NO: 1-337, and/or a functional homolog and/or fragment thereof. A component of the present invention also includes an antigen, a nucleic acid, a eukaryotic expression vector, a biological entity, a vaccine composition, a kit-of-parts, a method, a use, a compound, a cell, and/or an antibody as defined or described in the present invention.

As used herein, the expression “immunogenic” is used to describe an agent capable of eliciting at least one type of immune response directed against an HIV envelope polypeptide or fragment thereof. Thus, such an immune response may be any response, in particular a CTL response where CTLs are generated that are capable of recognising the HLA/polypeptide complex presented on cell surfaces resulting in cell lysis, i.e. the vaccine elicits the production in the vaccinated subject of effector T-cells having a cytotoxic effect against the host cells harbouring the retroviral vector or RNA thereof; as well as an antibody response giving rise to the production of anti-HIV antibodies.

Adjuvant: Any substance whose admixture with an administered immunogenic polypeptide/peptide/antigen/nucleic acid construct/biological entity increases or otherwise modifies the immune response to said determinant.

Antibody: Immunoglobulin molecules and active portions of immunoglobulin molecules. Antibodies are for example intact immunoglobulin molecules or fragments thereof retaining the immunologic activity.

Antigen: Any substance that can bind to a clonally distributed immune receptor (T-cell or B-cell receptor). Usually a peptide, polypeptide or a multimeric polypeptide. Antigens are preferably capable of eliciting an immune response.

APC: Antigen-presenting cell. An APC is a cell that displays foreign antigen complexed with MHC on its surface. T-cells may recognize this complex using their T-cell receptor (TCR). APCs fall into two categories: professional, (of which there are three types: Dendritic cells, macrophages and B-cells) or non-professional (does not constitutively express the Major histocompatibility complex proteins required for interaction with naive T cells; these are expressed only upon stimulation of the non-professional APC by certain cytokines such as IFN-γ).

Boost: To boost by a booster shot or dose is to give an additional dose of an immunizing agent, such as a vaccine, given at a time after the initial dose to sustain the immune response elicited by the previous dose of the same agent.

Cancer: Herein any preneoplastic or neoplastic disease, benign or malignant, where “neoplastic” refers to an abnormal proliferation of cells.

Carrier: Entity or compound to which for example antigens, polypeptides and/or biological entities are coupled to aid in the induction of an immune response.

Chimeric protein: A genetically engineered protein that is encoded by a nucleotide sequence made by a splicing together of two or more complete or partial genes or a series of (non)random nucleic acids.

Clinical condition: A condition that requires medical attention, herein especially conditions associated with the expression of HIV polypeptides such as envelope proteins. Examples of such conditions include: cancers and infections.

Complement: A complex series of blood proteins whose action “complements” the work of antibodies. Complement destroys bacteria, produces inflammation, and regulates immune reactions.

CTL: Cytotoxic T lymphocyte. A sub group of T-cells expressing CD8 along with the T-cell receptor and therefore able to respond to antigens presented by class I molecules.

Cytokine: Growth or differentiation modulator, used non-determinative herein, and should not limit the interpretation of the present invention and claims. In addition to the cytokines, adhesion or accessory molecules, or any combination thereof, may be employed alone or in combination with the cytokines.

Delivery vehicle: An entity whereby a nucleotide sequence or polypeptide or both can be transported from at least one media to another.

DC: Dendritic cell. (DCs) are immune cells and form part of the mammalian immune system. Their main function is to process antigen material and present it on the surface to other cells of the immune system, thus functioning as antigen-presenting cells (APCs).

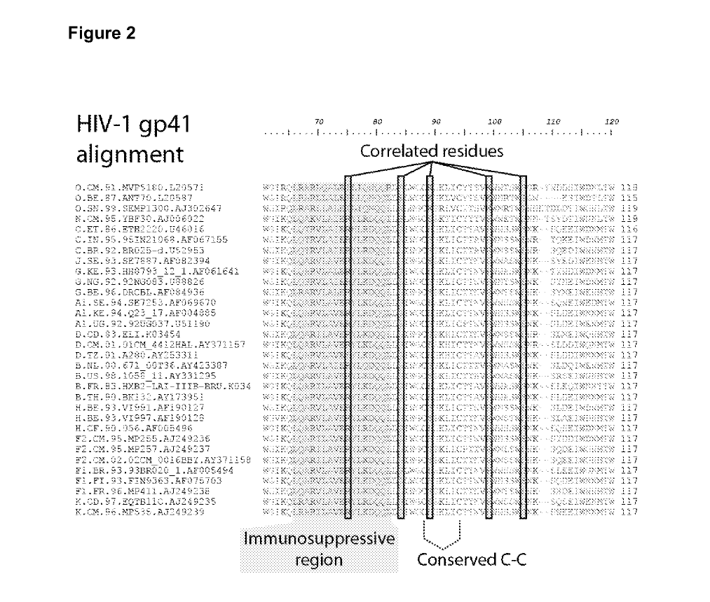

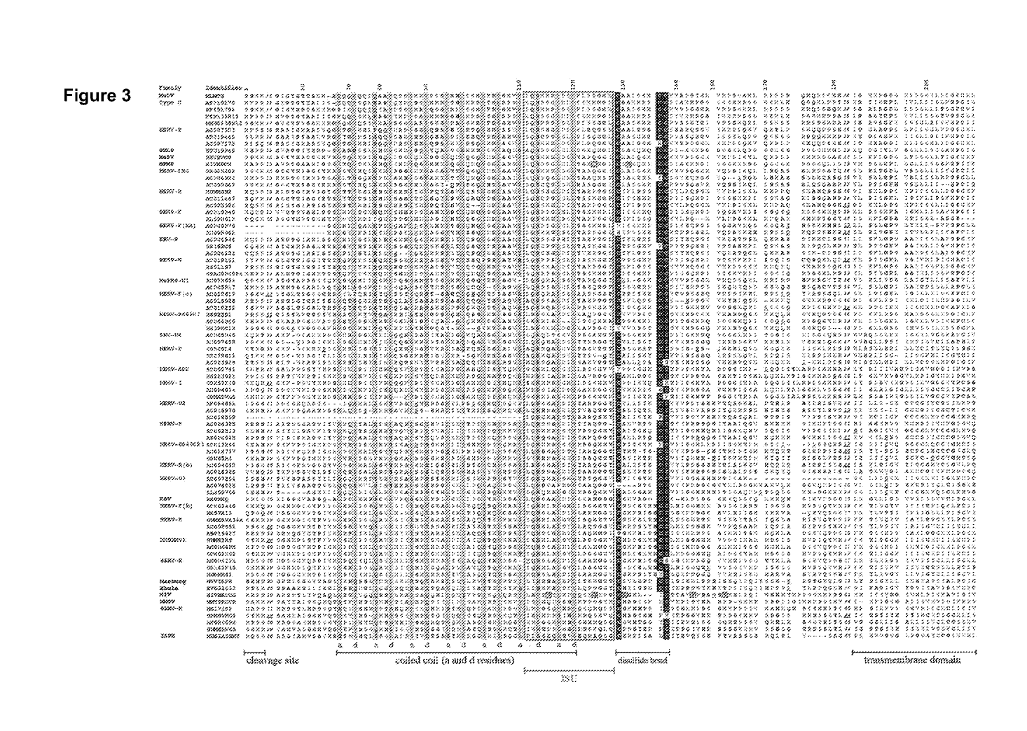

Envelope: Protein integrated in the membrane of the retroviral particle. HIV-1 envelope proteins are gp120 and gp41, examples of gp41 is represented by FIGS. 2 and 3, as well as SEQ ID NO: 74-76.

Fragment: is used to indicate a non-full length part of a nucleic acid or polypeptide. Thus, a fragment is itself also a nucleic acid or polypeptide, respectively.

Functional homologue: A functional homologue may be any nucleic acid/protein/polypeptide that exhibits at least some sequence identity with a wild type version/sequence of a given gene/gene product/protein/polypeptide and has retained at least one aspect of the original sequences functionality. Herein a functional homologue of HIV-1 envelope has the capability to induce an immune response to cells expressing HIV-1 envelope.

HIV: As used herein, the term “HIV” refers to all forms, subtypes and variations of the 2 5 HIV virus, and is synonymous with the older terms for HIV, such as HTLVIII and LAV. Various cell lines capable of propagating HIV or permanently infected with the HIV virus have been developed and deposited with the ATCC, including HuT 78 cells and the HuT 78 derivative H9, as well as those having accession numbers CCL 214, TIB 161, CRL 1552 and CRL 8543, which are described in U.S. Pat. No. 4,725,669 and Gallo, Scientific 3 0 American 256:46 (1987).

Immunostimulatory sequence: As used herein, the term “immunostimulatory sequence” or “ISS” refers to a genetic adjuvant, i.e. nucleotide sequence that is capable of enhancing the immune response in a mammal when administered in combination with an antigen. An ISS id for example a nucleic acid sequence comprising at least one unmethylated

Individual: Generally any species or subspecies of bird, mammal, fish, amphibian, or reptile, preferably a mammal, most preferably a human being.

Infection: Herein the term “infection” relates to any kind of clinical condition giving rise to an immune response and therefore includes infections, chronic infections, autoimmune conditions and allergic inflammations. In the context of the present invention, infection predominantly relates to HIV infection and related infections.

Isolated: used in connection with nucleic acids, polypeptides, and antibodies disclosed herein ‘isolated’ refers to these having been identified and separated and/or recovered from a component of their natural, typically cellular, environment. Nucleic acids, polypeptides, and antibodies of the invention are preferably isolated, and vaccines and other compositions of the invention preferably comprise isolated nucleic acids, polypeptides or isolated antibodies.

MHC: Major histocompatibility complex, two main subclasses of MHC, Class I and Class II exist.

Operative linker: A sequence of nucleotides or amino acid residues that bind together two parts of a nucleic acid construct or (chimeric) polypeptide in a manner securing the biological processing of the nucleic acid or polypeptide.

Pathogen: a specific causative agent of disease, especially a biological agent such as a virus, bacteria, prion or parasite that can cause disease to its host, also referred to as an infectious agent.

Peptide: Plurality of covalently linked amino acid residues defining a sequence and linked by amide bonds. The term is used analogously with oligopeptide and polypeptide. The natural and/or non-natural amino acids may be linked by peptide bonds or by non-peptide bonds. The term peptide also embraces post-translational modifications introduced by chemical or enzyme-catalyzed reactions, as are known in the art. The term can refer to a variant or fragment of a polypeptide.

Pharmaceutical carriers: also termed excipients, or stabilizers are non-toxic to the cell or individual being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

Plurality: At least two.

Promoter: A binding site in a DNA chain at which RNA polymerase binds to initiate transcription of messenger RNA by one or more nearby structural genes.

Signal peptide: A short sequence of amino acids that determine the eventual location of a protein in the cell, also referred to as sorting peptide.

Surfactant: A surface active agent capable of reducing the surface tension of a liquid in which it is dissolved. A surfactant is a compound containing a polar group which is hydrophilic and a non polar group which is hydrophobic and often composed of a fatty chain.

Treg: Regulatory T cells/T lymphocytes

Vaccine: A substance or composition capable of inducing an immune response in an animal. Also referred to herein as an immunogenic composition. An immune response being an immune response (humoral/antibody and/or cellular) inducing memory in an organism, resulting in the infectious agent, being met by a secondary rather than a primary response, thus reducing its impact on the host organism. A vaccine of the present invention may be given as a prophylactic and/or therapeutic medicament. The composition may comprise one or more of the following: HIV-1 envelope polypeptide(s), antigen(s), nucleic acid(s), vector(s), biological entities, adjuvants and pharmaceutical carriers.

Variant: a ‘variant’ of a given reference nucleic acid or polypeptide refers to a nucleic acid or polypeptide that displays a certain degree of sequence homology/identity to said reference nucleic acid or polypeptide but is not identical to said reference nucleic acid or polypeptide.

Vector: a genetically engineered nucleic acid construct. Typically comprising several elements such as genes or fragments of same, promoters, enhancers, terminators, polyA tails, linkers, polylinkers, operative linkers, multiple cloning sites (MCS), markers, STOP codons, other regulatory elements, internal ribosomal entry sites (IRES) or others. The present invention comprises expression vectors, such as eukaryotic or prokaryotic expression vectors, as well as vectors of retroviral origin.

Virosome: A virosome is a fusion between a virus and a liposome, for example a liposome with HIV-1 envelope polypeptides.

### HIV-1 Envelope Polypeptide

Due to the high error rate during replication of the HIV-1 genome, the genetic code for HIV-1 envelope is highly variable, both inside any one person and even more so across populations. The present invention relates to any variant of the HIV-1 envelope polypeptide, in particular any variant in which gp41, comprise an amino acid sequence of 50 amino acids, or part thereof, wherein the 50 amino acid sequence is defined as X(1-22)-C(23)-X(24-28)-C(29)-X(30-50). The number(s) in parentheses designate the number of each amino acid in the 50 amino acid sequence, i.e. C(23) specifies that amino acid number 23 is a cystein, and X(1-22) designates that amino acids 1 to 22 are any amino acids as defined above. Note however, that the term X(1-22) does not imply that amino acids 1-22 are the same amino acid; X merely indicates that each of the amino acids 1-22 are selected from all amino acids. Unless otherwise specified, the amino acids in the present invention are designated by their conventional single letter code, as defined above. The invariable cysteins in positions 23 and 29 of the 50 amino acid sequence may be used to align the amino acid sequence of the present invention with HIV-1 envelope subtypes, see e.g. FIG. 2.

Due to the variability in the gp41 sequence among different HIV-1 subtypes and clades, the present invention relates to any HIV-1 envelope polypeptide, which comprises a 50 amino acid sequence of the present invention, or part thereof. Non-limiting examples of sequences of HIV-1 envelopes are provided in SEQ ID NO: 74-119, 120-124, and 330-333. Thus, any HIV-1 envelope including those defined by SEQ ID NO: 74-119, 120-124, and 330-333 as well as functional homologes and/or fragments thereof as well as polypeptides with at least 70% identities to said homologes or fragments, such as at least 80% identities, for example at least 90% identities, such as at least 94, 95, 96, 97, 98, or at least 99% identity thereto are within the scope of the present invention.

The present invention, thus, relates to an HIV-1 envelope polypeptide comprising an amino acid sequence selected from the group of amino acid sequences consisting of: X(1-22)-C(23)-X(24-28)-C(29)-X(30-50) (identified as IGP1), or any part thereof, or functional homolog or any amino acid sequence with at least 90% identity to said amino acid sequence or part thereof. The 50 amino acid sequence is derived from any known HIV-1 envelope subtype, such as those identified by SEQ ID NO: 74-119. Thus, the specific amino acid in position X of the amino acid sequence above may be selected from an amino acid in the corresponding position of any of those and other HV-1 subtypes. Thus, in particular, the invention relates to an HIV-1 envelope polypeptide comprising an amino acid sequence selected from the group of amino acid sequences consisting of: X(1-22)-C(23)-X(24-28)-C(29)-X(30-50), or any part thereof, or functional homolog or any amino acid sequence with at least 90% identity to said amino acid sequence or part thereof, wherein the amino acid residues in the X(1-22)-C(23)-X(24-28)-C(29)-X(30-50) amino acid sequence are selected from the groups of residues consisting of:

### X(1): L, S, R, P, F, A, V, M, and I; and

### X(2): Q, R, K, H, L, M, and P; and

### X(3): A, T, V, H, S, R, Q, G, M, and E; and

### X(4): R, K, G, E, T, S, C, M, and H; and

### X(5): V, I, L, D, A, S, F, M, and G; and

### X(6): L, Q, V, M, P, W, T, and I; and

### X(7): A, S, T, V, L, G, F, D, M, and E; and

### X(8): V, L, I, M, A, W, K, G, and E; and

### X(9): E, K, G, D, A, V, M, and F; and

### X(10): X; and

### X(11): Y, L, F, H, C, I, T, M, and N; and

### X(12): L, I, V, M, Q, P, T, Y, and A; and

### X(13): K, R, Q, G, S, E, H, W, T, V, M, N, Z, Y, A, P, and C; and

### X(14): D, N, G, E, Y, V, S, H, A, M, and I; and

### X(15): Q, R, H, K, P, L, M, and N; and

### X(16): Q, K, R, T, H, E, S, P, M, and L; and

### X(17): L, F, I, R, V, P, S, M, and H; and

### X(18): L, M, P, I, H, and S; and

### X(19): X; and

### X(20): I, L, M, V, S, F, T, D, A, R, P, and J; and

### X(21): W, R, G, F, L, M, and T; and

### X(22): G, D, A, R, M, and C; and

### X(24): X; and

### X(25): G, R, E, N, A, M, and D; and

### X(26): K, R, N, E, Q, T, S, I, M, and G; and

### X(27): L, H, I, T, V, F, R, Q, S, P, A, J, M, and Y; and

### X(28): I, V, T, L, R, F, and M; and

### X(30): T, P, Y, A, N, S, I, V, R, L, M and H; and

### X(31): T, S, P, N, M and I; and

### X(32): A, N, T, S, D, R, F, Q, P, I, E, V, M, L, K, H, C, and B; and

### X(33): V, A, L, M, G, R, and C; and

### X(34): X; and

### X(35): W, R, G, L, M, and P; and

### X(36): N, S, D, B, K, E, R, Q, M, and G; and

### X(37): S, T, A, N, D, V, I, E, Y, K, L, R, G, P, M, F, W, H, Q, B, and C; and

### X(38): S, T, N, I, G, R, L, C, A, W, M and E; and

### X(39): W, G, A, R, E, C, Y, V, S, M, and H; and

### X(40): X; and

### X(41): N, G, K, S, D, E, T, R, H, P, A, B, V, Q, Y, M, and I; and

### X(42): K, R, N, D, S, T, G, E, I, V, Y, Q, P, H, A, W, M, and C; and

### X(43): S, T, N, K, I, R, D, E, P, L, A, W, G, M, H, Y, F, V, and C; and

### X(44): L, Y, Q, F, E, H, S, V, K, M, T, I, W, N, D, R, P, A, and G; and

### X(45): D, E, N, S, T, K, G, L, A, Q, H, I, Y, B, R, V, P, M, F, W, Z, and C; and

### X(46): E, D, Q, Y, K, N, T, S, A, W, H, M, R, I, G, L, V, Z, F, B, and P; and

### X(47): I, D, E, M, G, T, Q, S, W, L, N, Y, K, V, R, F, A, P, and H; and

### X(48): W, I, T, N, D, E, L, G, S, Y, R, V, K, H, A, Q, M, and F; and

### X(49): D, N, E, G, W, Q, K, H, L, B, S, I, Y, T, A, R, M, Z, and V; and

### X(50): N, D, T, K, S, H, L, G, E, W, I, Q, M, R, B, Y, P, and A.

According to the present invention, at least one of the amino acids in position 10, 19, 24, 34, and/or 40 affect the immunogenic properties of the HIV-1 envelope polypeptide. Thus, the amino acids in those positions alone or in combination affects the immunogenicity of the envelope polypeptide. Thus in a primary aspect, the present invention relates to any HIV-1 envelope polypeptide, such as any HIV-1 envelope as defined herein, comprising an amino acid sequence with a mutation in any one of the positions corresponding to amino acid residue position 10, 19, 24, 34 and/or 40 of any one of the amino acid sequences identified herein as IGP1-7, SEQ ID NO: 1-73, 125-329 and/or 330-337, and/or any amino acid sequence at least 60% identical thereto, such as at least 70%, for example at least 80% identical, such as at least 90%, such at least 91, 92, 93, 94, 95, 96, 97, 98, such as at least 99% identical to any of those amino acid sequences carrying mutations in one or more of the amino acid residue positions 10, 19, 24, 34 and/or 40. Thus, any HIV-1 envelope polypeptide comprising an amino acid sequence identified herein, which carries a mutation in a position such as positions 10, 19, 24, 34 and/or 40 of any one of IGP1-7, SEQ ID NO: 1-73, 125-329, and/or 334-337 are within the scope of the present invention, as well as any HIV-1 envelope comprising an amino acid sequence, which with respect to the other amino acid residues (i.e. amino acid residues 1-9, 11-18, 20-23, 25-33, 35-39 and 41-50) of for example any one of IGP1-7, SEQ ID NO: 1-73, 125-329, and/or 334-337 is at least 60% identical thereto, such as at least 70%, for example at least 80% identical, such as at least 90%, such at least 91, 92, 93, 94, 95, 96, 97, 98, such as at least 99% identical to any of those amino acid sequences.

In a specific embodiment, the amino acid residue in position X(10) is selected from the group consisting of: R, S, T, K, G, A, N, Q and/or I. In another specific embodiment, the amino acid residue in position X(19) is selected from the group consisting of: G, N, S, R, E, T, D, V, C and/or A. In another specific embodiment, the amino acid residue in position X(24) is selected from the group consisting of: S, K, T, R, A, P, Y, F, G, Q, I and/or H. In another specific embodiment, the amino acid residue in position X(34) is selected from the group consisting of: P, K, R, S, A, L, Q, E, H, T, I, V, and/or F. In another specific embodiment, the amino acid residue in position X(40) is selected from the group consisting of: S, N, G, T, R, I, V, K, W, A, P, Y, D, Q, H, E, and/or C.

More specifically, in a further embodiment, the amino acid residue in position X(10) is arginine (R), such that the HIV-1 envelope of the present invention comprises an amino acids sequence selected from the group of amino acid sequences consisting of: X(1-9)-R(10)-X(11-22)-C(23)-X(24-28)-C(29)-X(30-50), or any part thereof, or functional homolog or any amino acid sequence with at least 90% identity to said amino acid sequence or part thereof.

In another embodiment, the amino acid residue in position X(19) is glycine (G), such that the HIV-1 envelope of the present invention comprises an amino acids sequence selected from the group of amino acid sequences consisting of: X(1-18)-G(19)-X(20-22)-C(23)-X(24-28)-C(29)-X(30-50), or any part thereof, or functional homolog or any amino acid sequence with at least 90% identity to said amino acid sequence or part thereof.

In yet another embodiment, the amino acid residue in position X(24) is serine (S), such that the HIV-1 envelope of the present invention comprises an amino acids sequence selected from the group of amino acid sequences consisting of: X(1-22)-C(23)-S(24)-X(25-28)-C(29)-X(30-50), or any part thereof, or functional homolog or any amino acid sequence with at least 90% identity to said amino acid sequence or part thereof.

In a further embodiment, the amino acid residue in position X(34) is proline (P), such that the HIV-1 envelope of the present invention comprises an amino acids sequence selected from the group of amino acid sequences consisting of: X(1-22)-C(23)-X(24)-28)-C(29)-X(30-33)-P(34)-X(35-50), or any part thereof, or functional homolog or any amino acid sequence with at least 90% identity to said amino acid sequence or part thereof.

In another embodiment, the amino acid residue in position X(40) is serine (S), such that the HIV-1 envelope of the present invention comprises an amino acids sequence selected from the group of amino acid sequences consisting of: X(1-22)-C(23)-X(24-28)-C(29)-X(30-39)-S(40)-X(41-50), or any part thereof, or functional homolog or any amino acid sequence with at least 90% identity to said amino acid sequence or part thereof.

More specifically, in a further embodiment, the amino acid residue in position X(10) is threonine (T), such that the HIV-1 envelope of the present invention comprises an amino acids sequence selected from the group of amino acid sequences consisting of: X(1-9)-T(10)-X(11-22)-C(23)-X(24-28)-C(29)-X(30-50), or any part thereof, or functional homolog or any amino acid sequence with at least 90% identity to said amino acid sequence or part thereof.

In another embodiment, the amino acid residue in position X(19) is Asparagine (N), such that the HIV-1 envelope of the present invention comprises an amino acids sequence selected from the group of amino acid sequences consisting of: X(1-18)-N(19)-X(20-22)-C(23)-X(24-28)-C(29)-X(30-50), or any part thereof, or functional homolog or any amino acid sequence with at least 90% identity to said amino acid sequence or part thereof.

In yet another embodiment, the amino acid residue in position X(24) is lysine (K), such that the HIV-1 envelope of the present invention comprises an amino acids sequence selected from the group of amino acid sequences consisting of: X(1-22)-C(23)-K(24)-X(25-28)-C(29)-X(30-50), or any part thereof, or functional homolog or any amino acid sequence with at least 90% identity to said amino acid sequence or part thereof.

In a further embodiment, the amino acid residue in position X(34) is lysine (K), such that the HIV-1 envelope of the present invention comprises an amino acids sequence selected from the group of amino acid sequences consisting of: X(1-22)-C(23)-X(24)-28)-C(29)-X(30-33)-K(34)-X(35-50), or any part thereof, or functional homolog or any amino acid sequence with at least 90% identity to said amino acid sequence or part thereof.

In another embodiment, the amino acid residue in position X(40) is serine (S), such that the HIV-1 envelope of the present invention comprises an amino acids sequence selected from the group of amino acid sequences consisting of: X(1-22)-C(23)-X(24-28)-C(29)-X(30-39)-K(40)-X(41-50), or any part thereof, or functional homolog or any amino acid sequence with at least 90% identity to said amino acid sequence or part thereof.

In a specific embodiment, the HIV-1 envelope polypeptide of the present invention comprises an amino acid sequence selected from the group of amino acid sequences consisting of IGP1-7 and SEQ ID NO: 1-337, such as the group consisting of IGP1-7 and SEQ ID NO: 1-73, 125-329, or 334-337, any part thereof, functional homolog and/or any amino acid sequence with at least 90% identity to said amino acid sequence or part thereof. Each of those amino acids sequences, e.g. IGP1-7, SEQ ID NO: 1-73, 125-329, or 334-337 are also within the scope of the present invention and, consequently, an HIV-1 envelope polypeptide of the present invention, comprising any amino acid sequence selected from IGP1-7 or SEQ ID NO: 1-337, such as SEQ ID NO: 1-73, 125-329, or 334-337 are claimed as individual embodiments. Also, among HIV-1 envelopes a frequent polymorphism exist at the position corresponding to position 5 in SEQ ID NO: 4-68. For example the first 10 amino acids of SEQ ID NO: 5 are LQARILAVETYLKDQQLLNI, but the I in position 5 is replaced by V in many naturally found envelopes, and also the L in position 17 is replaced by R in some clades or subtypes. Thus, the present invention comprise HIV-1 envelope polypeptide of the present invention comprising either I or V in a position corresponding to position 5 and L or R in a position corresponding to position 17 of SEQ ID NO: 5, and I and V may replace each other in a position corresponding to position 5 and L and R may replace each other in a position corresponding to position 17 of SEQ ID NO: 5 in any of the amino acid sequences provided in the present invention, including any one of IGP1-7 and SEQ ID NO: 1-337.

In a specific embodiment, the HIV-1 envelope polypeptide of the present invention comprises an amino acid sequence selected from the group consisting of IGP1-7, SEQ ID NO: 1-19, or any part thereof, or functional homolog or any polypeptide with at least 90% identity to said polypeptide or part thereof. In another embodiment, the HIV-1 envelope polypeptide of the present invention comprises an amino acid sequence selected from the group consisting of SEQ ID NO: 20-63, or any part thereof, or functional homolog or any polypeptide with at least 90% identity to said polypeptide or part thereof. In another embodiment, the HIV-1 envelope polypeptide of the present invention comprises an amino acid sequence selected from the group consisting of SEQ ID NO: 64-68, or any part thereof, or functional homolog or any polypeptide with at least 90% identity to said polypeptide or part thereof.

As non-limiting examples of a specific embodiment of the present invention, is an HIV-1 envelope polypeptide comprising or consisting of the amino acid sequence LRARLLALETFIQNQQLLNLWGCKGNLICYTSVKWNDTWKGNSDTSLENIWDN (SEQ ID NO: 4), LQARIMAVERYLKDQQLLGIWGCSGKLICTTAVPWNASWSNKSLEQIWNH (SEQ ID NO: 20), QARILAVERYLKDQQLLRIWGCFGKLICTTAVPWNASWSNKSLEQIWNH (SEQ ID NO: 64), HIV G19R: LQARVLAVERYLKDQQLLRIWGC (SEQ ID NO: 132), HIV M/O chimera: LQARILAVETLIQNQQLLNLWGC (SEQ ID NO: 133) and/or any part thereof, or functional homolog or any polypeptide with at least 70%, such as at least 80%, for example at least 90% identity to said polypeptide or part thereof.

Specific examples of HIV-1 envelope polypeptides according to the present invention are represented by SEQ ID NO: 120-124, 325, and 330-333. Thus in one embodiment, the HIV-1 envelope of the present invention is selected from the group consisting of SEQ ID NO: 120-124, 325, and 330-333.

The polypeptides of the present invention may be free carboxyl- or amino groups, amides, acyls, acetyls or salts thereof, two or more of the Cys residues may form part of an intrachain- or interchain disulphide binding, a —S—(CH2)p-S— or a —(CH2)p-bridge wherein p=1-8, optionally intervened by one or more heteroatoms such as O, N or S. The C- and N-terminals ends of the polypeptide sequences could deviate from the natural sequences by modification of the terminal NH2-group and/or COOH-group. They may, for instance, be acylated, acetylated, amidated or modified to provide a binding site for a carrier or another molecule.

The polypeptides may be lyophilized, or dissolved or suspended in a suitable liquid buffer. Moreover, the HIV-1 envelope may be comprised in a composition, or integrated in a biological entity, for example in the membrane of a cell, or on the surface of a viral particle. Thus, the polypeptide or polypeptide antigen according to the invention is either in a free or in a carrier-bound form. The carrier or solid phase to which the peptide is optionally bound can be selected from a wide variety of known carriers. It should be selected with regard to the intended use of the immobilized polypeptide as a diagnostic antigen or as an immunizing component in a vaccine.

Examples of carriers that can be used for diagnostic purposes, for example, are magnetic beads or latex of co-polymers such as styrene-divinyl benzene, hydroxylated styrene-divinyl benzene, polystyrene, carboxylated polystyrene, beads of carbon black, non-activated or polystyrene or polyvinyl chloride activated glass, epoxy-activated porous magnetic glass, gelatine or polysaccharide particles or other protein particles, red blood cells, mono- or polyclonal antibodies or fab fragments of such antibodies, liposomes and/or nanoparticles.

Further embodiments of the invention include any vector, method, provirus, retroviral particle, use, composition, vaccine, vaccine composition or kit as described herein, comprising an HIV-1 envelope polypeptide of the present invention that has a sequence that is at least 36% identical to the amino acid sequence of any polypeptide of the present invention, or is a fragment of a sequence that is at least 36% thereto.

However, in other embodiments of the present invention the HIV-1 envelope polypeptide or a fragment thereof has a sequence that is for example at least 40%, such as at least 45%, for example at least 50%, such as at least 55%, for example at least 60%, such as at least 65%, for example at least 67%, such as at least 70%, for example at least 72%, such as at least 75%, for example at least 77%, such as at least 80%, for example at least 81%, such as at least 82%, for example at least 83%, such as at least 84%, for example at least 85%, such as at least 86%, for example at least 87%, such as at least 88%, for example at least 89%, such as at least 90%, for example at least 91%, such as at least 92%, for example at least 93%, such as at least 94%, for example at least 95%, such as at least 96%, for example at least 97%, such as at least 98%, for example at least 99% identical to the amino acid sequence of any polypeptide of the present invention.

### Production of Peptides

The polypeptides of the invention can be produced by any known method of producing a linear amino acid sequence either being synthesized as a synthetic polypeptide or produced by recombinant DNA techniques. A nucleic acid sequence which encodes a polypeptide of the invention or a multimer of the said polypeptides is introduced into an expression vector. Suitable expression vectors are for instance plasmids, cosmids, viruses and YAC (yeast artificial chromosome) which comprise necessary control regions for replication and expression. The expression vector may be stimulated to expression in a host cell. Suitable host cells are for example bacteria, yeast cells and mammal cells. Such techniques are well known in the art and described for instance by Sambrook et al., Molecular Cloning: A Laboratory Manual, Cold Spring Harbor Laboratory Press, Cold Spring Harbor, 1989. Other well-known techniques are degradation or synthesis by coupling of one amino acid residue to the next one in liquid phase or preferably on a solid phase (resin) for instance by the so-called Merrifield synthesis. See for instance Barany and Merrifield in the Peptides, Analysis, Synthesis, Biology, Vol. 2, E. Gross and Meinhofer, Ed. (Acad. Press, N.Y., 1980), Kneib-Coronier and Mullen Int. J. Peptide Protein Res., 30, p. 705-739 (1987) and Fields and Noble Int. J. Peptide Protein Res., 35, p. 161-214 (1990). In case a linked or cyclic peptide is desired, the amino acid sequence is subjected to a chemical oxidation step in order to cyclize or link the two cysteine residues within one or between two peptide sequences, when the appropriate linear amino acid sequences are synthesized, see Akaji et al., Tetrahedron Letter, 33, 8, p. 1073-1076, 1992.

### Antigen

One aspect of the present invention relates to an antigen, which comprises a peptide derived from an HIV-1 envelope polypeptide of the present invention. In particular, the antigen incorporates a part of HIV-1 envelope polypeptide, including any region of gp41 and/or gp120, such as for example the transmembrane domain (TM-domain)) or surface subunit (SU). Preferably, the antigen comprises at least one peptide with an amino acid sequence of an HIV-1 envelope polypeptide of the present invention, or a functional homolog thereof having at least 70%, such as at least 80%, for example at least 90% identity to said envelope polypeptide or an immunological active fragment comprising or consisting of a consecutive sequence of at least 10 amino acids selected from a region of said envelope polypeptide. In another embodiment, the antigen of the present invention comprises or consists of at least one peptide with an amino acid sequence comprising an immunological active fragment comprising or consisting of a consecutive sequence of at least 12 amino acids, such as at least 15 amino acids, for example at least 20 amino acids, such as 30 amino acids, such as at least 40 amino acids, for example at least 50 amino acids, such as 60 amino acids, such as at least 70 amino acids, for example at least 80 amino acids, such as 90 amino acids, such as at least 100 amino acids, such as at least 110 amino acids, for example at least 120 amino acids, such as 130 amino acids, such as at least 140 amino acids, for example at least 150 amino acids, such as 160 amino acids, such as at least 170 amino acids, for example at least 180 amino acids, such as 190 amino acids, such as at least 200 amino acids, such as at least 300 amino acids, for example at least 400 amino acids, such as 500 amino acids, such as at least 600 amino acids, for example at least 700 amino acids, such as 800 amino acids, such as at least 900 amino acids, for example at least 1000 amino acids selected from a region of an HIV-1 envelope polypeptide of the present invention, for example selected from a sequence according to any of SEQ ID NO: 1-326, IGP1-7 or SEQ ID NO: 327-337.

The present invention also relates to an antigen comprising or consisting of at least one peptide having at least 50%, such as at least 60%, for example at least 70% such as at least 80% for example at least 90%, such as at least 95%, such as at least 98%, for example at least 99% identity to any polypeptide according to the present invention, for example a sequence according to any of SEQ ID NO: 1-124 or SEQ ID NO: 125-326, IGP1-7 or SEQ ID NO: 327-337 or any part or functional homolog or immunological active fragment thereof, for example any part or functional homolog or immunological active fragment thereof comprising or consisting of at least 3, such as at least 5, such as at least 10, or such as at least 15 consecutive amino acid residues.

### Nucleic Acid

In one aspect, the present invention relates to a nucleic acid sequence, e.g. DNA and/or RNA, encoding at least one HIV-1 envelope polypeptide of the present invention, or a fragment thereof and/or a nucleic acid sequence encoding at least one antigen as defined herein. Thus in a specific embodiment, the present invention relates to a nucleic acid sequence encoding an HIV-1 envelope polypeptide comprising or consisting of an amino acid sequence selected from the group consisting of IGP1-7, SEQ ID NO:1-124, SEQ ID NO: 125-326 or SEQ ID NO: 327-337 and/or any part or functional homolog thereof. It is understood that a nucleic acid encoding any sequence selected from any of IGP1-7, SEQ ID NO: 1-124, SEQ ID NO: 125-326 or SEQ ID NO: 327-337 or part thereof, constitute a single embodiment, and is consequently claimed individually. In one embodiment, the nucleic acid sequence has been optimized by codon optimization, as explained elsewhere herein.

### Vectors

In one aspect, the present invention relates to an isolated expression vector comprising at least one nucleic acid sequence according to the present invention or a functional homolog or a fragment thereof, or a nucleic acid encoding a polypeptide with at least 70% identity thereto. The vector of the present invention is a prokaryotic expression vector or a eukaryotic expression vector, preferably a mammalian expression vector. Thus, in one embodiment, the present invention relates to an isolated eukaryotic expression vector comprising at least one nucleic acid sequence encoding at least one HIV-1 envelope polypeptide of the present invention, or a fragment thereof and/or a nucleic acid sequence encoding at least one antigen as defined herein.

Numerous vectors are available and the skilled person will be able to select a useful vector for the specific purpose. The vector may, for example, be in the form of a plasmid, cosmid, viral particle or artificial chromosome. The appropriate nucleic acid sequence may be inserted into the vector by a variety of procedures, for example, DNA may be inserted into an appropriate restriction endonuclease site(s) using techniques well known in the art. Apart from the nucleic acid sequence according to the invention, the vector may furthermore comprise one or more of a signal sequence, an origin of replication, one or more marker genes, an enhancer element, a promoter, and a transcription termination sequence. The vector may also comprise additional sequences, such as enhancers, poly-A tails, linkers, polylinkers, operative linkers, multiple cloning sites (MCS), STOP codons, internal ribosomal entry sites (IRES) and host homologous sequences for integration or other defined elements. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). The vector is preferably an expression vector, comprising the nucleic acid operably linked to a regulatory nucleic acid sequence directing expression thereof in a suitable cell. Within the scope of the present invention said regulatory nucleic acid sequence should in general be capable of directing expression in a mammalian cell, preferably a human cell, more preferably in an antigen presenting cell or a T-cell.

In one preferred embodiment the vector is a viral vector. The vector may also be a bacterial vector, such as an attenuated bacterial vector. Attenuated bacterial vectors may be used in order to induce lasting mucosal immune responses at the sites of infection and persistence. Different recombinant bacteria may be used as vectors, for example the bacterial vector may be selected from the group consisting of Salmonella, Lactococcus], and Listeria. In general, induction of immunity to the heterologous antigen HPV16 L1 or E7 could be shown, with strong CTL induction and tumor regression in mice. The vector may furthermore comprise a nucleic acid encoding a T-cell stimulatory polypeptide.

The vector of the present invention may be any eukaryotic expression vector, for example a mammalian expression vector, or a yeast vector. The vector may comprise at least one intron, which will facilitate the transport from the nucleus to the cytoplasma of the vector encoded RNA, for example in packaging cells. In another embodiment, the vector is capable of expressing RNA in the cytoplasm by cytoplasmic transcription, which can be translated into envelope polypeptide. The vector is also, in one embodiment, capable of expressing high levels of vector encoded RNA, which is transported to the cytoplasma to be translated into envelope polypeptide as encoded in the vector. Thus, in one embodiment the vector of the present invention is transcribed in the nucleus, thereby producing high levels of transcript, which after transport to the cytoplasm can be translated into envelope polypeptide. The vector of the present invention may be transfected into a packaging cell which is capable of producing viral particles comprising said lentiviral envelope polypeptide.

In one embodiment, the vector is a retroviral vector. The retroviral vector may be either replication deficient or replication competent.

The retroviral vector can be derived from any species of retroviridae. In one embodiment, the retroviral vector is derived from Orthoretrovirinae, comprising Alpharetrovirus, Betaretrovirus, and Gammaretrovirus. In a specific embodiment, the retroviral vector is derived from Avian carcinoma Mill Hill virus 2, Avian leukosis virus, Avian myeloblastosis virus, Avian myelocytomatosis virus 29, Avian sarcoma virus CT10, Fujinami sarcoma virus, Rous sarcoma virus, UR2 sarcoma virus or Y73 sarcoma virus. The alphaviruses are listed in table 1. Each of the alphaviruses specified above is intended to be an individual embodiment. Consequently, a retroviral vector according to the present invention derived from each of them is claimed individually.

TABLE 1
List of alpharetroviruses

| | |
|---|---|
| Alpharetrovirus | Avian carcinoma Mill Hill virus 2 |
| Alpharetrovirus | Avian leukosis virus |
| Alpharetrovirus | Avian myeloblastosis virus |
| Alpharetrovirus | Avian myelocytomatosis virus 29 |
| Alpharetrovirus | Avian sarcoma virus CT10 |
| Alpharetrovirus | Fujinami sarcoma virus |
| Alpharetrovirus | Rous sarcoma virus |
| Alpharetrovirus | UR2 sarcoma virus |
| Alpharetrovirus | Y73 sarcoma virus |

In another specific embodiment, the retroviral vector is derived from Jaagsiekte sheep retrovirus, Langur virus, Mason-Pfizer monkey virus, Mouse mammary tumor virus or Squirrel monkey retrovirus. The betaviruses are listed in table 2. Each of the betaviruses specified herein is intended to be an individual embodiment. Consequently, a retroviral vector according to the present invention derived from each of them may be claimed individually.

TABLE 2
List of betaretroviruses

| | |
|---|---|
| Betaretrovirus | Jaagsiekte sheep retrovirus |
| Betaretrovirus | Langur virus |
| Betaretrovirus | Mason-Pfizer monkey virus |
| Betaretrovirus | Mouse mammary tumor virus |
| Betaretrovirus | Squirrel monkey retrovirus |

In another embodiment the retroviral vector according to the present invention is derived from gammaretroviruses as shown in table 3 below.

TABLE 3
List of gammaretroviruses

```
    Avian (Reticuloendotheliosis) virus group
        Chick syncytial virus
        Reticuloendotheliosis virus
            Avian spleen necrosis virus
            Spleen necrosis virus
    Mammalian virus group
        Murine endogenous retrovirus
        Murine leukemia-related retroviruses
            Epicrionops marmoratus retrovirus
            Ichthyophis kohtaoensis retrovirus
            Osteolaemus tetraspis retrovirus
            Sericulus bakeri retrovirus
            Terdus iliacus retrovirus
            Tomistoma schlegelii retrovirus
            Viper berus retrovirus
            Xenotropic MuLV-related virus
            Monodelphis sp. retrovirus
        Replication competent viruses
            Feline leukemia virus
            Gibbon ape leukemia virus (GALV)
            Murine leukemia virus
            Porcine type-C oncovirus
        Replication defective viruses
            Abelson murine leukemia virus
            Gardner-Arnstein feline sarcoma virus
            Hardy-Zuckerman feline sarcoma virus
            Harvey murine sarcoma virus
            Kirsten murine sarcoma virus
            Moloney murine sarcoma virus
            Murine osteosarcoma virus
            Snyder-Theilen feline sarcoma virus
            Spleen focus-forming virus
            Woolly monkey sarcoma virus
    unclassified Gammaretrovirus
        Baboon endogenous virus
            Baboon endogenous virus strain M7
        Feline endogenous virus
            Feline endogenous virus ECE1
            Feline endogenous virus RD114
        Koala retrovirus
        Macaca mulatta type C retrovirus
            Macaca endogenous retrovirus
        MLV-related retrovirus
        Rat leukemia virus
        Rat sarcoma virus
        RD114 retrovirus
        Recombinant M-MuLV/RaLV retrovirus
Mammalian virus group
    Murine endogenous retrovirus
    Murine leukemia-related retroviruses
        Epicrionops marmoratus retrovirus
        Ichthyophis kohtaoensis retrovirus
        Osteolaemus tetraspis retrovirus
        Sericulus bakeri retrovirus
        Terdus iliacus retrovirus
        Tomistoma schlegelii retrovirus
        Viper berus retrovirus
        Xenotropic MuLV-related virus
            Xenotropic MuLV-related virus VP35
            Xenotropic MuLV-related virus VP42
            Xenotropic MuLV-related virus VP62
        Monodelphis sp. retrovirus
    Replication competent viruses
        Feline leukemia virus
            Feline leukemia provirus (clone CFE-16)
            Feline leukemia provirus (clone CFE-6)
            Feline leukemia provirus ftt
            Feline leukemia virus strain A/Glasgow-1
            Feline leukemia virus strain B/lambda-B1
            Feline leukemia virus strain C/FA27
            Feline leukemia virus strain C/FS246
            Feline leukemia virus strain C/Sarma
            Feline sarcoma virus
            Gardner-Arnstein feline leukemia oncovirus B
        Gibbon ape leukemia virus (GALV)
            Simian sarcoma-associated virus
        Murine leukemia virus
            AKR (endogenous) murine leukemia virus
            Friend murine leukemia virus
            Moloney murine leukemia virus
            Murine leukemia virus isolates
            unclassified Murine leukemia virus
        Porcine type-C oncovirus
            Porcine endogenous retrovirus
            Porcine endogenous type C retrovirus
    Replication defective viruses
        Abelson murine leukemia virus
        Gardner-Arnstein feline sarcoma virus
        Hardy-Zuckerman feline sarcoma virus
            Feline sarcoma virus (STRAIN HARDY-ZUCKERMAN 2)
            Feline sarcoma virus (STRAIN HARDY-ZUCKERMAN 4)
        Harvey murine sarcoma virus
        Kirsten murine sarcoma virus
        Moloney murine sarcoma virus
            Cas-NS-1 murine sarcoma virus
            FBJ murine osteosarcoma virus
            Moloney murine sarcoma virus (STRAIN HT-1)
            Moloney murine sarcoma virus (STRAIN M1)
            Moloney murine sarcoma virus (strain TS110)
            Murine sarcoma virus 3611
            Myeloproliferative sarcoma virus
            NS.C58 murine sarcoma virus
        Murine osteosarcoma virus
            FBR murine osteosarcoma virus
        Snyder-Theilen feline sarcoma virus
        Spleen focus-forming virus
            Friend spleen focus-forming virus
            Rauscher spleen focus-forming virus
        Woolly monkey sarcoma virus
```

Thus, the retroviral vector is in one embodiment derived from Chick syncytial virus, Feline leukemia virus, Finkel-Biskis-Jinkins murine sarcoma virus, Gardner-Arnstein feline sarcoma virus, Gibbon ape leukemia virus, Guinea pig type-C oncovirus, Hardy-Zuckerman feline sarcoma virus, Harvey murine sarcoma virus, Kirsten murine sarcoma virus, Moloney murine sarcoma virus, Murine leukemia virus (MLV), Porcine type-C oncovirus, Reticuloendotheliosis virus, Snyder-Theilen feline sarcoma virus, Trager duck spleen necrosis virus, Viper retrovirus or Woolly monkey sarcoma virus. See table 3 for a list of gammaviruses. Each of the gammaviruses specified herein is intended to be an individual embodiment. Consequently, a retroviral vector according to the present invention derived from each of them may be claimed individually. In a particularly preferred embodiment, the retroviral vector is derived from Murine Leukemia Virus (MLV) or Moloney Murine Leukemia Virus (MoMLV) or Akv MLV.

In a specific embodiment, the retroviral vector is derived from Avian (Reticuloendotheliosis) virus group such as Chick syncytial virus, Reticuloendotheliosis virus, Avian spleen necrosis virus, Spleen necrosis virus, Mammalian virus group, Murine endogenous retrovirus, Murine leukemia-related retroviruses, Epicrionops marmoratus retrovirus, Ichthyophis kohtaoensis retrovirus, Osteolaemus tetraspis retrovirus, Sericulus bakeri retrovirus, Terdus iliacus retrovirus, Tomistoma schlegelii retrovirus, Viper berus retrovirus, Xenotropic MuLV-related virus, Monodelphis sp. retrovirus, Replication competent viruses, Feline leukemia virus, Gibbon ape leukemia virus (GALV), Murine leukemia virus, Porcine type-C oncovirus, Replication defective viruses, Abelson murine leukemia virus, Gardner-Arnstein feline sarcoma virus, Hardy-Zuckerman feline sarcoma virus, Harvey murine sarcoma virus, Kirsten murine sarcoma virus, Moloney murine sarcoma virus, Murine osteosarcoma virus, Snyder-Theilen feline sarcoma virus, Spleen focus-forming virus, Woolly monkey sarcoma virus, unclassified Gammaretrovirus, Baboon endogenous virus, Baboon endogenous virus strain M7, Feline endogenous virus, Feline endogenous virus ECE1, Feline endogenous virus RD114, Koala retrovirus, Macaca mulatta type C retrovirus, Macaca endogenous retrovirus, MLV-related retrovirus, Rat leukemia virus, Rat sarcoma virus, RD114 retrovirus, Recombinant M-MuLV/RaLV retrovirus, Murine endogenous retrovirus, Murine leukemia-related retroviruses, Epicrionops marmoratus retrovirus, Ichthyophis kohtaoensis retrovirus, Osteolaemus tetraspis retrovirus, Sericulus bakeri retrovirus, Terdus iliacus retrovirus, Tomistoma schlegelii retrovirus, Viper berus retrovirus, Xenotropic MuLV-related virus, Xenotropic MuLV-related virus VP35, Xenotropic MuLV-related virus VP42, Xenotropic MuLV-related virus VP62, Monodelphis sp. retrovirus, Replication competent viruses, Feline leukemia virus, Feline leukemia provirus (clone CFE-16), Feline leukemia provirus (clone CFE-6), Feline leukemia provirus ftt, Feline leukemia virus strain A/Glasgow-1, Feline leukemia virus strain B/lambda-B1, Feline leukemia virus strain C/FA27, Feline leukemia virus strain C/FS246, Feline leukemia virus strain C/Sarma, Feline sarcoma virus, Gardner-Arnstein feline leukemia oncovirus B, Gibbon ape leukemia virus (GALV), Simian sarcoma-associated virus, Murine leukemia virus, AKR (endogenous) murine leukemia virus, Friend murine leukemia virus, Moloney murine leukemia virus, Murine leukemia virus isolates, unclassified Murine leukemia virus, Porcine type-C oncovirus, Porcine endogenous retrovirus, Porcine endogenous type C retrovirus, Replication defective viruses, Abelson murine leukemia virus, Gardner-Arnstein feline sarcoma virus, Hardy-Zuckerman feline sarcoma virus, Feline sarcoma virus (STRAIN HARDY-ZUCKERMAN 2), Feline sarcoma virus (STRAIN HARDY-ZUCKERMAN 4), Harvey murine sarcoma virus, Kirsten murine sarcoma virus, Moloney murine sarcoma virus, Cas-NS-1 murine sarcoma virus, FBJ murine osteosarcoma virus, Moloney murine sarcoma virus (STRAIN HT-1), Moloney murine sarcoma virus (STRAIN M1), Moloney murine sarcoma virus (strain TS110), Murine sarcoma virus 3611, Myeloproliferative sarcoma virus, NS.C58 murine sarcoma virus, Murine osteosarcoma virus, FBR murine osteosarcoma virus, Snyder-Theilen feline sarcoma virus, Spleen focus-forming virus, Friend spleen focus-forming virus, Rauscher spleen focus-forming virus or Woolly monkey sarcoma virus. Each of the gammaviruses mentioned above is intended to be an individual embodiment. Consequently, a retroviral vector according to the present invention derived from each of them is claimed individually.

In a further embodiment, the retroviral vector is derived from Bovine leukemia virus, Primate T-lymphotropic virus 1, Primate T-lymphotropic virus 2 or Primate T-lymphotropic virus 3. The deltaviruses are listed in table 4. Each of the deltaviruses mentioned herein is intended to be an individual embodiment. Consequently, a retroviral vector according to the present invention derived from each of them is claimed individually.

TABLE 4
List of deltaretroviruses

| | |
|---|---|
| Deltaretrovirus | Bovine leukemia virus |
| Deltaretrovirus | Primate T-lymphotropic virus 1 |
| Deltaretrovirus | Primate T-lymphotropic virus 2 |
| Deltaretrovirus | Primate T-lymphotropic virus 3 |

In yet a further embodiment, the retroviral vector is derived from Walleye dermal sarcoma virus, Walleye epidermal hyperplasia virus 1 or Walleye epidermal hyperplasia virus 2. The epsilonviruses are listed in table 5. Each of the epsilonviruses mentioned herein is intended to be an individual embodiment. Consequently, a retroviral vector according to the present invention derived from each of them may is individually.

TABLE 5
List of epsilonretroviruses

| | |
|---|---|
| Epsilonretrovirus | Walleye dermal sarcoma virus |
| Epsilonretrovirus | Walleye epidermal hyperplasia virus 1 |
| Epsilonretrovirus | Walleye epidermal hyperplasia virus 2 |

In a primary aspect of the present invention is provided a replication deficient retroviral vector. The vector comprises at least one heterologous nucleic acid sequence encoding an HIV-1 envelope polypeptide of the present invention or a fragment thereof. In one embodiment, the lentiviral envelope polypeptide or fragment thereof is derived from Bovine immunodeficiency virus, Caprine arthritis encephalitis virus, Equine infectious anemia virus, Feline immunodeficiency virus, Human immunodeficiency virus 1, Human immunodeficiency virus 2, Puma lentivirus, Simian immunodeficiency virus, Visna/maedi virus or hepatitis C. The lentiviruses, wherefrom the lentiviral envelope or fragment thereof can be derived are listed in table 6.

TABLE 6
List of lentiviruses from which a nucleic acid sequence encoding
an envelope polypeptide or fragment thereof is used according
to the present invention.

| | |
|---|---|
| Lentivirus | Bovine immunodeficiency virus |
| Lentivirus | Caprine arthritis encephalitis virus |
| Lentivirus | Equine infectious anemia virus |
| Lentivirus | Feline immunodeficiency virus |
| Lentivirus | Human immunodeficiency virus 1 |
| Lentivirus | Human immunodeficiency virus 2 |
| Lentivirus | Puma lentivirus |
| Lentivirus | Simian immunodeficiency virus |
| Lentivirus | Visna/maedi virus |

Each of the lentiviruses mentioned above is intended to be an individual embodiment. Consequently, a retroviral vector comprising at least one heterologous nucleic acid sequence encoding an HIV-1 envelope polypeptide of the present invention or a fragment thereof derived from each of them is claimed individually.

### Reporter Gene, Suicide Gene and Selectable Markers

The vector of the present invention may comprise at least one additional nucleic acid sequence, in addition to a nucleic acid encoding an HIV-1 envelope. In a particular embodiment of the present invention, the vectors and/or nucleic acids of the present invention comprise a reporter gene. Thus, in one embodiment, the at least one additional nucleic acid sequence of a vector of the present invention encodes a reporter gene. In the present context the term “reporter gene” refers to any reporter gene that can be used to evaluate whether a host cell harbours the vector, provirus, retroviral particle, composition and/or kit of the present invention. A number of reporter genes and systems for detection exist which will be appreciated by a person skilled in the art. For example the reporter gene of the present invention is selected from the group consisting of the enhanced green fluorescent protein (eGFP), lac Z, dsRed, enhanced yellow fluorescent protein (eYFP), enhanced cyan fluorescent protein (eCFP), enhanced blue fluorescent protein (eBFP) and the human alpha-1-antitrypsin (hAAT). It is understood that any of the enhanced green fluorescent protein (eGFP), lac Z, dsRed, enhanced yellow fluorescent protein (eYFP), enhanced cyan fluorescent protein (eCFP), enhanced blue fluorescent protein (eBFP) or the human alpha-1-antitrypsin (hAAT) there are also claimed in separate embodiments. In a preferred embodiment the eGFP gene is used.

In another embodiment of the present invention, the vectors and/or nucleic acids of the present invention comprise a suicide gene and/or a selection gene encoding a selective marker. The selection gene of the present invention may be any gene suitable for example for selecting cells harbouring the vector constructs of the present invention. Typically the selection gene is a gene that confers resistance to antibiotics or drugs. Examples of such selection genes are the puromycin resistance gene (Puro), the tetracycline resistance gene, the streptomycin resistance gene, the hygromycin B resistance gene (Hygro), the zeocin resistance gene (zeo), the neomycin resistance gene (neo), and the blasticidin resistance gene (Bst). Therefore, the selection gene of the present invention is selected from the group consisting of puromycin resistance gene (Puro), the tetracycline resistance gene, the streptomycin resistance gene, the hygromycin B resistance gene (Hygro), the zeocin resistance gene (zeo), the neomycin resistance gene (neo) and the blasticidin resistance gene (Bst). In a preferred embodiment the selection gene is selected from the group consisting of puromycin resistance gene (Puro), the hygromycin B resistance gene (Hygro), the zeocin resistance gene (zeo), the neomycin resistance gene (neo) and the blasticidin resistance gene (Bst). It is appreciated that the resistance gene is selected from any of puromycin resistance gene (Puro), the tetracycline resistance gene, the streptomycin resistance gene, the hygromycin B resistance gene (Hygro), the zeocin resistance gene (zeo), the neomycin resistance gene (neo) or the blasticidin resistance gene (Bst). In a preferred embodiment the resistance gene is the neomycin resistance gene. In another preferred embodiment, the selective marker is neomycin phosphotransferase II.

The suicide gene and/or a selection gene encoding a selective marker according to the present invention is in another embodiment Herpes simplex virus thymidine kinase (HSV-TK).

In a specific embodiment, the genes of the nucleic acids and/or vectors of the present invention are under the control of a constitutive promoter, however in another embodiment, the promoter is non-constitutive and may be activated.

### Constitutive Transport Element (CTE) and Rev Responsive Element (RRE)

The nucleic acids, including vectors and genomes of biological entities, of the present invention, in one embodiment, further comprise a constitutive transport element (CTE) that is characterised in that it serves as a signal of nuclear export of unspliced viral RNAs. The CTE of the present invention may be selected from the CTEs listed below in table 6A.

TABLE 6A
CTE elements

| Element | Origin | Ref |
|---|---|---|
| CTE | Mason-Pfizer Monkey Virus | Bray et al 1994 PNAS |
| RTE | Rodent intracisternal type | Wodrich et al 2001 J Virol |
| | A particle element (IAPE) | |
| CTE | Simian type D retrovirus | Tabernero et al 1996 J |
| | | Virol |
| WPRE | Woodchuck Hepatitis virus | Zufferey et al 1999 J Virol |
| RTEm26 | Rodent intracisternal type | Smulevitch et al 2005 J |
| | A particle element (IAPE) | Virol |
| DR | Rous Sarcoma Virus | Paca et al 2000 J Virol |

In a particular embodiment the CTE is derived from for Mason-Pfizer monkey virus, in another preferred embodiment the CTE is derived from the Woodchuck Hepatitis virus, for example the Woodchuck Hepatitis virus posttranscriptional regulatory element (WPRE).

Moreover, the vector may comprise a Rev responsive element (RRE). The presence of an RRE in the RNA transcript facilitates its transport from the nucleus to the cytoplasma, for example in a semipackaging cell, when REV/REX is coexpressed. In one specific embodiment, the vector is transcribed in the cytoplasm, thereby producing high levels of transcript, which can be translated into envelope polypeptide. This envelope polypeptide may then be incorporated into viral particles in a packaging/producer cell.

### Internal Ribosomal Entry Site (IRES)

The present invention also relates to a vector comprising at least one additional nucleic acid sequence, such as at least two nucleic acid sequences, such as at least three nucleic acid sequences, such as at least four nucleic acid sequences, such as at least five nucleic acid sequences. The additional nucleic acid sequences preferably comprise a translatable open reading frame. The nucleic acid sequences and/or vectors of the present invention are thus in one embodiment bi- or multicistronic. Therefore, in a preferred embodiment, the nucleic acids and/or vectors of the present invention further comprise at least one internal ribosomal entry site. Thus, the vector according to the present invention comprises, in one embodiment, two additional nucleic acid sequences, three, four, five or six additional nucleic acid sequences and at least two IRES elements, three, four, five or six IRES elements. In one embodiment, the at least one IRES are of different origin. Specifically, the nucleic acid sequence encoding an HIV-1 envelope polypeptide of the present invention or fragment thereof may be preceded by an IRES. Similarly, any of the at least one additional nucleic acid sequences encoding an HIV-1 envelope polypeptide or a part thereof, as mentioned herein, may be preceded by an IRES. In one embodiment of the present invention the HIV-1 envelope polypeptide or fragment thereof is not translated under the control of IRES. An IRES preceding a nucleic acid sequence results in the translation of said sequence under the control of the IRES. The IRES elements according to the present invention may be derived from picornaviridae, retroviridae or retrotransposons, mammalia or combinations thereof. In a specific embodiment, the IRES is selected from the IRES elements of encephalomyocarditis (ECMV) or another picornavirus. Specifically, the IRES according to the present invention can be derived from the IRES element of eIF4G. In further embodiments of the present invention, the vector comprises an IRES element, which is selected from any of the IRES elements listed in the tables below.

TABLE 7
IRES elements which can be used according to the present invention. RNA. 2006 Oct; 12(10): 1755-85
TABLE 1. Reported cellular IRES

| Gene name | Function | Organism | [symbol] seq pos | Reference |
|---|---|---|---|---|
| α-Cas [symbol] II | αsubunit | Rat | 203203 (200-431) | Pin [symbol] et al., 2001 |
| | Ca- [symbol] -dependent | | | |
| | kinase II | | | |
| ABETA | Amolaid β [symbol] precursor protein | Human | 341201 (899-1049) | Qin and Saman 2004 |
| AMU/RUNA1 | [symbol] factor | Human | 2944212 (8496-10040) | [symbol] ner et al., 2000 |
| Antp | A [symbol] | Drosophila | 16648961 (1-1209) | Oh et al., 1992 |
| Apaf-1 | Pro-apoptosis factor | Human | 2330014 (1-200) | Coldwell et al., 2000 |
| APC | Adenomatosis peptides coli gene | Human | 182396 (486-520) | Heppaner Goss et al., 2002 |
| ARC | Cytoskeleton association protein | Rat | 834413 (1-200) | Pin [symbol] et al., 2001 |
| ATIR | Angiotensin II type I | Human | 18490885 (1-273) | Mertin et al., 2003 |
| | receptor-Coprotein cocpied | | | |
| | receptor | | | |
| BAC-1 p36 | Anti-apoptosis factor | Human | 1143475 (1-413) | Coldwell et al., 2001 |
| Bcl-2 | Anti-apoptotic factor | Human | 129366 (313-1463) | Sherril et al., 2004 |
| [symbol] | ER protein chaperone | Human | 1143393 (1-225) | Macerak and Samow 1991 |
| βPix-b | Bak-interacting exchange factor | Mouse | 32288334 (1-375) | Rhes et al., 2004 |
| | isoform b | | | |
| Cat-1 | Cationic amino acid transporter | Rat | 18542233 (1-273) | Femander et al., 2001 |
| c-fos | Transcription factor | Chicken | 232221 (500-815) | Sehgal et al., 2000 |
| c-Myb | Transcription factor | Human | 45502012 (1-202) | Mitchell et al., 2005 |
| c-Myc | Transcription factor | Human | 11493193 (2501-2881, | Stoneley et al., 1998 |
| | | | 4506-4523) | |
| Connexin26 | Gap junction protein | Human | 1762120 (1472-1631, | Lahlou et al., 2005 |
| | | | 4780-4804) | |
| Connexin32 | Gap junction protein | Human | 974140 (404-529, 884-903) | Hudder and Werner 2000 |
| Connexin43 | Gap junction protein | Rat | 43393193 (1-232) | Sch [symbol] et al., 1999 |
| CCND1 | Cyclin D1 | Human | 22788593 (1380-3591) | S [symbol] et al., 2005 |
| DAP3 | Translation initiation factor | Human | 1903413 (1-357) | Henis-Konnenb [symbol] et al., 2000 |
| Dendrin | Putative modulator of the | Rat | 1252674 (1-151) | Pinkstaff et al., 2001 |
| | post-synaptic cytoskeleton | | | |
| elFAG | Translation initiation factor | Human | 21655145 (143-538) | Johnnes and Sar [symbol] 1998 |
| ERα | Estrogen receptor α | Human | 182192 (293-814) | Barraille et al., 1999 |
| FMR1 | RNA binding protein | Human | 1668818 (13698-13964) | Chiang et al., 2001 |
| FGF1a | Fibroblast growth factor | Human | 178226 (1-360) | Martineau et al., 2004 |
| | 1A - angiogenic factor | | | |
| FGF1a | Fibroblast growth factor | Mouse | 4321921 (865-1236) | Martineau et al., 2004 |
| | 1A - angiogenic factor | | | |
| FGF1b | Fibroblast growth factor | Human | 9125828 (35-185) | Martineau et al., 2004 |
| | 1B - angiogenic factor | | | |
| FGF1c | Fibroblast growth factor | Human | Poo [symbol] defined | Martineau et al., 2004 |
| | 1C - angiogenic factor | | | |
| FGF1d | Fibroblast growth factor | Human | 21595686 (10-93) | Martineau et al., 2004 |
| | 1D - angiogenic factor | | | |
| FGF1 | Fibroblast growth factor | Human | 31363 (486-809) | Martineau et al., 2004 |
| | | | | [symbol] et al., 2003. |
| HAP4 | Transcriptional activator | Yeast | 3762 (228-500) | [symbol] ka et al., 1994 |
| Haides | Transcription repressor | Fly | 157621 (686-1072) | Maier et al., 2002 |
| Htap2 | Anti-apoptosis protein | Human | 34367137 (1313-1465) | Wamakui [symbol] |
| | | | | et al., 2004 |
| HIF-1α | Trancription factor | Mouse | 12837319 (1-287) | Ling et al., 2002 |
| HnRNPA/B | Heterogenesis nuclear | Human | 33872827 (1-227) | Qin and Sanmow 2004 |
| | riboprotein A/B | | | |
| Hsp70 | Heat shock protein | Human | 184419 (274-491) | Rub [symbol] et al., 2003 [symbol] |
| | | | | Yuch and Schneder 2000 |
| Hsp70 | Heat shock protien | Fly [symbol] | 157720 (1514-1757) | Hemandez et al., 2004 |
| Hsp101 | Heat shock protein | Plant | 4584956 (290-438) | Dinkova et al., 2005 |
| IGF-II leader 1 | Insulin-like growth factor | Human | 26190552 (130580-130698, | Teerink et al., 1995 |
| | II-UTR leader 1 | | 139619-139838, | |
| | | | 141156-141399, | |
| | | | 153434-153443) | |
| IGF-II leader 2 | Insulin-like growth factor | Human | 6453816 (125-735) | Pedersen et al., 2002 |
| | II-UTR leader 2 | | | |
| IGF-IE | Growth factor receptor | Rat | 204227 (413-1358) | Cd et al., 2003 |
| Kv1.4 | Voltage-gated potassium channel | Mouse | 26331157 (1-1201) | Negulescu et al., 1998 |
| La1 | RNA binding protein--more | Human | 511006 (249-345, | Canter and Samow 2000 |
| | abudant trancript | | 2329-2340) | |
| La1⁺ | RNA binding protein-less | Human | 511006 (698-885, | Cader and Samow 2000 |
| | abudant trancript | | 2329-2340) | |
| LEF-1 | lymphoid enhancer factor | Human | 2285703 (1523-2703) | Limener et al., 2005 |
| L-myc | Lung myc | Human | 188906 (224-431, | lopling et al., 2004 |
| | | | 796-802) | |
| MAP2 | Cytoskeleton-associated protein | Rat | 987493 (1-120) | Pinkstaff et al., 2001 |
| M [symbol] | MAX binding protein-trancriptional | Human | 1041919 (33-215) | Soneiey et al., 2001 |
| | receptor | | | |
| M5 | Methionine synthase | Human | 1763268 (126-397) | Oltean and Banerjee 2005 |
| MTGSa (RUNX133) | Trancription factor | Human | 940399 (1-411) | Mitchell et al., 2005 |
| MYCHEX1 | Upstream open reading | Human | 11493393 (2223-2306) | Nanb [symbol] et al., 2001 |
| | frame on c-Myc trancript | | | |
| My12 | Myelin trancription factor 2 | Rat | 2246660 (997-1158) | Kim et al., 1998 |
| Nap11.1 | Nucleosome assembly | Human | 461207 (16-142) | Qin and Samow 2004 |
| | protein 1-like 1 | | | |
| NBS1 | Nijimegan breakage syndrome allele | Human | Undefined | Moiser et al., 2001 |
| Neurogranin (RC3) | Neural-specific regulator of CIMKII | Rat | 924645 (4217-4470) | Pinstaff et al., 2001 |
| | activity | | | |
| Nkxb.1 | Homodomain trancription factor | Mouse | 13338686 (2988-1959) | Wisade et al., 2000 |
| N-myc | Neuronal myc-trancription factor | Human | 11692795 (1-323) | lopling and Wills 2001 |
| Notch2 | Intercellular signaling receptor | Fly | 1622786 (1-238) | Lauding and Overbaugh 2000 |
| NFM1 | Nucleophosmin | Human | 2745708 (1222-1320) | Qin and Samow 2004 |
| NRF | NF-all repressing | Human | 2406601 (1-656) | Ou [symbol] et al., 2000 |
| | factor-trancription factor | | | |
| ODC | Om [symbol] | Rat | 205803 (1-426) | Pyronnet et al., 2000 |
| P [symbol] | Translation interaction factor | Yeast | 1323279 (14130-14641) | Zhou et al., 2001 |
| | homolog at etF4G | | | |
| P22 [symbol] | Cyclin-dependant kinase inhibitor | Mouse | 532221 (1-221) | Miskimins et al., 2001 |
| PKCδ | Protein kinase C delta | Rat | 206180 (2-363) | Monish and Rum [symbol] 2002 |
| PITS [symbol] RE | Cyclin-dependant kinase | Human | 507159 (946-1120) | Cornelis et al., 2000, |
| | | | | Tinten et al., 2003 |
| PP2Cβ | Protein phosphotase 2Cβ | Rat | 12666526 (1-400) | Se [symbol] et al., 2001 |
| Reaper | Pro-apoptosis protein | Fly, Dmt | 476009 (1-174) | Hermandez et al., 2004 |
| Runx2 Type I | Runt-related transcription | Mouse | 391766 (1-1018) | Kiao et al., 2003 |
| | factor 2-UTR2 | | | |
| Runx2 Type II | Runt-related transcription | Mouse | 390766 (1-207 | Kiao et al., 2003 |
| | factor 2-UTR1 | | | |
| Scampes | Calcium channel | Dog | 21553346 (263-368) | De [symbol] Toneil et al., 2003 |
| Smadi | Mediator of bone | Human | 4433529 (259-360) | St [symbol] et al., 2002 |
| | morphogenetic protein signaling | | | |
| SNM1 | Homolog of [symbol] sensitivity to | Human | 522302 (1-921) | Zhang et al., 2002 |
| | mitrogene mustand gene | | | |
| TIE2 | Tyrosine kinase with | Human | 20411198 | P [symbol] et al., 2003 |
| | immunoglobulin like and | | (28326-27857) | |
| | EGF-like domains 1 | | | |
| TFHO | Transcriptional activator | Yeast | 122898 ( [symbol] -275) | [symbol] zoka et al., 1994 |
| TFK8 | Neurotropsin receptor-tropomysin- | Human | 18369867 (3761-1940) | D [symbol] et al., 2003 |
| | [symbol] tyrosine kinase | | | |
| Ubx | Ultrabinding-hometic gene | Fly | 8294 (3518-4135) | Hart and Bienz 1996 |
| Unx | Upstream of M- [symbol] | Human | 32220548 (1-468) | C [symbol] et al., 2005 |
| Utr | Utropsin | Mouse | 74144053 (195-704) | Miuta et al., 2005 |
| Vlbr | Vasopressin V1b receptor | Rat | 945040 (35-544) | Kahadan-Dield et al., 2003 |
| VECF-A [symbol] A | Vascular endothelial | Human/mouse | 4154290 (2864-3403) | Stein et al., 1998; |
| | growth factor-A | | 1134964 (1218-2234) | Huex et al., 2001 |
| VEGF. [symbol] esii | Voscular endotheial growth factor-A | Human | 415290 (2363-2863) | Stein et al, 1990; |
| | | | | Huex et al, 2003 |
| Vinkentin | Stuctural protein | Human | 2437837 (1758-1982) | Qus and sarpow 2004 |
| KIAP | Apoposis inhibtor | Human | 28250426 (306-409) | Bolck et al, 1999 |
| YAPI | Yes-as-ocefed Protein transcriptional | Yeast | 4792 (297-333) | Zhou et al, 2001 |
| | activator | | | |

Sequences are either the minimal sequence of the fully functioning IRES of the begining of the known 5′-UTR. The sequences 3′ end includes the staft [symbol] position [symbol] . Some [symbol] in the published positions have been [symbol] upon comminications with the authors where possible. [symbol]
indicates data missing or illegible when filed

TABLE 8
Reported viral IRES elements which can be used according to the present
invention. RNA. 2006 Oct; 12(10): 1755-85.
TABLE 2 Reported viral IRES

| Virus | Viral name product | Viral host | GI (IRES seq. pos.) | Reference |
|---|---|---|---|---|
| Dioistroi [symbol] | | | | |
| [symbol] | | | | |
| CrPv | Cricket paralysis virus | Insect | 8895586 (1-211) | Wilson et al, 2008 |
| | ORF1-cons [symbol] protein | | | |
| CrPv | OSf2-stuctural proteins | Insect | [symbol] (6025-6216) | Wilson et al, 2008 |
| | | | contains c [symbol] stan codon | |
| | | | needed ior pseudokina | |
| DCV | Drosaphila C virus IRES1 | Insect | 2388672 (1-661) | Johnson and Christian 1998 |
| DCV | Drosaphila C virus IRES2 | Insect | 2388672 (6080-6266) | Johnson and Christian 1998; |
| | | | contains c [symbol] stan codon | Kanamed and Nakashuma 2001 |
| | | | needed for pseudokina | |
| PSIV | Plau [symbol] stall intentine virus - | Insect | 2344756 (5949-6195) | [symbol] and Nakahima 1999 |
| | capsod protein | | | |
| RhPV | Rhopa [symbol] phum padi ORF2 | Insect | 2911238 (6327-7112) | Domier et al, 2000 |
| | | | gca start codon | |
| TRV | Triatco [symbol] virus-5′-UTR | Insect | 6003484 (1-631) | Cabener et al, 2005 |
| TRV-IGR | Triatco [symbol] virus-intergenic region | Insect | 6003484 (5934-6311) | Cabener et al, 2005 |
| TSV | T [symbol] syndrome | S [symbol] | Contains reu start codon | Hafakeyama et al, 2004 |
| | virus-capsid protein | | needed for pseudokina | |
| Flaenvinid [symbol] | | | | |
| [symbol] | | | | |
| HCV | Hepatitis C virus | Human | 126311192-(1-144) | Tsukiyama-katara et al, 1992 |
| Flavi [symbol] | | | | |
| [symbol] | | | | |
| BVDV | Bovine viral dia [symbol] virus | Cow | [symbol] 36067 0-383 | [symbol] et al, 1995 |
| CSFV/HoCV | Classical swine leves | Pig | 1256482 (1-376) | Riphomad et al, 1997 |
| | virusbog [symbol] virus | | | |
| Flav [symbol] | | | | |
| Unclassified | | | | |
| GSV-B | Hepaditis virus [symbol] | Primates | 13162367 (23-448) | Grace et al, 1999 |
| | 8 inom patient GB | | | |
| Herpesvaidae, | | | | |
| [symbol] | | | | |
| (dsDNA) | | | | |
| KSHV | Kernnel- [symbol] -associated | Human | 2063326 | Bieieki and [symbol] 2000; |
| | [symbol] | | (223206-122709) | Gaundhojj and Garram 2001; |
| | | | | [symbol] et al, 2008) |
| Retravirible | | | | |
| F-MutV | friend murine leutamis virus | Mouse | 61544 (1-157) 61544 | [symbol] and Darlix 1995 |
| | [symbol] , and sag polyprotein | | (1-621) | |
| F-mutV | Friend murine lukemia | Mouse | 61544 (5492-5760) | Deiiski and Darlix 2008a |
| | virus-envelope gene | | | |
| HaMEV | Harvey murine [symbol] | gat | 207622 (25-533) | Hertioz et al, 1995 |
| | virus-Vt30 | | | |
| HTIV-1 | Human T-cell Lympho [symbol] | Human | 221866 (354-621) | Attai et al., 1995 |
| | virus 1-R and portial US region | | No staff codon | |
| MoMutV | Moitery murine leukemia virus | Mouse | 333923 (812-1041) | Vagnea et al, 1995b |
| RSV | Raus sarcoma virus-gag | Chicken | 2603459 (210-382) | Defauki and Darlix 2000b |
| RSV-src | Rous sarcoma virus-src | Chicken | 2801439 (7086-7131) | Defauki and Darlix 2000b |
| | | | No [symbol] than [symbol] spliced | |
| | | | UTR exists | |
| HIV | Human immunnodefiency virus | [symbol] | 14657 (607-1041) | O [symbol] mann et al, 2000 |
| Piomavirides | | | | |
| Aphthovirus | | | | |
| FMDV | Font and motiff disease virus | Mammais | 61070 (1252-716) | Nohn et al., 1990 |
| Piocommavirides | | | | |
| Candiovirus | | | | |
| EMCV | Encephaicmmy [symbol] virus | Human | 9626692 (260-836) | lang et al, 1998 |
| TMEV | Theier's murind | Mouse | 6205528-(876) | Pilipenko et al, 1994 |
| | encephafocmyetis, virus | | | |
| Pico [symbol] | | | | |
| [symbol] virus | | | | |
| CVB3 | C [symbol] b3 | Human | 5439992011-2433 | Yang et al. 1987 |
| EV71 | E [symbol] 71 strain B [symbol] | Human | 117112011-7460 | Bh [symbol] and Sa [symbol] 2003 |
| PV | Pediovirus | Human | 611234-2501 | [symbol] and Sonenberg 1966 |
| Picomavadae | | | | |
| Hepa [symbol] | | | | |
| HAV | Hepatitis A virus | Human | 329585 (150-720) | Brown et al. 1994 |
| Pico [symbol] | | | | |
| Rhinovirus | | | | |
| HRV | Human rhinovirus | Human | 220708 (20-625) | Borman and Jackson 1993 |
| Pico [symbol] | | | | |
| Techovirus | | | | |
| PPV-1 | [symbol] | Pig | 10111645 (146-414) | [symbol] et al. 2001 |
| | 1 stain [symbol] | | | |
| Leu [symbol] prima | | | | |
| [symbol] group | | | | |
| HIV | Human Immunodeficiency | Human | 4338520 838-808 | Back et al. 2001 |
| | virus type t gag | | [symbol] resides in CDS | |
| [symbol] | | | | |
| Pol [symbol] | | | | |
| PLRV | [symbol] virus | Plate ( [symbol] ) | 222301 (1513-1728) | Jaag et al, 2009 |
| | | | includes 21 | |
| Potyviridae | | | bases of CDS | |
| Podyvirus | | | | |
| CPMV | Cowpea mouse virus | Plant | 58910 (161-514) | Thoma et al. 1991 |
| PVY | Potato virus Y | Plant | 61450 (1-187) | Levis and Astict Manfacter 1993 |
| TEV | Tobacco etch virus | Plant | 335207 (2-147) | Carringon and [symbol] 1990 |
| Potyviridae | | | | |
| Avipoxodis | | | | |
| (dsDNA) | | | | |
| REV | Avian [symbol] | Bird | 28927608 (400-974) | Lopez Eastra et al. 1997 |
| | virus type A | | | |
| [symbol] | Tobaco mosaic virus | Plant | 486713 (3456-5600) | [symbol] et al. 1997 |
| criMV | [symbol] | | | |
| [symbol] | | | | |
| Gladiavirus | | | | |
| (dsDNA virus) | | | | |
| GLV | Glandia lamidia virus | Glandis lambida | 1352866 (8-368) | Garlapati and Wang 2005 |

Surgenece and either the munimal sequence of the fully [symbol] IRES or the beginning of the 5′UTR. The sequence's 3′-end includes the start codon. position [symbol] . Some error in the published positions have been corrected upon communications with the [symbol] where possible. [symbol]
indicates data missing or illegible when filed

TABLE 9
Reported minimal IRES modules. RNA. 2006 Oct;12(10): 1755-85
TABLE [symbol] Reported minimal IRES modules.

| [symbol] | Full name and product | [symbol] | Sequence | Reference |
|---|---|---|---|---|
| ARC-1 | Active iRNA complementary | Plant | AUAC [symbol] CCCCC | [symbol] |
| | sequence [symbol] | | G [symbol] GGACTT [symbol] GCACTGGA | |
| [symbol] | [symbol] | Human | ACUACAACACCCGA | [symbol] |
| (minimal | | | GCAAGGACGCG | |
| IRES) | | | ACTCT | |
| [symbol] | [symbol] | Mouse | CCGGCGGGT | [symbol] et al. 2000 |
| [symbol] | [symbol] RNA-binding protein | Mouse | UUUAGAAUGUCUUCU | [symbol] et al. 2001. |
| | | | UCGAGAA | [symbol] and [symbol] 2003 |
| synthetic | Short synthetic nucleotides | Human/yeast | Five positive | [symbol] and [symbol] [symbol] |
| | transcribed within IRES [symbol] | | [symbol] - [symbol] /56 positive | [symbol] et al. [symbol] |
| | | | 18- [symbol] | |
| [symbol] | [symbol] simplex virus [symbol] | Human | CCGUGCUGGCGUC [symbol] G | [symbol] and [symbol] [symbol] |
| | kinase mutant | | [symbol] [symbol] codon | |

[symbol]

indicates data missing or illegible when filed

TABLE 10
list of viral IRES elements, which can be used according to the present
invention. Curr Gene Ther. 2004 Mar; 4(1): 15-31.
Table 1A. List of Viral IRESe [symbol] Discovered Up to Date

| Viral IRES | Virus | 5′UTR Length | IRES Length | Reference |
|---|---|---|---|---|
| Picorn [symbol] Family | | | | |
| [symbol] | | | | |
| PV | Poliovirus | 742nt | | [Hellen et al., 1994] |
| CV-B (BB)* | Cossackievirus B (B3) | 743nt | | [Ray and Das, 2002] |
| EV (12)* | Echovirus (12) | 764nt | | [Bradrick et al., 2001] |
| SVDV | Swine Vesiculor Disease Virus | 742nt | | [Chen et al., 1993] |
| [symbol] | | | | |
| HRV (14)* | Human Rhinovirus (14) | 636nt | 550nt | [Rojao-Eiening et al., 1995] |
| Apthovirus | | | | |
| FMDV | Foot and Maxb Disease Virus | 828nt | 438nt | [Belcham, 1992] |
| Cardiovirus | | | | |
| EMCV | Encepholomyocattis Virus | 833nt | 431nt | [Jang and Winser, 1990] |
| TMEV (GDVII) | Thiller's Morine Encepholomyocattis Virus | 1067nt | | [Piligenko et al., 2000] |
| Heostovirus | | | | |
| HAV | Hepenitie A Virus | 730nt | 388nt | [Glass and Gammers, 1993] |
| Tesciavirus | | | | |
| PTV (Taltiue)* | Puncine Eesclovirus-1 (Taltiue) | 412nt | 405nt | [Kake et al., 2002] |
| Erbovirus | | | | |
| ERBV | Eqnire Plasmids B Virus | 920nt | 710nt | [Hinton and Crobb, 2001] |
| Other Viruses | | | | |
| HCV | Hepatitis C Virus | 341nt | | [Wong et al., 1994] |
| MHV | Morine Hepatitis Virus | | 286nt | [Jesdrack et al., 1994] |
| MMLV | Moloney Morine Leukemia Virus (gag)** | | 126nt | [Vagner et al., 1995] |
| FrMLV | Friend Morine Leukemia Virus (gag)** | 500nt | | [Deffsud and Ostlux, 2000] |
| HTLV-1 | Human T Cell Leukemia Virus 1 | 284nt | | [Attal et al., 1995] |
| RmPV | R [symbol] pedi Virus | 579nt | | [Woodway et al., 2001] |
| CSFV | Cladical Swine Fever Virus | 374nt | 350nt | [Sizone et al., 1998] |
| PSTV | Plonin Stat Inovessine Virus | | 250nt | [Sseski and [symbol] , 1998] |
| BVDV | Bovine Virus Disease Virus | 385nt | | [Chen et al., 1998] |
| CrPV | Cricker Pazolysis Virus | | 204nt | [Jan and Semonce, 2002] |
| TEV | Tabacco Etch Virus | 143nt | | [Gellie, 2001] |
| KSHV | Kaposile Sarcoma-associated Herpissina | | 233nt | [Biel [symbol] and Tailbox, 2001] |
| | (vCyclic)** | | | |

*Strain number or name indicated in parenthes.
**Gene name indicated in parantheses.
Table 1B. List of Cellular IRESes Discovered Up to Date

| | | | | |
|---|---|---|---|---|
| Cellular IRES | Gene Product | 5′UTR Length | IRES Length | References |

Growth Factors

| | | | | |
|---|---|---|---|---|
| VEGF | Vascular Endothelial Growth Factor | 1014nt | 163nt | [Sreia et al. 1998] |
| FGF2 | Fibroblost Growth Factor 2 | 318nt | 165nt | [Vagner et al., 1995a] |
| IGF II (5′UTR 1) | Insulin-like Growth Factor II | 598nt | | [ [symbol] et al., 1995] |
| IGF II (5′UTR2) | Insulin-like Growth Factor II | 400nt | 200nt | [Peterson et al., 2002] |
| Cyr81 | (A [symbol] -induced secreted protein) | 250nt | | [ [symbol] et al., 1999] |

Probe-oncogenes

| | | | | |
|---|---|---|---|---|
| c-myc | (A member of myc family) | 383nt | 270nt | [ [symbol] et al., 1997] |
| N-myc | (A member of myc family) | 320nt | | [Jopling and Willis, 2001] |
| PDGF-2/c- [symbol] | Platelet-derived Growth Factor 2 | 1022nt | 396nt | [Barnett et al., 1997] |
| ODC | [symbol] Decarbonylate | 303nt | 153nt | [Pyronet et al., 2000] |
| Pbn-I | (A serise-threonine protein kinase) | 380nt | | [Jo [symbol] et al., 1999] |
| c- [symbol] | Transcription Factor | 302nt | | [Seigal et al., 2000] |
| BAG-1 | Anti-apoptosis factor | 410nt | | [Coldwell et al., 2001] |

[symbol]

| | | | | |
|---|---|---|---|---|
| PITSLRE | (A Protein Nisase) | 100nt | 382nt (80%) | [Cornells et al., 200] |

Recestory/Channels/

[symbol]

| | | | | |
|---|---|---|---|---|
| IGF-IR | Insulin-like Growth Factor Receptor | 943nt | | [Gizaud et al., 2003] |
| Norcb2 | (A cell surface transmembrane receptors | 214nt | 235nt | [Lasting and |
| | protein) | | | Overbaugh, 2000] |
| Kv1AMCNA4 | (A Cardiac Voltage-gated Pot [symbol] | 1200nt | 200nt | [Negolesca et al., 1998] |
| | Channel) | | | |
| Connexid43 | (A gap [symbol] protein) | 208nt | 164nt | [Schdevi et al., 1999] |
| Connexid32 | (A gap [symbol] protein) | 150nt | | [Hudder and Warner, 2000] |
| Cat-1 | Cationic Amino Acid Transporte | 234nt | | [Fernandez et al. 2001] |
| Scoper | (A Sphingcaylphosphobolize Receptor) | 361nt | | [De Pletti Tonelli et al., |
| | | | | 2003] |

Transcriptional/Translational Factors

| | | | | |
|---|---|---|---|---|
| NRx6.1 | (A hemeodomain transcription factor) | 973nt | | [Wstads, et al, 2000] |
| AML1RUMK1 | (A mut decausan transcription factors) | 163nt | | [Pozzier et al, 2000] |
| HIF-1α | Hypossis-inducible Factor 1α | 265nt | | [Lund et al, 2002] |
| MYFZ | (A DNA-binding protein) | 1155nt | | [Kruez et al, 1998] |
| Ls1 | L0 Auttestige | 115nt | 80nt | [Carner and Sannow, 2000] |
| Ls1 | L0 Auttestige | 483nt | 109nt | [Carner and Sannow, 2000] |
| eIF4G | Eukaryotic [symbol] Factor 4G | 357nt | 101nt | [Gau et al, 1993] |
| PMR1 | (A RNA-binding protein) | 257nt | 204nt | [Chiang et al, 2009] |
| Fbm3 | (A RNA-binding protein) | 220nt | | [Chappell et al, 2001] |

Transcriptional Repressors

| | | | |
|---|---|---|---|
| Mn | (Mad family-related Transcriptional | 180nt | [ [symbol] et. al., 2001] |
| | repressor) | | |
| MEF | NP-XB Repressing Facter | 652nt | [C [symbol] d et. al., 2000] |
| Gtz | (A homeodomine protein) | 196nt | [Cl et. al., 2000] |

Signaling Molerbis

| | | | | |
|---|---|---|---|---|
| S [symbol] 5 | (Intracellular [symbol] or of RMP signaling | 320nt | 100nt | [Sh [symbol] ki et. al., 2002] |
| | J [symbol] ay) | | | |

An [symbol] -natutent Facters

| | | | | |
|---|---|---|---|---|
| Apaf-1 | Apoptonic Pro [symbol] erse Activiting Factor (Pto-) | 578nt | 233nt | [Cold [symbol] e [symbol] et. al., 2000] |
| DAP [symbol] NAT1 | Death-associated protein 5 (pro-) | 306nt | | [H [symbol] -Ko [symbol] et. al., |
| | | | | 2000] |
| XIAP | X-linked Inhibitor of Apoptosis ( [symbol] -) | 1007nt | 162nt | [H [symbol] et. al., 1999] |

Others

| | | | | |
|---|---|---|---|---|
| Hsp70 | Heas Shock Protein | 216nt | | [R [symbol] et. al., 2003] |
| p27 | (A cell cycle IE [symbol] ) | 217nt | | [Mis [symbol] et. al., 2001] |
| β-mRNA | B Subunit of Mitoxiondrial R-ATP | 150nt | | [Iequients and Cae [symbol] s. |
| | Syntase | | | 2000] |
| Eip | Ino [symbol] oglo [symbol] Heavy Clasic B [symbol] ling | 220nt | 91nt | [Yang and S [symbol] , 1997] |
| | Protein | | | |
| A [symbol] p | A [symbol] (A dr [symbol] gene) | 1735nt | | [Ye et. al., 1997] |
| ET [symbol] | U [symbol] (A frosophils gene) | 268nt | | [Ye et. al., 1997] |
| Heit [symbol] ess | (M [symbol] h R [symbol] go [symbol] ) | 130nt | | [Mrier et. al., 2003] |

Table 3B. Cellular IRESee and Signals that Regulate the IRES Activity

| | | | | |
|---|---|---|---|---|
| | Cell lines | | | |
| Cellular IRES | origin | Tested in | Regulation of activity | References |

Growth Factors

| | | | | |
|---|---|---|---|---|
| VEGF | Brain | C6, Neuro-24 | Hypoxia | [Axin et al., 1998, Enez et al., 1998; Stain et |
| | Kidney | 2P3, DO57 | Hypoglycernar | al., 1998, Wong et al., 2002] |
| | Cervix | HeLa, NB32 | | |
| FGF2 | Brain | SK-N-AS, SK-N-BE | Transformation | [Vagner et al., 1995a; Vagner et al., 1996] |
| | Kidney | COS7, 293 | Heat Shock | |
| | Liver | SK-Hep-5 | Serum starvasion & oxidatiive | |
| | | | stress | |
| | Skin | Harmone skin fibroblast | | |
| | Bone | Sros2 | | |
| | Ovary | CHO | | |
| IGF II (5′UTR I) | Cervix | HeLa | | [Teesick et al., 1995] |
| IGF II (5′UTR II) | | RD | | [Pedersen et al., 2002] |
| Cyrol | Cervix | HeLa | | [Jakannes et al., 1999] |

Proto-oncogenes

| | | | | |
|---|---|---|---|---|
| c-myc | Brain | Neuro-2a, SF539 | Cell cycle G2 [symbol] M phase | [ [symbol] et al., 1997, Swedey et al., 1998] |
| | Lung | MRC5 | Apoptosis | Pyro [symbol] et al., 2000; Stoneley et al., 2000] |
| | Liver | HepG2 | | HeLA-Kerebilt et al., 2002; Nerve et al., |
| | Kidney | 295, 29ST, COS7 | | 2002, Wong et al., 2002] |
| | Bladder | T24 | | |
| | Cervix | HeLa, KR31 | | |
| | Ovary | CHO | | |
| N-myc | Brain | SH-SY5Y, NB2a | Neuroual cell differentiation | [Jopling and Willis. 2001] |
| | Breast | MCF7 | | |
| | Kidney | 293 | | |
| | Cervix | HeLa | | |
| | Testicle | NT2 | | |
| PDGF-2k-bis | Blood | K582 | Differentiation | [Berostein et al., 1997; Sells et al, 1999] |
| ODC | Cervia | HeLa | Cell cycle G2/M phase | [Pyroceet et al., 2000] |
| Pim-3 | Cervia | HeLa | | [Jouannes et al., 1999] |
| c-jun | | 1′CEF | | [Seligal et al., 2000] |
| | | RAT1 | | |
| | | NIH BTB | | |
| BAG-1 | Cervix | HeLa | Heat Shock | [Coldiwell et al., 2001] |

Tumor Suporersor

| | | | | |
|---|---|---|---|---|
| PITGLRE | Kidney | 195T | Cell cycle G2M phase | [Coraeits et al., 2000] |
| | Blood | BaF3 | | |

Seropter/Charrdels/Transports

| | | | | |
|---|---|---|---|---|
| IGF-IR | Kidney | COST | | [Girsod et al., 2001] |
| Noxb2 | Cervix | HeLa | | [Lauring and Overbsogh 2000] |
| Kvl.4/KCNA4 | | NIH3T3 | | [Negulearn et al., 1999] |
| Connexin43 | Brain | Neuro2a | | [Schiavi et al., 1999] |
| | Cervix | HeLa | | |
| Connexin32 | Brain | Neuro-2a | | [Hudder and Wemer. 2000] |
| | Cervix | HeLa | | |
| Car-1 | Brain | C6 | Amino acid starvation | [Fermodel et al., 2001] |
| Scamper | Kidney | MDCK | | [De Pietri Touelli et al., 2003] |

Transcriptional/Transmitional Factors

| | | | | |
|---|---|---|---|---|
| Nrx6.1 | Process | β cells (β TC3, INS1) - | | [Wetada et al., 2000] |
| | | high activity | | |
| | Kidney | α cell (αTC1.6) - | | |
| | | suboptimal | | |
| | | COS7—low activity | | |
| AMLL/RUNX1 | Blood | K582, [symbol] SUP-TI - | Megskeryoxyik differentiation | [Pomer et al., 2000] |
| | | high activity | | |
| | | Bjah, SXY6.4 - low | | |
| | | activity | | |
| | | 293 | | |
| | | HeLa | | |
| HIF-1α | Kidney | 293 | Hypxia | [Lang et al., 2002] |
| | Cerviz | HeLa | Serun starvasion | |
| MYT2 | Brain | SVG | | [Kim et al., 1998] |
| | Cervix | HeLa | | |
| Lal | Cervix | HeLa | | [Cartex and Serum, 2000] |
| LaP | Cervix | HeLa | | [Cartex and Serum, 2000] |
| elF4G | Brain | Neuro-2a | | [Gen et al., Wong et al., 2002] |
| | Liver | HepG2 | | |
| | Kidney | 293 | | |
| | Blood | K562 | | |
| | Cervix | XB11 | | |

| | | | | |
|---|---|---|---|---|
| Cellular IRES | Gene Product | 5′UTR Length | IRES Length | References |

Transcriptional Processess

| | | | |
|---|---|---|---|
| Max | (Msd family-relsted transcriptional | 180nt | [Stoneley et al., 2001] |
| | repressor) | | |
| MRF | WF-kB Repressing Psctor | 653nt | [ [symbol] et al., 2000] |
| Gts | (A homeodomsin protein) | 190nt | [Chappess et al., 2000] |

Signaling Molecule

| | | | | |
|---|---|---|---|---|
| Start5 | (Intracellular issedistor of ESIP signaling | 321nt | 100nt | [Shireld et al., 2002] |
| | pathway) | | | |

Apoprosis-related Factor

| | | | | |
|---|---|---|---|---|
| Apaf-1 | Apoprotic Presses Actionsing Factor (pro-) | 578nt | 233nt | [Colorell et al., 2000] |
| DAP5pG7NAT1 | Deals-associated protein 5 (pro-) | 306nt | | [Heinis-KoreMix et al., 2000] |
| XIAP | X-linked Sadictor of Apoprosis (pro-) | 100nt | 16nt | [Holrik et al., 1999] |

Others

| | | | | |
|---|---|---|---|---|
| Hsp70 | Hear Shory Protein | 216nt | | [Enbrssors et al., 2003] |
| p27 | (A cell cycle registory) | 217nt | | [Maxmim et al., 2001] |
| βmRNA | B Submit of Mitchondrial H′-ATP | 150nt | | [Irqiends and Caervs, |
| | Synthase | | | 2000] |
| Bip | Immoglobulin Heavy Chain Binding | 120nt | 91nt | [Yang and Sarnow, 1997] |
| | Protein | | | |
| Amp | A [symbol] pedia (A drosphila gene) | 1735nt | | [Ye et al., 1997] |
| [symbol] | [symbol] (A drosphila gene) | 968nt | | [Ye et al., 1997] |
| Hairless | (Notch anagonist) | 139nt | | [Maiez et al., 2002] |

| | | | | |
|---|---|---|---|---|
| | Cell line | | | |
| Cellular IRES | origin | Tested in | Regulation of activity | References |

Growth Factors

| | | | | |
|---|---|---|---|---|
| VEGF | Brain | C6, Neuro-24 | Hypoxia | [Axin et al., 1998, Enez et al., 1998; Stain et |
| | Kidney | 293, COS7 | Hypoglycemia | al., 1998, Wong et al., 2002] |
| | Cervix | HeLa, NB32 | | |
| FGF2 | Brain | SK-N-AS, SK-N-BE | Transformation | [Vagner et al., 1995a; Vagner et al., 1996] |
| | Kidney | COS7, 293 | Heat Shock | |
| | Liver | SK-Hep-5 | Serum starvasion & oxidatiive | |
| | | | stress | |
| | Skin | Harmone skin fibroblast | | |
| | Bone | Sros2 | | |
| | Ovary | CHO | | |
| IGF II (5′UTR I) | Cervix | HeLa | | [Teesick et al., 1995] |
| IGF II (5′UTR II) | | RD | | [Pedersen et al., 2002] |
| Cyrol | Cervix | HeLa | | [Jakannes et al., 1999] |

Proto-oncogenes

| | | | | |
|---|---|---|---|---|
| c-myc | Brain | Neuro-2a, SF539 | Cell cycle G2 [symbol] M phase | [ [symbol] et al., 1997, Swedey et al., 1998] |
| | Lung | MRC5 | Apoptosis | Pyro [symbol] et al., 2000; Stoneley et al., 2000] |
| | Liver | HepG2 | | HeLA-Kerebilt et al., 2002; Nerve et al., |
| | Kidney | 295, 29ST, COS7 | | 2002, Wong et al., 2002] |
| | Bladder | T24 | | |
| | Cervix | HeLa, KR31 | | |
| | Ovary | CHO | | |
| N-myc | Brain | SH-SY5Y, NB2a | Neuroual cell differentiation | [Jopling and Willis. 2001] |
| | Breast | MCF7 | | |
| | Kidney | 293 | | |
| | Cervix | HeLa | | |
| | Testicle | NT2 | | |
| PDGF-2k-sis | Blood | K562 | Differentiation | [Bernstesis et al., 1997, Setta et al., 1999] |
| ODC | Cervix | HeLa | Cell cycle G12d phase | [P [symbol] et al., 2000] |
| Pim-3 | Cervix | HeLa | | [Johanses et al., 1999] |
| c-Jan | | [symbol] CEF | | [Sekgal et al., 2000] |
| | | RAT1 | | |
| | | NTHSTS | | |
| RAG-1 | Cervix | HeLa | Heat Stock | [Coldwell et. al., 2001] |

Tumor Suppressor

| | | | | |
|---|---|---|---|---|
| PITSLRE | Kidney | 193T | Cell cycle GIM phase | [Cornelle et al., 2000] |
| | Blood | BsF3 | | |

Receptors/Ch [symbol] /Transporters

| | | | | |
|---|---|---|---|---|
| IGF-IR | Kidney | COS7 | | [Gitsel et al., 2001] |
| Norcb2 | Cervix | HeLa | | [La [symbol] and Over [symbol] , 2000] |
| Kv1.4KCNA4 | | NIH3T3 | | [Ne [symbol] et al., 1998] |
| Connexed43 | Brain | Mem2a | | [Schiavi et al., 1999] |
| | Cervix | HeLa | | |
| Connexed32 | Brain | Neuro-2a | | [Holder and Werner, 2000] |
| | Cervix | HeLa | | |
| Cat-I | Brain | C6 | Amino acid [symbol] | [Fernander et al., 2000] |
| Scamper | Kidney | MDCK | | [De Pieri Teneii et al., 2003] |

Transcriptional/Transcriptional Factors

| | | | | |
|---|---|---|---|---|
| Nax6.1 | Pancreas | β cells (BTC3, INSI)- | | [Wetada et al., 2000] |
| | | high activity | | |
| | Kidney | α cell (αTC1.6)- | | |
| | | suboptional | | |
| | | COS7 - low activity | | |
| AMLIRUNX: | Blood | K562, [symbol] SUP-T1- | Meg [symbol] differentiation | [Pomer et al., 2000] |
| | | high activity | | |
| | Kidney | Bjxb. SKW6.4 - low | | |
| | Cervix | activity | | |
| | | 283 | | |
| | | HeLa | | |
| HIF-1α | Kidney | 293 | Hyposis | [Lang et al., 2002] |
| | Cervix | HeLa | Serum [symbol] | |
| MYT2 | Brain | SVG | | [Kim et al., 1998] |
| | Cervix | HeLa | | |
| LsF | Cervix | HeLa | | [Caster and Se [symbol] , 2000] |
| LSF | Cervix | HeLa | | [Caster and Se [symbol] , 2000] |
| eIF4G | Brain | Mem-2a | | [Gan et al., 1998, Wang et al., 2002] |
| | Liver | HepG2 | | |
| | Kidney | 293 | | |
| | Blood | N562 | | |
| | Cervix | KBI3 | | |
| FMR1 | Brain | SK-N-MC | | [Chinmug et al., 2001] |
| Rbm3 | Brain | SK-Hemo-2a, C6 | Hypothema | [Chappell et. al., 2001] |

Transcription at Restressors

| | | | |
|---|---|---|---|
| Mur | Cervix | HeLa | [Stertey et al., 2001] |
| MEF | Kidney | BHK | [Ornament et al., 2010] |
| | Cervis | HeLa | |
| | Ovary | CHO | |
| Gre | Brain | SK-N-SH, Neuro-2a, Co | [He et al., 1999; Chappell et al., 2009] |
| | Kidney | NRK | |
| | Muscle | C₃Cu | |
| | Pindsory | AiT-20 | |
| | gland | K563 | |
| | Blood | | |

Signaling Malenole

| | | | |
|---|---|---|---|
| Stand5 | Embrysek | ATDC3 | [Stusok et. al., 2002] |
| | Muscle | C₃Cu | |
| | Bone | MC3T3BI | |
| | Brain | Ghoma - low activity | |
| | Liver | HepG2 - low activity | |
| | Kidney | 293, 2993, COS7 - low | |
| | Cervex | activity | |
| | | HeLa - low activity | |

Apoptosis-related Factors

| | | | | |
|---|---|---|---|---|
| Apef-3 | Cervix | HeLa - higher activity | Apoptosis | [Coldwell et al., 2000; Heris Kerenbilt et al., |
| | Breast | MCF7 | | 2002, Mevine et al., 2002] |
| | Lung | MRC3 | | |
| | Liver | HepG2 | | |
| | Kidney | 293, 29ST, COS7 | | |
| DAP [symbol] p97NAF1 | Kidney | 293, 293T | Apoptosis | [Heria-Kerenblit et al., 2000; Henis-Ketebilt |
| | Cervix | HeLa, HFB | | et al., 2002; Nevises et al., 2002] |
| | Ovary | CHO | | |
| MIAP | Brain | SP539 | γ-irradiation | [Holcik et al., 1999; Holick et al., 2000; |
| | Lung | H661, H520 | Serun starvasion | Helis-Nore [symbol] et al., 2002; Hevina et al, 2002; |
| | Cervix | HeLa | Apoptosis | Holick et al., 2003] |
| | Ovary | CHO, SHOV3 | | |
| | Kidney | 293, 29ST | | |
| | Bladder | T24 | | |
| O [symbol] | | NIH3T3 | | |
| Hsp20 | Kidney | 203 | | [ [symbol] et al., 2003] |
| | Brain | TE571 | | |
| p27 | Brain | DoPIT | Cell cycle | [M [symbol] et al., 2001] |
| β-mRNA | — | In vitro translation | | [legrierdo and C [symbol] , 2000] |
| Bip | Brain | Neuro-2a | Heat Shock | [Yang and [symbol] , 1997; Kiim and Jang, 2002; |
| | Kidney | 203 | | Nevins et al., 2002] |
| | Bladder | T24 | | |
| | Cervix | HeLa, KB31 | | |
| Amp | Drosophlis | Schneider SL2 | | [Ye et al., 1997] |
| U [symbol] | Drosophlis | Schneider SL2 | | [Ye et al., 1997] |
| Hairless | Drosophlis | S2 | Cell cycle G2/M phase | [Meier et al., 2002] |

[symbol]

indicates data missing or illegible when filed

In a specific embodiment of the present invention, the retroviral vector or nucleic acid comprises an IRES, wherein said IRES is located in the 3′-LTR or the 5′-LTR, or in a region flanked by the 3′-LTR and the 5′-LTR. In particular, said IRES may be located in the R region of both the 5′-LTR and/or 3′-LTR. In another embodiment, the IRES is located in the U3 region of the 3′-Long Terminal Repeat or the U5 region of the 5′-LTR. In yet another specific embodiment of the present invention, said IRES is located in the U3 region between the inverted repeats and the transcription regulatory elements.

### Preferred Vectors

A central aspect of the present invention relates to a eukaryotic expression vector comprising at least one heterologous nucleic acid sequence encoding an HIV-1 envelope polypeptide of the present invention or a fragment thereof. In one embodiment, said vector comprises an intron. The intron is deleted by splicing, thus, facilitating the transport from the nucleus to the cytoplasm of said vector encoded RNA in packaging cells. As an alternative to an intron, the vectors of the present invention may also comprise a constitutive transport element (CTE). The CTE facilitates the transport of the vector encoded RNA from the nucleus to the cytoplasm of the semipackaging cell. In another approach, the vector may comprise a Rev responsive element (RRE). The presence of an RRE in the RNA transcript facilitates its transport from the nucleus to the cytoplasma of the semipackaging cell, when REV/REX is coexpressed. In one specific embodiment, the vector is transcribed in the cytoplasm, thereby producing high levels of transcript, which can be translated into envelope polypeptide. This envelope polypeptide may then be incorporated into viral particles in a packaging/producer cell.

In a specific embodiment, the vector of the present invention is a retroviral vector. Retroviral vectors include both replication deficient retroviral vectors, and replication competent retroviral vectors. In one embodiment, the present invention relates to a vector, which comprises at least one heterologous nucleic acid sequence encoding an HIV-1 envelope polypeptide of the present invention or a fragment thereof. In one embodiment, the nucleic acid sequence has been modified codon optimization to eliminate RNA elements, which may reduce the RNA expression level. Such codon optimization are known to those of skill within the art. The vector may further comprise at least one additional nucleic acid sequence and/or at least one internal ribosomal entry site (IRES). In one embodiment, at least one heterologous nucleic acid sequence encoding an HIV-1 envelope polypeptide of the present invention or fragment thereof is preceded by an IRES, and in another embodiment, the at least one additional nucleic acid sequence is preceded by an IRES. The vectors of the present invention are preferably derived from gamma-retroviruses, for example Murine Leukemia Virus (MLV), Moloney Murine Leukemia Virus (MoMLV), or Akv MLV. Preferably, the at least one heterologous nucleic acid sequence encoding an HIV-1 envelope polypeptide of the present invention or a fragment thereof is selected from the nucleic acid sequences encoding envelope polypeptides derived from HIV-1, HIV-2, or SIV. In a specific embodiment, the at least one heterologous nucleic acid sequence encoding an HIV-1 envelope polypeptide of the present invention or a fragment thereof encodes HIV-1 envelope polypeptide, for example an HOV envelope polypeptide as defined in any of SEQ ID NO: 1-326, 327-337 or a fragment thereof. In another specific embodiment, the at least one heterologous nucleic acid sequence encoding an HIV-1 envelope polypeptide of the present invention or a fragment thereof encodes a C-terminally truncated HIV-1 envelope, or a fragment thereof. In yet another specific embodiment, the at least one heterologous nucleic acid sequence encoding an HIV-1 envelope polypeptide of the present invention or a fragment thereof encodes an SIV envelope.

The vectors of the present invention, also encompass vectors as defined above, wherein said at least one additional nucleic acid sequence is a reporter gene. Different embodiments of reporter genes according to the present invention include reporter genes encoding enhanced green fluorescent protein (eGFP), lac Z, dsRed, enhanced yellow fluorescent protein (eYFP), enhanced cyan fluorescent protein (eCFP), enhanced blue fluorescent protein (eBFP) and the human alpha-1-antitrypsin (hAAT). It is understood that any of the enhanced green fluorescent protein (eGFP), lac Z, dsRed, enhanced yellow fluorescent protein (eYFP), enhanced cyan fluorescent protein (eCFP), enhanced blue fluorescent protein (eBFP) or the human alpha-1-antitrypsin (hAAT). However, the at least one additional nucleic acid sequence may also encode a selective marker, such as neomycin phosphotransferase II, and/or a suicide gene. Moreover, the at least one additional nucleic acid sequence may encode an immunomodulating polypeptide or peptide, such as an immunostimulating polypeptide, a genetic adjuvant, cytokines and/or hormones.

Important embodiments of the present invention include vectors, wherein said IRES is selected from the IRES elements of picornaviridae, retroviridae or retrotransposons, mammalia or combinations thereof. In a specific embodiment, said IRES is selected from the IRES elements of picornavirus. In another specific embodiment, said IRES is selected from the IRES elements of encephalomyocarditis (ECMV). The IRES may be inserted at different locations in the retroviral vector. In one embodiment, the IRES is located in a region flanked by the 3′-LTR and the 5′-LTR. In another embodiment, the IRES is located in the 3′-Long Terminal Repeat (LTR) or the 5′-LTR. In yet another embodiment, the IRES is located in the U3 region of the 3′ LTR. In a further embodiment, the IRES is located in the U3 region between the inverted repeats and the transcription regulatory elements.

The design of the vectors of the present invention allows the vectors to be used for vaccination purposes. Thus, in one embodiment, the lentiviral envelope encoded by the vectors as defined by the present invention is capable of inducing an immunogenic response in a host animal. For example, said immunogenic response is an antibody response and/or cytotoxic T Lymphocyte (CTL) response.

In one embodiment, the immunogenic response of a vector according to the present invention is directed against a retroviral particle expressing said lentiviral envelope. In a specific embodiment, the lentiviral envelope is incorporated in said retroviral particle. The immunogenic response may thus be directed against the retroviral particle in the host animal. Thus, the retroviral particle is not required to be infectious and/or fusogenic. In a preferred embodiment, the envelope polypeptide as described herein is expressed and displayed on the surface of said retroviral particle. The present invention, thus relates to a vector, wherein said envelope is incorporated in said retroviral particle.

In another embodiment, the immunogenic response is a CTL response, wherein said vector is integrated into the genome of a host cell.

In one embodiment, the retroviral vector of the present invention is derived from Akv MLV, and comprises a first nucleic acid sequence encoding eGFP and an additional nucleic acid sequence encoding an HIV-1 envelope polypeptide (e.g. any of SEQ ID NO: 1-326, 327-338, IGP1-7 or part thereof) preceded by an ECMV IRES element. In another preferred embodiment, the retroviral vector of the present invention is derived from Akv MLV, and comprises a first nucleic acid sequence encoding neomycin phosphotransferase II and an additional nucleic acid sequence encoding an HIV-1 envelope polypeptide (e.g. any of SEQ ID NO: 1-326, 327-337, IGP1-7 or part thereof), preceded by an ECMV IRES element.

### Biological Entity

In a primary aspect, the present invention provides a biological entity such as a eukaryotic cell, a prokaryotic cell, a viral particle, and/or a retroviral particle, wherein said biological entity comprises at least one HIV-1 envelope polypeptide as defined by the present invention and/or nucleic acid according to the present invention. In a preferred embodiment, the biological entity of the present invention is a retroviral particle and/or a provirus. However, in another preferred embodiment the biological entity is a prokaryotic cell and/or a eukaryotic cell, such as a mammalian cell. In yet another embodiment the biological entity is a liposome, a nanoparticle, a virosome, a protzoa, and/or enterobacteria. Specifically, the biological entity may be prokaryotic and/or eukaryotic cells in the intestinal tract and/or other parts of the digestive system. Moreover, the biological entity may be a nanoparticle or a liposome with synthetic envelope polypeptides.

Thus, in one embodiment, the present invention provides a biological entity comprising at least one HIV-1 envelope as defined elsewhere herein or part thereof, and/or at least one antigen as defined elsewhere herein, and/or at least one nucleic acid as defined elsewhere herein, and/or at least one vector as defined elsewhere herein.

In a preferred embodiment the HIV-1 envelope or part thereof is expressed on the surface of the biological entity of the present invention. Specifically, the HIV-1 envelope polypeptide or part thereof of said biological entity comprises gal-alfa1-3Galbeta1-4GlcNAc-R epitopes. In a specific embodiment, the biological entity of the present invention is not capable of mediating fusion of said biological entity and cells expressing receptors for HIV. However in another specific embodiment, the biological entity of the present invention is capable of mediating fusion of said biological entity and cells expressing receptors for HIV. In another embodiment, the biological entity of the present invention is capable of infecting a CD4 positive cell. Moreover, the biological entity is in one embodiment capable of inducing an immunogenic response in a host animal. In particular, said immunogenic response may be directed towards said biological entity in said host animal. The host animal may be any animal, however, preferably a mammal, more preferably a human being. However, in one preferred embodiment, the biological entity of the present invention is capable of infecting mammalian cells, such as preferably human cells. In the present context, the term “human cells” comprise any sort of human cell including mutated human cells pathogenic cells as well as any type of human stem cell.

Further embodiments of the invention include any biological entity comprising an HIV-1 envelope polypeptide that has a sequence that is at least 70% identical to the amino acid sequence shown in any one of IGP1-7, SEQ ID NO.: 1-124 or SEQ ID NO: 125-326, 327-337 or is a fragment of a sequence that is at least 70%, such as at least 80%, for example 90%, such as at least 95%, such as 100% identical to the amino acid sequence shown in SEQ ID NO.: 1-124 or SEQ ID NO: 125-326, 327-337, IGP1-7 or a part thereof, for example a part thereof comprising or consisting of at least 3, such as 4, 5, 6, 7, 8, 9, such as at least 10, for example at least 15 amino acids. In another aspect, the present invention relates to a biological entity comprising an RNA transcribed from any of the embodiments of the retroviral vectors described herein, or an RNA encoding any of the HIV envelope polypeptides of the present invention.

In a preferred embodiment, the HIV-1 envelope polypeptide as described herein is expressed on the surface of the biological entity. Cell surface expression of envelope polypeptides may be detected by persons skilled in the art. One method of detecting surface expression of ENV is provided in example 3 below. Moreover, incorporation of ENV into retroviral particles may in one example be detected by the method shown in example 4

In a specific embodiment, the biological entity of the present invention comprise an HIV-1 envelope polypeptide as defined elsewhere herein, wherein said envelope polypeptide is glycosylated. In a preferred embodiment, said envelope polypeptide comprises alpha-gal. In a specific embodiment, the biological entity of the present invention comprises an HIV-1 envelope polypeptide or fragment thereof as defined elsewhere herein, which comprises gal-alfa1-3Galbeta1-4GlcNAc-R epitopes on the envelope protein. Thus, in a preferred embodiment, the packaging/producer cells of the present invention comprise alpha-galactosidase enzyme, which make them capable of producing biological entities, such as retroviral particles comprising alpha-gal labeled envelope polypeptides.

In a specific embodiment, the biological entity is a nanoparticle, for example a nanoparticle with synthetic envelope peptides, a liposome, for example a virosome or any combination thereof. Thus, in a specific embodiment, the present invention relates to a liposome comprising any component of the present invention, including an HIV-1 envelope polypeptide as defined herein and/or fragments thereof.

### Retroviral Particles and Producer Cells

The retroviral particle of the present invention is derived from any retrovirus, including any of the retroviruses, wherefrom the retroviral vectors of the present invention may be derived, as describes above. In a preferred embodiment, the retroviral particle is a gamma-retroviral particle.

In one embodiment, the retroviral particles of the present invention are fusogenic and/or infectious. However, in another embodiment, the retroviral particles of the present invention are non-fusogenic and/or non-infectious. Specifically, in one embodiment, the retroviral particle of the present invention is not capable of mediating fusion of said retroviral particle and cells expressing receptors for HIV. However, in another embodiment, the retroviral particle of the present invention is capable of mediating fusion of said retroviral particle and cells expressing receptors for HIV. A number of different assays are known for the skilled practitioner for detecting fusogenicity and/or infectiousness of a retroviral particle. Example 5 provides one such methodology for detecting fusogenicity of retroviral particles.

The retroviral vector of the present invention is in one embodiment constructed as a replication-defective vector based on any of the retroviruses mentioned elsewhere herein. A replication-defective retroviral vector is characterised in that, one or more genes essential for virus replication, packaging of viral RNA and/or formation of infective particle, have been deleted from the retroviral vector. Thus, to reconstitute the viral life cycle and generate viral particles comprising such replication defective vectors a specialised producer cell providing the deleted genes is needed. Such producer cell are constructed by transducing a cell with DNA constructs encoding the genetic information of the retroviral proteins, which are essential for packaging a retroviral vector genome and generating viral particles.

In the presence of a retroviral vector genome (the process known as transduction) a producer cell will generate infectious viral particles which comprise the retroviral genome derived from the vector. The viral particles produced in this manner will be released and can infect another producer cell. Such producer cells are also designated as packaging cells. In one embodiment, the retroviral particle of the present invention is obtained by transfection of a producer cell with a retroviral vector or part thereof, or an RNA or part thereof according to the present invention. In another embodiment, the retroviral particle of the present invention is obtained by transfection of a semipackaging cell with a vector or part thereof, or an RNA or part thereof according to the present invention.

In one aspect, the present invention relates to a producer cell comprising a vector as provided by the present invention. In one example, a producer cell of the present invention does not comprise lentiviral tat or rev and/or rex originating from HTLV.

Packaging/producer cells are often produced by transfecting cells with genetic information and/or genes essential for retroviral particle formation. The culture of packaging cells is subsequently supertransfected with the vector DNA. However, the genetic information and/or genes essential for retroviral particle formation and the vector DNA may also be introduced in a single round of transfection. Supertransfection here describes another or a second transfection event, namely the transfection of the packaging cell with the vector. The resulting supertransfected packaging cell will subsequently produce infectious viral particles comprising the vector RNA genome. Said particles, which will be released from the packaging cell, can be isolated. It should be noted that only supertransfected packaging cells produce infectious viral particles. Accordingly, the transduction efficiency directly correlates with the amount of infectious viral particles produced.

However, a problem of low transduction efficiency is overcome by the present invention. A replication-defective retroviral vector is provided, said vector comprising a gene encoding an HIV-1 envelope polypeptide of the present invention or fragment thereof. According to one embodiment of the present invention, a nucleic acid sequence encoding an HIV-1 envelope polypeptide of the present invention or fragment thereof is under translational control of a heterologous IRES. Alternatively, a nucleic sequence encoding an HIV-1 envelope polypeptide of the present invention or fragment thereof may be present as in the vector backbone and is expressed in the transcript of the viral RNA and translated by the host cell machinery. Since the vector according to one embodiment of the present invention encodes itself an envelope, a packaging cell needs only to provide the proteins encoded by gag and/or pol. Such packaging cells comprising a gag and/or pol encoding DNA construct, but no env encoding DNA construct, are known as semi-packaging cell. Advantageously, this semi-packaging cell is not resistant to superinfection since these cells do not express envelope protein prior to transfection with the retroviral particle. Consequently, no envelope protein binds to the cellular receptor and thus, no resistance is mediated in said cell. Consequently, viral particles are only generated and released after transfection of the semi-packaging cell with the vector. The released retroviral particles comprising a gene of a functional lentiviral envelope polypeptide or fragment thereof are able to infect further semi-packaging cells in culture. Thus, the vector of the present invention is in one embodiment, replication-competent in the semi-packaging cells and thus allows for an easy and highly efficient production of the retroviral particles in high titers. Infection of target cells (also known as host cells) lacking gag and/or pol, by infectious particles produced in these semi-packaging cells result in the transfer of the genetic information of the vector only. Since this vector according to this embodiment does not comprise the gag and/or pol, no further replication of the vector in the target cell is observed. Accordingly, said replication-defective vector is a safe vector e.g. for use in gene therapy or for use as a vaccine as described herein. However, the present invention also encompasses semi-packaging cells, which are capable of producing non-infectious retroviral particles.

In a particular embodiment of the invention, the producer cell does not comprise lentiviral tat or rev for example HIV-1 tat or rev. Also the rex gene originating from HTLV is in one embodiment not present in the producer cell.

The semi-packaging cells of the present invention are selected from all types of eukaryotic cells e.g. mammalian cells, any type of eukaryotic cell that will survive in the colon and/or digenstive system, yeast cells. In a further embodiment, cells from old world monkeys and humans for production of infectious viral particles. For viral particle production capable of activating the innate immune system, all eukaryotic cells except cells derived from old world monkeys and humans may be used in the as semi-packaging cells. Examples of cells used are Human embryonic Kidney cells HEK 293, HEK 293T, Human rhabdomysarcoma cells TE671, Canine osteosarcome cells D17, murine fibroblast cells NIH3T3. In a particular embodiment the semi-packaging cells are 293T cells comprising Mo-MV GagPol DNA and/or protozoa.

In another embodiment, the retroviral particle according to the present invention comprises vesicular stomatitis virus envelope (VSV-G), the amphotropic murine leukemia virus envelope, Mutated SL3-2 envelope, Xenotropic murine leukaemia virus envelope, 10A1 virus envelope, Hepatitis virus C envelope, gibbon ape leukaemia virus. Human T-cell lymphotropic virus, or envelopes of endogenous human retroviruses. In one embodiment, the retroviral particle comprises the G glycoprotein of the vesicular stomatitis virus envelope (VSV-G).

The retroviral particles are preferably capable of infecting animal cells, such as mammalian cells, preferably human cells. In a specific embodiment, the retroviral particles are capable of infecting stem cells. In another specific embodiment, the retroviral particles are capable of infecting a CD4 positive cell. Moreover, the retroviral particles of the present invention are capable of inducing an immunogenic response in a host animal, preferably a human being. In a preferred embodiment, the immunogenic response is directed towards a retroviral particle of the present invention in said host animal. Thus, it is not required that the retroviral particle is infectious and/or fusogenic. The immunogenic response may directed against the retroviral particle it self. In one embodiment, the host animal is a human being.

In a specific embodiment, the retroviral packaging cells or semipackaging cells producing retroviral particles are encapsulated and/or any other biological entity of the present invention, which express an HIV envelope polypeptide as defined herein is encapsulated, wherein the capsules have a porous capsule wall which is permeable to said retroviral particles and/or said biological entity.

### Target Cells (Host Cells)

A primary aspect of the present invention relates to a biological entity, such as a retroviral particle, as defined herein, capable of infecting specific target cells. Infection of a target cell can occur by receptor mediated endocytosis and/or membrane fusion, as described elsewhere.

One aspect of the present invention relates to a host cell comprising a vector or part thereof of the present invention. Another aspect of the present invention relates to a host cell comprising a biological entity, such as a retroviral particle, of the present invention.

One embodiment of the present invention relates to any component of the present invention including a vector, a nucleic acid, a biological entity, such as a retroviral particle, a composition, and/or a kit-of-parts described herein comprising a retroviral envelope polypeptide, capable of mediating infection of a cell, by use of the CD4-CXCR4/CCR5 receptor pathway. Thus, embodiments of the present invention comprise any components of the present invention comprising an HIV-envelope polypeptide as defined herein, capable of mediating infection of a CD4-positive cell, and/or a CXCR4/CCR5-positive cell.

CD4 (cluster of differentiation 4) is a glycoprotein that is found primarily on the surface of helper T cells, as well as regulatory T cells and dendritic cells. CD4 is a member of the immunoglobulin superfamily. It has four immunoglobulin domains (D1 to D4) that are exposed on the extracellular surface of the cell: D1 and D3 resemble immunoglobulin variable (IgV) domains, while D2 and D4 resemble immunoglobulin constant (IgC) domains. On T cells, CD4 is the co-receptor for the T cell receptor (TCR) and recruits the tyrosine kinase Ick.

CD4 is also a primary receptor used by HIV-1 to gain entry into host T cells. The HIV-1 envelope gp120 protein attaches to CD4, creating a shift in the conformation of the viral gp120 protein, which allows HIV-1 to bind to two other cell surface receptors on the host cell (the chemokine receptors CCR5 and CXCR4). Following another change in shape of a different HIV-1 envelope protein (gp41), HIV inserts a fusion peptide into the host T cell that allows the outer membrane of the virus to fuse with the T-cell membrane. HIV infection leads to a progressive reduction in the number of T cells possessing CD4 receptors and, therefore, the CD4 count is used as an indicator to help physicians decide when to begin treatment in HIV-infected patients.

The terms “CD4-positive cell” or “CXCR4/CCR5-positive cell” as used herein, relates to any cells expressing CD4 receptor or CXCR4/CCR5 receptor, respectively. The CD4 receptor and/or CXCR/CCR5 receptor may be accessible from the extracellular space at the cellular membrane.

In one embodiment, the retroviral particle, wherein an HIV-1 envelope polypeptide of the present invention is expressed from a retroviral vector, comprises the G glycoprotein of the vesicular stomatitis virus envelope (VSV-G), the amphotropic murine leukemia virus envelope, Mutated SL3-2 envelope, Xenotropic murine leukaemia virus envelope, 10A1 virus envelope, Hepatitis virus C envelope, gibbon ape leukaemia virus, human T-cell lymphotropic virus or envelopes of endogenous human retroviruses.

In a particular embodiment of the present invention, the producer cell does not comprise lentiviral tat or rev, for example HIV-1 tat or rev. Also the rex gene originating from HTLV is not present in the host cell.

### Infections

The word infection as used herein relates to any kind of clinical condition giving rise to an immune response, such as an inflammation, and therefore includes infectious diseases, chronic infections, autoimmune conditions and allergic inflammations. Thus, infections, such as infectious diseases, chronic infections, autoimmune conditions and allergic inflammations are all clinical conditions of relevance for the present invention, and are dealt with in turn hereunder. Furthermore, the terms infection and inflammation are used interchangeably herein.

Inflammation is the complex biological response of vascular tissues to harmful stimuli, such as pathogens, damaged cells, or irritants. It is a protective attempt by the organism to remove the injurious stimuli as well as initiate the healing process for the tissue. Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

An infectious diseases may be caused by a virus, and viral diseases against which the vaccine composition of the present invention may be administered in the treatment of HIV, AIDS, AIDS Related Complex (ARC); thus it is an object of the present invention to administer a component if the present invention as the treatment of or as part of the treatment of these viral infections.

### Infectious Disease Combination Treatment

It is further provided for that a treatment of any infectious disease by the administration of the vaccine composition according to the present invention may be given in conjunction with a further (second) active ingredient or in combination with a further treatment such as antibiotic treatment, chemotherapy, treatment with immunostimulating substances, treatment using dendritic cells, antiviral agents anti parasitic agents and so forth.

Examples of a second active ingredient that may be used in the treatment of an infectious disease in combination with the vaccine of the present invention include, and are not limited to antibiotics. The term antibiotics herein refers to substances with anti-bacterial, anti-fungal, anti-viral and/or anti-parasitical activity; examples of relevance to the present invention include, but are not limited to: Amikacin, Gentamycin, Kanamycin, Neomycin, Netilmicin, Paromomycin, Streptomycin, Tobramycin, Ertapenem, Imipenem, Meropenem, Chloramphenicol, Fluoroquinolones, Ciprofloxacin, Gatifloxacin, Gemifloxacin, Grepafloxacin, Levofloxacin, Lomefloxacin, Moxifloxacin, Norfloxacin, Ofloxacin, Sparfloxacin, Trovafloxacin, Glycopeptides, Vancomycin, Lincosamides, Clindamycin, Macrolides/Ketolides, Azithromycin, Clarithromycin, Dirithromycin, Erythromycin, Cefadroxil, Cefazolin, Cephalexin, Cephalothin, Cephapirin, Cephradine, Cefaclor, Cefamandole, Cefonicid, Cefotetan, Cefoxitin, Cefprozil, Cefuroxime, Loracarbef, Cefdinir, Cefditoren, Cefixime, Cefoperazone, Cefotaxime, Cefpodoxime, Ceftazidime, Ceftibuten, Ceftizoxime, Ceftriaxone, Cefepime, Monobactams, Aztreonam, Nitroimidazoles, Metronidazole, Oxazolidinones, Linezolid, Penicillins, Amoxicillin, Amoxicillin/Clavulanate, Ampicillin, Sulbactam, Bacampicillin, Carbenicillin, Cloxacillin, Dicloxacillin, Methicillin, Mezlocillin, Nafcillin, Oxacillin, Penicillin G, Penicillin V, Piperacillin, Piperacillin/Tazobactam, Ticarcillin, Ticarcillin/Clavulanate, Streptogramins, Quinupristin, Dalfopristin, Sulfonamide/Sulfamethoxazole, Trimethoprim, Tetracyclines, Demeclocycline, Doxycycline, Minocycline, Tetracycline, Azole antifungals Clotrimazole Fluconazole, Itraconazole, Ketoconazole, Miconazole, Voriconazole, Amphotericin B, Nystatin, Echinocandin, Caspofungin, Micafungin, Ciclopirox, Flucytosine, Griseofulvin, and Terbinafine. Of further relevance are antivirals such as Vidarabine, Acyclovir, Gancyclovir and Valcyte (valganciclovir), Nucleoside-analog reverse transcriptase inhibitors (NRTI): AZT (Zidovudine), ddI (Didanosine), ddC (Zalcitabine), d4T (Stavudine), 3TC (Lamivudine), Non-nucleoside reverse transcriptase inhibitors (NNRTI): Nevirapine, Delavirdine, Protease Inhibitors Saquinavir, Ritonavir, Indinavir, Nelfinavir, Ribavirin, Amantadine/Rimantadine, Relenza and Tamiflu, Pleconaril, Interferons.

In an embodiment, the present invention regards a vaccine composition comprising at least one HIV-1 envelope polypeptide and/or a functional homologs or fragment thereof as defined elsewhere herein, for the treatment of an infectious disease, such as HIV, AIDS and/or ARC in combination with at least one antibiotic. Preferably, the vaccine composition of the present invention is used for the treatment of chronic infections e.g. HIV, AIDS and/or ARC and therefore is used in combination with any of the above listed antibiotics such as anti-viral agents.

### Vaccine Composition

The present invention regards pharmaceutical compositions capable of treating, reducing the risk of and/or preventing a clinical disorder associated with HIV infection in an individual; in other words the pharmaceutical composition of the present invention is a vaccine composition, and is accordingly referred to as such. However, the vaccine composition of the present invention may also be referred to as a pharmaceutical composition. The vaccine compositions of the present invention may be “traditional” vaccine compositions comprising antigens such as proteins, polypeptides and/or nucleic acid molecules. They may also be in the form of compositions comprising cells, such as modified cells originating from the individual and later processed, or to compositions comprising complex molecules such as antibodies or TCRs. In particular, the vaccine compositions of the present invention may comprise viral particles such as retroviral particle as defined elsewhere herein. All vaccine compositions of the present invention are claimed for use as a medicament.

Generally, a vaccine is a substance or composition capable of inducing an immune response in an individual. The composition may comprise one or more of the following: an “active component” such as an antigen(s) (e.g. protein, polypeptides, peptides, nucleic acids and the like), nucleic acid constructs comprising one or more antigens amongst other elements, cells, (e.g. loaded APC, T cells for adoptive transfer aso.), complex molecules (Antibodies, TCRs and MHC complexes and more), carriers, adjuvants and pharmaceutical carriers. The various components of a vaccine composition according to the present invention are disclosed in more detail elsewhere herein.

In a broadest aspect, the present invention relates to a vaccine composition comprising
a) an HIV-1 envelope polypeptide as defined elsewhere herein, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined elsewhere herein, and/or
c) a nucleic acid as defined elsewhere herein, and/or
d) a vector as defined elsewhere herein, and/or
e) a biological entity as defined elsewhere herein, and
f) an adjuvant

In one embodiment, the immunologically active peptide fragment of the vaccine composition above consists of a consecutive sequence of in the range of from 10 to 50 amino acids. However in another embodiment, the immunologically active peptide fragment of the vaccine composition above consists of a consecutive sequence of at least 10 amino acids, such as at least 20 amino acids, such as at least 30, such as at least 40, such as at least 50, for example at least 60, such as at least 70, for example at least 80, such as at least 90, for example at least 100 amino acids. In another embodiment, the immunologically active peptide fragment of the vaccine composition above consists of 8 to 10 or 18 to 25 consecutive amino acids of a polypeptide as defined herein, for example any peptide selected from any of SEQ ID NO: 1-124, or SEQ ID NO: 125-326, 327-337.

In another embodiment of the vaccine composition of the present invention, the HIV-1 envelope polypeptide or immunologically active peptide fragment thereof consists of the vaccine composition above consists at the most 50 amino acid residues, for example at the most 45 amino acid residues, such as at the most 40 amino acid residues, for example at the most 35 amino acid residues, such as at the most 30 amino acid residues, for example at the most 25 amino acid residues, such as 20 to 25 amino acid residues. However, in another embodiment, the HIV-1 envelope polypeptide or fragment thereof consists of at the most 20 amino acid residues, for example at the most 19 amino acid residues, such as at the most 18 amino acid residues, for example at the most 17 amino acid residues, such as at the most 16 amino acid residues, for example at the most 15 amino acid residues, such as at the most 14 amino acid residues, for example at the most 13 amino acid residues, such as at the most 12 amino acid residues, for example at the most 11 amino acid residues, such as 8 to 10 amino acid residues.

The vaccine composition of the invention is capable of eliciting an immune response against an HIV-1 envelope polypeptide of the present invention, or a functional homologue thereof having at least 70% identity to any sequence selected from SEQ ID NO 1-124 or SEQ ID NO: 125-326, 327-337, when administered to an individual. In another embodiment, the vaccine composition of the invention is capable of eliciting an immune response against an antigen presenting cell expressing an HIV-1 envelope of the present invention, and/or against an antigen as defined in the present invention, and/or against a biological entity of the present invention, when administered to an individual. In one embodiment the individual is infected with HIV. The vaccine composition of the invention is in one embodiment capable of eliciting a cellular immune response in the individual.

For example, the vaccine composition is capable of eliciting the production in a vaccinated individual of effector T-cells having a cytotoxic effect against HIV-1 infected cells in a subject. In another embodiment, the vaccine composition is capable of eliciting the production in a vaccinated individual of regulatory T-cells having a cytotoxic effect against cells expressing HIV-1 envelope polypeptide or part thereof, and/or antigen presenting cells expressing HIV-1 envelope or part thereof. In another embodiment, the vaccine composition of the present invention is capable of initiating an antibody response in an individual and/or a biological entity.

In a particular embodiment the vaccine composition is to be given against infection with HIV, in particular HIV-1. The present invention therefore also pertains to a vaccine composition which is administered to an animal including a human being, in which the vaccine is capable of eliciting an immune response against a disease caused by a lentivirus, in particular HIV-1. Thus, a vaccine composition of the present invention is capable of eliciting a clinical response in a subject, wherein the clinical response is characterised by a reduced susceptibility, resistance, stabilisation, remission or curing/recovery of an HIV infection and/or AIDS.

One embodiment combines any one of the components of the present invention, including an HIV-1 envelope polypeptide, an antigen, a nucleic acid, a eukaryotic expression vector, and/or a biological entity of the present invention with various at least one adjuvant to produce a vaccine composition.

Examples of adjuvants and immunomodulating peptides are described elsewhere herein. Adjuvants, broadly defined, are substances which promote immune responses. Frequently, the adjuvant of choice is Freund's complete or incomplete adjuvant, or killed B. pertussis organisms, used e.g. in combination with alum precipitated antigen. A general discussion of adjuvants is provided in Goding, Monoclonal Antibodies: Principles & Practice (2nd edition, 1986) at pages 61-63. Goding notes, however, that when the antigen of interest is of low molecular weight, or is poorly immunogenic, coupling to an immunogenic carrier is recommended. Examples of such carrier molecules include keyhole limpet haemocyanin, bovine serum albumin, ovalbumin and fowl immunoglobulin. Various saponin extracts have also been suggested to be useful as adjuvants in immunogenic compositions. Recently, it has been proposed to use granulocyte-macrophage colony stimulating factor (GM-CSF), a well known cytokine, as an adjuvant (WO 97/28816).

The vaccine compositions according to the invention preferably comprise an adjuvant and/or a carrier and/or at least one immunomodulating peptide. Examples of useful adjuvants and carriers are given elsewhere herein. Thus, the biological components of the present invention, such as retroviral particles present in the composition can be associated with a carrier such as e.g. a protein or an antigen-presenting cell to a T cell.

In particular the use of adjuvants is desired when the immunogenic agents of the present invention are used to boost the immune response due to the ability of the biological entities of the present invention, such as retroviral particles are able to infect, integrate and display a lentiviral envelope or fragments thereof on the surface of a host cell, thereby boosting or enhancing the immune response, in particular the CTL response as discussed elsewhere herein.

Adjuvants are any substance whose admixture into the vaccine composition increases or otherwise modifies the immune response of the biological entity coated with the HIV-1 envelope polypeptide or fragment thereof as defined elsewhere herein. Carriers are scaffold structures, for example a polypeptide or a polysaccharide, to which the biological entity coated with the HIV-1 envelope polypeptide or fragment thereof is capable of being associated.

Desirable functionalities of adjuvants capable of being used in accordance with the present invention are listed in the below table.

TABLE 11
Modes of adjuvant action

| Action | Adjuvant type | Benefit |
|---|---|---|
| 1. Immunomodulation | Generally small molecules or | Upregulation of immune |
| | proteins which modify the | response. Selection of Th1 or |
| | cytokine network | Th2 |
| 2. Presentation | Generally amphipathic molecules | Increased neutralizing antibody |
| | or complexes which interact with | response. Greater duration of |
| | immunogen in its native | response |
| | conformation | |
| 3. CTL | Particles which can bind or | Cytosolic processing of protein |
| induction | enclose immunogen and | yielding correct class 1 |
| | which can fuse with or disrupt | restricted peptides |
| | cell membranes | |
| | w/o emulsions for direct | Simple process if promiscuous |
| | attachment of peptide to cell | peptide(s) known |
| | surface MHC-1 | |
| 4. Targeting | Particulate adjuvants which | Efficient use of adjuvant and |
| | bind immunogen. Adjuvants | immunogen |
| | which saturate Kupffer cells | |
| | Carbohydrate adjuvants which | As above. May also determine |
| | target lectin receptors on | type of response if targeting |
| | macrophages and DCs | selective |
| 5. Depot | w/o emulsion for short term | Efficiency |
| Generation | Microspheres or nanospheres for | Potential for single-dose |
| | long term | vaccine |

Source: Cox, J. C., and Coulter, A. R. (1997). Vaccine 15, 248-56.

A vaccine composition according to the present invention may comprise more than one different adjuvant. Furthermore, the invention encompasses a therapeutic composition further comprising any adjuvant substance including any of the above or combinations thereof. It is also contemplated that the retroviral particle coated with the lentiviral envelope polypeptide or fragment thereof, and the adjuvant can be administered separately in any appropriate sequence.

A carrier may be present independently of an adjuvant. In particular, the inclusion of a carrier is relevant in connection with using a biological entity of the present invention are used to boost the immune response due to the ability of the retroviral particles of the present invention to infect, integrate and display the HIV-1 envelope or fragments thereof on the surface of a host cell, thereby boosting or enhancing the immune response, in particular the CTL response as discussed elsewhere herein.

The function of a carrier can for example be to increase the molecular weight of in particular peptide fragments in order to increase their activity or immunogenicity, to confer stability, to increase the biological activity, or to increase serum half-life. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans, for example sepharose.

In one embodiment, the vaccine composition of the present invention, further comprises an immunogenic polypeptide or peptide fragment selected from a polypeptide or peptide fragment, which is not derived from HIV-1 envelope.

In another specific embodiment, the vaccine composition of the present invention comprises antigen presenting biological entity of the present invention, as specified elsewhere herein. In yet another embodiment, the vaccine composition comprises a eukaryotic expression vector of the present invention, as defined elsewhere.

Accordingly, the invention encompasses a therapeutic composition further comprising an adjuvant substance including any of the above or combinations thereof. It is also contemplated that for example an antigen or a retroviral particle of the invention and the adjuvant can be administered simultaneously or separately in any appropriate sequence.

The pharmaceutical compositions may be prepared and administered using any conventional protocol known by a person skilled in the art. It will be appreciated by the person skilled in the art that the protocol may be easily adapted to any of the vaccine compositions described herein.

In on embodiment of the invention, the vaccine compositions of the invention are useful for the prophylaxis of HIV infection or for treatment of HIV infection in a human being, where the human being is receiving treatment for the infection. In a further embodiment, the pharmaceutical compositions, vaccines and vaccine compositions of the invention are suitable for the treatment, amelioration and/or prevention of a lentiviral infection, such as HIV infection, including HIV-1 infection, and AIDS.

The choice of components in the vaccine composition of the invention will depend on parameters determinable by the person of skill in the art. The composition of the invention may also contain a combination of two or more HIV-1 envelope polypeptides, antigens, nucleic acids, mammalian vectors and/or biological entities. Thus, as examples, the vaccine composition may contain any combination of those components of the present invention. Also, the composition may comprise a combination of a peptide restricted by a HLA-A molecule and a peptide restricted by a HLA-B molecule, e.g. including those HLA-A and HLA-B molecules that correspond to the prevalence of HLA phenotypes in the target population, such as e.g. HLA-A2 and HLA-B35. Additionally, the composition may comprise a peptide restricted by an HLA-C molecule.

In the case of peptide-based vaccines, epitopes can be administered in an ‘MHC-ready’ form, which enables presentation through exogenous loading independently of antigen uptake and processing by host antigen-presenting cells. The peptides of the present invention comprise both peptides in a short ‘MHC-ready’ form and in a longer form requiring processing by the proteasome thus providing a more complex vaccine composition that can target multiple tumor antigens. The more different HLA groups are targeted by a vaccine, the higher likelihood of the vaccine functioning in diverse populations.

The present invention regards in a preferred embodiment a vaccine composition comprising any HIV-1 envelope polypeptide or a functional homologue thereof having at least 70% identity to any of SEQ ID NOs: 1-124, SEQ ID NO: 125-326, 327-337 or an immunogenically active peptide fragment comprising or consisting of a consecutive sequence of at least 3, such as at least 5, such as at least 10 amino acids of said HIV-1 envelope or said functional homologue thereof or a nucleic acid encoding said HIV-1 envelope or said peptide fragment; in combination with an adjuvant for use as a medicament. The vaccine composition may be administered to treat, prevent, or reduce the risk associated with a clinical condition in an individual.

### Adjuvants

A vaccine composition according to the present invention may comprise one or more than one adjuvant. Immunostimulatory adjuvants augment antigen-specific immune responses by physical localization and improved presentation of antigen, and by provocation of inflammatory or innate immune responses (Petrovsky N, Aguilar J C. Vaccine adjuvants: current state and future trends. Immunol Cell Biol 2004; 82(5):488-96). A key feature in the innate immune system is its capability to detect foreign organisms using a set of cell receptors termed pattern-recognition receptors (PRR). One family of PRRs are Toll-Like Receptors, such as Toll-Like Receptor 9 (TLR9). TLR9 detects unmethylated CpG dinucleotides, which are relative common in the genomes of most bacteria and DNA viruses.

Use of CpG oligodeoxynucleotides (ODNs) as adjuvants has been tested in several vaccine trials. Cooper (2005) used CpG 7909 as an adjuvant to a hepatitis B vaccination schedule in HIV patients and after 12 months seroprotective titres were found in 100% of subjects in the CpG group compared to 63% in the control group (p=0.008). In a recent study immunotherapy with a ragweed-toll-like receptor δ agonist vaccine for allergic rhinitis appeared to offer long-term clinical efficacy in the treatment of ragweed allergic rhinitis (Creticos P S, Schroeder J T and Hamilton R G, et al. Immunotherapy with a ragweed-toll-like receptor δ agonist vaccine for allergic rhinitis. N Engl J Med 2006; 355(14):1445-55). TLR9-receptor agonists are also currently being evaluated as adjuvant to novel malaria vaccine candidates but they are also being used in a number of cancer trials.

Some of the shortcomings of regular vaccination are:
need for several boosts to achieve protection
delay in rise of protective antibodies
prevalence of vaccine non-responders (as outlined above—this is particularly a problem for immune-compromised individuals)
cost of antigen and vaccine production which is a very significant limitation in the development of new conjugated pneumococcal vaccines.
poorly protective antibodies with low affinity—this has been observed in a number of trials with pneumococcal vaccines in HIV-infected individuals.
Fall in antibody titre over time.

These shortcomings can be overcome by the effects of TLR9-agonists:
Reduce number of vaccinations required to achieve seroprotection (this was demonstrated in the Engerix and CpG7909 trial (Cooper C L, Davis H L and Angel J B, et al. CPG 7909 adjuvant improves hepatitis B virus vaccine seroprotection in antiretroviral-treated HIV-infected adults. AIDS 2005; 19(14):1473-9))
Accelerate seroconversion, possibly permitting post-exposure vaccination
Reduce non-responders rate
Reduce amount of antigen required
Increase antibody avidity and protective activity
More sustained antibody levels

Thus in one embodiment, the vectors, methods, proviruses, retroviral particles, uses, compositions, vaccines, vaccine compositions and kits according to the present invention comprise an adjuvant and/or a nucleic acid sequence encoding an adjuvant.

In one embodiment, the adjuvant of the present invention is an immunostimulatory adjuvant. Examples of such adjuvants are toll-like receptor agonists, such as agonists for TLR9, including CpG ODNs.

TLR agonist may in one embodiment be utilized as a mean of attracting and activating antigen presenting cells (APC; primarily Monocyte/macrophages and dendritic cells). This allows to selectively targeting the infection of pseudotyped MLV particles to the activated APC and thus promoting immunological cross-talk.

In yet another embodiment, the components of the present invention, including the vectors, nucleic acids, biological entities, compositions and kits, comprises at least one immunomodulating peptide and/or at least one nucleic acid sequence encoding an immunomodulating peptide. For example, the at least one additional nucleic acid sequence of a vector of the present invention encodes an immunomodulating polypeptide. Specifically, said immunomodulating peptide may be an immunostimulating polypeptide, an immunodominant polypeptide and/or a genetic adjuvant. Such embodiments include components of the present invention including vectors, nucleic acids, retroviral particles, compositions, and kit-of-parts comprising at least one cytokine and/or hormone, and/or at least one nucleic acid sequence encoding a cytokine and/or a hormone. Examples of cytokines include without restriction Interleukin-2 (IL-2), Interleukin-4 (IL-4), Interleukin-10 (IL-10), Granulocyte-macrophage colony stimulating factor (GM-CSF), Vascular endothelial growth factor (VEGF), Interleukin-12 (IL-12), Fibroblast growth factor (FGF), Interleukin-7 (IL-7), Interleukin-6 (IL-6), Tumor Necrosis Factor-alpha (TNF-a), Tumor Necrosis Factor-beta (TNF-b), Lymphotactin, Interferon-alpha (IFN-a), Interferon-beta (IFN-b), Interferon-gamma (IFN-g), Tumor Necrosis Factor (TNF), Interleukin-15 (IL-15), Interleukin-5 (IL-5), Interleukin-13 (IL-13), Interleukin-1a (IL-1 alfa), Interleukin-1b (IL-1beta), Interleukin-18 (IL-18), MCP-1, MIP-1a, MIP-1b, RANTES, TCA-3, CD80, CD86, CD40L, CCL3, CCL4, CCL5, Lymphocyte Chemotactic Factor (LCF), Erythropoietin (EPO), Prothymosin-alpha, Thymopoietin, Thymosin-alpha-1. Each of the cytokines mentioned herein is intended to be an individual embodiment. Consequently, a vector, method, provirus, use, composition, vaccine, vaccine composition and/or kit comprising at least one heterologous nucleic acid sequence encoding a cytokine polypeptide or a fragment thereof derived from each of them are claimed individually.

Immunomodulating polypeptides may also be expressed by the vector by including at least one nucleic acid sequence encoding an immunomodulating polypeptide. A number of immunomodulating peptides are known to the person skilled in the art. The immunomodulating polypeptides may be immunostimulatory adjuvants, immunostimulatory cytokines or immunostimulant polypeptides other than cytokines. Non-limiting examples of immunomodulating polypeptides according to the present invention are shown in table 6a, table 6b and table 6c.

TABLE 6a
List of preferred immunostimulatory adjuvants which can be used as
immunostimulatory adjuvants according to the present invention.
Protein and peptides

| | |
|---|---|
| Alpha-1,3- | J. Virol. 2006 Jul; 80(14): 6943-51. |
| galactosyltransferase | Increased immunogenicity of human |
| | immunodeficiency virus gp120 |
| | engineered to express Galalpha1- |
| | 3Galbeta1-4GlcNAc-R epitopes. Abdel- |
| | Motel U, Wang S, Lu S, Wigglesworth K, |
| | Galili U. |
| bacillus Calmette- | Cancer Immunol. Immunother. 2002 |
| Guérin (BCG) | Nov; 51(10): 521-31. Epub 2002 Sep 20. |
| | Adjuvants and the promotion of Th1-type |
| | cytokines in tumour immunotherapy. |
| | Dredge K, Marriott JB, Todryk SM, |
| | Dalgleish AG. |
| Mycobacterium vaccae | Cancer Immunol. Immunother. 2002 |
| (SRL 172) | Nov; 51(10): 521-31. Epub 2002 Sep 20. |
| | Adjuvants and the promotion of Th1-type |
| | cytokines in tumour immunotherapy. |
| | Dredge K, Marriott JB, Todryk SM, |
| | Dalgleish AG. |
| Flagellin (ligand to | Hum. Gene Ther. 2006 Nov; 17(11): 1051-61. |
| TLR5) | DNA vaccines: recent developments |
| | and future possibilities. Liu MA, Wahren B, |
| | Karlsson Hedestam GB. |
| Antrax proteins | Hum. Gene Ther. 2006 Nov; 17(11): 1051-61. |
| | DNA vaccines: recent developments |
| | and future possibilities. Liu MA, Wahren B, |
| | Karlsson Hedestam GB. |

TABLE 6b
List of immunostimulatory cytokines, which can be used as
immunostimulatory adjuvants according to the present invention. Genetic adjuvants
other than cytokines and conventional adjuvants.

| Category | Classification | Name | References |
|---|---|---|---|
| Genetic adjuvants | Confirmulatory molecules | CD80 | [22, 23] |
| | | CD86 | [22, 23] |
| | | CD154 (CD40L) | [25, 26] |
| | Chemokines | TCA-3 | [24] |
| | | RANTES | [34] |
| | | MIP1-a | [33] |
| | Complement | C3d | [28] |
| | Heat shock protein | Hsp70 | [29] |
| | Apoplosis inducer | Fas | [27] |
| | | Caspase | [30, 32] |
| | Transcriptional factors | TRFs | [31] |
| Conventional adjuvants | Mineral salts | Aluminum phosphate | |
| | | Aluminum hydoxide | [38] |
| | Bacteria-derived adjuvants | Monophosphoryl lipid A | [41, 43] |
| | | Cholers foxin | [44] |
| | | Muramyl peptides | |
| | Lipid particles | Cationic liposomes | [35-37] |
| | | Mannan-coated liposomes | [39] |
| | Emulsifier-based adjuvants | QS-21 | [42] |
| | Synthetic adjuvants | Ubenimex | [40] |

(From Methods. 2003 November; 31(3):243-54. Adjuvant formulations and delivery systems for DNA vaccines. Sasaki S, Takeshita F, Xin K Q, Ishii N, Okuda K.)

TABLE 6c
List of immunostimulatory cytokines, which can be used as immunostimulatory
adjuvants according to the present invention. (Vaccine. 2001 Mar. 21; 19(17-
19): 2647-56. Genetic adjuvants for DNA vaccines. Scheerlinck J Y.)

| | | Species^{b}, | Effects^{d} | | | | | |
|---|---|---|---|---|---|---|---|---|
| Cytokines | Ag/model^{a} | route^{c} | Protection | Ab/IgG | CTL | IgG1/IgG2a | DTH | Reference |
| MCP-1 | BAB/No Ag | R, i.m. | ↑ | ↑^{e} | n.d. | n.d. | n.d. | [51, 52] |
| MIP-1α | BAB/No Ag | R, i.m. | ↑ | ↑^{e} | n.d. | n.d. | n.d. | [51, 52] |
| MIP-1α | HIV-1 | M, i.m. | n.d. | n.d. | ↑ | ↑ | n.d. | [55] |
| MIP-1α | HIV-1 | M, i.m./i.n. | n.d. | ↑ | ↑ | ↓ | ↑ | [56] |
| MIP-1β | BAB/No Ag | R, i.m. | ↓ | ↑^{e} | n.d. | n.d. | n.d. | [51] |
| RANTES | HIV-1 | M, i.m. | n.d. | n.d. | ↑ | n.d. | n.d. | [55] |
| RANTES | BAB/No Ag | R, i.m. | = | ↑^{e} | n.d. | n.d. | n.d. | [51] |
| TCA-3 | HIV-1 | M, i.m. | n.d. | ↓ | ↑ | ↓ | ↑ | [57] |
| CD80 | HSV-2 | M, i.m. | = | = | n.d. | = | = | [54] |
| CD80 | HSV-2 | M, i.m. | ↑ | = | n.d. | = | ↑ | [54] |
| CD80 | HIV-1 | M, i.m. | n.d. | = | = | n.d. | = | [58, 59] |
| CD80^{f} | Influenza | M, i.m. | n.d. | n.d. | = | n.d. | n.d. | [29] |
| CD86 | HSV-3 | M, i.m./i.d. | = | = | n.d. | = | = | [54] |
| CD86 | HIV-1 | M, i.m. | n.d. | = | ↑ | = | ↑ | [58, 60, 53, 59] |
| CD86 | HIV-1 | C, i.m. | n.d. | n.d. | ↑ | n.d. | n.d. | [60] |
| CD86^{f} | Influenza | M, i.m. | n.d. | n.d. | ↑ | n.d. | n.d. | [29] |
| CD40L | β-gal | M, i.m. | ↑ | ↑ | ↑ | ↓ | n.d. | [46] |

Adjuvants could for example be selected from the group consisting of: AlK(SO₄)₂, AlNa(SO₄)₂, AlNH₄ (SO₄), silica, alum, Al(OH)₃, Ca₃ (PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see US 58767 and U.S. Pat. No. 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. Imidazochinilines are yet another example of preferred adjuvants. The most preferred adjuvants are adjuvants suitable for human use.

Montanide adjuvants (all available from Seppic, Belgium), may be selected from the group consisting of Montanide ISA-51, Montanide ISA-50, Montanide ISA-70, Montanide ISA-206, Montanide ISA-25, Montanide ISA-720, Montanide ISA-708, Montanide ISA-763A, Montanide ISA-207, Montanide ISA-264, Montanide ISA-27, Montanide ISA-35, Montanide ISA 51F, Montanide ISA 016D and Montanide IMS, preferably from the group consisting of Montanide ISA-51, Montanide IMS and Montanide ISA-720, more preferably from the group consisting of Montanide ISA-51. Montanide ISA-51 (Seppic, Inc.) is oil/surfactant based adjuvants in which different surfactants are combined with a non-metabolizable mineral oil, a metabolizable oil, or a mixture of the two. They are prepared for use as an emulsion with an aqueous solution comprising HIV-1 envelope polypeptide or peptide fragment thereof. The surfactant is mannide oleate. QS-21 (Antigenics; Aquila Biopharmaceuticals, Framingham, Mass.) is a highly purified, water-soluble saponin that handles as an aqueous solution. QS-21 and Montanide ISA-51 adjuvants can be provided in sterile, single-use vials.

The well-known cytokine GM-CSF is another preferred adjuvant of the present invention. GM-CSF has been used as an adjuvant for a decade and may preferably be GM-CSF as described in WO 97/28816.

In a preferred embodiment, the vaccine composition adjuvant is selected from the group consisting of bacterial DNA based adjuvants, oil/surfactant based adjuvants, viral dsRNA based adjuvants, imidazochinilines, and/or incomplete Freund's adjuvant (IFA). In another preferred embodiment, the vaccine composition adjuvant is a Montanide ISA adjuvant. In another preferred embodiment the adjuvant is Montanide ISA 51 or Montanide ISA 720. In another preferred embodiment, the adjuvant is Montanide ISA 51. In yet another embodiment, the adjuvant is GM-CSF.

### Carriers

The vaccine composition of to the present invention may comprise any adjuvant substance and/or carrier including any of the above or combinations thereof. It is also contemplated that any of the immunogenic agents of the vaccine composition and the adjuvant and/or carrier can be administered simultaneously or separately and/or repetitively in any appropriate sequence.

A carrier may be present independently of an adjuvant. The function of a carrier can for example be to increase the molecular weight of in particular peptide fragments in order to increase their activity or immunogenicity, to confer stability, to increase the biological activity, or to increase serum half-life. Furthermore, a carrier may aid in presenting the HIV-1 envelope polypeptide, variant or peptide fragments thereof to T-cells. The carrier may be any suitable carrier known to a person skilled in the art, for example a protein or an antigen presenting cell. A carrier protein could be, but is not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

Thus it is an aspect of the present invention that the immunogenic agent, such as an HIV-1 envelope polypeptide or fragment or variant thereof or peptide derived here from present in the vaccine composition is associated with a carrier such as e.g. a protein of the above or an antigen-presenting cell such as e.g. a dendritic cell (DC).

Accordingly, the invention encompasses a therapeutic composition further comprising an adjuvant substance including any of the above or combinations thereof. It is also contemplated that the antigen, i.e. the peptide of the invention and the adjuvant can be administered simultaneously or separately in any appropriate sequence.

### Dosis and Administration

The amount of the immunogenic HIV1-envelope polypeptide, biological entity, and/or antigen of the invention in the vaccine composition may vary, depending on the particular application. However, a single dose of the HIV1-envelope polypeptide, biological entity, and/or antigen is preferably anywhere from about 1 μg to about 5000 μg, more preferably from about 50 μg to about 2500 μg such as about 100 μg to about 1000 μg. Modes of administration include intradermal, subcutaneous and intravenous administration, implantation in the form of a time release formulation, etc. Any and all forms of administration known to the art are encompassed herein. Also any and all conventional dosage forms that are known in the art to be appropriate for formulating injectable immunogenic peptide composition are encompassed, such as lyophilized forms and solutions, suspensions or emulsion forms containing, if required, conventional pharmaceutically acceptable carriers, diluents, preservatives, adjuvants, buffer components, etc.

The vaccine compositions may be prepared and administered using any conventional protocol known by a person skilled in the art. In examples 3-5 non-limiting examples of preparation of a vaccine composition according to the invention is given as well as a non-limiting example of administration of such as a vaccine. It will be appreciated by the person skilled in the art that the protocol may be easily adapted to any of the vaccine compositions described herein. In a further embodiment of the invention, the pharmaceutical composition of the invention is useful for treating an individual suffering from a clinical condition characterized by expression of HIV-1 envelope, such as HIV, AIDS and/or ARC.

The immunoprotective effect of the composition of the invention can be determined using several approaches known to those skilled in the art. A successful immune response may also be determined by the occurrence of DTH reactions after immunization and/or the detection of antibodies specifically recognizing the peptide(s) of the vaccine composition.

Vaccine compositions according to the invention may be administered to an individual in therapeutically effective amounts. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the methods of prophylaxis and treatment with the vaccine composition.

For example, the vaccine compositions can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the vaccine, comprising any of the herein described compounds can be employed as a prophylactic or therapeutic agent. Also any and all conventional dosage forms that are known in the art to be appropriate for formulating injectable immunogenic peptide composition are encompassed, such as lyophilized forms and solutions, suspensions or emulsion forms containing, if required, conventional pharmaceutically acceptable carriers, diluents, preservatives, adjuvants, buffer components, etc.

Preferred modes of administration of the vaccine composition according to the invention include, but are not limited to systemic administration, such as intravenous or subcutaneous administration, intradermal administration, intramuscular administration, intranasal administration, oral administration, rectal administration, vaginal administration, pulmonary administration and generally any form of mucosal administration. Furthermore, it is within the scope of the present invention that the means for any of the administration forms mentioned in the herein are included in the present invention.

A vaccine according to the present invention can be administered once, or any number of times such as two, three, four or five times. Administering the vaccine more than once has the effect of boosting the resulting immune response. The vaccine can further be boosted by administering the vaccine in a form or body part different from the previous administration. The booster shot is either a homologous or a heterologous booster shot. A homologous booster shot is a where the first and subsequent vaccinations comprise the same constructs and more specifically the same delivery vehicle especially the same viral vector. A heterologous booster shot is where identical constructs are comprised within different viral vectors.

The present invention also in one aspect, relates to a method of producing a vaccine composition of the present invention as defined elsewhere herein, comprising combining
a) an HIV-1 envelope polypeptide as defined elsewhere herein, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined elsewhere herein, and/or
c) a nucleic acid as defined elsewhere herein, and/or
d) a vector as defined elsewhere herein, and/or
e) a biological entity as defined elsewhere herein, and
f) an adjuvant

### Antibody

It is one aspect of the present invention to provide antibodies or functional equivalents thereof, such as antigen binding fragments or recombinant proteins specifically recognising and binding an HIV-1 envelope polypeptide, such as an HIV envelope polypeptide encoded by a gene selected from the group consisting of SEQ ID NO: 74-119. In one embodiment, the antibody, antigen binding fragment, recombinant protein or functional homologue thereof is capable of inhibiting binding of said polypeptide to a native cellular interaction partner. The antibody or functional homologue thereof specifically recognizes an epitope or a functional homologue thereof. The epitope may be any of the epitopes mentioned herein.

The antibody or functional equivalent thereof may be any antibody known in the art, for example a polyclonal or a monoclonal antibody derived from a mammal or a synthetic antibody, such as a single chain antibody or hybrids comprising antibody fragments. Furthermore, the antibody may be mixtures of monoclonal antibodies or artificial polyclonal antibodies. In addition functional equivalents of antibodies may be antibody fragments, in particular epitope binding fragments. Furthermore, antibodies or functional equivalent thereof may be small molecule mimetics, mimicking an antibody. Naturally occurring antibodies are immunoglobulin molecules consisting of heavy and light chains. In preferred embodiments of the invention, the antibody is a monoclonal antibody.

Monoclonal antibodies (Mab's) are antibodies, wherein every antibody molecule are similar and thus recognises the same epitope. Monoclonal antibodies are in general produced by a hybridoma cell line. Methods of making monoclonal antibodies and antibody-synthesizing hybridoma cells are well known to those skilled in the art. Antibody producing hybridomas may for example be prepared by fusion of an antibody producing B lymphocyte with an immortalized B-lymphocyte cell line. Monoclonal antibodies according to the present invention may for example be prepared as described in Antibodies: A Laboratory Manual, By Ed Harlow and David Lane, Cold Spring Harbor Laboratory Press, 1988. Said monoclonal antibodies may be derived from any suitable mammalian species, however frequently the monoclonal antibodies will be rodent antibodies for example murine or rat monoclonal antibodies. It is preferred that the antibodies according to the present invention are monoclonal antibodies or derived from monoclonal antibodies.

Polyclonal antibodies is a mixture of antibody molecules recognising a specific given antigen, hence polyclonal antibodies may recognise different epitopes within said antigen. In general polyclonal antibodies are purified from serum of a mammal, which previously has been immunized with the antigen. Polyclonal antibodies may for example be prepared by any of the methods described in Antibodies: A Laboratory Manual, By Ed Harlow and David Lane, Cold Spring Harbor Laboratory Press, 1988. Polyclonal antibodies may be derived from any suitable mammalian species, for example from mice, rats, rabbits, donkeys, goats, sheeps, cows or camels. The antibody is preferably not derived from a non-mammalian species, i.e. the antibody is for example preferably not a chicken antibody. The antibody may also for example be an artificial polyclonal antibody as for example described in U.S. Pat. No. 5,789,208 or U.S. Pat. No. 6,335,163, both patent specifications are hereby incorporated by reference into the application in their entirety.

The antibodies according to the present invention may also be recombinant antibodies. Recombinant antibodies are antibodies or fragments thereof or functional equivalents thereof produced using recombinant technology. For example recombinant antibodies may be produced using a synthetic library or by phage display. Recombinant antibodies may be produced according to any conventional method for example the methods outlined in “Recombinant Antibodies”, Frank Breitling, Stefan Dübel, Jossey-Bass, September 1999.

Human monoclonal antibodies of the invention can be produced by a variety of techniques, including conventional monoclonal antibody methodology, e.g., the standard somatic cell hybridization technique of Kohler and Milstein, Nature 256:495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes.

### Antibody Targets

The antibody, antigen binding fragment or recombinant protein thereof is specific for a translational gene product of/polypeptide encoded by HIV-1, such as an envelope polypeptide encoded by a gene selected from the group consisting of SEQ ID NO: 74-119, 120-124, 330-333 or a part or functional homolog of said polypeptide, and/or any envelope polypeptide comprising a nucleic acid sequence selected from the group consisting of SEQ ID NO: 1-337 or part thereof. In one embodiment, the antibody, antigen binding fragment or recombinant protein thereof is capable of specifically inhibiting binding of a native protein interaction partner to a polypeptide or part thereof encoded by a gene of the present invention. Said inhibition of binding is suitable for treatment of a clinical condition as defined elsewhere herein, such as HIV-1 or AIDS.

In one embodiment of the present invention, the antibody, antigen binding fragment or recombinant protein thereof is capable of specifically recognizing and binding an HIV-1 envelope polypeptide, such as an HIV-1 envelope polypeptide selected from any one of SEQ ID NO: 74-119, 120-124, 330-333.

Specifically, the antibody, antigen binding fragment or recombinant protein thereof according to the present invention, is capable of specifically recognizing and binding to an HIV-1 derived peptide as defined herein, such as any one of SEQ ID NO: 1-329, 330-337. In particular, the antibody, antigen binding fragment or recombinant protein thereof according to the present invention, is capable of specifically recognizing and binding to an epitope consisting of 3 to 10 amino acid residues, such as 3 to 8 amino acid residues, such as 3 to 6 amino acid residues selected from an HIV-1 envelope polypeptide sequence such as SEQ ID NO: 74-119. However, even more specifically the antibody, antigen binding fragment or recombinant protein thereof according to the present invention, is capable of specifically recognizing and binding to an epitope consisting of 3 to 10 amino acid residues, such as 3 to 8 amino acid residues, such as 3 to 6 amino acid residues of an HIV-1 derived peptide selected from SEQ ID NO: 1-73 and/or 120-329 and/or 330-337.

Another aspect of the present invention relates to antibodies and functional homologues thereof, which are able to specifically recognize and bind, and modulate the activity of a polypeptide encoded by a gene of the present invention, preferably an HIV-1 envelope polypeptide sequence such as SEQ ID NO: 74-119, 120-124, 330-333, or a functional homolog or part thereof.

Importantly, the present invention encompasses use of an antibody as defined herein, for the manufacture of a medicament for the treatment of a clinical condition as defined herein, such as HIV-1 and/or AIDS.

Also, the present invention encompasses methods of treatment of a clinical condition as defined herein, such as HIV-1 and/or AIDS comprising administration of an antibody as described herein to a person in need thereof. The invention also relates to an antibody as defined herein for treatment of said clinical condition.

Thus, one aspect of the present invention relates to an antibody, antigen binding fragment or recombinant protein thereof, which is specific for an HIV-1 envelope polypeptide or part thereof as defined herein, and/or a nucleic acid as defined herein, and/or an antigen as defined herein, and/or a biological entity as defined herein. In one embodiment, the antibody, antigen binding fragment or recombinant protein thereof is capable of initiating an immune response against HIV-1 retroviral particles.

The HIV-1 envelope polypeptide or part thereof is any HIV-1 envelope polypeptide, or any polypeptide derived therefrom described herein above, for example selected from any one of SEQ ID NO: 1-329, 330-337, or part thereof.

In another aspect, the present invention relates to an antibody obtainable by immunizing a host with an HIV-1 envelope polypeptide or part thereof as defined herein, such as a polypeptide identified by SEQ ID NO: 1-329, 330-337 or part thereof, and/or a nucleic acid as defined herein, and/or an antigen as defined herein, and/or a biological entity as defined herein, a vaccine composition as defined herein, and/or a kit-of-parts as defined herein.

The present invention also relates to a method of producing antibody of the present invention, said method comprising the steps of
a) administering an antigen, HIV-1 envelope polypeptide or part thereof as defined herein, and/or a nucleic acid as defined herein, and/or an antigen as defined herein, and/or a biological entity as defined herein, a vaccine composition as defined herein, and/or a kit-of-parts as defined herein to an animal
b) obtaining said antibody from said animal
   Specific methodologies for the production of antibodies are described herein above. The antigen, HIV-1 envelope polypeptide or part thereof used for producing an antibody is preferably a polypeptide selected from any one of SEQ ID NO: 1-73 and/or SEQ ID NO: 120-329, 330-337. In a preferred embodiment, the antigen, HIV-1 envelope polypeptide or part thereof comprise one or more epitopes of the present invention.

Thus in one aspect, the present invention relates to the use of an antigen, HIV-1 envelope polypeptide or part thereof of the present invention, such as a polypeptide according to any one of SEQ ID NO: 1-329, 330-337 for producing an antibody. In one example said use relates to the use for producing an antibody for the treatment of a clinical condition such as HIV-1 and/or AIDS.

### Functional Homologues

Functional homologues of polypeptides according to the present invention is meant to comprise any polypeptide sequence which is capable of associating with an envelope receptor protein and/or which renders a cell fusogenic when expressing said functional homolog, and/or which is capable of eliciting an immune response, when said functional homolog is presented on the surface of a cell, particle and/or other physical entity. Examples 1-5 provides different examples of methods for testing immunosuppression/immunogenicity, surface expression, and fusogenicity between two human cell lines.

Functional homologues according to the present invention comprise polypeptides with an amino acid sequence, which are sharing at least some homology with the predetermined polypeptide sequences as outlined herein. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the predetermined polypeptide sequences as outlined herein above. The homology between amino acid sequences may be calculated using well known algorithms such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such a substitution may be a conservative amino acid substitution or it may be a non-conservative substitution. A conservative amino acid substitution is a substitution of one amino acid within a predetermined group of amino acids for another amino acid within the same group, wherein the amino acids within predetermined groups exhibit similar or substantially similar characteristics. Within the meaning of the term “conservative amino acid substitution” as applied herein, one amino acid may be substituted for another within groups of amino acids characterised by having
i) polar side chains (Asp, Glu, Lys, Arg, His, Asn, Gln, Ser, Thr, Tyr, and Cys)
ii) non-polar side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and
xi) amino acids being monoamino-dicarboxylic acids or monoamino-monocarboxylic-monoamidocarboxylic acids (Asp, Glu, Asn, Gln).

Non-conservative substitutions are any other substitutions. A non-conservative substitution leading to the formation of a functional homologue would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions. The addition or deletion of an amino acid may be an addition or deletion of from 2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention. The polypeptides according to the present invention, including any variants and functional homologues thereof, may in one embodiment comprise more than 5 amino acid residues, such as more than 10 amino acid residues, for example more than 20 amino acid residues, such as more than 25 amino acid residues, for example more than 50 amino acid residues, such as more than 75 amino acid residues, for example more than 100 amino acid residues, such as more than 150 amino acid residues, for example more than 200 amino acid residues.

Additional factors may be taken into consideration when determining functional homologues according to the meaning used herein. For example functional homologues may be capable of associating with antisera which are specific for the polypeptides according to the present invention.

In a further embodiment the present invention relates to functional equivalents which comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted. The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the polypeptide compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with Met-Leu-Phe. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Homologues of nucleic acid sequences within the scope of the present invention are nucleic acid sequences, which encodes an RNA and/or a protein with similar biological function, and which is either
a) at least 50% identical, such as at least 60% identical, for example at least 70% identical, such as at least 75% identical, for example at least 80% identical, such as at least 85% identical, for example at least 90% identical, such as at least 95% identical
b) or able to hybridise to the complementary strand of said nucleic acid sequence under stringent conditions.

Stringent conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Homologous of nucleic acid sequences also encompass nucleic acid sequences which comprise additions and/or deletions. Such additions and/or deletions may be internal or at the end. Additions and/or deletions may be of 1-5 nucleotides, such as 5 to 10 nucleotide, for example 10 to 50 nucleotides, such as 50 to 100 nucleotides, for example at least 100 nucleotides.

### Second Active Ingredient

It is an aspect of the present invention that the vaccine composition herein provided is used in combination with a second active ingredient. The administration of the vaccine composition and the second active ingredient may be sequential or combined. Examples of second active ingredients are given elsewhere herein. It is a further aspect that the vaccine composition may be used in combination with other therapy of relevance for the given clinical condition to be treated. Such therapy may include surgery and/or gene therapy, immunostimulating substances or antibodies; a person skilled in the art is able to determine the appropriate combination treatment for a given scenario.

In some cases it will be appropriate to combine the treatment method of the invention with a further medical treatment such as treatment with immunostimulating substances, gene therapy, treatment with antibodies and/or antibiotics and treatment using dendritic cells.

### Monitoring Immunization

In preferred embodiments, the pharmaceutical composition of the invention is a vaccine composition. It is therefore of interest, and an aspect of the present invention to monitor the immunization in an individual to whom the vaccine composition of the present invention is administered. The pharmaceutical composition may thus be an immunogenic composition or vaccine capable of eliciting an immune response to HIV infection and/or AIDS. As used herein, the expression “immunogenic composition or vaccine” refers to a composition eliciting at least one type of immune response directed against HIV-1 envelope expressing biological entities, such as mammalian cells, APCs or DCs and/or retroviral particles. Thus, such an immune response may be any of the following: A CTL response where CTLs are generated that are capable of recognizing the HLA/peptide complex presented on cell surfaces resulting in cell lysis, i.e. the vaccine elicits the production in the vaccinated subject of effector T-cells having a cytotoxic effect against the cancer cells; a B-cell response giving rise to the production of anti-HIV antibodies; and/or a DTH type of immune response. It is on object of the present invention to monitor the immunization of an individual by monitoring any of the above reactions subsequent to administering the composition of the present invention to said individual.

In one aspect the invention relates to methods of monitoring immunization, said method comprising the steps of
a) providing a blood sample from an individual,
b) providing an HIV-1 envelope polypeptide or a fragment thereof, an antigen, a nucleic acid, a biological entity, and/or a vaccine composition, and/or a kit-of-parts to an animal, wherein said HIV-1 envelope polypeptide or a fragment thereof, an antigen, a nucleic acid, a biological entity, and/or a vaccine composition, and/or a kit-of-parts may be any of the HIV-1 envelope polypeptides or fragments thereof, antigens, a nucleic acid, biological entities, and/or vaccine compositions, and/or kits-of-parts described herein, and
c) determining whether said blood sample comprises antibodies or T-cells comprising T-cell receptors specifically binding an HIV-1 envelope polypeptide or a fragment thereof, an antigen and/or biological entity as defined be the present invention, and
d) thereby determining whether an immune response to said protein or peptide has been raised in said individual.

The individual is preferably a human being, for example a human being that has been immunized with an HIV-1 envelope polypeptide or a fragment thereof, an antigen, a nucleic acid, a biological entity, and/or a vaccine composition, and/or a kit-of-parts of the present invention.

### CTL Response

The components of the present invention including HIV-1 envelope polypeptides and/or fragments thereof, vectors, nucleic acids, biological entities, compositions, and kit-of-parts is capable of inducing an immunogenic response in a host animal, for example in a human. The immunogenic response may be divided into to two types of responses, the antibody response and the cytotoxic T lymphocyte (CTL) response.

In the antibody response, specific antibodies are important in and may protect against viral infections. The most effective type of antiviral antibody is “neutralizing” antibody—this is antibody which binds to the virus, usually to the viral envelope of the virus particle or capsid proteins, and which blocks the virus from binding and gaining entry to the host cell. Virus specific antibodies may also act as opsonins in enhancing phagocytosis of virus particles—this effect may be further enhanced by complement activation by antibody-coated virus particles e.g. through production of the viral particles in eukaryotic cells e.g. mouse cells that ads gal-alfa1-3Galbeta1-4GlcNAc-R epitopes on the envelope protein. In addition, in the case of some viral infections, viral proteins are expressed on the surface of the infected cell. These may act as targets for virus-specific antibodies, and may lead to complement-mediated lysis of the infected cell, or may direct a subset of natural killer cells to lyse the infected cell through a process known as antibody-directed cellular cytotoxicity (ADCC). At mucosal surfaces (such as the respiratory and gastrointestinal tracts), virus infection may induce the production of specific antibodies of the IgA isotype, which may be protective against infection at these surfaces. Not all antibodies to viruses are protective, however, and in certain cases an antibody to the virus may facilitate its entry into a cell through Fc receptor-mediated uptake of the antibody coated particle. Such antibodies are called enhancing antibodies.

During the course of a viral infection, antibody is most effective at an early stage, before the virus has gained entry to its target cell. In this respect, antibody is relatively ineffective in primary viral infections, due mainly to the lag phase in antibody production. Preformed antibody, particularly neutralising antibody, however, is an effective form of protective immunity against viral infections, as witnessed by the success of many viral vaccines, which work by stimulating virus-neutralising antibody responses.

The principal effector cells which are involved in clearing established viral infections are the virus specific cytotoxic T lymphocytes (CTL), for example the CD8+ cytotoxic T lymphocytes. These cells recognise (viral) antigens which have been synthesised within cell's nucleus or cytosol, and which have been degraded. They are presented at the cell's surface as short peptides associated with self class I MHC molecules. The recognition of antigen by CD8+ T cells is, therefore, distinct from that of CD4+ T cells in several respects. It requires synthesis of the target antigen within the cell (and is therefore restricted largely to virally infected or tumour cells); it is “restricted” by class I MHC molecules (as opposed to MHC class II restriction for CD4+ T cells); MHC class I molecules are expressed on almost all somatic cells, so virtually any cell, on infection with virus, can act as a “target” cell for antigen specific CTL (contrasts with the limited tissue distribution of class II MHC); recognition of an antigen presenting cell (APC) by an antigen-specific CTL usually results in the destruction of the APC.

In the context of the present invention the immune response is produced against the HIV-1 envelope polypeptide or fragment thereof which is displayed on the host cells or the virus particles carrying the envelope. The virus particles carrying an HIV envelope may be given to an animal, including a human, as a vaccine. The vaccine is produced as described herein using a vector of the present invention and/or a retroviral particle and given to an animal for example a human being. The retroviral particle produced according to the present invention may infect a host cell and upon integration of the vector of the present invention into the genome of the host cell, transcription and translation by the host cell, the HIV envelope polypeptide is presented on the surface of the host cell. The host cell targeted in this manner will be subject to a CTL response.

In one embodiment of the present invention a component, such as a vector and/or a biological entity described herein is able to induce an immune response. The response may be an antibody response following vaccination with retroviral particles using the vector of the present invention. However, in yet another embodiment the immune response is a CTL response. In a specific embodiment, the immunogenic response is a CTL response, wherein said vector, RNA, mRNA of the present invention is integrated into the genome of a host cell.

In one embodiment, the vector or retroviral particle is able to infect, integrate and display the HIV-1 envelope or fragments thereof on the surface of a host cell the lentiviral envelope polypeptide, thus providing a means of boosting or enhancing the immune response as compared to viral vaccines that are not able to infect human cells. In a specific embodiment of the present invention, however, is provided non-infectious retroviral particles, which express an HIV-1 envelope as described herein and display said envelope on its surface, thereby allowing the immune system to recognize said envelope or fragment thereof.

The components of the present invention including HIV-1 envelope polypeptides and/or fragments thereof, vectors, nucleic acids, biological entities, retroviral particles, compositions, and kits-of-parts can thus be used alone or in combination with other vaccines directed against lentiviruses as defined elsewhere herein, for example HIV.

During the course of a viral infection with for example HIV, antibody is most effective at an early stage, before the virus has gained entry to its target cell. In this respect, antibody is relatively ineffective in primary viral infections, due mainly to the lag phase in antibody production. Preformed antibody, particularly neutralising antibody, however, is an effective form of protective immunity against viral infections, as witnessed by the success of many viral vaccines, which work by stimulating virus-neutralising antibody responses. In contrast to existing vaccines against lentiviruses, the present invention thus provides an additional feature which renders the vaccine capable for eliciting a CTL response.

An immunogenic composition of the invention is effective in enhancing an immune response, for example, enhanced beta-chemokine and/or IL15, IFN, IL2, TNFa production, increased HIV-specific CD4 helper cells, IgG2b antibody production, HIV specific cytotoxic T lymphocyte (CTL) production, IFNy production by CD4+ cells and CD8 T cells, and the like, in a mammal administered the composition. As described in U.S. application Ser. No. 09/565,906, filed May S. 2000, and WO 00/67787, each of which is incorporated herein by reference, and in Examples I and III, below, production of the beta-chemokine RANTES can be detected and quantitated using an ELISA assay of supernatants of T cells (such as lymph node cells or peripheral blood cells) from mammals administered the composition. In order to determine antigen-specific beta-chemokine production, T cells from an immunized mammal can be stimulated with HIV antigen in combination with antigen-presenting thymocytes, and the beta-chemokine levels measured in the supernatant. In order to determine non-specific beta-chemokine production, either T cell supernatant or a blood or plasma sample from an immunized mammal can be assayed. Similarly, production of other beta-chemokines, such as MIP-1α and MIP-1,B, can be detected and quantitated using commercially available ELISA assays, according to the manufacturer's instructions. Methods of measuring cytokine production, including inteferon, ILLS, IL2, TNFa, IL10 and IL7, by ELISPOT, ELISA, or intracellular cytokine staining are well known to those skilled in the art (see, for example, Robbins et al., AIDS 17:1121-1126 (2003)).

An immunogenic composition of the invention can further be capable of enhancing HIV-specific IgG2b antibody production in a mammal administered the composition. High levels of IgG2b antibodies, which are associated with a Th1 type response, are correlated with protection against HIV infection and progression to AIDS. Thus, the invention provides compositions that can increase a TH1 response. An immunogenic composition of the invention can further be capable of enhancing HIV-specific cytotoxic T lymphocyte (CTL) responses in a mammal administered the composition. An immunogenic composition of the invention can increase IFN-y production by both CD4+ T cells and CD8+ T cells. IFN-γ production by CD4+ T cells is characterized as a classic CD4 helper 2 5 response important to cell-mediated immunity. CD4+ T cells producing both IFN and IL2 may be most effective. IFN-production by CD8+ T cells is representative of a cytotoxic-=T lymphocyte (CTL) response, and is highly correlated with cytolytic activity. Cells producing both IFN and TNFa may be most effective. CTL activity is an important component of an effective prophylactic or therapeutic anti-HIV immune response. Methods of determining whether a CTL response is enhanced following administration of an immunogenic composition of the invention are well known in the art, and include cytolytic assays and LPA assays (described, for example, in Deml et al. supra (1999); see Example III), and ELISA and ELISPOT assays for CD8-specific IFN-y production (see U.S. application Ser. No. 09/565,906 and WO 00/67787 and Examples I and II below), intracellular staining and FACS analysis using a myriad of antibodies against cell surface markers.

### Kit of Parts

As used herein, the term “kit-of-parts” refers to components packaged or marked for use together. For example, a kit-of-parts can contain any component of the present invention, including HIV-1 envelope polypeptides and/or fragments thereof, vectors, nucleic acids, biological entities, retroviral particles, and/or vaccine compositions. Alternatively, a kit-of-parts can contain any two components in one container, and a third component and any additional components in one or more separate containers. Optionally, a kit-of-parts further contains instructions for combining the components so as to formulate an immunogenic composition suitable for administration to a mammal. The components of the kit-of-parts are preferably comprised in individual compositions, it is however within the scope of the present invention that the components of the kit-of-parts all are comprised within the same composition. The components of the kit-of-parts may thus be administered simultaneously or sequentially in any order.

One aspect of the present invention, relates to a kit-of-parts comprising the vaccine composition as defined previously herein, and a second active ingredient. The kit-of-parts preferably comprises an adjuvant and/or a carrier. Examples of useful adjuvants are given elsewhere herein. Thus, the vaccine composition may in a kit-of-parts of the present invention be associated with an adjuvant and/or a carrier. As specified previously, adjuvants are any substance whose admixture into the vaccine composition increases or otherwise modifies the immune response to an HIV-1 envelope polypeptide or a peptide fragment thereof, as defined herein. Carriers are scaffold structures, for example a polypeptide or a polysaccharide, to which the HIV-1 envelope or peptide fragment thereof is capable of being associated and which aids in the presentation of especially the peptides of the present invention. Examples of carriers are provided elsewhere herein. Some of the peptide fragments of the invention are relatively small molecules and it may therefore be required in compositions as described herein to combine the peptides with various materials such as adjuvants and/or carriers, to produce vaccines, immunogenic compositions, etc. Adjuvants, broadly defined, are substances which promote immune responses.

A carrier may be present independently of an adjuvant. The function of a carrier can for example be to increase the molecular weight of in particular peptide fragments in order to increase their activity or immunogenicity, to confer stability, to increase the biological activity, or to increase serum half-life. Furthermore, a carrier may aid in presenting the HIV-1 envelope polypeptide, variant or peptide fragments thereof to T-cells. The carrier may be any suitable carrier known to a person skilled in the art, for example a protein or an antigen presenting cell. A carrier protein could be, but is not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

Thus it is an aspect of the present invention that the HIV-1 envelope polypeptide, fragment, or variant derived here from present in the composition is associated with a carrier such as e.g. a protein of the above or an antigen-presenting cell such as e.g. a dendritic cell (DC).

One aspect of the present invention relates to a kit-of-parts comprising a therapeutically effective amount of a vector, provirus, retroviral particle, composition, vaccine, and/or vaccine composition of the present invention.

In one embodiment, the second active ingredient in the kit-of-parts of the present invention is an immunostimulating composition. Examples of immunostimulatory agents are provided elsewhere herein, however, in one preferred embodiment, the immunostimulating composition comprises one or more interleukins. For example, the interleukins are selected from the group consisting of IL-2 and/or IL-21.

In another embodiment, the second active ingredient in the kit-of-parts of the present invention is an antibiotic, such as an antibiotic is selected from the group consisting of amoxicillin, penicillin, acyclovir and/or vidarabine.

The compositions provided by the kits-of-parts of the present invention are to be administered simultaneously or sequentially.

The invention also relates to a kit-of-parts comprising
any of the vaccine compositions described herein and/or
an HIV-1 envelope polypeptide or variant hereof and/or
any of the HIV-1 envelope polypeptides or fragments or variant thereof, and/or peptides derived therefrom as described herein, for example as defined by any of SEQ ID NO: 1-124 or SEQ ID NO: 125-326, 327-337 and/or
any of the nucleic acids encoding the proteins of the above two bullet points and/or eukaryotic expression vectors comprising said nucleic acid and/or
any biological entity as defined herein, comprising a polypeptide or nucleic acid of the present invention

The invention also relates to a kit-of-parts comprising
any of the vaccine compositions described herein and/or
an HIV-1 envelope polypeptide or variant hereof and/or
any of the HIV-1 envelope polypeptides or fragments or variant thereof, and/or peptides derived therefrom as described herein, for example as defined by any of SEQ ID NO: 1-124 or SEQ ID NO: 125-326, 327-337 and/or
any of the nucleic acids encoding the proteins of the above two bullet points and/or eukaryotic expression vectors comprising said nucleic acid and/or
any biological entity as defined herein, comprising a polypeptide or nucleic acid of the present invention
   and instructions on how to use the kit of parts.

The invention also relates to a kit-of-parts comprising
any of the vaccine compositions described herein and/or
an HIV-1 envelope polypeptide or variant hereof and/or
any of the HIV-1 envelope polypeptides or fragments or variant thereof, and/or peptides derived therefrom as described herein, for example as defined by any of SEQ ID NO: 1-124 or SEQ ID NO: 125-326, 327-337 and/or
any of the nucleic acids encoding the proteins of the above two bullet points and/or eukaryotic expression vectors comprising said nucleic acid and/or
any biological entity as defined herein, comprising a polypeptide or nucleic acid of the present invention
   and a second active ingredient.

Preferably, the second active ingredient is chosen in correspondence with the clinical condition to be treated so that the second active ingredient is chosen among other clinical agents suitable for treatment of the specific clinical condition, for example HIV, AIDS and/or ARC, which is known the person skilled in the art. For example, if treating a microbial/viral infection, the second active ingredient is preferably an anti-biotic and/or an anti-viral agent.

The components of the kit can be combined ex viva to produce an immunogenic composition, or alternatively, any two components can be combined ex vivo, and administered with a third component, such that an immunogenic composition forms in vivo. For example, an HIV envelope polypeptide can be emulsified in, dissolved in, mixed with, or adsorbed to an adjuvant and injected into a mammal, preceded or followed by injection of a second component, such as an adjuvant and/or a carrier. Likewise, each component of the kit can be administered separately. Those skilled in the art understand that there are various methods of combining and administering an the components of the kit-of-parts, so as to enhance the immune response in a mammal. The kit-of-parts can be administered by the same routes of administration as a vaccine composition of the present invention, for example it can be administered locally or systemically by methods well known in the art, including, but not limited to, intramuscular, intradermal, intravenous, subcutaneous, intraperitoneal, intranasal, oral or other mucosal routes.

### Medical Applications

The present invention provides a number of therapeutical applications. All components of the present invention including HIV-1 envelope polypeptides and/or fragments thereof, antigens, vectors, nucleic acids, biological entities, such as retroviral particles, compositions, and kit-of-parts are claimed for use as a medicament. It is understood that the components of the present invention including HIV-1 envelope polypeptides and/or fragments thereof, antigens, vectors, nucleic acids, biological entities, such as retroviral particles, compositions, and kit-of-parts may be used for treating a medical condition. Thus, one aspect of the present invention relates to the use of any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for the manufacture of a medicament for the treatment, prevention and/or amelioration of a clinical condition.

Another aspect relates to any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for treating, ameliorating and/or preventing a clinical condition.

In a preferred embodiment, said clinical condition is infection, such as HIV infection, such as HIV-1 infection and/or AIDS and/or ARC. In another preferred embodiment, said treatment is prophylactic treatment, for example by reducing the susceptibility of lentiviral infection, such as HIV infection and/or AIDS and/or ARC.

Thus, one aspect of the present invention relates to the use of any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for the manufacture of a medicament for the treatment, prevention and/or amelioration of an infection, such as lentiviral infection, such as HIV infection and/or AIDS and/or ARC. Also, the present invention relates to any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for treating, ameliorating and/or preventing lentiviral infection, including HIV infection and/or AIDS and/or ARC. The present invention also relates to use of any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for the manufacture of a medicament for lentiviral vaccination, such as HIV vaccination, such as HIV-1 vaccination.

Another aspect of the present invention relates to any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for use as a medicament. In one embodiment, the components, when administered to an animal including a human being, is capable of eliciting an immune response against a disease caused by lentivirus, for example HIV, such as HIV-1 or HIV-2, or SIV.

In one aspect, the present invention relates to the use of any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for the manufacture of a medicament for gene therapy. Another aspect relates to the use of any component of the present invention for the manufacture of a medicament for immune therapy.

One aspect of the present invention relates to a method of treating, preventing or ameliorating a clinical condition, said method comprising administering to an individual suffering from said clinical condition an effective amount of any component of the present invention, including an HIV-1 envelope polypeptide and/or fragments thereof, an antigen, a vector, a nucleic acid, a vector, a biological entity, such as a retroviral particle, a vaccine composition and/or a kit-of-parts. In one embodiment, the clinical condition is an infection, and/or more specifically HIV infection and/or AIDS and/or ARC. Moreover, the individual suffering from HIV infection and/or AIDS is preferably a human being. In one embodiment, said human being is HIV seronegative. However, in another embodiment, said human being is HIV seropositive. In one embodiment of the methods of treating, preventing or ameliorating a clinical condition according to the present invention, a component of the present invention, including an HIV-1 envelope polypeptide and/or fragments thereof, an antigen, a vector, a nucleic acid, a vector, a biological entity, such as a retroviral particle, a vaccine composition and/or a kit-of-parts and/or components thereof is administered to said organism two or more times.

Any use of a component of the present invention for the manufacture of a medicament, and/or methods of treating, preventing and/or ameliorating a clinical condition comprising administering a component of the present invention may be combined with a further treatment. In one example, the further treatment is selected from the group consisting of treatment with immunostimulating substances, gene therapy, treatment with antibodies, treatment using dendritic cells and/or treatments against infections.

Another aspect of the present invention relates to a pharmaceutical composition comprising a therapeutically effective amount of the vector, the producer cell, a retroviral particle and/or a host cell according to the present invention.

The invention also relates to a method for introducing a nucleotide sequence into target cells, said method comprising infection of target cells with a retroviral particle as defined elsewhere herein. This method may for example be used for the production of transgenic animals, said method comprising infection or transduction of embryonic stem cells with a retroviral particles or a vector of the present invention.

One aspect of the present invention relates to any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for treating, ameliorating or preventing a clinical condition.

Another aspect relates to a pharmaceutical composition comprising any component of the present invention, including an HIV-1 envelope polypeptide and/or fragments thereof, an antigen, a vector, a nucleic acid, a vector, a biological entity, such as a retroviral particle, a vaccine composition and/or a kit-of-parts for treating, ameliorating or preventing a clinical condition.

In one embodiment, any component of the present invention or treating, ameliorating or preventing a clinical condition, as well as a pharmaceutical composition comprising any of said components for treating, ameliorating or preventing a clinical condition, the clinical condition is an infection. In another embodiment, the clinical condition is HIV infection and/or acquired immunodeficiency syndrome (AIDS) and/or ARC.

In another aspect, the present invention relates to a method of reducing the risk of an individual encountering a clinical condition, said method comprising administration of at least one component of the present invention, including an HIV-1 envelope polypeptide and/or fragments thereof, an antigen, a vector, a nucleic acid, a vector, a biological entity, such as a retroviral particle, a vaccine composition and/or a kit-of-parts to said individual in an amount sufficient to generate a protective immune response. In one embodiment, the clinical condition is infection with HIV.

### EXAMPLES

### Example 1

### Cells and Cell Based Assays for Immunosuppression

### Ref.:

Immunology Letters. 19 (1988) 7-14
Human retrovirus-related synthetic peptides inhibit T lymphocyte proliferation
George J. Cianciolol, Hal Bogerd and Ralph Snyderman

### Virus Preparation

Supernatant from virus producing cultures will either be purified by chromatography and inactivated by psoralene/UV light treatment or purified by sucrose gradient centrifugation. The partckles purified by sucrose gradient centrifugation will either be used directly, inactivated by UV treatment or disrupted by 0.6M KCL and 0.5% triton X-100 and clarified by centrifugation at 60,000 to 100,000 g for 1 hour.

Human mononuclear cells will be isolated from healthy laboratory volunteers by density gradient centrifugation of heparinized (10 units/ml) blood using lymphocyte separation medium (LSM; Litton Bionetics, Charleston, S.C.). The cells will be resuspended to 2×10⁶ lymphocytes/ml in RPMI 1640 (Hazelton, Denver, Pa.) supplemented with 100 units/ml of penicillin, 100 μg/ml of streptomycin, 2 mM L-glutamine, 1% non-essential amino acids, 1 mM sodium pyruvate, and 2% fetal bovine serum (FBS; Hyclone) for assays of stimulated blastogenesis. Murine CTLL-2 cells were obtained from the American Type Culture Collection and maintained in RPM1 1640 containing 5% fetal bovine serum and 5% human interleukin 2 (IL-2) (Electronucleonics, Silver Spring, Md.).

### Murine Cytotoxic T Lymphocyte (CTL) Proliferation Assay

CTL-2 cells (5×10 s cells per well in 96-well culture plates) will be incubated in the presence or absence of the indicated amount of peptide for 20 h at 37° C. in RPMI 1640 culture medium supplemented with 100 units/ml penicillin, 100 μg/ml streptomycin, 2 mM L-glutamine, 1 mM sodium pyruvate, 1% non-essential amino acids, 2% fetal bovine serum, and 1% partially purified human IL-2 (Electronucleonics). One μCi of [3H]thymidine was added to each well and the incubation continued for an additional 4 h. Cultures were harvested onto glass fiber filters and incorporated [3H]thymidine will be measured by liquid scintillation counting. Each sample will be tested in quadruplicate and standard errors were less than 5% of the mean. The average incorporation by untreated cells was 50000-75000 cpm/well.

### Anti-CD3 Stimulated Proliferation

Human mononuclear cells will be isolated from healthy volunteers by density gradient centrifugation of blood using LSM. The cells were resuspended to 2×10⁶ lymphocytes/ml in supplemented RPMI 1640 with 2% fetal bovine serum. One-tenth ml of cell suspension was added to each well of a 96-well tissue culture plate with 50 μl of either media or the indicated amount of peptide and 50 μl containing 2 ng of anti-CD3 (OKT3) antibody (Ortho Pharmaceutical) and the plate incubated for ca. 68 h at 37° C. Fifty μl of media containing 1.0 μCi [3H]thymidine (New England Nuclear; 6-7 Ci/mmole) will be added for an additional 4 h at 37° C., the cells harvested by filtration onto glass fiber filters, and incorporated radioactivity determined by liquid scintillation spectrophotometry. All samples will be run in quadruplicate. Anti-CD3 stimulated human mononuclear cells in the presence of media alone.

### Human Two-Way Mixed Lymphocyte Reactions (MLR)

Human mononuclear cells will be isolated and suspended in media as described for anti-CD3 stimulated proliferation. Fifty μl (containing 1×10⁵ cells) of cell suspension from each of two individuals was added to each well of a 96-well tissue culture plate, an additional 50 μl of media added, and the cultures incubated for 120 h at 37° C. Fifty μl of media containing the indicated amount of peptide will be added, the cultures incubated an additional 20 h and then pulsed with 1.0 μCi [3H]thymidine and incorporated radioactivity determined as described above. All samples will be tested in quadruplicate.

### Human B-Cell Proliferation

Human mononuclear cells will be isolated as described above and resuspended at 1×10 v lymphocytes/ml in supplemented RPMI 1640 media. Fifty ul of cell suspension will be added to each of quadruplicate cultures in 96-well flat-bottom plates. Fifty μl of media or media containing peptide or BSA* control will be added to each well. An additional 50/zl of BCGF (B cell growth factor, purified from pbytohemagglutinin-stimulated human T lymphocytes and free of inducing agent as well as immune interferon and T cell growth factor; Cellular Products, I no., Buffalo, N.Y.) was added to give a final concentration of 10% (v/v). After addition of 50 μl of rabbit anti-human IgG (IgG fraction; Cooper Biomedical, Inc., Malvern, Pa.; 1:100 [v/v] in media) the cultures will be incubated for 72 h at 37° C. in humidified 5% CO 2 and DNA synthesis measured by [3H]thymidine incorporation as described above.

### Calculation of Inhibition

Percent inhibition will be calculated as: % Inh.=[control (stim.)−control (unstim.)]−[exper. (stim.)−exper. (unstim.)]/[control (stim.)−control (unstim.)]×100.

### Example 2

### Immunogenicity of Retroviral Particles

The development of a broad and neutralising antibody response is vital for a protective HIV-1 vaccine (Mascola et al 2000, Nat Medicine. Protection of macaques against vaginal transmission of a pathogenic HIV-1/SIV chimeric virus by passive infusion of neutralizing antibodies). In addition, the induction of specific and effective cytotoxic T lymphocytes has been shown to be required for infection control (Schmitz et al 1999, Science. Control of Viremia in Simian Immunodeficiency Virus Infection by CD8+ Lymphocytes).

Thus, the development of vaccine strategies that encompass both arms of the immune system are thus important. Differential MHC I and II antigen presentation is a key factor for initiation of a potent immune response. There appear to be short-comings in antigen presentation when antigens are administered solely as peptides or DNA. In contrast cross-talk between antigen presenting cells and T helper lymphocytes are promoted in vaccine strategies based on either infectious or non-infectious viral particles. A potent induction of cell mediated immunity can be achieved even with whole-killed viral particles (McBurney et al 2007, Virology. Membrane embedded HIV-1 envelope on the surface of a virus-like particle elicits broader immune responses than soluble envelopes). This is likely a consequence of improved antigen uptake and systemic immunestimulation in macrophages and dendritic cells (Buonaguro et al 2006, J. Virol. Baculovirus-derived human immunodeficiency virus type i virus-like particle activate dendritic cels and induce ex vivo t-cell responses).

To achieve potent immunogenic retroviral particles, γ-retroviral particles may be produced with functional HIV-1 envelope trimers on the surface. These particles can function as a superior immunogenic particle avoiding any risk associated with viral inactivation procedures.

### Example 3

### Detection of Cell Surface Expression of ENV by Flow Cytometry

The cells are labeled with anti HIV-ENV antibodies through incubation of the cells with the Ab for 45 min on ice. Followed by washing of the unbound Ab with PBS. The cell-anti envelope Ab complex is subsequently incubated with a fluorescent labled Ab against the primary ENV-binding Ab for 45 min. on ice followed by a second PBS wash. A flow cytometer will be used to detect fluorescence associated with the cells, which is indicative of ENV expression.

### Example 4

### Detection of Incorporation of ENV into Retroviral Particles by Flow Cytometry

Supernatant containing retroviral particles is incubated with cells expressing the CD4 receptor for 45 min. on ice followed by PBS wash. The cells are subsequently labeled with anti HIV-ENV antibodies through incubation of the cells with the Ab for 45 min on ice. followed by washing of the unbound Ab with PBS. The cell-anti envelope Ab complex is subsequently incubated with a fluorescent labled Ab against the primary ENV-binding Ab for 45 min. on ice followed by a second PBS wash. A flow cytometer will be used to detect fluorescence associated with the cells, which is indicative of ENV expression.

### Example 5

### Detection of Fusogenicity of the ENV by Syncytia Assay

An Env-expressing plasmid is transfected into 293T cells. Two days later, the ENV-expressing cells are co-cultivated with D17 cells expressing CD4 (10,000 cells pr. square centimeter) and one or more of the HIV co-receptors (ie. CXCR4, CCR5 etc. 10,000 cells pr. square centimeter). Fusogenicity of the ENV protein can be detected by examination of the level of cell-cell fusion in a microscope.

### Example 6

### Detection of Fusogenicity of the ENV by Syncytia Assay

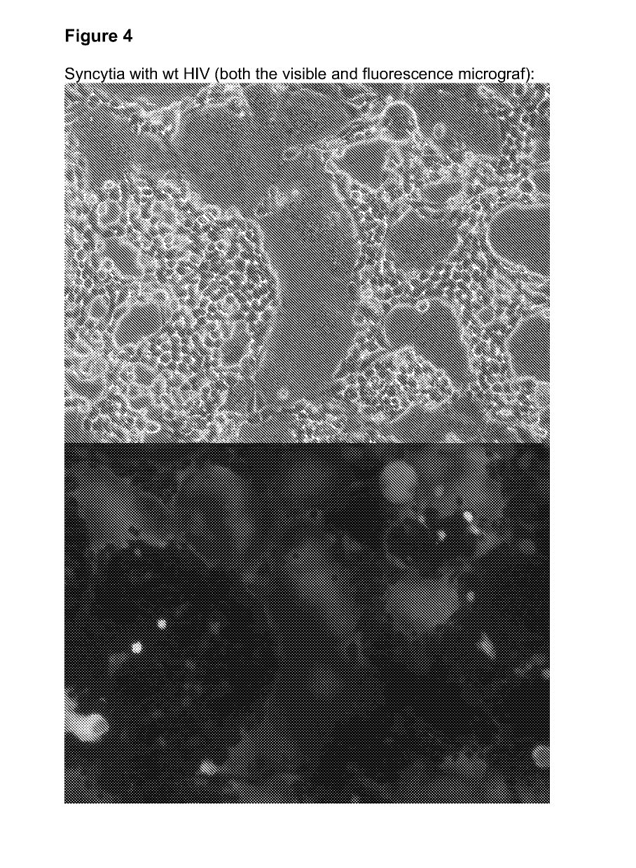

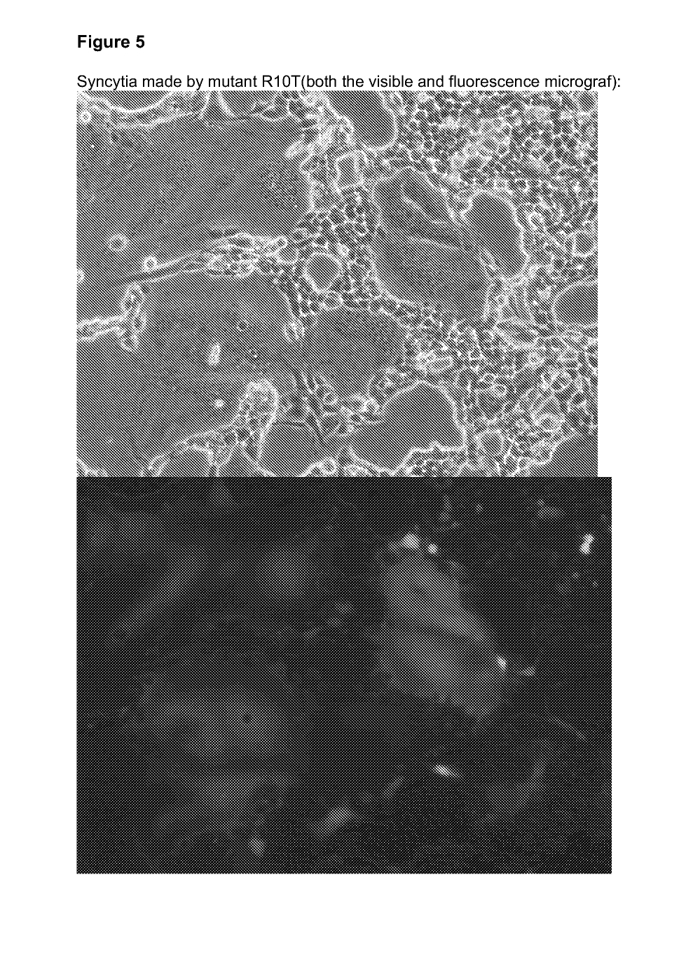

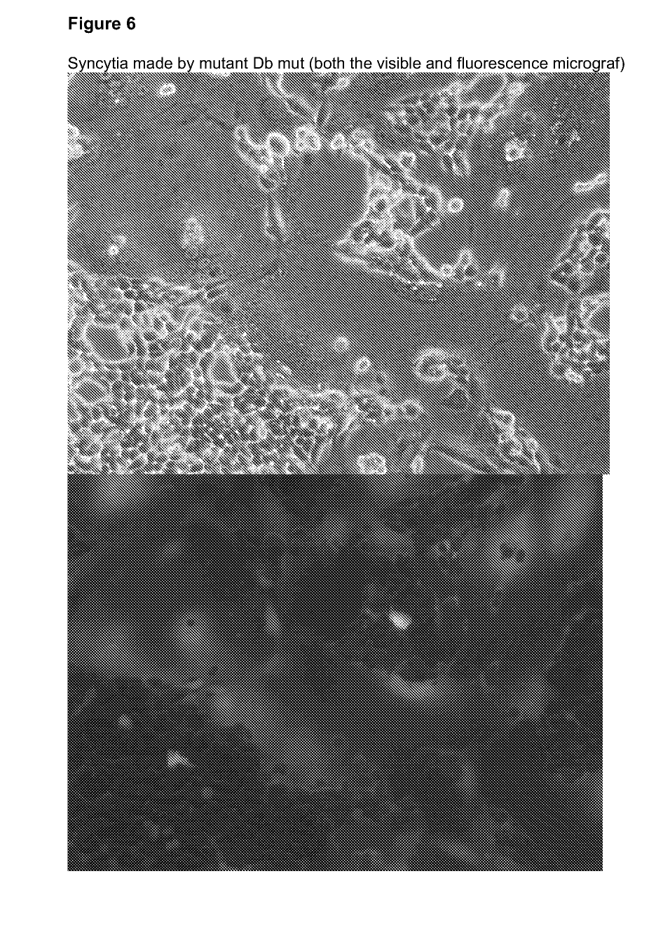

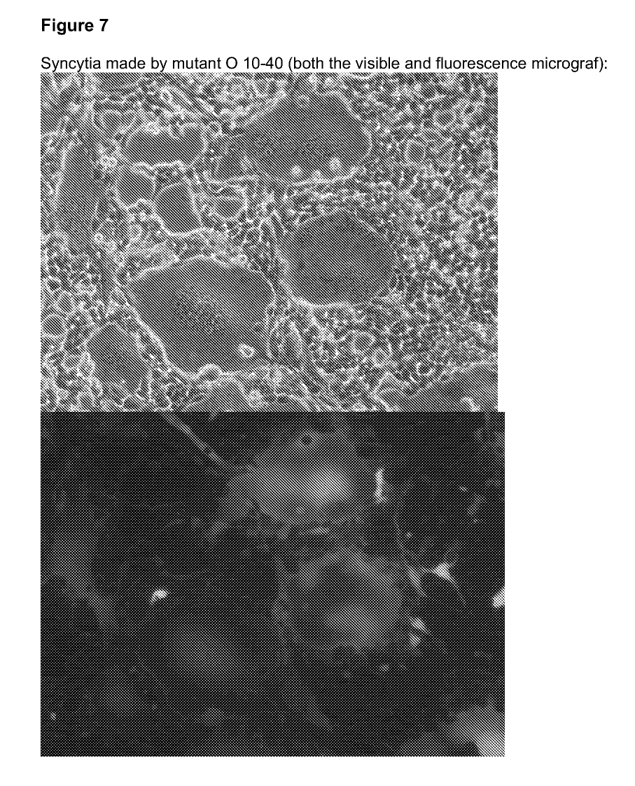

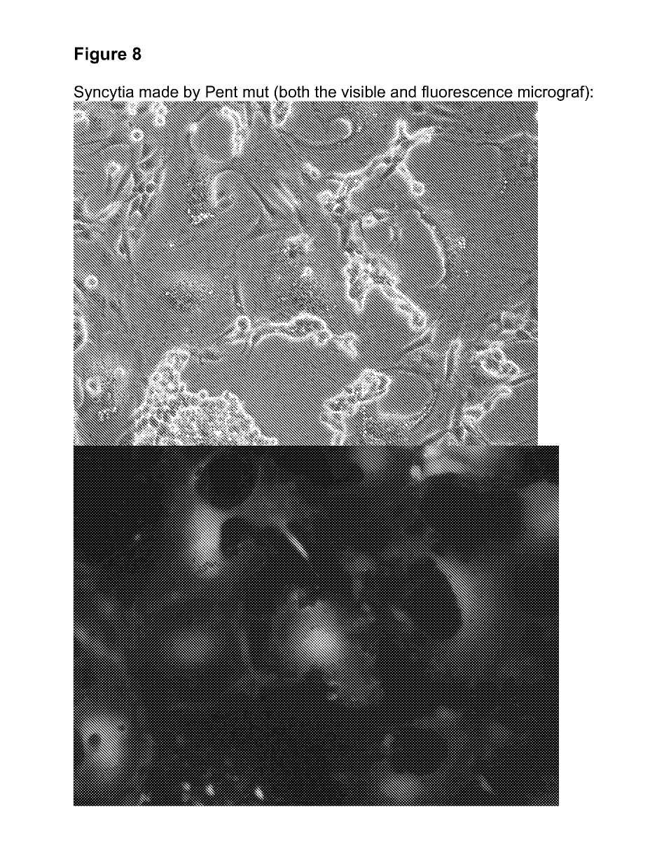

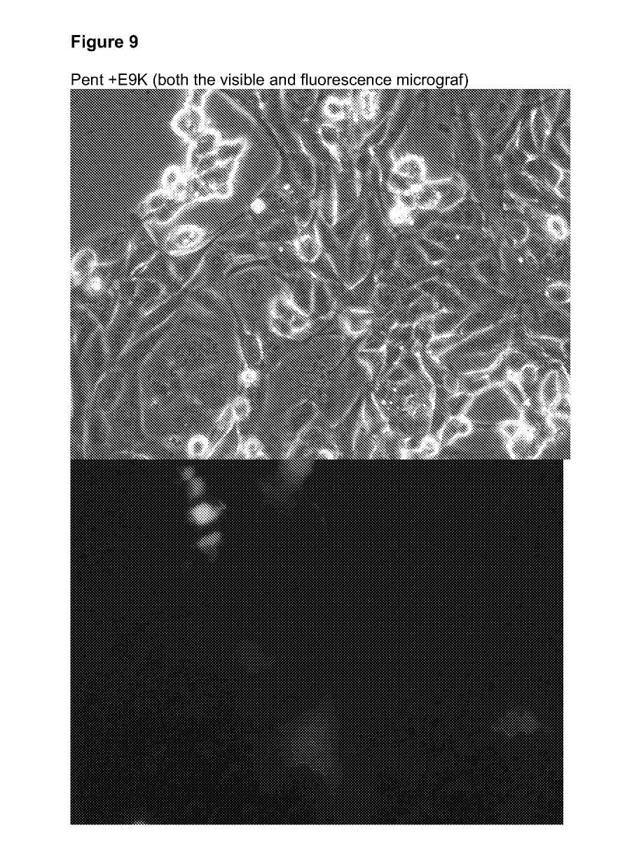

An Env-expressing plasmid (which also expresses egfp marker) is transfected into 293T cells. Two days later, the ENV-expressing cells are co-cultivated with D17 cells expressing CD4 (10,000 cells pr. square centimeter) and one or more of the HIV co-receptors (ie. CXCR4, CCR5 etc. 10,000 cells pr. square centimeter). Fusogenicity of the ENV protein can be detected by examination of the level of cell-cell fusion in a microscope, either in visible light or by green fluorescence found in the cells., se FIG. 4 to FIG. 9

| | |
|---|---|
| Wt | +++++ |
| R10T (#3) SEQ ID NO: 127 | +++++ |
| Db mut (#4) SEQ ID NO: 128 | +++++ |
| O 10-40 (#1) SEQ ID NO: 125 | +++ |
| Pent mut (#2) SEQ ID NO: 126 | ++++ |
| Pent + E9K (#5) SEQ ID NO: 129 | (+) |

### Example 7

### Immunosuppression of Selected Peptides

### Experiment Design

The experiment utilizes Human Peripheral Blood Mononuclear Cells (PBMC) prepared fresh from healthy donors. These are stimulated by Con A (5 ug/mL) concomitant to peptide addition at the indicated concentrations. Cultures are maintained and lymphocyte proliferation is measured 72 hrs later by EdU incorporation and Click-iT labelling with Oregon Green (Invitrogen, Denmark) as recommended by the manufacturer. The degree of activated lymphocytes is proportional to the fluorescence detection.

### Results

The peptides employed are:

| | |
|---|---|
| CKS-17: LQNRRGLDLLFLKEGGLC | (SEQ ID NO: 130) |

This peptide is derived from Murine Leukemia viruses and contains immunosuppressive activity associated with the envelope protein.

| | |
|---|---|
| CS-3: LQARVLAVERYLKDQQLLGIWGC, | (SEQ ID NO: 131) |

This peptide is derived from HIV-1 group M and contains immunosuppressive activity associated with the envelope protein.

| | |
|---|---|
| HIV G19R: LQARVLAVERYLKDQQLLRIWGC | (SEQ ID NO: 132) |

This peptide corresponds to CS-3 with a single point mutation at position 19 (indicated in bold).

(SEQ ID NO: 133)
HIV M/O chimera: LQARILAVETLIQNQQLLNLWGC

This peptide is a chimera between the CS3 peptide and the corresponding peptide in the envelope protein of HIV-1 clade O, that is the first 10 amino acid residues are derived from clade M sequence (CS-3, in bold) and the rest from Clade O.

The peptides are dimerized via the C-terminal Cysteines and added at the concentrations indicated below.

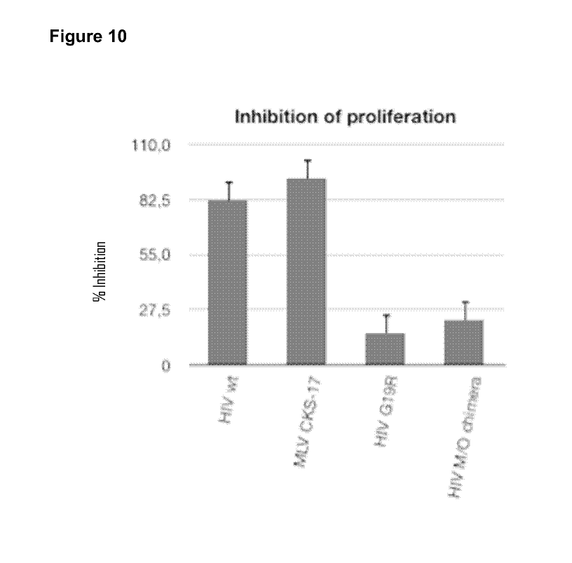

In FIG. 10 the inhibition of lymphocyte proliferation is depicted as percentage relative to the Con A stimulated cells without peptide addition. With the two reference peptides CKS-17 (from MLV) and CS-3 (from HIV-1 group M) inhibition of 93% and 82% respectively is observed at peptide concentration of 50 uM. In contrast the two variants HIV G19R and HIV M/O chimera inhibits the lymphocyte proliferation 15 and 22% respectively. This demonstrates that the peptides HIV G19R (SEQ ID NO: 132) and HIV M/O chimera (SEQ ID NO: 133) of the present invention are significantly less immunosuppressive than the wild type HIV derived CS-3 and MLV derived CKS-17 peptides.

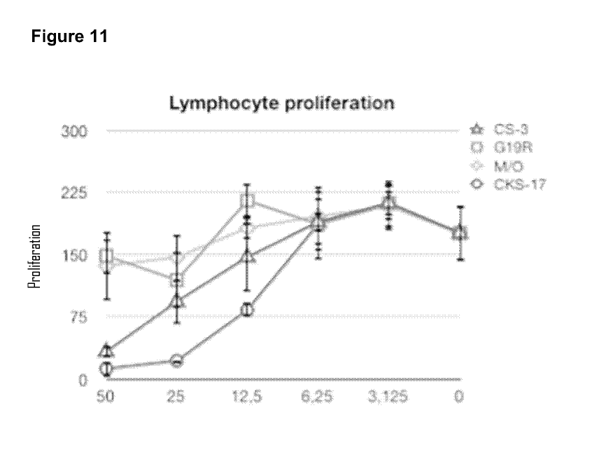

FIG. 11 depicts the proliferation at various peptide concentrations. The inhibition profiles for the two variants (HIV G19R and HIV M/O chimera) are less steep indicating a less pronounced immunosuppressive effect.

These assays clearly demonstrate that an HIV envelope, an antigen, vector, retroviral particle, entity, vaccine composition or kit-of-parts of the present invention comprising or consisting of HIV G19R and/or HIV M/O chimera peptides or nucleic acid molecules encoding said peptides are particularly suitable for prophylactic treatment and vaccination, since the peptides are less immunosuppressive than wild type HIV envelope peptides.

### Example 8

### Mixed Leukocyte Reaction

### Experiment Design

The experiment utilizes Human PBMC prepared fresh from healthy donors. These are stimulated by adding a human T cell line (Jurkat) at a 1:1 ratio. The jurkat cells have been irreversible arrested by incubating them with 50 ug/mL Mitomycin C for 1 hour prior to PBMC stimulation. Cultures are maintained and lymphocyte proliferation is measured 96 hrs later by EdU incorporation and Click-iT labelling with Oregon Green (Invitrogen, Denmark) as recommended by the manufacturer. The degree of activated lymphocytes is proportional to the flourescence detection.

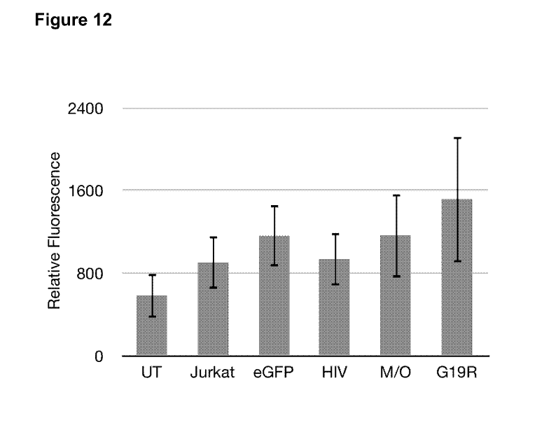

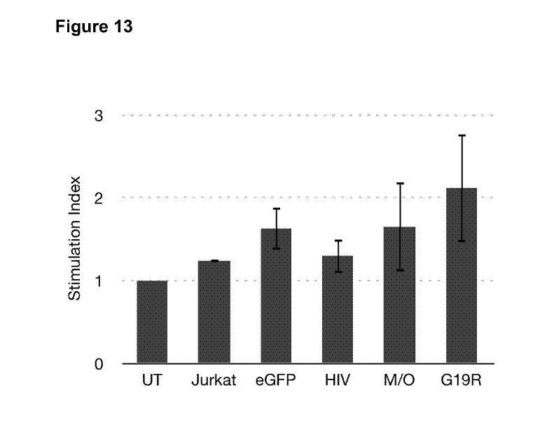

The jurkat cells have been transduced by a murine leukemia virus vector expressing either an eGFP marker gene alone (eGFP in FIG. 12 and FIG. 13) or the eGFP marker gene in addition to a HIV envelope variant: HIV (WT), M/O, or HIV G19R. Thus, Jurkat cells indicated by eGFP in FIG. 12 and FIG. 13 contain a retroviral vector without HIV envelope.

### Results

The mutations within the immunosuppressive domain in an HXB2 envelope background are indicated in bold and underlined:

HIV WT-HXB2:
(SEQ ID NO: 327)
WGIKQLQARVLAVERYLKDQQLLGIWGCSGKLICTTAVPWNASWSNKSLE.
HIV G19R-HXB2:
(SEQ ID NO: 328)
WGIKQLQARVLAVERYLKDQQLLRIWGCFGKLICTTAVPWNASWSNKSLE.
HIV M/O-HXB2:
(SEQ ID NO: 329)
WGIKQLQARVLAVETLIQNQQRLNLWGCKGKLICYTSVKWNTSWSNKSLE.

The column indicated “jurkat” in FIG. 12 and FIG. 13 are non-transduced jurkat cells.

In FIG. 12 lymphocyte proliferation is depicted. The fluorescence is proportional to proliferation.

Activation of PBMC is examined in the presence of jurkat stimulator cells. The “eGFP” column contain no HIV envelope have a higher activation level. When jurkat cells express the HIV wt envelope, a decrease in proliferation is observed. This decrease is relieved by introducing either the point mutation G19R or having an HIV M/O chimera, thus indicating that an envelope polypeptide carrying the point mutation G19R or the HIV M/O chimera display an immunostimulatory effect.

FIG. 13 depicts the stimulation index which is calculated as the proliferation in question/proliferation in UT.

### Example 9

### Cytokine Modulation

To verify the effect of immunosuppressive peptides of the present invention stimulated PBMC culture supernatant was analysed for cytokine secretion, as a measure of the cells immune response.

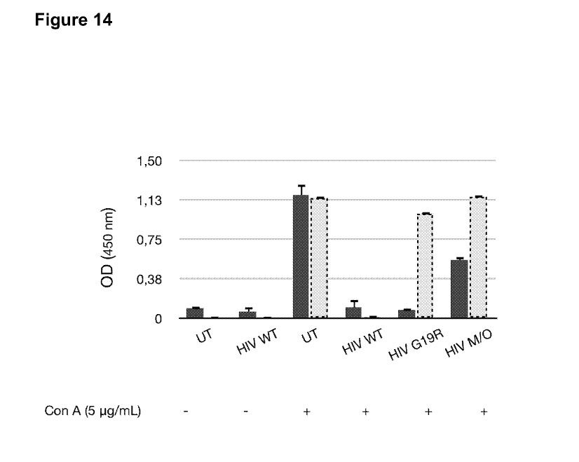

Donor PBMC's were maintained at 10⁵ cells/well in a 96-well. Stimulated by +/−Concanavalin A (ConA) and +/−50 uM peptide (HIV WT, HIV G19R, or HIV M/O). After 72 hrs, lymphocyte proliferation was measured and supernatants analysed by ELISA for levels of the proinflammatory cytokines, IFN-gamma (FIG. 14 grey bar) and TNF-alpha (FIG. 14 black bar). The phenotypic effect verified in example 8 is supported by data of inflammatory cytokines secreted from these stimulated PBMC cultures.

The present example shows the immunemodulatory properties of different peptides, and the results are shown in FIG. 14 for IFN-γ (grey bar) and TNF-α (black bar). Cytokine secretion is evaluated for PBMC untreated (UT), and in the presence of HIV WT (CS-3 (SEQ ID NO: 131)), HIV G19R (SEQ ID NO: 132), or HIV M/O peptide (SEQ ID NO: 133), as indicated. HIV WT significantly reduces both IFN-γ and TNF-α secretion upon Con A stimulation of PBMC's. The mutant G19R downregulates TNF-α but not IFN-γ. Thus, the results in FIG. 14 clearly identify HIV WT peptide as a potent blocker of both IFN-gamma and TNF-alpha, while the point mutation G19R only blocks TNF-alpha.

### Sequences

In the amino acid sequences below, the amino acids are designated by their conventional single letter code.

### IGP1

### General Immunogenic Peptide 1 (IGP1):

An HIV-1 envelope polypeptide comprising an amino acid sequence selected from the group of amino acid sequences consisting of: X(1-22)-C(23)-X(24-28)-C(29)-X(30-50):

XXXXXXXXXXXXXXXXXXXXXXCXXXXXCXXXXXXXXXXXXXXXXXXXXX

### IGP2

### General Immunogenic Peptide 2 (IGP2):

The amino acid sequence as defined by IGP1, wherein the amino acid residues in said amino acid sequence are selected from the groups of residues consisting of:

### X(1): L, S, R, P, F, A, V, M, and I; and

### X(2): Q, R, K, H, L, M, and P; and

### X(3): A, T, V, H, S, R, Q, G, M, and E; and

### X(4): R, K, G, E, T, S, C, M, and H; and

### X(5): V, I, L, D, A, S, F, M, and G; and

### X(6): L, Q, V, M, P, W, T, and I; and

### X(7): A, S, T, V, L, G, F, D, M, and E; and

### X(8): V, L, I, M, A, W, K, G, and E; and

### X(9): E, K, G, D, A, V, M, and F; and

### X(10): X; and

### X(11): Y, L, F, H, C, I, T, M, and N; and

### X(12): L, I, V, M, Q, P, T, Y, and A; and

### X(13): K, R, Q, G, S, E, H, W, T, V, M, N, Z, Y, A, P, and C; and

### X(14): D, N, G, E, Y, V, S, H, A, M, and I; and

### X(15): Q, R, H, K, P, L, M, and N; and

### X(16): Q, K, R, T, H, E, S, P, M, and L; and

### X(17): L, F, I, R, V, P, S, M, and H; and

### X(18): L, M, P, I, H, and S; and

### X(19): X; and

### X(20): I, L, M, V, S, F, T, D, A, R, P, and J; and

### X(21): W, R, G, F, L, M, and T; and

### X(22): G, D, A, R, M, and C; and

### X(24): X; and

### X(25): G, R, E, N, A, M, and D; and

### X(26): K, R, N, E, Q, T, S, I, M, and G; and

### X(27): L, H, I, T, V, F, R, Q, S, P, A, J, M, and Y; and

### X(28): I, V, T, L, R, F, and M; and

### X(30): T, P, Y, A, N, S, I, V, R, L, M and H; and

### X(31): T, S, P, N, M and I; and

### X(32): A, N, T, S, D, R, F, Q, P, I, E, V, M, L, K, H, C, and B; and

### X(33): V, A, L, M, G, R, and C; and

### X(34): X; and

### X(35): W, R, G, L, M, and P; and

### X(36): N, S, D, B, K, E, R, Q, M, and G; and

### X(37): S, T, A, N, D, V, I, E, Y, K, L, R, G, P, M, F, W, H, Q, B, and C; and

### X(38): S, T, N, I, G, R, L, C, A, W, M and E; and

### X(39): W, G, A, R, E, C, Y, V, S, M, and H; and

### X(40): X; and

### X(41): N, G, K, S, D, E, T, R, H, P, A, B, V, Q, Y, M, and I; and

### X(42): K, R, N, D, S, T, G, E, I, V, Y, Q, P, H, A, W, M, and C; and

### X(43): S, T, N, K, I, R, D, E, P, L, A, W, G, M, H, Y, F, V, and C; and

### X(44): L, Y, Q, F, E, H, S, V, K, M, T, I, W, N, D, R, P, A, and G; and

### X(45): D, E, N, S, T, K, G, L, A, Q, H, I, Y, B, R, V, P, M, F, W, Z, and C; and

### X(46): E, D, Q, Y, K, N, T, S, A, W, H, M, R, I, G, L, V, Z, F, B, and P; and

### X(47): I, D, E, M, G, T, Q, S, W, L, N, Y, K, V, R, F, A, P, and H; and

### X(48): W, I, T, N, D, E, L, G, S, Y, R, V, K, H, A, Q, M, and F; and

### X(49): D, N, E, G, W, Q, K, H, L, B, S, I, Y, T, A, R, M, Z, and V; and

### X(50): N, D, T, K, S, H, L, G, E, W, I, Q, M, R, B, Y, P, and A;

Preferred variants of IDS2 include:

### IGP3

The amino acid sequence as defined by IGP2, wherein the amino acid residue in position X(10) are selected from the group consisting of: R, S, T, K, G, A, N, Q and I.

### IGP4

The amino acid sequence as defined by IGP2, wherein the amino acid residue in position X(19) are selected from the group consisting of: G, N, S, R, E, T, D, V, C and A.

### IGP5

The amino acid sequence as defined by IGP2, wherein the amino acid residue in position X(24) are selected from the group consisting of: S, K, T, R, A, P, Y, F, G, Q, I and H.

### IGP6

The amino acid sequence as defined by IGP2, wherein the amino acid residue in position X(34) are selected from the group consisting of: P, K, R, S, A, L, Q, E, H, T, I, V, and F.

### IGP7

The amino acid sequence as defined by IGP2, wherein the amino acid residue in position X(40) are selected from the group consisting of: S, N, G, T, R, I, V, K, W, A, P, Y, D, Q, H, E, and C.

SEQ ID NO: 1
Group O derived peptide
wgikqlgarilaveXLIQNQQRLXLWGCXGKLICYTSVWNTSWXnksle
SEQ ID NO: 2
Group O derived peptide
wgikqlgarilaveXLIQNQQRLXLWGCXGKLICYTSVWNTSWYnksle
SEQ ID NO: 3
Group O derived peptide
wgikqlgarvlaveXLIQNQQLLYLWGCXGKLICYTSVXWNTSWThksle
SEQ ID NO: 4-68:
Examples of peptides of the present invention.

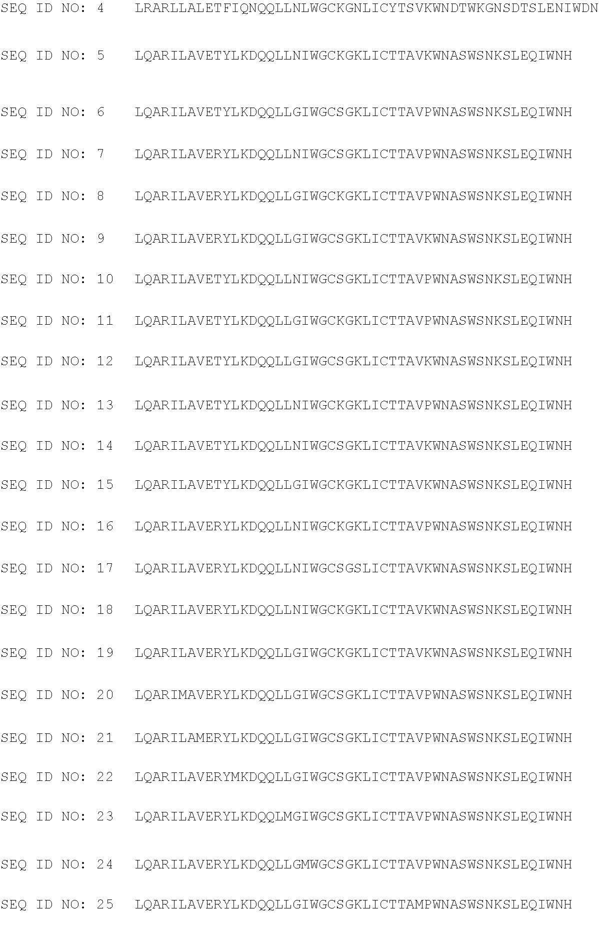

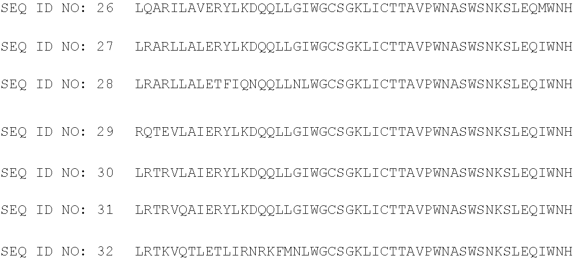

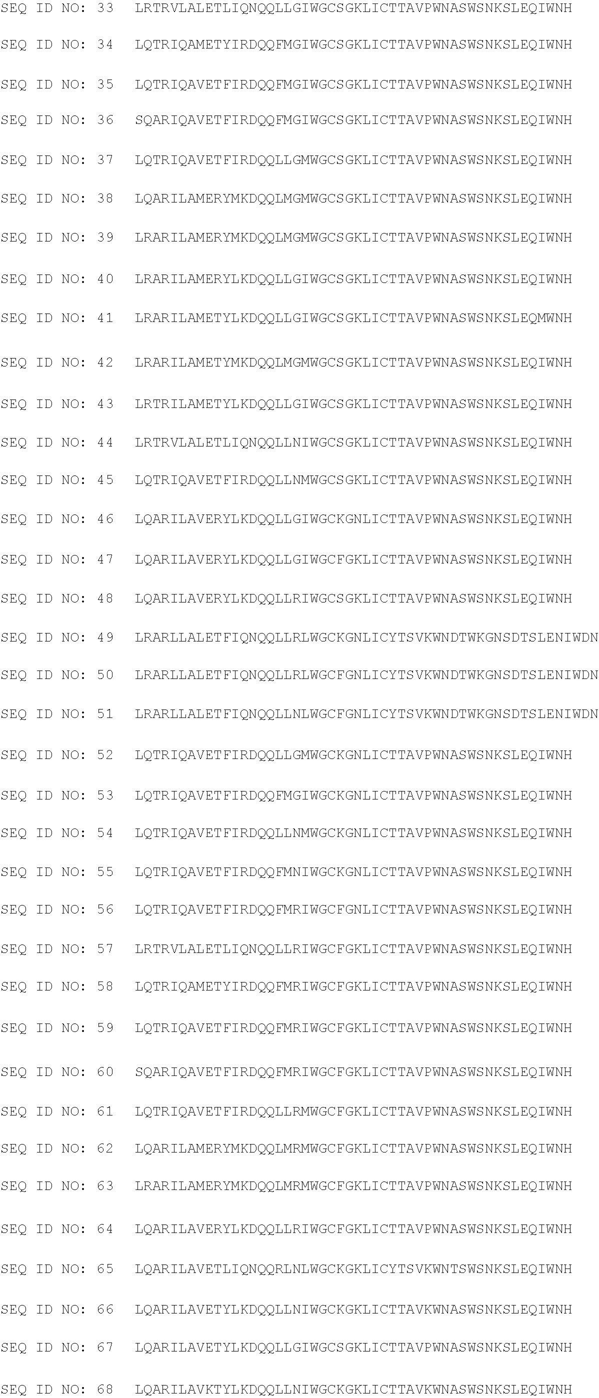

SEQ ID NO: 69
Peptide derived from SEQ ID NO: 120
LQARILAVETLIQNQQRLNLWGCKGKLICYTSVKWNTSWSNKSLEQIWNH
SEQ ID NO: 70
Peptide derived from SEQ ID NO: 121

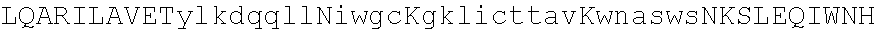

SEQ ID NO: 71
Peptide derived from SEQ ID NO: 122

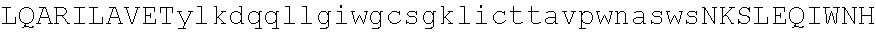

SEQ ID NO: 72
Peptide derived from SEQ ID NO: 123

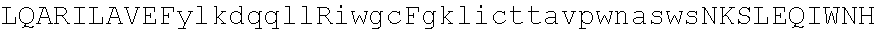

SEQ ID NO: 73
Peptide derived from SEQ ID NO: 124

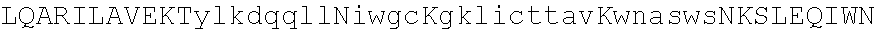

SEQ ID NOs: 74-119:
HIV envelope polypeptide prototypes for different HIV-1 subtypes.
SEQ ID NO: 74
>A1.AU.x.PS1044_Day0.DQ676872
MRAKGIQMNLHCLLKWGTMILGMILICSAAEQRWVTVYYGVPVWKDAETTLFCASDAKAYDTE
VHNVWATHACVPTDPNPQEINLXNVTEEFNMWKNNMVEQMQEDIISLWDQSLKPCVKLTPLC
VTLNCSHEVIFNSTLNNSTHSNKTLNNNTIEMKEEVRNCSYNVTTVLRDKKQKIYSLFYRLDVVP
IGNNSDSEYILINCNTSTITQACPKVSFEPIPIHYCTPAGYAILKCNDKDFNGTGPCKNVSTVQCT
HGIKPVVTTQLLLNGSLAENRTMIRSKNITDNKENIIVQLTEPVNITCIRPNNNTRKSVRIGPGQTF
YATGEIIGDIRKAHCVVNKTEWNKNLKKVVVQLRTYFKNKTISFTNHSGGDPEVTTHSFNCGGE
FFYCNTSELFNRTWNATDQLNSTEDSTALNETIILPCRIKQVINMWQTPGQAMYAPPIRGAIRCE
SNITGLILTRDGGNDNTSTNETFRPGGGDMRDNWRSELYKYKVVRIEPLGIAPTTAKRRVVQRE
KRAVGIGAVFIGFLGAAGSTMGAASITLTVQARQLLSGIVQQQSNLLRAIEAQQHMLKLTVWGIK
QLQARVLALERYLKDQQLLGIWGCSGKLICTTNVPWNNTWSNKNKSEIWDKMTWLQWDKEIS
NYTQIIYNLIEESQTQQEINEQELLALDKWANLWNWFDISQWLWYIKIFIMIVGGLIGLRIVFAVLSI
ISRVRQGYSPLSFQTHTPNPEGLDRPGRTEEEGGEQGRDRSIRLVSGFLALAWDDLRSLCLFS
YHRLRDLLSIVTRTVELLGHSSLKGLRLGWEGLKYLWNLLVYWSQELKISAVNLYDTIAIAVAGW
TDRVIEIGQGICRAILNIPRRIRQGLERALL*
SEQ ID NO: 75
>A1.KE.94.Q23_17.AF004885
MRVMGIQRNCQHLLTWGIMILGTIIFCSAVENLWVTVYYGVPVWRDADTTLFCASDAKAYETEK
HNVWATHACVPTDPNPQEIHLDNVTEKFNMWKNNMVEQMHTDIISLWDQSLKPCVKLTPLCVT
LHCTNVTSVNTTGDREGLKNCSFNMTTELRDKRQKVYSLFYRLDIVPINENQGSEYRLINCNTS
AITQACPKVSFEPIPIHYCTPAGFAILKCKDEGFNGTGLCKNVSTVQCTHGIKPVVSTQLLLNGSL
AEKNITIRSENITNNAKIIIVQLVQPVTIKCIRPNNNTRKSIRIGPGQAFYATGDIIGDIRQAHCNVTR
SRWNKTLQEVAEKLRTYFGNKTIIFANSSGGDLEITTHSFNCGGEFFYCNTSGLFNSTWYVNST
WNDTDSTQESNDTITLPCRIKQIINMWQRAGQAMYAPPIPGVIKCESNITGLLLTRDGGKDNNV
NETFRPGGGDMRDNWRSELYKYKVVEIEPLGVAPTRAKRRVVEREKRAVGIGAVFLGFLGAA
GSTMGATSITLTVQARQLLSGIVQQQNNLLRAIEAQQHLLKLTVWGIKQLQARVLAVERYLRDQ
QLLGIWGCSGKLICTTNVPWNSSWSNKSLDEIWNNMTWLQWDKEINNYTQLIYRLIEESQNQQ
EKNEKELLELDKWANLWSWFDISNWLWYIKIFIIIVGGLIGLRIVFAVLSVINRVRQGYSPLSFQTH
TPNPRGLDRPERIEEEDGEQGRGRSIRLVSGFLALAWDDLRSLCLFSYHRLRDFILIAARTVELL
GHSSLKGLRLGWEGIKYLWNLLSYWGRELKISAINLVDTIAIAVAGWTDRVIEIAQRIGRAILHIPV
RIRQGLERALL*
SEQ ID NO: 76
>A1.RW.92.92RW008.AB253421
MRVKGIQRNCQCLLTWGTMILGILIICRATENLWVTVYYGVPVWKDAKTTLFCASDAKAYETEK
HNVWATHACVPTDPNPQEIHLENVTEDFNMWKNNMVEQMHTDIISLWDQSLKPCVKLTPLCVT
LHCSNVTGANSTGTGGEEIKNCSYNITTELRDKRKKVYSLFYRLDIVQLSSNNSNSNEYRLINCN
TSAITQACPKVSFEPIPIHYCAPAGFAILKCKDEEFNGTGPCKNVSTVQCTHGIKPVVSTQLLLN
GSLAKEKVIIRSENITNNVKTIIVQLVKPVKINCTRPNNNTRTSIRIGPGQSFHATGDIIGDIRQAHC
NVSRSEWNEALRQVVEQLRGHFGNKTIIFTNSSGGDIEITTHSFNCGGEFFYCDSSGLFNSTW
DNTNITQPNSTGSNDTITLQCRIKQIINMWQRAGQAMYAPPIPGVISCVSNITGLLLTRDGGITSA
NETFRPGGGDMRDNWRSELYKYKVVKLEPLGVAPTRARRRVVEREKRAVGIGAVFIGFLGAA
GSTMGAASMTLTVQARQLLSGIVQQQSNLLRAIEAQQHLLKLTVWGIKQLQARVLAVESYLRD
QQLLGIWGCSGKLICTTTVPWNASWSNKSYSEIWENMTWLQWDKEISNYTNLIYGLIEESQNQ
QEKNEQDLLALDKWANLWSWFEISNWLWYIKIFIMIVGGLIGLRIVFAVLSIINRVRQGYSPLSFQ
THTPNPGGPDRPGRIEEEDGELGRGRSIRLVNGFLALAWDDLRSLCLFSYHRLRDFILIAARTV
ELLGHSSLKGLRLGWEGLKYLWNLLVYWSRELRISATSLVDTIAIVVAGWTDRVIEIVQGIGRAIL
HIPRRIRQGLERALL*
SEQ ID NO: 77
>A1.UG.92.92UG037.AB253429
MRVMGIERNYPCWWTWGIMILGMIIICNTAENLWVTVYYGVPIWKDANTTLFCASDAKAYDTEV
HNVWATHACVPTDPSPQELKMENVTEEFNMWKNNMVEQMHTDIISLWDQSLKPCVQLTPLCV
TLDCSYNITNNITNSITNSSVNMREEIKNCSFNMTTELRDKNRKVYSLFYKLDVVQINNGNNSSN
LYRLINCNTSALTQACPKVTFEPIPIHYCAPAGYAILKCNDKEFNGTGLCKNVSTVQCTHGIRPV
VSTQLLLNGSLAEGKVMIRSENITNNVKNIIVQLNESVTINCTRPNNNTRRSVRIGPGQTFYATG
DIIGDIRQAHCNVSGSQWNKTLHQVVEQLRKYWNNNTIIFNSSSGGDLEITTHSFNCAGEFFYC
NTSGLFNSTWVNGTTSSMSNGTITLPCRIKQIINMWQRVGQAMYAPPIQGVIKCESNITGLILTR
DGGVNSSDSETFRPGGGDMRDNWRSELYKYKVVKIEPLGVAPTKARRRVVEREKRAVTLGAV
FIGFLGTAGSTMGAASITLTVQARKLLSGIVQQQSNLLRAIEAQQHLLKLTVWGIKQLQARVLAV
ERYLRDQQLLGIWGCSGKLICPTNVPWNSSWSNKSLDEIWENMTWLQWDKEISNYTIKIYELIE
ESQIQQERNEKDLLELDKWASLWNWFDISKWLWYIKIFIMIVGGLIGLRIVFAVLSVINRVRQGYS
PLSFQTHTPNPRGLDRPGRIEEEGGEQDRGRSIRLVSGFLALAWDDLRNLCLFSYHRLRDFILI
AARTVELLGHSSLKGLRLGWEGLKYLGNLLLYWGRELKISAINLLDTIAIAVAGWTDRVIETVQR
LGRAILNIPRRIRQGFERALL*
SEQ ID NO: 78
>A2.CD.97.97CDKS10.AF286241
MRVMGTQTSYQHLWRWGILILGMLIICKATDWWVTVYYGVPVWKDAETTLFCASDDKAYETEA
HNVWATHACVPTDPNPQEVNLKNVTEDFNMWKNNMVEQMHEDIISLWDQSLKPCVKLTPLCV
TLNCSNANTNSTNSTSAPSMGPGEIKNCSFNVTTEVRDKEKKVYALFYKLDVVQINESDSNSTK
DSTQYRLINCNTSAITQACPKVSFEPIPIHYCAPAGFAILKCEDPRFNGTGPCNNVSSVQCTHGI
MPVASTQLLLNGSLAEKEVMIRSENITNNAKNIIVQFNESVPITCIRPNNNTRKGIPIGPGQVFYT
SDIIGDIRQAYCSINKTKWDASLQKVAEQLRKHFPNKTINFTKPSGGDLEITTHSFNCGGEFFYC
NTTSLFNSTWKNGATIQENSTETNGIMTLPCRIKQIVDMWQEVGQAMYAPPIAGVIYCTSNITGII
LTRDGGSSNTNSEIFRPGGGDMRDNWRSELYKYKVVKIEPLGVAPSRAKRRVVEREKRAVGIG
AVFLGFLGAAGSTMGAASITLTVQARQLLSGIVQQQSNLLKAIEAQQHLLKLTVWGIKQLQARVL
ALERYLQDQQLLGIWGCSGKLICTTTVPWNSSWSNKTYEEIWNNMTWLQWDREIDNYTNIIYN
LLEESQNQQEKNEQDLLALDKWASLWNWFSITNWLWYIRIFIMIVGGLIGLRIVMAIISVVNRVR
QGYSPLSFQIPTPNPEGLDRHGRIEEGGGEQDRTRSIRLVSGFLGLAWDDLRSLCLFSYHRLR
DCILIVARTVELLGHSSLKGLRLGWEGLKYLGNLLLYWGRELKNSAISLLNSTAIAVAEWTDRVIE
IGQRACRAILNIPRRIRQGFERALL*
SEQ ID NO: 79
>A2.CD.97.97CDKTB48.AF286238
TRVMGTQRNCQKWWEWGILVFGMIMMCKAADLWVTVYYGVPVWRDADTTLFCASDAKAYAT
EKHNVWATHACVPTDPNPQEVNLANVTEDFNMWKNNMVEQMHADIISLWDQSLKPCVKLTPL
CVTLNCSNANTTNTNSTEEIKNCSYNMPTELKDKTQKVYSLFYELDVVLLNRSKNSSYSTYRLIS
CNTSVITQACPKVSFEPIPIHYCAPAGYAILKCKDKEFNGKGSCSNVSSVQCAHGIRPVASTQLL
LNGSLAEGKVMIRSENITDNAKNIIVQFNKPVPINCTRPNNNTRKSIRFGPGQAFYTNNNIIGDIR
QAHCNISITEWNATLKKVVEQLREHFPNKTIIFNSSSGGDLEITTHSFNCGGEFFYCNTTGLFNS
TWENGTNKQNYTESNDTITLQCRIKQIINMWQRVGRAMYAPPIAGVIKCTSNITGMILTRDGGK
NSINETFRPGGGDMRDNWRSELYKYKVVKIEPLGIAPTEARRRVVQREKRAVGLGAVFLGFLG
AAGSTMGAASITLTVQARQLLTGIVQQQSNLLKAIEAQQQMLRLTVWGIKQLQARVLALERYLQ
DQQLLGIWGCSGKLICATDVRWNSSWSNKTQEQIWKNMTWLQWDKEISTYTDIIYMLLEESQN
QQEKNEQDLLALDKWANLWNWFDITRWLWYIKIFIMIVGGLIGLRIVIAIISVVKRVRQGYSPLSF
QIPTPNPEGLDRPGRIEEEGGEQGRDRSIRLVSGFLALAWDDLRSLCLFSYHRLRDCILIAARIV
ELVGHSSLKGLRLGWEGLKHLWNLLVYWGQELKTSAIRLLDTIAVAVAEWTDRVIEIGQRACRA
IRNIPRRIRQGLERALL*
SEQ ID NO: 80
>A2.CY.94.94CY017_41.AF286237
MRVMGTQRNYQHLWRGGILILGMLIMCKATDLWVTVYYGVPVWKDADTILFCASDAKAYDTEV
HNVWATHACVPTDPNPQEINLENVTENFNMWKNNMVEQMQEDIISLWDQSLKPCVKLTPLCVI
LNCSNANTSTHSNSSSTQSPINEEIKNCSYNTTTILRDKTQKVYSLFYRLDVVQLDESENKNTSG
SNTLYRLINCNTSTITQACPKVTFEPIPIHYCAPAGFAILKCKDPRFNGTGSCKNVSSVQCTHGIK
PVASTQLLLNGSLAEGGKIMIRSENITNNAKNIIVQFTKPVLITCIRPNNNTRKSIRFGPGQAFYTN
EIIGDIRQAHCNINKTLWNDTLQKVAEQLREKFPKKTIIFTNSSGGDPEITTLSFNCAGEFFYCNT
TGLFNGTWWNNGTWNGPYTPNNTNGSIILPCRIKQIINMWQRVGRAMYAPPIAGIIKCTSNITGII
LTRDGGNNGTNETFRPGGGDMRDNWRSELYKYKVVKLEPLGVAPTRAKRRVVEREKRAVGL
GAVFLGFLGAAGSTMGAASLTLTVQARQLLSGIVQQQSNLLQAIEAQQHLLKLTVWGIKQLQAR
VLAVERYLKDQQLLGIWGCSGKLICATTVPWNTSWSNKSQDEIWDNMTWLQWDKEISNYTNII
YRLLEESQNQQEKNEQDLLALDKWADLWSWFNISHWLWYIRIFIMIVGGLIGLRIVFAIITVVNRV
RQGYSPVSFQIPTPSPEGPDRPRGTEEGGGEQGRDRSIRLVNGFFALAWDDLRSLCLFSYHR
LRDCILIAARTVELLGHCSLKGLRLGWEGLKNLWNLLLYWGRELKNSAISLFDTIAVAVAEWTDR
VIEIGQRAFRAILNIPRRIRQGLERALL*
SEQ ID NO: 81
>B.FR.83.HXB2_LAI_IIIB_BRU.K03455
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKVVNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ
LQARILAVERYLKDQQLLGIWGCSGKLICTTAVPWNASWSNKSLEQIWNHTTWMEWDREINNY
TSLIHSLIEESQNQQEKNEQELLELDKWASLWNWFNITNWLWYIKLFIMIVGGLVGLRIVFAVLSI
VNRVRQGYSPLSFQTHLPTPRGPDRPEGIEEEGGERDRDRSIRLVNGSLALIWDDLRSLCLFS
YHRLRDLLLIVTRIVELLGRRGWEALKYWWNLLQYWSQELKNSAVSLLNATAIAVAEGTDRVIE
VVQGACRAIRHIPRRIRQGLERILL*
SEQ ID NO: 82
>B.NL.00.671_00T36.AY423387
MKVKGIRKNYQLLWRWGIMLLGTLMICSATENLWVTVYYGVPVWKEATTTLFCASDAKAYETE
VHNVWATHACVPTDPNPQELVLENVTENFNMWKNNMVEQMHEDIISLWDESLKPCVKLTPLC
VTLNCTDANITSSNNITGSNNNSNLEQMAREISNCSFNITTTIKNKRQREFALLSKLDIVPIDNDS
YSYMLINCNTSVITQACPKVSFQPIPIHYCTPAGFAILKCNDKKFNGTGPCKNVSTVQCTHGIRP
VVSTQLLLNGSLAEEDVVIRSKNFTDNTKTIIVQLKESVEINCTRPNNNTRKSIHIGPGRAFYATG
EIIGDIRQAHCNLSRAKWNDTLNQIVGKLRELYKNKTIVFNSSSGGDPEIVMHSFNCRGEFFYCN
TTQLFNSTWDVNATGNGTTEPNSTITLPCRIKQIINRWQEVGKAMYAPPIAGQISCSSNITGLLLT
RDGGGGENNSTEIFRPGGGDMRDNWRSELYKYKVVKIEPLGVAPTKAKRRVVQREKRAITLG
AMFLGFLGAAGSTMGAASMALTVQARQLLSGIVQQQNNLLRAIEAQQHLLQLTVWGIKQLQAR
VLAIERYLQDQQLLGIWGCSGKLICTTTVPWNASWSNKSLDQIWENMTWMQWEREIDNYTSLI
YTLIEDSQKQQEKNEQELLALDTWASLWNWFSITNWLWYIKIFIMIVGGLVGLRIVFIVLSIVNRV
RKGYSPLSFQTHLPAPRGPDRPEGIEEEGGERDRDGSGPLVNGFLAIIWVDLRSLCLFSYHRL
RDLLLIVVRIVELLGRRGWEALKYWWNLLQYWIQELRGSAVSLFNAIAIAVAEGTDRVIETIQRAF
RAILHIPRRIRQGLERILL*
SEQ ID NO: 83
>B.TH.90.BK132.AY173951
MRVKEIRKNCQHLWRWGILLLGILMISSAAENLWVTVYYGVPVWKEATTTLFCASDAKAYDTEV
HNVWATHACVPTDPNPQEVVLVNVTENFXMWTNNMAEQMHEDIISLWDQSLKPCVKLTPLCV
TLNCTDLRNTTNTNSTAEEMEAKGEMKNCSFNITTSIRNKLQKEYALFYKLDIVPINNDNTSYRLI
SCNTSVITQACPKVSFEPIPIHYCAPAGFAILKCNDKKFSGNGPCKNVSTVQCTHGIKPVVSTQL
LLNGSLAEEEVVIRSENFTDNAKTIIVQLKEPVEINCTRPNNYTRKRITMGPGRVYYTTGEIIGDIR
RAHCNISSTKWNNTLGQIVKKLKEQFNNNTIVFKKSSGGDPEIVMHSFICGGEFFFCNSTKLFNS
TWNSTEGNDDGEERNITLPCRIKQIVNMWQEVGKAMYAPPIGGQIRCTSNITGLLLTRDGGNQ
NGTNETEIFRPGGGNMRDNWRSELYKYKVVKIEPLGVAPTKAKRRVVQREKRAVGIGAVFLGF
LGAAGSTMGAASVTLTVQARLLLSGIVQQQNNLLRAIEAQQHLLQLTVWGIKQLQARVLAVERY
LKDQQLLGIWGCSGKLICTTAVPWNASWSNKSLDEIWNNMTWMQWEREINNYTGLIYTLIEES
QNQQEKNELDLLQLDKWASLWNWFDITNWLWYIKIFIMIVGGLVGLRIIFTVLSIVNRVRQGYSP
LSFQTHLPAPRGPDRPGGIEEEGGERDRDTSGRLVDGFLAIFWVDLRNLCLFSYHRLRDLLLIV
TRIVELLGRRGWEALKYWWNLLQYVVSQELKNSAVSLLNATAIAVAEGTDRVIEVLQRVYRAILNI
PTRIRQGLERALL*
SEQ ID NO: 84
>B.US.98.1058_11.AY331295
MRVKGIRRNCQHSWRWGTTLTMLLGILMICRAAEQLWVTVYYGVPVWREAKTTLFCASDAKA
YDTEVHNVWATHACVPTDPNPQELVLVNVTENFNAWENNMVEQMHEDIISLWDQSLKPCVKL
TPLCVTLNCNDLNTTTSNTTGTEGLTMDKGEMKNCSFNITTDISNKKQKQYALFYKLDVVQMN
NNNNSYRLISCNTSVITQACPKVSFEPIPIYYCAPAGFAILKCNDKSFSGKGECKNVSTVQCTHG
IRPVVSTQLLLNGSLAEEDVIIRSDNFTDNAKTIIVQLNETVDIHCIRPNNNTRKRITMGPGKVYYT
TGQIIGDIRQAHCNLSEAKWNNTLRRVVRKLREKFNKTIVFNQSSGGDPEIVMHTFNCGGEFFY
CNSTKLFNSIWDNNKDSTKTNEPNDGKNITLPCRIKQIINMWQGVGKAMYAPPIRGQIRCTSNIT
GLLLTRDGGKNNGTNGTEVFRPGGGNMKDNWRSELYKYKVVKIEPLGVAPTTAKRRVVQREK
RAVTLGALFLGFLGAAGSTMGAASMTLTVQARLLLSGIVQQQNNLLRAIEAQQHLLQLTVWGIK
QLQARVLAVERYLEDQQLLGIWGCSGKLICTTAVPWNASWSNKSRSEIWNNMTWMQWDKEIH
NYTNLIYTLIGESQIQQEKNEQELLGLDKWASLWNWFDITKWLWYIKIFIMIVGGLIGLRIVFTVLSI
MNRVRQGYSPLSFQTRLPTQRGPDRPEGTEEEGGERDRDRSGPLVDGFLAIIWVDLRSLCLFL
YHRLRDLLLIVTRTLELLGRRGWEILKYWWNLLQYWSQELKNSAVSLLNATAIAVAEGTDRVIEI
VQRTFRAILHIPVRIRQGLERALL*
SEQ ID NO: 85
>B.US.98.15384_1.DQ853463
MKVKETRRNYQHLWRWGTMLLGMLMICRAAENLWVTVYYGVPVWKEATTTLFCASDAKAYE
TEVHNVWATHACVPTDPNPQEIVLENVTENFNMWKNNMVEQMHEDIISLWDQSLKPCVKLTPL
CVTLNCTDYLGNTTKTNTTSAPTTSTTTTNTTNNKGELKNCSFQVTTGIGDRTKKEYALFYKHD
VVPIDNDNNKTNNSNFILIHCNSSVITQACPKVSFEPIPIHYCAPAGFAILKCKDKKFNGTGPCKN
VSTVQCTHGIRPVVSTQLLLNGSLAEEEIVIRSQNFTDNVKSIIVQLNETVKINCTRPNNNTRKAI
RIGRGRAIYATDRIIGDIRQAYCNISRTKWNDTLGQIATKLREQFGNKTIVFNSSSGGDPEIVMHS
FNCGGEFFYCNTTQLFNGMWHANGTWNSTWNDTGGSNDTIRLPCRIKQIVNMWQEVGKAMY
APPIKGQIQCSSNITGLILTRDGGNSTNETGEVFRPGGGNMKDNWRSELYKYKVVEIEPLGVAP
TKAKRRVVQREKRAVGLIGAVFLGFLGAAGSTMGAASIALTVQTRHLLSGIVQQQNNLLRAIEA
QQHLLQLTVWGIKQLQARILAVERYLRDQQLLGIWGCSGKLICPTAVPWNASWSNKSLEEIWE
NMTWREWEREIDNYTGKIYDLLAKSQNQREMNEQELLKLDKWADLWNWFDITQWLWYIKIFIM
IVGGLIGLRIIFAVISIVNRVRQGYSPLSLQTLLPTQRGPDRPEGIEEEGGERDRDRSIRLVEGFS
ALIWDDLRSLFLFSYHRLRDLLLIVTRIVELLGRRGWEALKYWWNLLQYWIQELKNSAINLLNTT
AIVVAEGTDRVIEVVLRAYRAILHIPRRIRQGLERLLL*
SEQ ID NO: 86
>C.BR.92.BR025_d.U52953
MRVEGIQRNWKQWWIWGILGFWMVMIYNVRGNLWVTVYYGVPVWKEAKTTLFCASDAKAYD
AEVHNVWATHACVPTDPNPQEMVLENVTENFNMWENDMVEQMHQDIISLWDQSLKPCVKLT
PLCVTLHCSNRTIDYNNRTDNMGGEIKNCSFNMTTEVRDKREKVHALFYRLDIVPLKNESSNTS
GDYRLINCNTSAITQACPKVSFDPIPIHYCAPAGYAILKCNNKTFNGTGPCNNVSTIQCTHGTKP
VVSTQLLLNGSLAEEEIIIRSKNLTDNVKTIIVHLNESVEINCTRPNNNTRKSIRIGPGQAFYATGEI
IGDIRQAHCNISRTAWNKTLQEVGKKLAEHFPNKAIKFAKHSGGDLEITTHSFNCRGEFFYCNTS
SLFNSTYTPNSTENITGTENSIITIPCRIKQIINMWQGVGRAMYAPPIEGILTCRSNITGLLLTRDG
GTGMHDTEIFRPEGGDMRDNWRSELYKYKVVEIKPLGIAPTKAKRRVVEREKRAVGIGAVFLG
FLGAAGSTMGAASITLTVQVRQLLSGIVQQQSNLLRAIEAQQHMLOLTVWGIKOLQTRVLAIER
YLRDQQLLGIWGCSGKLICTTAVPWNSSWSNRSQEDIWNNMTWMQWDREISNYTNTIYRLLE
DSQNQQEKNEQDLLALDKWQNLWTWFGITNWLWYIKIFIKIVGGLIGLRIIFAVLSIVNRVRQGY
SPLSFQTLTPNPRGPDRLGGIEEEGGEQDRDRSIRLVSGFLALAWDDLRSLCLFSYHRLRDLILI
AARAVELLGRSSLRGIQRGWEILKYLGGLVQYWSLELKKSAISLFDTIAIAVAEGTDRIIEVIQGIW
RAICNIPRRIRQGFEAALQ*
SEQ ID NO: 87
>C.ET.86.ETH2220.U46016
MKVMGIQRNCQQWWIWGILGFWMLMICNGMGNLWVTVYYGVPVWKDASPTLFCASDAKAYD
TEVHNVWGTFACVPTDPSPQELGLENVTENFNMWKNDMVEQMHQDIISLWDQGLKPCVKLTP
LCVTLNCNAIKNNTKVTNNSINSANDEMKNCSFNITTELRDKKRKAYALFYKLDIVPLNNGSTDY
RLINCNTSTITQACPKVSLDPIPIHYCAPAGYAILKCRDKTFTGTGPCHNVSTVQCTHGIKPVVST
QLLLNGSIAEGETIIRFENLTNNAKIIIVQLNESVEITCTRPSNNTRESIRIGPGQTFYATGDIIGDIR
QAHCNISEEKWNKTLQKVKEKLQKHFPNKTIEFKPSSGGDLEITTHSFNCGGEFFYCNTSNLFN
STKLELFNSSTNLNITLQCRIKQIINMWQGVGRAMYAPPIEGIIMCRSNITGLLLTRDGAKEPHST
KEIFRPEGGDMRDNWRSELYKYKVVEIKPLGVAPTKPKRRVVEREKRAALGALFLGFLGAAGS
TMGAASITLTVQARQLLSGIVQQQSNLLKAIEAQQHMLQLTVWGIKQLQTRVLAIERHLRDQQLL
GIWGCSGKLICTTAVPWNSSWSNKSQEEIWDNMTWMQWDREISNYTDIIYNLLEVSQNQQDK
NEKDLLALDKWENLWNWFNITNWLWYIKIFIMIVGGVIGLRIIFAVLSIVNRVRQGYSPLSFQTLIP
HPRGPDRLGGIEEEGGEQGRDRSIRLVNGFLAIFWDDLRSLCLFSYHRLRDLILIAARTVELLGR
SSLKGLQRGWETLKYLGSLVQYVVGLELKKSAINLLNTTAIVVGEGTDRFIELIQRIWRAFCNIPR
RIRQGLEAALQ*
SEQ ID NO: 88
>C.IN.95.95IN21068.AF067155
MRVRGILRNYQQWWIWGVLGFWMLMICNVVGNLWVTVYYGVPVWKEANTTLFCASDAKAYE
KEVHNVWATHACVPTDPNPQEIVMENVTENFNMWKNDMVNQMHEDVISLWDQSLKPCVKLT
PLCVTLECRNVNSTGNGTHSKTYNESMKEIKNCSFNATTVIKDKKQTVYALFYKLDIVPLDNEE
QENDSNSSGYYRLINCNTSALTQACPKVTFDPIPIHYCAPAGYAILKCNNKTFNGTGPCHNVST
VQCTHGIKPVVSTQLLLNGSLAEGGIIIRSENLTNNVKTIIVHLNQPVEIMCTRPDNNTRKSIRIGP
GQTFYATGDIIGDIRQAHCNISEDKWNETLQNVSKKLAEHFPNKTIIFNSSSGGDLEITTHSFNCR
GEFFYCNTSGLFNRTYMPNDTKSNSSSNPNANITIPCRIKQIINMWQEVGRAMYAPPIEGKITCR
SNITGLLLVRDGGEDKNNTETNKTETFRPGGGDMRDNWRSELYKYKVVEVKPLGVAPTTAKR
RVVEREKRAVGIGAVFLGFLGAAGSTMGAASITLTVQARQLLSGIVQQQSNLLRAIEAQQHLLQ
LTVWGIKQLQTRVLAIERYLKDQQLLGIWGCSGKLICTTAVPWNSSWSNRTQKEIWDNMTWM
QWDREINNYTNTIYRLLEESQNQQEENEKDLLALDSWKNLWNWFDITKWLWYIKIFIIIVGGLIGL
RIIFAVISIVNRVRQGYSPLSFQTLTPNPGGPDRLGRIEEEGGEQDKDRSIRLVSGFLALFWDDL
RNLCLFSYHRLRDFILVAARVLELLGRRSLRGLQRGWEALKYLGSLVQYWGLELKKSAINLLDRI
AIAVAEGTDRILELVQRICRAIRNIPRRIRQGFEAALQ*
SEQ ID NO: 89
>C.ZA.04.SK164B1.AY772699
MRVRGILRNWPQWWIWGILGFWMIIICRGEENSWVTVYYGVPVWTEAKTTLFCASDAKAYEKE
VHNVWATHACVPTDPSPQELVLENVTESFNMWENDMVDQMHEDIIGLWDESLKPCVKLTPLC
VTLNCNTTSHNNSSPSPMTNCSFNATTELRDKTQKVNALFYRSDIVPLEKNSSEYILINCNTSTIT
QACPKVSFDPIPIHYCAPAGYAILKCNNKTFNGTGPCSNVSTVQCTHGIKPVVSTQLLLNGSLAE
GEIIIRSENLTDNAKTIIVHLNKSVAIVCTRPNNNTRKSIRIGPGQVFYTNEIIGNIRQAHCNISREL
WNNTLEQVKKKLKEHFQNKTIEFQPPAGGDLEVTTHSFNCRGEFFYCNTSNLFNITASNASDA
NNNTITLPCKIKQIINMWQEVGRAMYAPPIAGNITCNSSITGLLLTRDGGNNNDTGNNNDTEIFR
PGGGNMKDNWRSELYKYKVVEIKPLGIAPTKAKRRVVEREKRAVGLGAVLLGFLGTAGSTMGA
ASITLTVQARQLLSGIVQQQSNLLRAIEAQQHMLQLTVWGIKQLQARVLAIERYLKDQQLLGLW
GCSGKLICTTAVHWNSSWSNKSQDYIWGNMTWMQWDREINNYTDIIYTLLEESQSQQEKNEK
DLLALDSWNNLWNWFSITKWLWYIKIFIMIVGGLIGLRIILGVLSIVKRVRQGYSPLSFQTLPPNP
RGPDRLRGIEEEGGEQDKDRSIRLVSGFLALVWEDLRSLCLFSYHRLRDFILIAGRAAELLGRSS
LRGLQTGWQALKYLGSLVQYWGLELKKSAINLFDTTAIVVAEGTDRLIEGLQGIGRAIYNIPRRIR
QGFEAALL*
SEQ ID NO: 90
>D.CD.83.ELI.K03454
MRARGIERNCQNWWKWGIMLLGILMTCSAADNLWVTVYYGVPVWKEATTTLFCASDAKSYET
EAHNIWATHACVPTDPNPQEIALENVTENFNMWKNNMVEQMHEDIISLWDQSLKPCVKLTPLC
VTLNCSDELRNNGTMGNNVTTEEKGMKNCSFNVTTVLKDKKQQVYALFYRLDIVPIDNDSSTN
STNYRLINCNTSAITQACPKVSFEPIPIHYCAPAGFAILKCRDKKFNGTGPCTNVSTVQCTHGIRP
VVSTQLLLNGSLAEEEVIIRSENLTNNAKNIIAHLNESVKITCARPYQNTRQRTPIGLGQSLYTTR
SRSIIGQAHCNISRAQWSKTLQQVARKLGTLLNKTIIKFKPSSGGDPEITTHSFNCGGEFFYCNT
SGLFNSTWNISAWNNITESNNSTNTNITLQCRIKQIIKMVAGRKAIYAPPIERNILCSSNITGLLLTR
DGGINNSTNETFRPGGGDMRDNWRSELYKYKVVQIEPLGVAPTRAKRRVVEREKRAIGLGAM
FLGFLGAAGSTMGARSVTLTVQARQLMSGIVQQQNNLLRAIEAQQHLLQLTVWGIKQLQARILA
VERYLKDQQLLGIWGCSGKHICTTNVPWNSSWSNRSLNEIWQNMTWMEWEREIDNYTGLIYS
LIEESQTQQEKNEKELLELDKWASLWNWFSITQWLWYIKIFIMIIGGLIGLRIVFAVLSLVNRVRQ
GYSPLSFQTLLPAPRGPDRPEGTEEEGGERGRDRSVRLLNGFSALIWDDLRSLCLFSYHRLRD
LILIAVRIVELLGRRGWDILKYLWNLLQYWSQELRNSASSLFDAIAIAVAEGTDRVIEIIQRACRAV
LNIPRRIRQGLERSLL*
SEQ ID NO: 91
>D.CM.01.01CM_4412HAL.AY371157
MRVMGIERNYQHSWKWGTMLLGMLLMTYSAAGNLWVTVYYGVPVWKEAKTTLFCASDAKSY
KTEAHNIWATHACVPTDPDPQELVLDNVTENFNMWKNNMVEQMHEDIISLWDQSLKPCVKLTP
LCVTLNCSNPNITNNSSANNISIVKKMKNCSFNTTTILKDKQKQEYALFYILDIVGIDNSSNRLINC
TTSVITQACPKITSEPIPIHYCAPAGFAILKCNDKLFNGTGPCRNVSAVQCTHGIKPVVSTQLLLN
GSLAEEVMVRSENLTDNAKNIIVQLNNTINITCVRPNSNTRKSINLGPGQAFYATYATNIIGNIRQ
AHCNLSATQWNKTLHQVAQKLGKLLNKTKINFNSSSGGDPEITTHSFVCGGEFFYCNTSGLFN
GTWDNGTWTWNTSAVPNETITIPCRIKQIINMWQGVGRAMYAPPIEGLIKCSSNITGLLLTRDG
GNTSDSAETFRPGGGDMRDNWRSELYKYKVVKIEPLGVAPTRAKRRVVEREKRAIGLGAMFL
GFLGAAGSTMGAASVTLTVQARQLLSGIVQQQNNLLRAIEAQQHLLQLTVWGIKQLQARVLAV
ERYLKDQQLLGIWGCSGKHICTTNVPWNSSWSNRSLDDIWQNMTWMQWEREIENYTGVIYSL
IEESQIQQEKNEKELLELDKWASLWNWFSISNWLWYIRIFIMIVGGLIGLRIVFAVLSMVRRVRQ
GYSPLSFQTLLPAPRGPDRPEGIEEEGGEQDRGRSIRLVNGFSALIWDDLRNLCLFSYHRLRDL
ILIAARIVDLLGRRGWEALKYLWNLLRYWSQELKNSAINLLNTTAIAVAEGTDRVIEIIQRAGRAVL
HIPRRIRQGFERALL*
SEQ ID NO: 92
>D.TZ.01.A280.AY253311
MRVMETQRNYQHLWRWGIMLLGMWMTYSVAEQLWVTIYYGVPVWREANTTLFCASDAKSFD
TEAHNIWATHACVPTDPNPQEMDLVNVSENFNMWKNNMVEQMHEDIISLWEESLKPCVKLTP
LCVTLHCSDANTTNSGNGTNTTDPRLIEKGEMKNCSFNITTEIRDKRKQVQALFYKLDVVPIDKK
NNNSYTLMHCNTSAIKQACPKVSFEPIPIHYCAPAGFAILKCKDKKFNGTGPCKKVSTVQCTHGI
RPVVSTQLLLNGSLAGEEIIIRSENLTNNVKTIIVQLNETVKINCTRPNNNTRKGIRIGPGQTFFTA
EVTGDIRKAYCNISGAEWDKTLQQVATKLGDLLNKTIINFSPSSGGDPEITTHSFNCGGEFFYCN
TSLLFNTTWIKGTQNNTETNNSTITIPCRIKQIINMWQGVGKAMYAPPIAGLIRCTSNITGLLLTRD
GGNVNNSREEIFRPGGGDMRDNWRSELYKYKVVRIEPIGVAPTRAKRRVVEREKRAIGLGAMF
LGFLGAAGSTMGAASLTLTVQARQLLSGIVQQQNNLLRAIEAQQHLLQLTVWGIKQLQARVLAV
ESYLKDQQLLGIWGCSGKHICTTAVPWNSSWSNKSLDDIWNNMTWMEWEKEIDNYTGVIYSLI
EESQVQQEKNEKELLELDKWASLWNWFSITKWLWYIKLFIMIVGGLIGLKIVFTVFSLVNRVRQG
YSPLSLQTLLPASRGPDRPEGIEEEGGEQGRGRSIRLVNGFSALIWDDLRNLCLFSYRHLRDLIL
IATRIVGLLGHRGWEAIKYLWNLLQYVVIQELKNSAISLLNVTAIAVAEGTDRIIEIIQRAFRAVLHIP
RRVRQGLERALL*
SEQ ID NO: 93
>D.UG.94.94UG114.U88824
MRVRETKRNYQHLWKWGTMLLGMLMICSVTGKSWVTVYYGVPVWKEATTTLFCASDAKAYK
AEAHNIWATHACVPTDPNPQEIKLENVTENFNMWKNNMVEQMHEDIISLWDQSLKPCVKLTPL
CVTLNCTNWVTDTTNTTGMANCSFNITTEIRDKKKQVQALFYKLDVVKINDNDSDNTSYRLINC
NTSAITQACPKMTFEPIPIHYCAPAGFAILKCNEKKFNGTGPCKNVSTVQCTHGIKPVVSTQLLL
NGSLAEEEIIIRSENLTNNAKIIIVQLNESVPINCIRPYNNTRQSTRIGPGQALFTTKVIGDIRQAHC
NISGAGWNKTLQQVAEKLGNLLNQTTIIFKPSSGGDPEITTHSFNCGGEFFYCNTTRLFNSTWK
RNNSEWRSDNTPDETITLQCRIKQIINMWQEVGKAMYAPPIEGFINCSSNITGLLLTRDGGAINS
SQNETFRPGGGDMRNNWRSELYKYKVVKLEPIGLAPTAAKRRVVEREKRAIGLGALFLGFLGT
AGSTMGAVSLTLTVQARQVLSGIVQQQNNLLRAIEAQQHLLQLTVWGIKQLQARILAVESYLKD
QQLLGIWGCSGKHICTTNVPWNSSWSNRSVDEIWNNMTWMEWEREIDNYTELVYSLLEVSQI
QQEKNEQELLKLDTWASLWNWFSITQWLVVYIKIFIMIVGGLIGLRIVFAVLSVVNRVRQGYSPLS
FQTLLPAPREPDRPEGIEEEGGERDRGRSIRLVNGLSALIWDDLRNLCLFSYHRLRDLILIAARIV
ELLGRRGWEAIKYLWNLLQYWIQELKNSAVSLFNTIAIAVAEGTDRAIELVQRAVRAILNIPVRIR
QGLERALL*
SEQ ID NO: 94
>F1.BE.93.V1850.AF077336
MRVRGMQRNWQHLGKWGLLFLGILIICNAADNLWVTVYYGVPVWKEATTTLFCASDAKAYER
EAHNVWATHACVPTDPNPQEVFLKNVTENFDMWKNNMVEQMHTDIISLWDQSLKPCVKLTPL
CVTLNCTNATNNSQEKPGAMQNCSFNMTTEVRDKKLKLSALFYRLDIVPIGNNNSSEYRLINCN
TSTITQACPKVSWDPIPIHYCAPAGYAILKCNDKRFNGTGPCKNVSTVQCTHGIKPVVSTQLLLN
GSLAEEGIVIRSQNISNNAKTIIVHLNESVQINCTRPNNNTRKGIHLGPGQTFYATGAIIGDIRKAH
CNISGTQWNNTLEYVKAELKSHFPNNTAIKFNQSSGGDLEITMHSFNCRGEFFYCDTSGLFND
TGSNNGTITLPCRIKQIVNMWQGVGRAMYTSPIAGNITCNSNITGLLLTRDGGNESNIETFRPEG
GNMKDNWRSELYKYKVVEIEPLGVAPTKAKRQVVQREKRAAGLGALFLGFLGDSREHMGAAS
ITLTVQARQLLSGIVQQQNNLLRAIEAQQHLLQLTVWGIKQLQARVLAVERYLKDQQLLGIWGC
SGKLICTTNVPWNSSWSNKSQEEIWNNMTWMEWEKEISNYSNIIYKLIEESQNQQEKNEQELL
ALDKWASLWNWFDISNWLWYIKIFIMIVGGLIGLRIVFAVLSIVNRVRKGYSPLSLQTLIPSPRGP
DRPEGIEEGGGEQGKDRSVRLVTGFLALAWDDLRNLCLFSYRHLRDFILIAARIVDRGLRRGWE
ALKYLGNLTRYWSQELKNSAISLFNTTAIVVAEGTDRIIEVLQRAGRAVLNIPRRIRQGAERALL*
SEQ ID NO: 95
>F1.BR.93.93BR020_1.AF005494
MRVRGMQRNWQHLGKWGLLFLGTLIICNAAENLWVTVYYGVPVWKEATTTLFCASDAKSYEK
EAHNVWATHACVPTDPNPQEVVLENVTERFNMWENNMVEQMHTDIISLWDQSLKPCVKLTPL
CVTLDCRNIATNGTNDTIAINDTLKEDPEAIQNCSFNTTTEIRDKQLKVHALFYKLDIVQINKDDN
RTYRLINCDASTITQACPKVSWDPIPIHYCAPAGYAILKCNEKNFTGTGSCKNVSTVQCTHGIKP
VVSTQLLLNGSLAEGEIVIRSQNISDNAKTIIVHLNESVQINCTRPNNNTRKRISLGPGRVFYTTG
EIIGDIRKAHCNVSGTQWRNTLAKVKAKLGSYFPNATIKFNSSSGGDLEITRHNFNCMGEFFYC
NTDELFNDTKFNDTGFNGTITLPCRIKQIVNMWQEVGRAMYANPIAGNITCNSNITGLLLTRDGG
LNSTNETFRPGGGNMKDNWRSELYKYKVVEIEPLGVAPTKAKRQVVKRERRAVGLGALFLGFL
GAAGSTMGAASITLTVQARQLLSGIVQQQSNLLRAIEAQQHLLQLTVWGIKQLQARVLAVERYL
KDQQLLGLWGCSGKLICTTNVPWNSSWSNKSLEEIWGNMTWMEWEKEVSNYSKEIYRLIEDS
QNQQEKNEQELLALDKWASLWNWFDITQWLWYIKIFIMIVGGLIGLRIVFTVLSIVNRVRKGYSP
LSFQTHIPSPREPDRPEGIEEGGGEQGKDRSVRLVTGFLALAWDDLRNLCLFSYRHLRDFILIAA
RIVDRGLKRGWEALKYLGNLTQYWGQELKNSAISLLNATAIAVAEWTDRVIEALQRAGRAILNIP
RRIRQGLERALL*
SEQ ID NO: 96
>F1.FI.93.FIN9363.AF075703
MRVRGMQRNWQHLGKWGLLFLGMLIICKAADDLWVTIYYGVPVWKEANTTLFCASDAKSYEK
EVHNVWATHACVPTDPNPQEVALNVTENFNMWENDMVEQMHKDIISLWDQSLKPCVKLTPLC
VTLNCTNATTTNDTLSDQSSTLKEEPGAIQNCSFNMTTEVEDKKQKVHALFYRLDIEPISNNNS
REEYRLITCNTSTITQACPKVSWDPIPIHYCAPAGYAILKCKDKRFNGTGPCRNVSTVQCTHGIR
PVVSTQLLLNGSLSEGGIIIRSQNLSDNAKTIIVHLNESVQINCTRPNNNTRKSIRIGPGQSFYATG
EIIGDIRKAHCNISGEQWNKTLDRVKAELKLHFNKTIQFNSSSGGDLEITMHSFNCRGEFFYCNT
SLLFNNTVPNNGTITLPCRIKQFVNMWQEVGRAMYAAPIAGNITCNSNITGLLLTRDGGQSNNS
DSETFRPGGGDMKDNWRSELYKYKVVEIEPLGVAPTRPKRPVVRRERRAVAIGAVFLGFLSAA
GSTMGAASLTLTVQARQLLSGIVQQQNNLLQAIEAQQHMLQLTVWGIKQLQARVLAVERYLKD
QQLLGLWGCSGKLICTTNVPWNSSWSNKSQDEIWNNMTWMQWEKEISNYSKTIYMLIEKSQS
QQERNEQELLELDKWDSLWSWFDITNWLWYIKIFIMIVGGLIGLRIVFAVLSIVNRVRKGYSPLSL
QTLIPAPTEPDRPEGIEEGGGEQGKDRSVRLVNGFLALVWDDLRNLCLFSYRHLRDFILIAARIV
DRGLRRGWEALKYLGNIIQYVVSQELKNSAISLFNTTAIVVAEGTDRVIEALQRAVRAVLNIPRRIR
QRVERALI*
SEQ ID NO: 97
>F1.FR.96.MP411.AJ249238
MRVRVMQRNWQHLGKWGLLFLGILIICSAADNLWVTVYYGVPVWKEATTTLFCASDAKGYER
EVHNVWATHACVPTDPNPQEIWLKNVTENFDMWKNNMVEQMHEDIISLWDQSLKPCVKLTPL
CVTLHCSDVNITSNATTTNDTSTPEESGAIQNCSFNMTTEVKDKKLRVNALFYKLDIIPINNSSSS
DYRLINCNTSTIKQACPKVSWDPIPIHYCAPAGYAILKCRDPRFNGTGPCKNVSTVQCTHGIRPV
VSTQLLLNGSLAEEDIIIRSQNISDNAKTIIVHLNESVQINCTRPNNNTRKSIHLGPGQAFYATGDII
GDIKKAYCEINGTQWSKTKTQVQEKLRALFNKTIKFNQSSGGDLEITMHSFNCRGEFFYCDTSG
LFNESEKYNGTIILPCKIKQIINMWQGVGQAMYSAPIAGRINCNSTITGLLLTRDGGQSNDTNRT
ETFRPEGGNMKDNWRNELYKYKVVEIEPLGVAPTKARRRVVQRERRAVGIGALFLRFLGAAGS
NIGAASITLTVQARQLLSGIVQQQNNLLRAIEAQQHLLQLTVWGIKQLQARVLAVERYLKDQQLL
GIWGCSGKLICTTNVPWNTSWSNKSHDEIWNNMTWMQWEKEINNYSNTIYRLIEESQNQQEK
NEQELLALDKWASLWSWFDISNWLWYIKIFIMIVGGMIGLRIVFAVLSIVNRVRKGYSPLSLQTLI
PSPRGPDRPEGIEEGGGEQDRNRSVRLVNGFLSLVWDDLRNLCLFSYRHLRDFILIAARTVDR
GLTRGWETLKYLWNLAQYWSQELKNSAISLLNTTAIVVAEGTDRVIEVLQRAGRAVLNVPRRIR
QGLERSLL*
SEQ ID NO: 98
>F2.CM.02.02CM_0016BBY.AY371158
MRVRGMQRNWQHLGKWGFLFLGILIICNAADNLWVTVYYGVPVWKEATTTLFCASDAKAYEK
EAHNVWATHACVPTDPDPQEIFLDNVTENFNMWKNNMVDQMHEDIISLWDQSLKPCVKITPLC
VTLHCSDVNITANNTNQPNNITLEEQGEIKNCSFNITTEIKDKRKNEFATFYKHDIVPIEKNTTSYR
LTSCNTSTVTQACPKVSFDPIPIYYCAPAGYAILKCNDKRFNGKGLCTNVSTVQCTHGIKPVVST
QLLLNGSLAEKNIIIRSENITDNAKTIIVQFNESVKINCTRPNNNTRKSIRIGPGQVFYATGEIIGDIR
KAHCTINGTLWNATLNRVAAEVKNLTNITIKFEPSSGGDLEVTTHSFSCGGEFFYCDTTALFNTT
LLNTTMDNNGTIIIPCRIKQIVNVWQRVGRAMYAPPIAGKIQCNSNITGLLLTRDGGSKANNTDIL
RPIGGEMRDNWRSELYKYKVVQIQPLGIAPSRAKRQVVKRERRAVGIGAVFLGFLGAAGSTMG
AASITLTVQARQLLSGIVQQQNNLLKAIEAQQHLLQLTVWGIKQLQARILAVERYLKDQQLLGIW
GCSGKLICTTNVPWNSSWSNKSQEEIWGNMTWMQWEKEIDNYTDTIYRLIEEAQNQQEKNEQ
DLLALDKWDSLWSWFTITNWLWYIRIFIMVVGGLIGLRIVFAVLSIINRVRQGYSPLSLQTLIPSPR
GPDRPGGIEEEGGEQDKDRSVRLVSGFLALAWDDLRSLCLFSYRHLRDFILIAARTVDRGLKG
GWEVLKYLWNLAQYWGRELKISAISLLNTTAIVVAEGTDRVIEILQRAGRAILHIPRRIRQGFERA
LL*
SEQ ID NO: 99
>F2.CM.95.MP255.AJ249236
MRVREMQRNWQHLGKVVGLLFLGILIICNANATDDLWVTVYYGVPVWKEPTTTLFCASDAKAYD
PEVHNVWATYACVPTDPNPQELVLGNVTENFNMWENNMVDQMHLDIISLWDQSLKPCVKSTP
LCVTLNCTDVNITMSDINGTSLKEDQGEIKNCSFNVTTELKDKKRKQQALFYRLDVEPIKNSSNI
YKLISCNMSTVTQACPKVSFDPIPIHYCAPAGYAILKCNDKRFNGTGPCEKVSTVQCTHGIRPVV
STQLLLNGSLAQEDIIIRSKNITDNTKNIIVQFNRSVIIDCRRPNNNTRKGIRIGPGQTFFATGEIIG
DIRKAYCNINRTLWNETLKNVSGEFKKHFNFSVAFNSSSGGDVEITTHSFNCRGEFFYYNTSGL
FNETEVANNTNENITLPCRIRQFVNMWQRIGRAMYAPPIEGEIQCTSNITGLLLTRDGSKDIDGK
EILRPIGGDMRDNWRSELYKYKVVRIEPVGVAPTKAKRRVVQRAKRAVGMGAVLFGFLGAAGS
TMGAAAITLTAQARQLLSGIVQQQSNLLKAIEAQQHLLQLTVWGIKQLQARILAVERYLKDQQLL
GIWGCSGKLICTTNVRWNSSWSNKSYDDIWDNMTWMQWEKEIDNYTKTIYSLIEDAQNQQER
NEQELLALDKWDSLWSWFSITNWLWYIKIFIMIVGGLIGLRIVFAVLSVVNRVRQGYSPLSLQTLI
PNPRGPDRPGGIEEEGGEPDRDRSMRLVSGFLPLTWDDLRSLCSFSYRHLRDLLLIAARTVDR
GVKGGWEALKYLWNLTQHWGRELKNSAISLFDTIAIAVAEGTDRIIEVLQRAGRAVLHIPRRIRQ
GAERFLL*
SEQ ID NO: 100
>F2.CM.95.MP257.AJ249237
MRVREMQRNWQHLGRWGLLFLGILIICSAADKLWVTVYYGVPVWKEATTTLFCASDAKAYERE
VHNVWATYACVPTDPSPQELVLGNVSEKFNMWKNNMVDQMHEDIISLWDESLKPCVKLTPLC
VTLNCTKAIINVTSSNNTTLAPNVTISEEMKNCSFNITTEIRDKQKKEYALFYKLDVVQINNSNTSY
RLINCNTSTLTQACPKVSFDPIPIHYCAPAGFAILKCNNKTFNGTGLCRNVSTVQCTHGIKPVVS
TQLLLNGSLAEEKMIIRSENISDNTKTIIVQFKNPVKINCTRPNNNTRRSIHIGPGRAFYATGEIIGD
TRKAHCNISEKQWYDTLIKIATEFKDQYNKTVGFQPSAGGDLEITTHSFNCRGEFFYCNTTILFN
HTRVNDILSNNHTRENDTITLPCRIKQIVNMWQRVGQAMYAPPIAGKIQCNSNITGLLLTIDGGE
GNESETLRPGGGDMRDNWRSELYKYKVVKIEPLGVAPTKAKRQVVQREKRAVGMGAMFLGF
LGAAGSTMGAASITLTVQARNLLSGIVQQQSNLLKAIEAQQHLLQLTVWGIKQLQARILAVERYL
KDQQLLGIWGCSGKLICPTTVPWNLSWSNKSQDEIWGNMTWMEWEKEIGNYTDTIYRLIESAQ
NQQEKNEQDLLALDKWDNLWNWFSITRWLWYIEIFIMIIGSLIGLRIVFTVLSIINRVRQGYSPLSL
QTLIPNSRGPERPGGIEEEGGEQDKDRSIRLVSGFLALAWDDFRSLCVFSYHCLRNFILIAARTV
DKGLKRGWEVLKYLWNLAQYWGQELKNSAISLLDRTAIAVAEGTDRIIEILQRAGRAVLNIPRRI
RQGLERALL*
SEQ ID NO: 101
>F2.CM.97.CM53657.AF377956
MRVREMQRNWQHLGKWGLLFLGILIICNAADNLWVTVYYGVPVWKEATTTLFCASDAKAYERE
IHNVWATYACVPTDPNPQELVLGNVTENFNMWKNNMVDQMHEDIISLWDQSLKPCVQITPLCV
TLNCTDVPVNITNGNSTLDNITLEEQGEIKNCSFNITTEINDIKKKESAIFYRLDVVPINNSTSEYRL
LSCNTSTVTQACPKVSFDPIPIHYCAPAGFAILKCNDKEFNGTGLCRNVSTVQCTHGIKPVVSTQ
LLLNGSLAEGDIVIRSENISDNAKTIIVQFNRSVAINCTRPTNITRRSMRIGPGRVFYATGTVLGDI
RKAYCTINGTLWNKTLEGVAKEVQSHLNKSITFAPSSGGDLEVTTHSFNCRGEFFYCNTVALFN
ATNMTNAMNRSNGIITLPCRIRQIVNMWQRVGRAMYAAPIAGQIQCNSSITGLILTRDGGKNNT
NNDTLRPGGGDMRDNWRSELYKYKVVKIEPLGVAPTKAKRQVVKREREKRAVGIGAVLLGFL
GAAGSTMGAASMTLTVQARQLLSGIVQQQNNLLKAIEAQQHLLQLTVWGIKQLQARILAVERYL
KDQQLLGIWGCSGKLICTTNVPWNSSWSNKSQNEIWENMTWMQWEKEISNYTGTIYKLIENAQ
NQQEKNEQDLLALDKWDNLWSWFTITNWLWYIKLFIMIVGGLIGLRIVFAVLAVINRVRQGYSPL
SLQTLTPSRREPERPGGIEEEGGEQDKTRSVRLVSGFLALAWDDLRSLCLFSYRHLRDFILIAA
RTVNKGLIRGWEILKYLGNLAQYWGREIKNSAIDLLNTTAIVVAEGTDRIIEVLQRAGRAILHIPRR
IRQGAERALL*
SEQ ID NO: 102
>G.BE.6.DRCBL.AF084936
MRVKGIQRNWQHLWNWGILILGLVIICSAEKLWVTVYYGVPVWEDANAPLFCASDAKAHSTES
HNIWATHACVPTDPSPQEINMRNVTENFNMWKNNMVEQMHEDIISLWDESLKPCVKLTPLCVT
LNCTEINNNSTRNITEEYRMTNCSFNMTTELRDKKKAEYALFYRTDVVPINEMNNENNGTNST
WYRLTNCNVSTIKQACPKVTFEPIPIHYCAPAGFAILKCVDKKFNGTGTCNNVSTVQCTHGIKPV
VSTQLLLNGSLAEKDIIISSENISDNAKVIIVHLNRSVEINCTRPNNNTRRSVAIGPGQAFYTTGEVI
GDIRKAHCNVSWTKWNETLRDVQAKLQEYFINKSIEFNSSSGGDLEITTHSFNCGGEFFYCNTS
GLFNNSILKSNISENNDTITLNCKIKQIVRMWQRVGQAMYAPPIAGNITCRSNITGLILTRDGGDN
NSTSEIFRPGGGDMKNNWRSELYKYKTVKIKSLGIAPTRARRRVVEREKRAVGVGAIFLGFLGT
AGSTMGAASITLTVQVRQLLSGIVQQQSNLLRAIEAQQHLLQLTVWGIKQLRARVLALERYLKD
QQLLGIWGCSGKLICTTNVPWNTSWSNKSYNEIWENMTWIEWEREIDNYTYHIYSLIEQSQIQQ
EKNEQDLLALDQWASLWSWFSISNWLWYIRIFVMIVGGLIGLRIVFAVLSIVNRVRQGYSPLSFQ
TLLHHQREPDRPAGIEEGGGEQDRDRSIRLVSGFLALAWDDLRSLCLFSYHRLRDFILIAARTVE
LLGRNSLKGLRLGWEALKYLWNLLLYWARELKNSAINLLDTIAIAVANWTDRVIEVAQRAGRAVL
NIPRRIRQGLERALL*
SEQ ID NO: 103
>G.KE.93.HH8793_12_1.AF061641
MRVKGIERNWQHLWKWGTLILGLVIICSASNNLWVTVYYGVPVWEDAKTTLFCASDAKAYSTE
RHNIWATHACVPTDPDPQEIPLGNVTENFNVWKNDMVEQMHEDIISLWDESLKPCVKLTPLCV
TLNCTDANVTTVANESVSAQEIKNCSFNITTEIRDRKRKEYALFYRLDVIPINDDSSNSTGNYSN
YRLINCNVSTIKQACPKVDFDPIPIHYCAPGGFAILKCKEKEFNGTGPCQNVSTVQCTHGIKPVV
STQLLLNGSLAEGEIIIKSENITDNTKVIIVQLNETVEITCVRPNNNTRKSIHLGPGQALYATGDIIG
NIRQAHCDVSGRNWSNMIEKVKAQLRKIFNKTITFDSSAGGDLEITTHSFNCRGEFFYCNTSGL
FNNETISNGTITLPCGXKQIVRLWQRVGQAMYSPPIARNITCKSNITGLLLTRDGGNANNASETE
TFRPAGGNMKDNWRNELYKYKVVKIKPLGVAPTKARRRVVGREKRAVGVGAVFLGFLGAAGS
TMGAASITLTVQVRQLLSGIVQQQSNLLRAIEAQQHLLQLTVWGIKQLQARVLALERYLRDQQL
LGIWGCSGKLICTTNVPWNASWSNKTYNDIWDNMTWIQWDREISNYTQQIYSLIEESQNQQEK
NEQDLLALDNWASLWTWFDITKWLWYIKIFIMIVGGLISLKIIFAVLSIVNRVRKGYSPLSFQTLTH
HQREPDRPERIEEEGGEQDKDRSIRLVSGFLALAWDDLRSLCLFSYHRLRDFILIAARTVELLGH
NSLKGLRLGWEGLKYLWNLLLYWGRELKNSAINLLDTIAIAVANWTDRVIEIVQRAFRAFLNIPTR
IRQGLERALL*
SEQ ID NO: 104
>G.NG.92.92NG083.U88826
MRVKGIQRNWQHLWKWGTLILGLVIICSASDNLWVTVYYGVPVWEDADTPLFCASDAKSYSSE
KHNVWATHACVPTDPNPQEIAIENVTENFNMWKNNMVEQMQEDIISLWEESLKPCVKLTPLCIT
LNCTNVNSANHTEANNTVENKEEIKNCSFKITTERGGKKKEEYALFYKLDVVPISNGNKTSYRLI
HCNVSTIKQACPKVNFDPIPIHYCAPAGFAILKCRDKEYNGTGPCKNVSTVQCTHGIKPVVSTQL
LLNGSLAEEDIRIRSENFTDNTKVIIVQLNNSIEINCIRPNNNTRKSIPIGPGQAFYATGDIIGDIRQA
HCNVSRIKWREMLKNVTAQLRKIYNNKNITFNSSAGGDLEITTHSFNCRGEFFYCNTSGLFNNN
ISNINNETITLPCKIKQIVRMWQKVGQAMYALPIAGNLVCKSNITGLILTRDGGNNNDSTEETFRP
GGGDMRDNWRSELYKYKTVKIKSLGVAPTRARRRVVEREKRAVGLGAVFLGFLGAAGSTMGA
ASITLTAQVRQLLSGIVQQQSNLLRAIEAQQHLLQLTVWGIKQLQSRVLAIERYLKDQQLLGIWG
CSGKLICTTNVPWNTSWSNKSYNEIWDNMTWLEWEREIHNYTQHIYSLIEESQNQQEKNEQDL
LALDKWASLWNWFDISNWLWYIRIFIMIVGGLIGLRIVFAVLSIVNRVRQGYSPLSFQTLTHHQR
EPDRLGKTEEGGGEQDRDRSTRLVSGFLALAWDDLRSLCLFSYHRLRDLVLIAARTVELLGRS
SLKGLRLGWEGLKYLWNLLLYWGRELKNSAINLLDTIAIATANGTDRVIEVAQRAYRAILNVPTRI
RQGLERALL*
SEQ ID NO: 105
>G.PT.x.PT2695.AY612637
MRVRGTQTNWQLLWKWGTLILGLVIICSASNNLWVTVYYGVPVWEDADTTLFCASDAKAYSTE
SHNVWATHACVPTDPNPQEIPMKNVTENFNMWKNNMVEQMHEDIISLWDESLKPCVKLTPLC
VTLTCTNVTINVTSSSNSSVTSTSTVGSTTSTSTVGSTASTSTVGSTAGYREELKNCSFNITTEIK
DRRKQEYALFYKLDIVPINDGRNSSANNYRLINCNVSTIKQACPKVSFDPIPIHYCAPAGFAILKC
RDKEFNGTGLCKNVSTVQCTHGIKPVVSTQLLLNGSLAEGEIMIRSENITNNAKNIIVQLNETVPI
TCARPSNNTRKSIRFGPGQAFYATDAIIGDIRQAHCNISRIRWENMKQNVTAKLEKIFKKNITFNP
PAGGDLEITTHSFICRGGFFYCNTSALFNSSSLSNSNSSNDTITLPCRIKQIVRMWQRVGQAMY
APPIEGNITCMSNITGLLLTRDGGENNGTNETEIFRPVGGDMRDNWRSELYKYKVVKIKPLGVT
PTRARRRVVGREKRAVGLGAVLLGFLGTAGSTMGAASITLTVQVRQLLSGIVQQQSNLLRAIEA
QQHLLQLTVWGIKQLQARVLAVERYLKDQQLLGIWGCSGRLICTTNVPWNASWSNKSYNQIW
DNLTWVQWEREISNYTQQIYTLLEESQNQQEKNEQDLLALDKWADLWNWFDISRWLWYIKIFI
MIVGGLIGLRIVFAVLSIINRVRKGYSPLSFQTLTHHQREPDRPGRIEEGDGEQDRDKSIRLVSG
FLALAWDDLRSLCLFSYHHLRDFILIAARTVELLGRSSLKGLSLGWEGLKYLWNLLLYVVGRELK
NSAINLIDTVAIAVANWTDRAIEVVQRVGRAILNIPVRIRQGLERLLL*
SEQ ID NO: 106
>H.BE.93.VI991.AF190127
TRVMETQRNYPSLWRWGTLILGMLLICSVVGNLWVTVYYGVPVWKEAKTTLFCASDAKAYDTE
RHNVWATHACVPTDPNPQEMVLENVTETFNMWVNDMVEQMHTDIISLWDQSLKPCVKLTPLC
VTLDCSSVNATNVTKSNNSTDINIGEIQEQRNCSFNVTTAIRDKNQKVHALFYRADIVQIDEGER
NKSDNHYRLINCNTSVIKQACPKVSFEPIPIHYCAPAGFAILKCNGKKFNGTGPCTNVSTVQCTH
GIRPVVSTQLLLNGSLAEVEEVIIRSKNITDNTKNIIVQLNEPVQINCTRTGNNTRKSIRIGPGQAF
YATGDIIGDIRRAYCNISGKQWNETLHKVITKLGSYFDNKTIILQPPAGGDIEIITHSFNCGGEFFY
CNTTKLFNSTWTNSSYTNDTYNSNSTEDITGNITLQCKIKQIVNMWQRVGQAMYAPPIRGNITCI
SNITGLILTFDRNNTNNVTFRPGGGDMRDNWRSELYKYKVVKIEPLGVAPTEARRRVVEREKR
AVGMGAFFLGFLGAAGSTMGAASITLTVQARQLLSGIVQQQSNLLRAIQAQQHMLQLTVWGIK
QLQARVLAVERYLKDQQLLGIWGCSGKLICTTNVPWNSSWSNKSLDEIWDNMTWMEWDKQIN
NYTDEIYRLLEVSQNQQEKNEQDLLALDKWANLWNWFSITNWLWYIRIFIMIVGGIIGLRIVFAVL
SIVNRVRQGYSPLSLQTLIPNQRGPDRPREIEEEGGEQDRDRSIRLVNGFLPLVWEDLRNLCLF
SYRRLRDLLSIVARTVELLGRRGWEALKLLGNLLLYWGQELKNSAISLLNTTAIAVAEGTDRIIEL
VQRAWRAILHIPRRIRQGFERALL*
SEQ ID NO: 107
>H.BE.93.VI997.AF190128
TRVMRNYPQWWRGGILLLGMLLIYSAAGNLWVTVYYGVPVWKEAKTTLFCASDAKAYEPEKH
NVWATHACVPTDPSPQEMVLANVTENFNMWDNDMVEQMQTDIISLWDQSLKPCVKLTPLCVT
LDCSNITRNDTNSSSTVNATSSPSANELTNCSFNVTTVIRDKQQRVHALFYRLDVVPIDETSNN
NNSNSTKYRLINCNTSVITQACPKVSFDPIPIHYCAPAGFAILKCNNKTFNGTGPCTNVSTVQCT
HGIKPVVSTQLLLNGSLAEGQVIIRSKNISDNTKNIIVQLDSPIEITCTRPNNNTRKGIHFGPGQAF
YATGDIIGNIRQAHCNVSEEKWNKTLQQIATQLSKYFVNRTLIFKPHSGGDLEVTTHSFNCRGEF
FYCNTSGLFNSSWTGDNINMPNDTGKNITLPCRIKQIVNMWQRVGQAMYAPPIKGSITCVSNIT
GLILTYDEDKGNNDNVTFRPGGGDMRDNWRSELYKYKVVKIEPLGVAPTEARRRVVEREKRA
VGMGAFFLGFLGAAGSTMGAASITLTVQARQLLSGIVQQQSNLLRAIQAQQHMLQLTVWGVKQ
LQARVLAVERYLKDQQLLGIWGCSGKLICTTNVPWNSTWSNKSLAEIWDNMTWMEWDRQIDN
YTEVIYRLLELSQTQQEQNEQDLLALDKWDSLWNWFSITNWLWYIKIFIIIVGALIGLRIIFAVLSIV
GRVRQGYSPLSFQTLIPNPRGPDRPEGIEEEGGEQDRGRSVRLVNGFLPIVWDDLRSLCLFSY
RLLRDSLLIVIRTVELLGRRGREALKYLWNLLQYWGQELKNSAINLLNTTAIVVAEGTDRIIEIVQR
AWRAVLHIPRRIRQGLERILL*
SEQ ID NO: 108
>H.CF.90.056.AF005496
TRVMETQRNYPSLWRWGTLILGMLLICSAAQNLWVTVYYGVPVWKEAKTTLFCASDAKAYETE
KHNVWATHACVPTDPNPQEMVMENVTESFNMWENNMVEQMHTDIISLWDQSLKPCVKLTPL
CVTLNCTNVRNNTSNSTSSMEAGGELTNCSFNVTTVLRDKQQKVHALFYRLDVVPIDNNSTQY
RLINCNTSVITQACPKVSFEPIPIHYCAPAGFAILKCNNKTFNGTGLCTNVSTVQCTHGIRPVVST
QLLLNGSLAEEQIIIRTKNISDNTKNIIVQLKTPVNITCTRPNNNTRTSIHLGPGRAFYATGDIIGDIR
QAHCNISRTDWNKTLHQVVTQLGIHLNNRTISFKPNSGGDMEVRTHSFNCRGEFFYCNTSGLF
NSSWEMHTNYTSNDTKGNENITLPCRIKQIVNMWQRVGRAMYAPPIQGNIMCVSNITGLILTIDE
GNASAENYTFRPGGGDMRDNWRSELYKYKVVKIEPLGIAPTKTRRRVVEREKRAVGMGASFL
GFLGAAGSTMGAASITLTVQARQLLSGIVQQQSNLLRAIQARQHMLQLTVWGIKQLQARVLAVE
RYLRDQQLLGIWGCSGKLICTTNVPWNSSWSNKSQSEIWDNMTWMEWDKQISNYTEEIYRLL
EVSQTQQEKNEQDLLALDKWASLWTWFDISHWLWYIKIFIMIVGGLIGLRIIFAVLSIVNRVRQGY
SPLSFQTLVPNPRGPDRPEGTEEGGGEQDRDRSVRLVNGFLPVVWDDLRSLSLFSYRLLRDL
LLIVVRTVELLGRRGREALKYLWNLLQYWGQELKNSAIDLLNTTAIAVAEGTDGIIVIVQRAWRAI
LHIPRRIRQGFERSLL*
SEQ ID NO: 109
>J.CD.97.J_97DC_KTB147.EF614151
TKVMETQMNWKNLWKWGLMIFGMLMICNGAEKLWVTVYYGVPVWKDAKTTLFCASDAKSYS
TEKHNVWATHACVPTDPNPREITMENVTENFNIWKNDMVEQMHDDIISVWDESLKPCVKITPL
CVTLNCTEATINNSTDNSNTTSPSPTITNPLGMKNCSFNITTEIGDRRKKEYALFYKQDVVSIDN
SNSSYILINCNTSVIKQACPKVSFEPIPIHYCAPAGFAILKCNDNKFNGTGPCKNVSTVQCTHGIK
PVVSTQLLLNGSIAEKEVIIKSKNISDNAKTIIVQLNQTVEINCTRPANNTRKGIPIGPGQVLYATG
AVIGNIRQAHCNISGVKWNNTLHKVAEELREQEHFKNKTIVFAPANSGGDIEIMMHTFNCGGEF
FYCNTSILFNSSWDENSTVTINETVTLPCKIRQIVRMWQRVGQAIYAPPIAGNITCTSNITGLLLT
RDGGNTKESNSSETFRPTGGDMKDNWRNELYKYKIVKVEPLGVAPTRAKRRVVEREKRAIVG
MGAVFLGFLGTAGSTMGAASIALTVQARQLLSGIVQQQSNLLKAIEAQQHLLRLTVWGIKQLQA
RILAVERYLKDQQLLGIWGCSGKLICTTNVPWNSSWSNKSHDEIWNNMTWVEWEREIDNYTRII
YNLIEVSQNQQEKNEQDLLALDKWTSLWSWFKISNWLWYIRIFIMIVGGLIGLRIIFAVLAIVNRVR
QGYSPLSLQTLIPNPTGVDRPGEIEEGGGEQGRTRSIRLVSGFLALAWDDLRSLCLFSYHRLRD
FVLIVARTVETLGRRGWEILKYLGNLVCYWGQELKNSAISLLNATAIAVAAGTDRIIEIVQGIFRAIL
HIPRRIRQ
SEQ ID NO: 110
>J.SE.93.SE7887.AF082394
TRVMETQKNWQTLWRGGLMIFGMLMICKAKEDLWVTVYYGVPVWKDAKTTLFCASDAKAYST
EKHNVWATHACVPTDPSPQEMNLPNVTENFNMWKNDMVDQMQEDIISVWDESLKPCVKITPL
CVTLNCSNITSNSNTTSNSSVSSPDIMTNCSFNITTEIRNKRKQEYALFYRQDVVPIDSNNKNYIL
INCNTSVIKQACPKVSFQPIPIHYCAPAGFAILKCNDKNFNGTGSCKNVSTVQCTHGIKPVVSTQ
LLLNGSIAEGDIIIRSENISDNAKNIIVQLNKTVEIVCYRPNNNTRKGIHMGPGQVLYATGEIIGNIR
ETHCNISERDWSNTLRRVATKLREHFNKTINFTSPSGGDIEIVTHSFNCGGEFLYCNTSKLFNSS
WDKNSIEATNDTSXATITIPCKIKQIVRMWQRTGQAIYAPPIAGNITCTSNITGLLLTRDGGNRGN
GSENGTETFRPTGGNMKDNWRSELYKYKVVEIEPLGVAPTKAKRRVVEREKRAVGIGAVFLGF
LGTAGSTMGAASITLTVQVRQLLSGIVQQQSNLLKAIEAQQHLLKLTVWGIKQLQARVLAVERYL
KDQQLLGIWGCSGKLICTTNVPWNASWSNKSYEDIWENMTWIQWEREINNYTGIIYSLIEEAQN
QQENNEKDLLALDKWTNLWNWFNISNWLWYIKIFIMIIGGLIGLRIIFAVLAIVNRVRQGYSPLSF
QTLIPNPTEADRPGGIEEGGGEQGRTRSIRLVNGFLALAWDDLRNLCLFSYHRLRDFVLIAART
VGTLGLRGWEILKYLVNLVWYWGQELKNSAISLLNTTAIAVAEGTDRIIEIAQRAFRAILHIPRRIR
QGLERALL*
SEQ ID NO: 111
>J.SE.94.SE7022.AF082395
TRVMETQTSWLSLWRWGLMIFGMLMICSARENLWVTVYYGVPVWRDAKTTLFCASDAKAYST
EKHNVWATHACVPTDPNPQEMSLPNVTENFNMWKNDMVDQMQEDIISVWDESLKPCVKITPL
CVTLNCSDVNSNNSTDSNSSASNNSPEIMKNCSFNVTTEIRNKRKQEYALFYRQDVVPINSDN
KSYILINCNTSVIKQACPKVSFQPIPIHYCAPAGFAILKCNNKTFNGTGPCKNVSTVQCTHGIKPV
VSTQLLLNGSVAEGDIIIRSENISDNAKNIIVQLNDTVEIVCTRPNNNTRKGIHMGPGQVLYATGEI
IGDIRKAYCNISRKDWNNTLRRVAKKLREHFNKTIDFTSPSGGDIEITTHSFNCGGEFFYCNTST
LFNSSWDENNIKDTNSTNDNTTITIPCKIKQIVRMWQRTGQAIYAPPIAGNITCKSNITGLLLTRD
GGNRNGSENGTETFRPTGGNMKDNWRSELYKYKVVELEPLGVAPTKAKRRVVEREKRAVGIG
AVFLGFLGTAGSTMGAASITLTVQVRQLLSGIVQQQSNLLKAIXAQQHLLKLTVWGIKQLQARVL
AVERYLKDQQLLGIWGCSGKLICTTNVPWNASWSNKSYEDIWENMTWIQWEREINNYTGIIYSL
IEEAQNQQETNEKDLLALDKWTNLWNWFNISNWLWYIKIFIMIIGGLIGLRIIFAVLAIVNRVRQGY
SPLSFQTLIPNPTEADRPGGIEEGGGEQGRTRSIRLVNGFLALAWDDLRSLCLFSYHRLRDFVLI
AARTVGTLGLRGWEILKYLVNLVWYWGQELKNSAISLLNTTAIAVAEGTDRIIEIAQRAFRAILHIP
RRIRQGLERALL*
SEQ ID NO: 112
>K.CD.97.EQTB11C.AJ249235
MRAREIQRNWQHLGKRGILFLGILIICSAANNLWVTVYYGVPVWKEATTTLFCASDAKAYETEV
HNVWATHACVPTDPNPQEVVLENVTENFNMWKNNMVEQMHTDIISLWDESLKPCVKLTPLCV
TLTCTNVTNNRTNANKNDTNINATVTSTDEIKNCSFNITTELKDKKKRVSALFYKLDIVQIKQSEIN
QSESEDRLINCNTSTVTQACPKVSFEPIPIHYCAPAGFAILKCNNNTCNGTGPCTNVSTVQCTH
GIKPVVSTQLLLNGSLAEEEIIIRSEDITKNTKNIIVQLNEAVEINCTRPSNNTRKSIHIGPGRAFYA
TGDIIGDIRQAHCNISGGQWNKTVNQVKKELGKHFNKTIIFQPSSGGDPQVTRHIFNCRGEFSY
CDTTDTVDDTEEEEDTTITIPCRIKQIINMWQKVGQAIYAPPTAGNITCRSNITGMILTRDGGNDN
NTRTEETFRPGGGDMRDNWRSELYKYKVVQIEPLGIAPTRARRRVVQREKRAVGIGALFLGFL
GAAGSTMGAASITLTVQARQLLSGIVQQQNNLLRAIEAQQQMLQLTVWGIKQLRARVLAVERYL
RDQQLLGIWGCSGKLICTTNVPWNSSWSNKSQSEIWENMTWMQWEKEISNHTSTIYRLIEESQ
IQQEKNEQDLLALDKWASLWNWFDISNWLWYIKIFIMIVGGLIGLRIVFTVLSVVNRVRQGYSPL
SFQTLTPSPRGPDRPEGIEEGGGEQDKDRSVRLVSGFLALAWDDLRNLCLFSYRHLRDLVLIAT
RILDRGLKGSWEALKYLWNLILYWGQEIKNSAINLLNTTAIAVAEGTDRIIEIVYRAFRALLHIPRRI
RQGFERLLL*
SEQ ID NO: 113
>K.CM.96.MP535.AJ249239
MRVRGMQRNWQTLGNWGILFLGILIICSNADKLWVTVYYGVPVWKEATPTLFCASDAKAYEKE
VHNVWATHACVPTDPNPQEVEMENVTENFNMWKNNMVEQMHTDIISLWDESLKPCVELTPLC
VTLNCTDYKGTNSTNNATSTVVSPAEIKNCSFNITTEIKDKKKKESALFYRLDVLPLNGEGNNSS
TEYRLINCNTSTITQTCPKVTFEPIPIHYCAPAGFAILKCKDKRFNGTGPCKNVSTVQCTHGIKPV
VSTQLLLNGSLAEEEIIIRSENITDNTKNIIVQLNETVQINCTRPNNNTRKSIHMGPGKAFYTTGDII
GDIRQAHCNISGEKWNMTLSRVKEKLKEHFKNGTITFKPPNPGGDPEILTHMFNCAGEFFYCNT
TKLFNETGENGTITLPCRIKQIINMWQKVGKAIYAPPIAGSINCSSNITGMILTRDGGNNTHNETF
RPGGGDMRDNWRSELYKYKVVQIEPLGIAPTRARRRVVQREKRAVGLGAVFFGFLGAAGSTM
GAASITLTVQARQLLSGIVQQQSNLLRAIEAQQHLLQLTVWGIKQLRARILAVERYLKDQQLLGI
WGCSGKLICTTNVPWNSSWSNKSWEEIWNNMTWMEWEKEIGNYSDTIYKLIEESQTQQEKNE
QDLLALDKWASLWNWFDITKWLWYIKIFIMIIGGLIGLRIAFAVLSVVNRVRQGYSPLSFQTLIPTS
RGADRPEGIEEEGGEQDKNRSVRLVSGFLALAWDDLRNLCLFSYRQLRNLILIVTRILERGLRG
GWEALKYLWNLVQYWSQELKNSAISLLNTTAIAVAGGTDRIIEIGQRAFRALLHIPRRIRQGLER
ALL*
SEQ ID NO: 114
>N.CM.02.DJO0131.AY532635
VRMMGMQIGWPFFCLMISLTIGSKKYWATVYYGVPVWRDVETVLFCASDAKAHSTEAHNIWAT
QACVPTDPNPQEVPLDNVTEPFNMWENKMAEQVQEDIISLWEQSLKPCVKLTPLCVTMNCSN
SNGNRTTDEKEKPGNGTDLEARHMKNCSFNITTEIHDKKKQAYSLFYVEDVVPLNDGNNSTYR
LINCNTTAVTQACPKTTFEPIPIHYCAPPGFAIMKCNEANFNGTGECKNVSTVQCTHGIKPVIST
QLILNGSLDKDIVIRNNSGGNLLVQWNEIVTMNCTRPGNNTGGQVQIGPAMTFYNIEKIVGDIRQ
AHCNVSNEWRSMWNKTKEKIKSLLGNNITFKAQEKNGGDPEVTHLMFNCGGEFIYCNTSRLFN
ESMNTNGTNGTITLPCRIRQIVNLWTRVGKGIYAPPIRGNLTCNSTITGLILEHSGGSNGTVYPT
GGNMVNLWRQELYKYKTVSIEPIGVAPGKAKRRTVSREKRAAFGLGALFLGFLGAAGSTMGAA
SITLTVQARTLLSGIVQQQNNLVRAIEAQQHLLQLSIWGIKQLRAKVLAIERYLRDQQILSLWGCS
GKTICYTTVPWNETWSNNTSYDXIWGNLTWQQWDRKVRNYSGVIFELIXKAQEQQNTNEKSLL
ELDQWASLWNWFSITNWLWYIKIAIMVVAGIIGIRIISVIITIIARVRQGYSPLSLQTLIPTTTTRGPD
RPEETEEGVGGQGRDRSVRLVSGFLTLVWEDFRNLLIFLYHRLTDSLLILQRTLELLGRSLIRGL
QLLNELRTRLWGIIAYWGKELKDSAISLLNTIAIVVAEGTDRLIELAQRIGRGILHIPRRIRQGLERA
LL*
SEQ ID NO: 115
>N.CM.95.YBF30.AJ006022
MKVMGMQSGWMGMKSGWLLFYLLVSLIKVIGSEQHWVTVYYGVPVWREAETTLFCASDAKA
HSTEAHNIWATQACVPTDPNPQEVLLPNVTEKFNMWENKMADQMQEDIISLWEQSLKPCVKLT
PLCVTMLCNDSYGEERNNTNMTTREPDIGYKQMKNCSFNATTELTDKKKQVYSLFYVEDVVPI
NAYNKTYRLINCNTTAVTQACPKTSFEPIPIHYCAPPGFAIMKCNEGNFSGNGSCTNVSTVQCT
HGIKPVISTQLILNGSLNTDGIVIRNDSHSNLLVQWNETVPINCTRPGNNTGGQVQIGPAMTFYNI
EKIVGDIRQAYCNVSKELWEPMWNRTREEIKKILGKNNITFRARERNEGDLEVTHLMFNCRGEF
FYCNTSKLFNEELLNETGEPITLPCRIRQIVNLWTRVGKGIYAPPIRGVLNCTSNITGLVLEYSGG
PDTKETIVYPSGGNMVNLWRQELYKYKVVSIEPIGVAPGKAKRRTVSREKRAAFGLGALFLGFL
GAAGSTMGAASITLTVQARTLLSGIVQQQNILLRAIEAQQHLLQLSIWGIKQLQAKVLAIERYLRD
QQILSLWGCSGKTICYTTVPWNETWSNNTSYDTIWNNLTWQQWDEKVRNYSGVIFGLIEQAQ
EQQNTNEKSLLELDQWDSLWSWFGITKWLWYIKIAIMIVAGIVGIRIISIVITIIARVRQGYSPLSLQ
TLIPTARGPDRPEETEGGVGEQDRGRSVRLVSGFSALVWEDLRNLLIFLYHRLTDSLLILRRTLE
LLGQSLSRGLQLLNELRTHLWGILAYWGKELRDSAISLLNTTAIVVAEGTDRIIELAQRIGRGILHI
PRRIRQGLERALI*
SEQ ID NO: 116
>N.CM.97.YBF106.AJ271370
RRVMGMQSGWPFFCLLISLTIGSDPHWVTVYYGVPVWRDAETVLFCASDAKAHSTEAHNIWA
TQACVPTDPNPQEVLLTNVTEYFNMWENKMAEQMQEDIISLWEQSLKPCVKLTPLCVTMLCNN
SNGNSAGNSTTNRTEDLEDRQMKNCSFNITTEIRDRKKQVYSLFYVEDVVPIKDGTDNNTYRLI
NCNTTAVTQACPKTTFEPIPIHYCAPPGFAIMKCNEGNFSGNGSCTNVSTVQCTHGIKPVISTQL
ILNGSLDTDDIVIRHHGGNLLVQWNETVSINCTRPGNNTGGQVQIGPAMTFYNIEKIVGDVRQA
YCNVSEEWGSMWNKTKKKIKRLLGNNTTFKAQDKNGGDLEVTHLMFNCXGEFFYCNTSRLFN
ESENKTNKTIILPCRIKQIVXLWTRVXKGIYAPPIRGNLSCXSSITGLILEHSGENGNKTVYPSGG
NMVNLWRQELYKYKVVSIEPIGVAPGKAKRRTVSREKRAAFGLGALFLGFLGAAGSTMGAASIT
LTVQARTLLSGIVQQQNNLLRAIEAQQHLLQLSIWGIKQLRAKVLAIERYLRDQQILSLWGCSGK
TICYTTVPWNDXWSSNTSYDTIWXNLTWQQWDRKVRNYSGVIFDLIEQAQEQQNTNEKALLEL
DQWASLWNWFDITKWLWYIKIAIMVVAGIIGIRIISAIITIIARVRQGYSPLSLQTLIPTAARGPDRP
EETEEGVGGQDRGRSVRLVSGFLALIWEDLRNLLIFLYHRLADSLLIIRRTLEILGQSLSRGLQLL
NELRIRLWGIIAYWGKELKDSAISLLNTTAIVVAEGTDRFIELAQRIGRGILHIPRRIRQGLERALL*
SEQ ID NO: 117
>O.BE.87.ANT70.L20587
MKAMEKRNKKLWTLYLAMALITPCLSLRQLYATVYAGVPVWEDATPVLFCASDANLTSTEKHNI
WASQACVPTDPTPYEYPLHNVTDDFNIWKNYMVEQMQEDIISLWDQSLKPCVQMTFLCVQME
CTNIAGTTNENLMKKCEFNVTTVIKDKKEKKQALFYVSDLMELNETSSTNKTNSKMYTLTNCNS
TTITQACPKVSFEPIPIHYCAPAGYAIFKCNSTEFNGTGTCRNITVVTCTHGIRPTVSTQLILNGTL
SKGKIRMMAKDILEGGKNIIVTLNSTLNMTCERPQIDIQEMRIGPMAWYSMGIGGTAGNSSRAA
YCKYNATDWGKILKQTAERYLELVNNTGSINMTFNHSSGGDLEVTHLHFNCHGEFFYCNTAKM
FNYTFSCNGTTCSVSNVSQGNNGTLPCKLRQVVRSWIRGQSGLYAPPIKGNLTCMSNITGMIL
QMDNTWNSSNNNVTFRPIGGDMKDIWRTELFNYKVVRVKPFSVAPTRIARPVISTRTHREKRA
VGLGMLFLGVLSAAGSTMGAAATTLAVQTHTLLKGIVQQQDNLLRAIQAQQQLLRLSXWGIRQL
RARLLALETLLQNQQLLSLWGCKGKLVCYTSVKWNRTWIGNESIWDTLTWQEWDRQISNISSTI
YEEIQKAQVQQEQNEKKLLELDEWASIWNWLDITKWLWYIKIAIIIVGALVGVRVIMIVLNIVKNIR
QGYQPLSLQIPNHHQEEAGTPGRTGGGGGEEGRPRWIPSPQGFLPLLYTDLRTIILWTYHLLS
NLASGIQKVISYLRLGLWILGQKIINVCRICAAVTQYWLQELQNSATSLLDTLAVAVANWTDGIIA
GIQRIGTGIRNIPRRIRQGLERSLL*
SEQ ID NO: 118
>O.CM.91.MVP5180.L20571
MKVMKKNNRKSWSLYIAMALLIPCLSYSKQLYATVYSGVPVWEEAAPVLFCASDANLTSTEQH
NIWASQACVPTDPNPHEFPLGNVTDNFDIWKNYMVDQMHEDIISLWEQSLKPCEKMTFLCVQ
MNCVDLQTNKTGLLNETINEMRNCSFNVTTVLTDKKEQKQALFYVSDLSKVNDSNAVNGTTYM
LTNCNSTIIKQACPKVSFEPIPIHYCAPTGYAIFKCNDTDFNGTGLCHNISVVTCTHGIKPTVSTQL
ILNGTLSREKIRIMGKNITESAKNIIVTLNTPINMTCIREGIAEVQDIYTGPMRWRSMTLKRSNNTS
PRSRVAYCTYNKTVWENALQQTAIRYLNLVNQTENVTIIFSRTSGGDAEVSHLHFNCHGEFFYC
NTSGMFNYTFINCTKSGCQEIKGSNETNKNGTIPCKLRQLVRSWMKGESRIYAPPIPGNLTCHS
NITGMILQLDQPWNSTGENTLRPVGGDMKDIWRTKLYNYKVVQIKPFSVAPTKMSRPIINIHTPH
REKRAVGLGMLFLGVLSAAGSTMGAAATALTVRTHSVLKGIVQQQDNLLRAIQAQQHLLRLSV
WGIRQLRARLQALETLIQNQQRLNLWGCKGKLICYTSVKWNTSWSGRYNDDSIWDNLTWQQ
WDQHINNVSSIIYDEIQAAQDQQEKNVKALLELDEWASLWNWFDITKWLWYIKIAIIIVGALIGIRV
IMIILNLVKNIRQGYQPLSLQIPVPHRQEAETPGRTGEEGGEGDRPKWTALPPGFLQQLYTDLR
TIILWTYHLLSNLISGIRRLIDYLGLGLWILGQKTIEACRLCGAVMQYWLQELKNSATNLLDTIAVS
VANWTDGIILGLQRIGQGFLHIPRRIRQGAERILV*
SEQ ID NO: 119
>O.SN.99.SEMP1300.AJ302647
MKVMEKRNRKLGILCMVMALITPCLSHNQHYATVYAGVPVWEEATPVLFCASDVNLTSTEQHN
IWASQACVPTDPSPYEYPLKNVTDNFNIWENYMVEQMQEDIISLWEQSLKPCVQMTFLCVQMN
CTNVNDETNSSVKNDTSSSENLMKKCEFNVTTVLKDKKEKQQALFYVSDLMKVNENNDTMYT
LINCNSTTIKQTCPKVSFEAIPIHYCAPAGYAIFKCNNTGFNGTGPCTNVTVVTCTHGIRPTVSTQ
LILNGTISEGKIRIMGKNISDTGKNIIVTINSTINMTCERPGNQTVQKILTGPVAWYSMGLKNNLTN
SRAASCKYNSSVWEEALKQTAERYLELMNNTNTVNITFNHSTGGDPEVTHLHFNCHGEFFYCN
TSQMFNYTFSCTRTNCIRQSNSSINGTISCRIKQVVRSWIQGGSGLYAPPRPGYLTCNSSITGMI
LQLDKTWNRTNNSESTFRPIGGDMKDIWRTELFKYKVVKIKPFSVAPTKIARPVIGTGTRREKRA
VGLGMLFLGVLSAAGSTMGAAATTLAVQTHTLMKGIVQQQDNLLRAIQAQQQLLRLSVWGIRQ
LRARLLALETLIQNQQLLNLWGCKGRLVCYTSVKWNRTWTNNNTDLDTIWGNLTWQEWDQQI
SNISATIYDEIQKAQVQQEHNEKKLLELDEWASIWNWLDITKWLWYIKIAIIIVGALIGVRIVMIVLN
LVRNIRHGYQPLSFQTPTHHQQPEAQAPGGTGEGGGERDRLRSIPSPQGFLPLLYTDLRTIILW
SYHLLSNLASGIQTVISHLGLGLWILGQKIISACRICIAVIQYWLQELQNSATSLLDTLAVAVANWT
DGIILGLQRIGRGILNIPRRIRQGLERALL*
SEQ ID NO: 120
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ
LQARILAVETLIQNQQRLNLWGCKGKLICTYSVKWNTSESNKSLEQIWNHTTWMEWDREINNYTSLI
HSLIEESQNQQEKNEQELLELDKWASLWNWFNITNWLWYIKLFIMIVGGLVGLRIVFAVLSIVNR
VRQGY
SEQ ID NO: 121
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

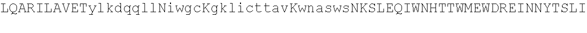

HSLIEESQNQQEKNEQELLELDKWASLWNWFNITNWLWYIKLFIMIVGGLVGLRIVFAVLSIVNR
VRQGY
SEQ ID NO: 122
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

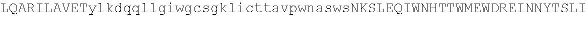

HSLIEESQNQQEKNEQELLELDKWASLWNWFNITNWLWYIKLFIMIVGGLVGLRIVFAVLSIVNR
VRQGY
SEQ ID NO: 123
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

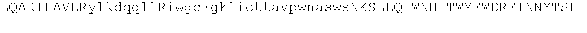

HSLIEESQNQQEKNEQELLELDKWASLWNWFNITNWLWYIKLFIMIVGGLVGLRIVFAVLSIVNR
VRQGY
SEQ ID NO: 124
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

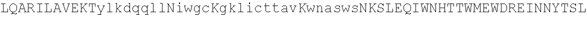

IHSLIEESQNQQEKNEQELLELDKWASLWNWFNITNWLWYIKLFIMIVGGLVGLRIVFAVLSIVNR
VRQGY
SEQ ID NO: 125
1. Group O 10-40:
wgikqlgarilaveTLIQNQQRLNLWGCGKLICYTSVKWNTSWSnksle
→
GGGGGTGCAAAGGTAAGCTAATTTGTTATACTAGTGTTAAATGGAACACCTCATGGAGC
AA TAAATCTCTG GAACAGATTT G
SEQ ID NO: 126
2. Group O pentamutant

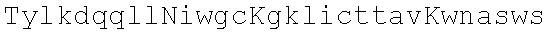

SEQ ID NO: 127
3. R10T single mutant:

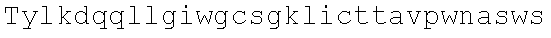

SEQ ID NO: 128
4. Heidmann double mutant

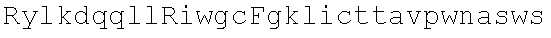

SEQ ID NO: 129
5. Pentamutants + E9K:

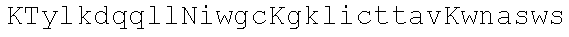

SEQ ID NO: 130
CKS-17: LQNRRGLDLLFLKEGGLC
SEQ ID NO: 131
CS-3: LQARVLAVERYLKDQQLLGIWGC,
SEQ ID NO: 132
HIV Gl9R: LQARVLAVERYLKDQQLLRIWGC,
and
SEQ ID NO: 133
HIV M/O chimera: LQARILAVETLIQNQQLLNLWGC
SEQ ID NO: 134-323
Examples of peptides of the present invention.

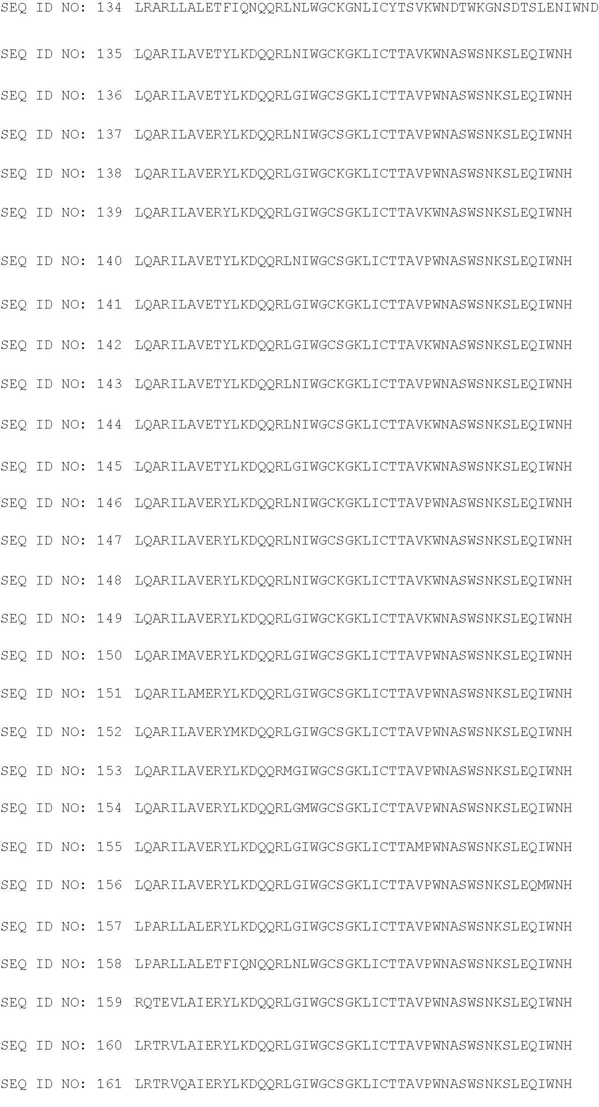

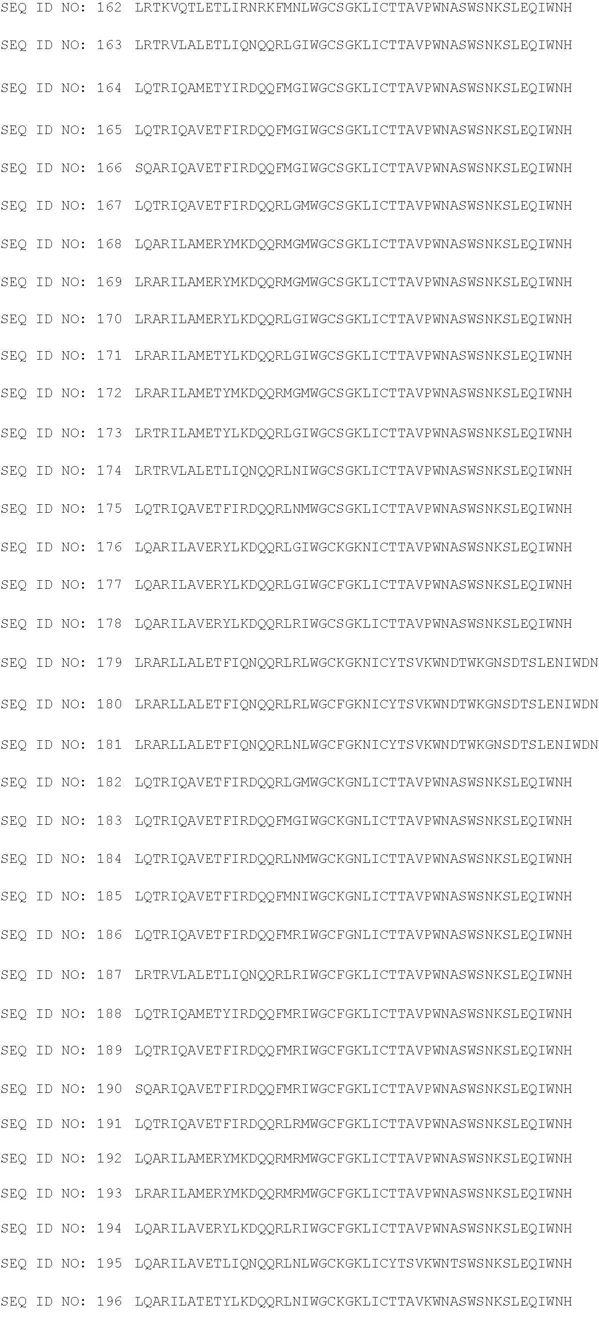

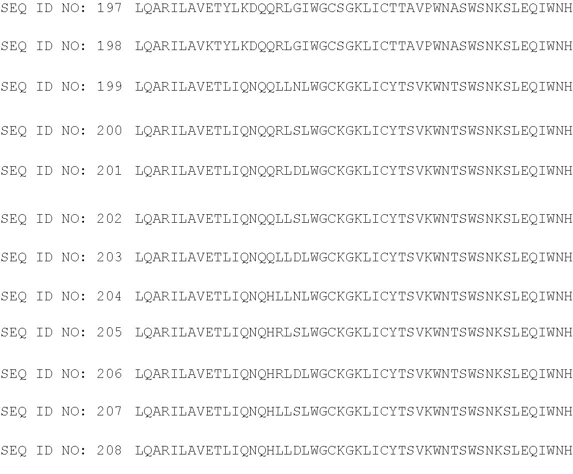

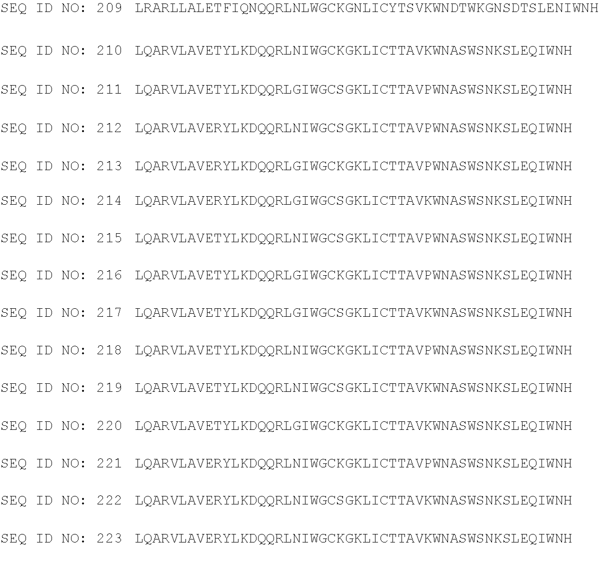

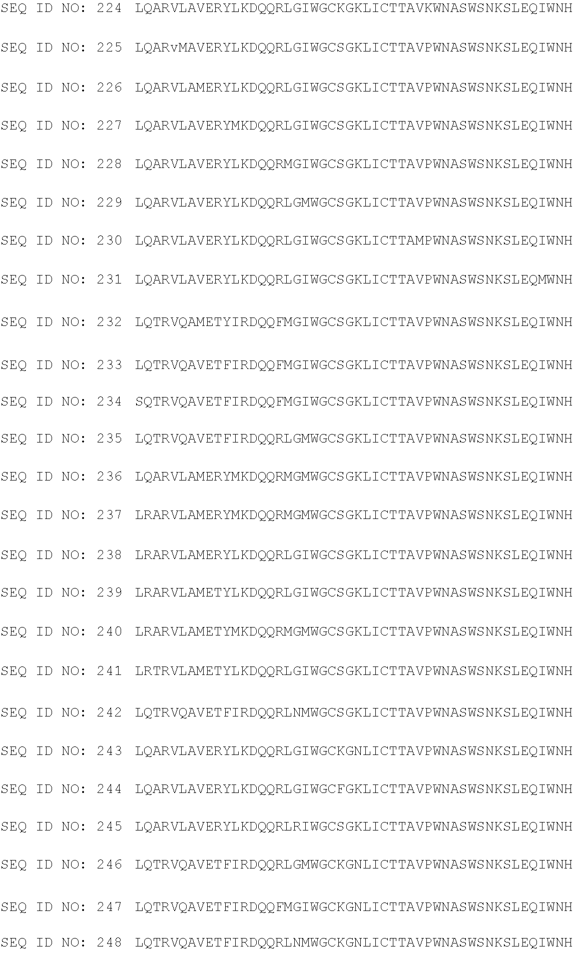

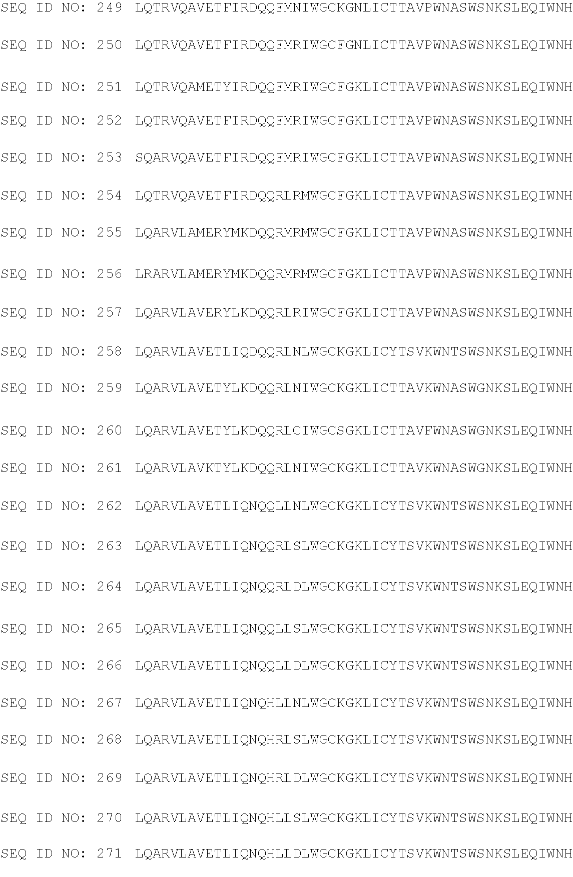

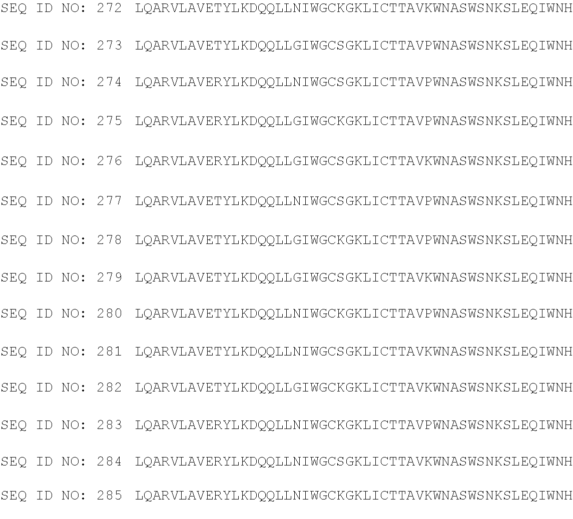

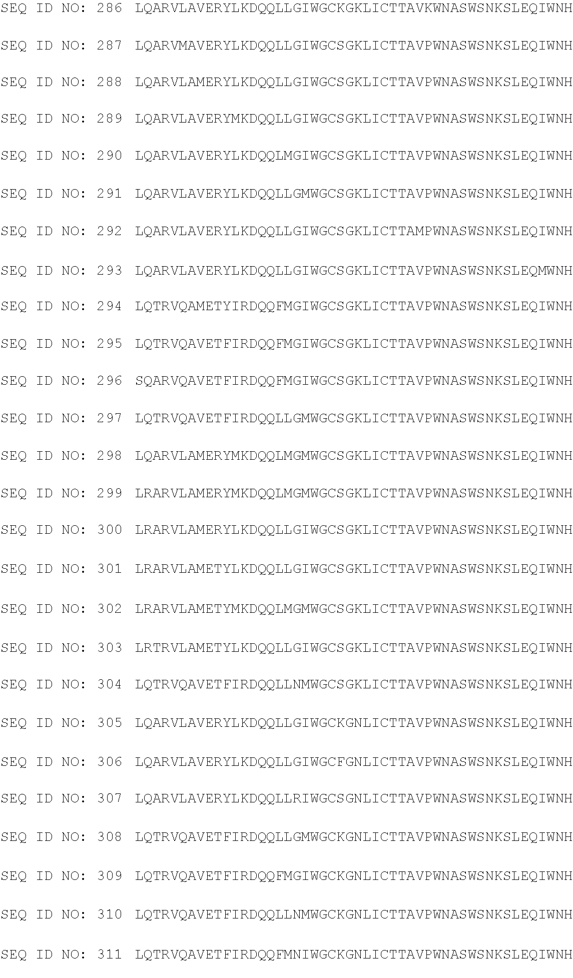

SEQ ID NO: 324
MRVKEKYQHLWRWGWRWGTMLLGMLMICSATEKLWVTVYYGVPVWKEATTTLFCA
SDAKAYDTEVHNVWATHACVPTDPNPQEVVLVNVTENFNMWKNDMVEQMHEDIISL
WDQSLKPCVKLTPLCVSLKCTDLKNDTNTNSSSGRMIMEKGEIKNCSFNISTSIRGKV
QKEYAFFYKLDIIPIDNDTTSYKLTSCNTSVITQACPKVSFEPIPIHYCAPAGFAILKCNN
KTFNGTGPCTNVSTVQCTHGIRPVVSTQLLLNGSLAEEEVVIRSVNFTDNAKTIIVQLN
TSVEINCTRPNNNTRKRIRIQRGPGRAFVTIGKIGNMRQAHCNISRAKWNNTLKQIASK
LREQFGNNKTIIFKQSSGGDPEIVTHSFNCGGEFFYCNSTQLFNSTWFNSTWSTEGS
NNTEGSDTITLPCRIKQIINMWQKVGKAMYAPPISGQIRCSSNITGLLLTRDGGNSNNE
SEIFRPGGGDMRDNWRSELYKYKVVKIEPLGVAPTKAKRRVVQREKRAVGIGALFLG
FLGAAGSTMGAASMTLTVQARQLLSGIVQQQNNLLRAIEAQQHLLQLTVWGIKQLQA

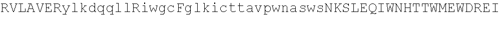

NNYTSLIHSLIEESQNQQEKNEQELLELDKWASLWNWFNITNWLWYIKLFIMIVGGLVG
LRIVFAVLSIVNRVRQGY
SEQ ID NO: 325
MRVKEKYQHLWRWGWRWGTMLLGMLMICSATEKLWVTVYYGVPVWKEATTTLFCA
SDAKAYDTEVHNVWATHACVPTDPNPQEVVLVNVTENFNMWKNDMVEQMHEDIISL
WDQSLKPCVKLTPLCVSLKCTDLKNDTNTNSSSGRMIMEKGEIKNCSFNISTSIRGKV
QKEYAFFYKLDIIPIDNDTTSYKLTSCNTSVITQACPKVSFEPIPIHYCAPAGFAILKCNN
KTFNGTGPCTNVSTVQCTHGIRPVVSTQLLLNGSLAEEEVVIRSVNFTDNAKTIIVQLN
TSVEINCTRPNNNTRKRIRIQRGPGRAFVTIGKIGNMRQAHCNISRAKWNNTLKQIASK
LREQFGNNKTIIFKQSSGGDPEIVTHSFNCGGEFFYCNSTQLFNSTWFNSTWSTEGS
NNTEGSDTITLPCRIKQIINMWQKVGKAMYAPPISGQIRCSSNITGLLLTRDGGNSNNE
SEIFRPGGGDMRDNWRSELYKYKVVKIEPLGVAPTKAKRRVVQREKRAVGIGALFLG
FLGAAGSTMGAASMTLTVQARQLLSGIVQQQNNLLRAIEAQQHLLQLTVWGIKQLQA

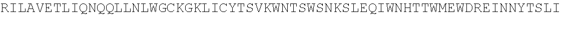

HSLIEESQNQQEKNEQELLELDKWASLWNWFNITNWLWYIKLFIMIVGGLVGLRIVFAV
LSIVNRVRQGY
SEQ ID NO: 326
1. Group O 10-40:

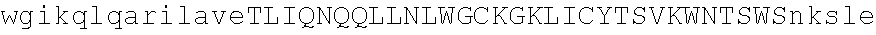

SEQ ID NO: 327
HIV WT-HXB2: WGIKQLQARVLAVERYLKDQQLLGIWGCSGKLICTTAVPWNASWSNKSLE
SEQ ID NO: 328
HIV G19R-HXB2: WGIKQLQARVLAVERYLKDQQLLRIWGCFGKLICTTAVPWNASWSNKSLE
SEQ ID NO: 329
HIV M/O-HXB2: WGIKQLQARVLAVETLIQNQQRLNLWGCKGKLICYTSVKWNTSWSNKSLE
SEQ ID NO: 330
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

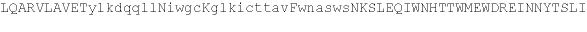

HSLIEESQNQQEKNEQELLELDKWASLWNWFNITNWLWYIKLFIMIVGGLVGLRIVFAVLSIVNR
VRQGY
SEQ ID NO: 331
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

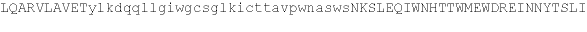

HSLIEESQNQQEKNEQELLELDKWASLWNWFNITNWLWYIKLFIMIVGGLVGLRIVFAVLSIVNR
VRQGY
SEQ ID NO: 332
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

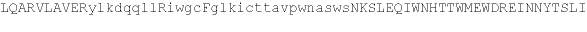

HSLIEESQNQQEKNEQELLELDKWASLWNWFNITNWLWYIKLFIMIVGGLVGLRIVFAVLSIVNR
VRQGY
SEQ ID NO: 333
MRVKEKYQHLWRWGWRWGTMLLGMLMICSATEKLWVTVYYGVPVWKEATTTLFCASDAKAY
DTEVHNVWATHACVPTDPNPQEVVLVNVTENFNMWKNDMVEQMHEDIISLWDQSLKPCVKLT
PLCVSLKCTDLKNDTNTNSSSGRMIMEKGEIKNCSFNISTSIRGKVQKEYAFFYKLDIIPIDNDTT
SYKLTSCNTSVITQACPKVSFEPIPIHYCAPAGFAILKCNNKTFNGTGPCTNVSTVQCTHGIRPV
VSTQLLLNGSLAEEEVVIRSVNFTDNAKTIIVQLNTSVEINCTRPNNNTRKRIRIQRGPGRAFVTI
GKIGNMRQAHCNISRAKWNNTLKQIASKLREQFGNNKTIIFKQSSGGDPEIVTHSFNCGGEFFY
CNSTQLFNSTWFNSTWSTEGSNNTEGSDTITLPCRIKQIINMWQKVGKAMYAPPISGQIRCSSN
ITGLLLTRDGGNSNNESEIFRPGGGDMRDNWRSELYKYKVVKIEPLGVAPTKAKRRVVQREKR
AVGIGALFLGFLGAAGSTMGAASMTLTVQARQLLSGIVQQQNNLLRAIEAQQHLLQLTVWGIKQ

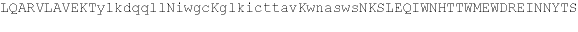

LIHSLIEESQNQQEKNEQELLELDKWASLWNWFNITNWLWYIKLFIMIVGGLVGLRIVFAVLSIVN
RVRQGY
SEQ ID NO: 334

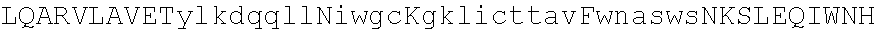

SEQ ID NO: 335

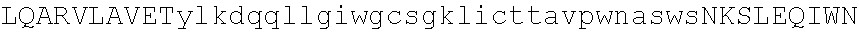

SEQ ID NO: 336

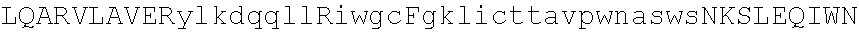

SEQ ID NO: 337

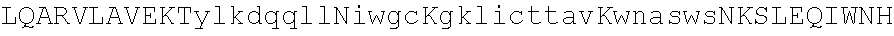

### Items

The following items 1-132 define preferred aspects and embodiments of the present invention.

Item 1. An HIV-1 envelope polypeptide comprising an amino acid sequence selected from the group of amino acid sequences consisting of: X(1-22)-C(23)-X(24-28)-C(29)-X(30-50), or any part thereof, or functional homolog or any amino acid sequence with at least 90% identity to said amino acid sequence or part thereof.

Item 2. The HIV-1 envelope polypeptide according to Item 1, wherein the amino acid residues in said amino acid sequence are selected from the groups of residues consisting of:

### X(1): L, S, R, P, F, A, V, M, and I; and

### X(2): Q, R, K, H, L, M, and P; and

### X(3): A, T, V, H, S, R, Q, G, M, and E; and

### X(4): R, K, G, E, T, S, C, M, and H; and

### X(5): V, I, L, D, A, S, F, M, and G; and

### X(6): L, Q, V, M, P, W, T, and I; and

### X(7): A, S, T, V, L, G, F, D, M, and E; and

### X(8): V, L, I, M, A, W, K, G, and E; and

### X(9): E, K, G, D, A, V, M, and F; and

### X(10): X; and

### X(11): Y, L, F, H, C, I, T, M, and N; and

### X(12): L, I, V, M, Q, P, T, Y, and A; and

### X(13): K, R, Q, G, S, E, H, W, T, V, M, N, Z, Y, A, P, and C; and

### X(14): D, N, G, E, Y, V, S, H, A, M, and I; and

### X(15): Q, R, H, K, P, L, M, and N; and

### X(16): Q, K, R, T, H, E, S, P, M, and L; and

### X(17): L, F, I, R, V, P, S, M, and H; and

### X(18): L, M, P, I, H, and S; and

### X(19): X; and

### X(20): I, L, M, V, S, F, T, D, A, R, P, and J; and

### X(21): W, R, G, F, L, M, and T; and

### X(22): G, D, A, R, M, and C; and

### X(24): X; and

### X(25): G, R, E, N, A, M, and D; and

### X(26): K, R, N, E, Q, T, S, I, M, and G; and

### X(27): L, H, I, T, V, F, R, Q, S, P, A, J, M, and Y; and

### X(28): I, V, T, L, R, F, and M; and

### X(30): T, P, Y, A, N, S, I, V, R, L, M and H; and

### X(31): T, S, P, N, M and I; and

### X(32): A, N, T, S, D, R, F, Q, P, I, E, V, M, L, K, H, C, and B; and

### X(33): V, A, L, M, G, R, and C; and

### X(34): X; and

### X(35): W, R, G, L, M, and P; and

### X(36): N, S, D, B, K, E, R, Q, M, and G; and

### X(37): S, T, A, N, D, V, I, E, Y, K, L, R, G, P, M, F, W, H, Q, B, and C; and

### X(38): S, T, N, I, G, R, L, C, A, W, M and E; and

### X(39): W, G, A, R, E, C, Y, V, S, M, and H; and

### X(40): X; and

### X(41): N, G, K, S, D, E, T, R, H, P, A, B, V, Q, Y, M, and I; and

### X(42): K, R, N, D, S, T, G, E, I, V, Y, Q, P, H, A, W, M, and C; and

### X(43): S, T, N, K, I, R, D, E, P, L, A, W, G, M, H, Y, F, V, and C; and

### X(44): L, Y, Q, F, E, H, S, V, K, M, T, I, W, N, D, R, P, A, and G; and

### X(45): D, E, N, S, T, K, G, L, A, Q, H, I, Y, B, R, V, P, M, F, W, Z, and C; and

### X(46): E, D, Q, Y, K, N, T, S, A, W, H, M, R, I, G, L, V, Z, F, B, and P; and

### X(47): I, D, E, M, G, T, Q, S, W, L, N, Y, K, V, R, F, A, P, and H; and

### X(48): W, I, T, N, D, E, L, G, S, Y, R, V, K, H, A, Q, M, and F; and

### X(49): D, N, E, G, W, Q, K, H, L, B, S, I, Y, T, A, R, M, Z, and V; and

### X(50): N, D, T, K, S, H, L, G, E, W, I, Q, M, R, B, Y, P, and A;

wherein the amino acids are designated by their conventional single letter code.

Item 3. The HIV-1 envelope polypeptide according to any of the preceding, wherein the amino acid residue in position X(10) is selected from the group consisting of: R, S, T, K, G, A, N, Q and I.

Item 4. The HIV-1 envelope polypeptide according to any of the preceding, wherein the amino acid residue in position X(19) is selected from the group consisting of: G, N, S, R, E, T, D, V, C and A.

Item 5. The HIV-1 envelope polypeptide according to any of the preceding, wherein the amino acid residue in position X(24) is selected from the group consisting of: S, K, T, R, A, P, Y, F, G, Q, I and H.

Item 6. The HIV-1 envelope polypeptide according to any of the preceding, wherein the amino acid residue in position X(34) is selected from the group consisting of: P, K, R, S, A, L, Q, E, H, T, I, V, and F.

Item 7. The HIV-1 envelope polypeptide according to any of the preceding, wherein the amino acid residue in position X(40) is selected from the group consisting of: S, N, G, T, R, I, V, K, W, A, P, Y, D, Q, H, E, and C.

Item 8. The HIV-1 envelope polypeptide according to any of the preceding, wherein the amino acid residue in position X(10) is threonine (T).

Item 9. The HIV-1 envelope polypeptide according to any of the preceding, wherein the amino acid residue in position X(19) is asparagine (N).

Item 10. The HIV-1 envelope polypeptide according to any of the preceding, wherein the amino acid residue in position X(24) is lysine (K).

Item 11. The HIV-1 envelope polypeptide according to any of the preceding, wherein the amino acid residue in position X(34) is lysine (K).

Item 12. The HIV-1 envelope polypeptide according to any of the preceding, wherein the amino acid residue in position X(40) is serine (S).

Item 13. The HIV-1 envelope polypeptide according to any of the preceding, wherein the amino acid sequence is selected from the group consisting of SEQ ID NO: 2-68 or SEQ ID NO: 130-326, or any part thereof, or functional homolog or any polypeptide with at least 80% identity to said polypeptide or part thereof.

Item 14. The HIV-1 envelope polypeptide according to any of the preceding, wherein the amino acid sequence is selected from the group consisting of SEQ ID NO: 4-19, or any part thereof, or functional homolog or any polypeptide with at least 90% identity to said polypeptide or part thereof.

Item 15. The HIV-1 envelope polypeptide according to any of the preceding, wherein the amino acid sequence is selected from the group consisting of SEQ ID NO: 20-63, or any part thereof, or functional homolog or any polypeptide with at least 90% identity to said polypeptide or part thereof.

Item 16. The HIV-1 envelope polypeptide according to any of the preceding, wherein the amino acid sequence is selected from the group consisting of SEQ ID NO: 64-68, or any part thereof, or functional homolog or any polypeptide with at least 90% identity to said polypeptide or part thereof.

Item 17. The HIV-1 envelope polypeptide according to any of the preceding, wherein the amino acid sequence is selected from the group consisting of SEQ ID NO: 130-133, or any part thereof, or functional homolog or any polypeptide with at least 90% identity to said polypeptide or part thereof.

Item 18. The HIV-1 envelope polypeptide according to any of the preceding, wherein the amino acid sequence is selected from the group consisting of SEQ ID NO: 134-323, or any part thereof, or functional homolog or any polypeptide with at least 90% identity to said polypeptide or part thereof.

Item 19. The HIV-1 envelope polypeptide according to any of the preceding, wherein the amino acid sequence is LRARLLALETFIQNQQLLNLWGCKGNLICYTSVKWNDTWKGNSDTSLENIWDN (SEQ ID NO: 4), or any part thereof, or functional homolog or any polypeptide with at least 90% identity to said polypeptide or part thereof.

Item 20. The HIV-1 envelope polypeptide according to any of the preceding, wherein the amino acid sequence is LQARIMAVERYLKDQQLLGIWGCSGKLICTTAVPWNASWSNKSLEQIWNH (SEQ ID NO: 20) or any part thereof, or functional homolog or any polypeptide with at least 90% identity to said polypeptide or part thereof.

Item 21. The HIV-1 envelope polypeptide according to any of the preceding, wherein the amino acid sequence is LQARILAVERYLKDQQLLRIWGCFGKLICTTAVPWNASWSNKSLEQIWNH (SEQ ID NO: 64) or any part thereof, or functional homolog or any polypeptide with at least 90% identity to said polypeptide or part thereof.

Item 22. The HIV-1 envelope polypeptide according to any of the preceding, wherein the amino acid sequence is HIV G19R: LQARVLAVERYLKDQQLLRIWGC (SEQ ID NO: 132) or any part thereof, or functional homolog or any polypeptide with at least 90% identity to said polypeptide or part thereof.

Item 23. The HIV-1 envelope polypeptide according to any of the preceding, wherein the amino acid sequence is HIV M/O chimera: LQARILAVETLIQNQQLLNLWGC (SEQ ID NO: 133) or any part thereof, or functional homolog or any polypeptide with at least 90% identity to said polypeptide or part thereof.

Item 24. The HIV-1 envelope polypeptide according to any of the preceding with an amino acid sequence selected from the group consisting of SEQ ID NO: 120-124 or SEQ ID NO: 324-326, or any part thereof, or functional homolog or any amino acid sequence with at least 90% identity to said amino acid sequence or part thereof.

Item 25. The HIV-1 envelope polypeptide according to any of the preceding with an amino acid sequence of SEQ ID NO: 120, or any part thereof, or functional homolog or any amino acid sequence with at least 90% identity to said amino acid sequence or part thereof.

Item 26. The HIV-1 envelope polypeptide according to any of the preceding, wherein said envelope polypeptide has been produced synthetically.

Item 27. An antigen comprising at least one peptide with an amino acid sequence of an HIV-1 envelope polypeptide as defined in any of Item 1 to Item 26, or a functional homolog thereof having at least 70% identity to said envelope polypeptide or an immunological active fragment comprising a consecutive sequence of at least 10 amino acids selected from a region of said envelope polypeptide.

Item 28. A nucleic acid sequence encoding at least one HIV-1 envelope polypeptide with an amino acid sequence as defined in any of Item 1 to Item 26 or a fragment thereof and/or a nucleic acid sequence encoding at least one antigen as defined in Item 27.

Item 29. The nucleic acid according to Item 28, wherein the nucleic acid sequence has been modified by codon optimization.

Item 30. An isolated eukaryotic expression vector comprising at least one nucleic acid sequence as defined in any of Item 28 and/or Item 29 or a fragment thereof.

Item 31. The vector according to Item 30, wherein said is a mammalian expression vector.

Item 32. The vector according to any of Item 30 to Item 31, wherein said vector comprise at least one intron.

Item 33. The vector according to any of Item 30 to Item 32, which is transcribed in the nucleus, thereby producing high levels of transcript, which after transport to the cytoplasm can be translated into envelope polypeptide.

Item 34. The vector according to any of Item 30 to Item 32, which is transcribed in the cytoplasm, thereby producing high levels of transcript, which can be translated into envelope polypeptide.

Item 35. The vector according to any of Item 30 to Item 34, wherein said vector comprises a constitutive transport element (CTE).

Item 36. The vector according to any of Item 30 to Item 35, wherein said vector comprises a Rev responsive element (RRE).

Item 37. The vector according to Item 30, wherein said vector is a retroviral vector.

Item 38. The vector according to Item 37, wherein said vector is a replication deficient retroviral vector.

Item 39. The vector according to Item 37, wherein said vector is a replication competent retroviral vector.

Item 40. The isolated vector according to any of Item 30 to Item 39, wherein the vector is derived from gamma-retroviruses.

Item 41. The vector according to Item 40, wherein the vector is derived from Murine Leukemia Virus (MLV).

Item 42. The vector according to Item 41, wherein the vector is derived from Moloney Murine Leukemia Virus (MoMLV).

Item 43. The vector according to Item 42, wherein the vector is derived from Akv MLV.

Item 44. The vector according to any of Item 30 to Item 43, further comprising at least one additional nucleic acid sequence.

Item 45. The vector according to Item 44, further comprising at least one internal ribosomal entry site (IRES).

Item 46. The vector according to Item 45, wherein said IRES is selected from the IRES elements of picornaviridae, retroviridae or retrotransposons, mammalia or combinations thereof.

Item 47. The vector according to Item 46, wherein said IRES is selected from the IRES elements of picornavirus.

Item 48. The vector according to Item 46, wherein said IRES is selected from the IRES elements of encephalomyocarditis (ECMV).

Item 49. The vector according to any of the preceding Item 45 to Item 48, wherein said IRES is located in a region flanked by the 3′-LTR and the 5′-LTR.

Item 50. The vector according to any of the preceding Item 49, wherein said IRES is located in the 3′-Long Terminal Repeat (LTR) or the 5′-LTR.

Item 51. The vector according to Item 50, wherein said IRES is located in the U3 region of the 3′ LTR.

Item 52. The vector according to Item 50, wherein said IRES is located in the U3 region between the inverted repeats and the transcription regulatory elements.

Item 53. The vector according to any of Item 44 to Item 52, wherein said at least one additional nucleic acid sequence is a reporter gene.

Item 54. The vector according to Item 53, wherein said reporter gene encodes enhanced green fluorescent protein (eGFP), lac Z, dsRed, enhanced yellow fluorescent protein (eYFP), enhanced cyan fluorescent protein (eCFP), enhanced blue fluorescent protein (eBFP) and the human alpha-1-antitrypsin (hAAT). It is understood that any of the enhanced green fluorescent protein (eGFP), lac Z, dsRed, enhanced yellow fluorescent protein (eYFP), enhanced cyan fluorescent protein (eCFP), enhanced blue fluorescent protein (eBFP) or the human alpha-1-antitrypsin (hAAT).

Item 55. The vector according to any of Item 44 to Item 52, wherein said at least one additional nucleic acid sequence encodes a selective marker.

Item 56. The vector according to Item 55, wherein said selective marker is neomycin phosphotransferase II.

Item 57. The vector according to Item 55, wherein said at least one additional nucleic acid sequence encodes a suicide gene.

Item 58. The vector according to any of Item 44 to Item 52, wherein said at least one additional nucleic acid sequence encodes an immunomodulating polypeptide.

Item 59. The vector according to Item 58, wherein said immunomodulating polypeptide is an immunostimulating polypeptide.

Item 60. The vector according to Item 58, wherein said immunomodulating polypeptide is an genetic adjuvant.

Item 61. The vector according to Item 59, wherein said immunostimulating polypeptide is selected from the group consisting of cytokines or hormones.

Item 62. A biological entity comprising at least one HIV-1 envelope as defined in any of Item 1 to Item 26, and/or at least one antigen as defined in Item 27, and/or at least one nucleic acid as defined in any of Item 28 to Item 29, and/or at least one vector as defined in any of Item 30 to Item 61.

Item 63. The biological entity according to Item 62, wherein said HIV-1 envelope or part thereof is expressed on the surface of the biological entity.

Item 64. The biological entity according to any of Item 62 to Item 63, wherein said HIV-1 envelope or part thereof comprises gal-alfa1-3Galbeta1-4GlcNAc-R epitopes.

Item 65. The biological entity according to any of Item 62 to Item 64, which is not capable of mediating fusion of said biological entity and cells expressing receptors for HIV.

Item 66. The biological entity according to any of Item 62 to Item 64, which is capable of mediating fusion of said biological entity and cells expressing receptors for HIV.

Item 67. The biological entity according to any of Item 62 to Item 66, wherein said biological entity is capable of infecting a CD4 positive cell.

Item 68. The biological entity according to any of Item 62 to Item 67, wherein said biological entity is capable of inducing an immunogenic response in a host animal.

Item 69. The biological entity according to any of Item 62 to Item 68, wherein said immunogenic response is directed towards said biological entity in said host animal.

Item 70. The biological entity according to any of Item 62 to Item 69, wherein said host animal is a human being.

Item 71. The biological entity according to any of Item 62 to Item 70, which is capable of infecting human cells.

Item 72. The biological entity according to any of Item 62 to Item 71, which is capable of infecting stem cells.

Item 73. The biological entity according to any of Item 62 to Item 72, wherein said biological entity is selected from the group consisting of a viral particle, a retroviral particle, a eukaryotic cell, a prokaryotic cell, a liposome and/or a nanoparticle.

Item 74. The biological entity according to any of Item 62 to Item 73, wherein said biological entity is a liposome, a nanoparticle, a virosome, a protzoa, an enterobacteria, and/or a nanoparticle or a liposome with synthetic envelope polypeptides.

Item 75. The biological entity according to any of Item 62 to Item 74, wherein said biological entity biological entity is a nanoparticle or a liposome with at least one synthetic HIV-1 envelope polypeptide as defined in Item 26.

Item 76. The biological entity according to any of Item 62 to Item 73, wherein said biological entity is a retroviral particle.

Item 77. The biological entity according to any of Item 62 to Item 76, wherein said biological entity is a provirus.

Item 78. The biological entity according to Item 76, wherein said retroviral particle is a gammaretroviral particle.

Item 79. A vaccine composition comprising

a) an HIV-1 envelope polypeptide as defined in any of Item 1 to Item 26, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined in Item 27, and/or
c) a nucleic acid as defined in any of Item 28 to Item 29, and/or
d) a vector as defined in any of Item 30 to Item 61, and/or
e) a biological entity as defined in any of Item 62 to Item 78, and
f) an adjuvant
for use as a medicament.

Item 80. The vaccine composition according to Item 79, wherein said immunogenically active peptide fragment consists of a consecutive sequence of in the range of from 10 to 50 amino acids.

Item 81. The vaccine composition according to any of Item 79 to Item 80, wherein the adjuvant is selected from the group consisting of bacterial DNA based adjuvants, oil/surfactant based adjuvants, viral dsRNA based adjuvants and imidazochinilines, incomplete Freund's adjuvant (IFA).

Item 82. The vaccine composition according to any of Item 79 to Item 80, wherein said adjuvant is selected from the group consisting of AlK(SO4)2, AlNa(SO4)2, AlNH4 (SO4), silica, alum, Al(OH)3, Ca3(PO4)2, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), poly GC, wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see US 58767 and U.S. Pat. No. 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Preferred adjuvants to be used with the invention include oil/surfactant based adjuvants such as Montanide adjuvants (available from Seppic, Belgium), preferably Montanide ISA-51.

Item 83. The vaccine composition according to any of Item 79 to Item 81, wherein the adjuvant is a Montanide ISA adjuvant.

Item 84. The vaccine composition according to Item 83, wherein the adjuvant is Montanide ISA 51 or Montanide ISA 720.

Item 85. The vaccine composition according to Item 84, wherein the adjuvant is Montanide ISA 51.

Item 86. The vaccine composition according to any of Item 79 to Item 81, wherein the adjuvant is GM-CSF.

Item 87. The vaccine composition according to any of Item 79 to Item 86, wherein the vaccine composition is capable of eliciting an immune response against HIV envelope and/or against an antigen presenting cell expressing an HIV-1 envelope as defined in any of Item 1 to Item 25, and/or against an antigen as defined in Item 27, and/or against a biological entity as defined in any of Item 62 to Item 78 when administered to an individual.

Item 88. The vaccine composition according to any of Item 79 to Item 87, wherein said vaccine composition is capable of eliciting a cellular immune response in the individual.

Item 89. The vaccine according to Item 88, wherein said individual is infected with HIV.

Item 90. The vaccine composition according to any of Item 79 to Item 89, comprising a peptide fragment, which is restricted by a MHC Class I molecule.

Item 91. The vaccine composition according to any of Item 79 to Item 90, comprising a peptide fragment, which is restricted by a MHC Class II molecule.

Item 92. The vaccine composition according to any of Item 79 to Item 91, wherein the HIV-1 polypeptide or fragment thereof consists of at the most 50 amino acid residues, for example at the most 45 amino acid residues, such as at the most 40 amino acid residues, for example at the most 35 amino acid residues, such as at the most 30 amino acid residues, for example at the most 25 amino acid residues, such as 20 to 25 amino acid residues.

Item 93. The vaccine composition according to any of Item 79 to Item 92, where the vaccine elicits the production in a vaccinated individual of regulatory T-cells having a cytotoxic effect against cells expressing HIV-1 envelope polypeptide or part thereof, and/or antigen presenting cells expressing HIV-1 envelope or part thereof.

Item 94. The vaccine composition according to any of Item 79 to Item 93, wherein the vaccine composition is capable of eliciting a clinical response in a subject, wherein the clinical response is characterised by a reduced susceptibility, resistance, stabilisation, remission or curing/recovery of an HIV infection and/or AIDS.

Item 95. The vaccine composition according to any of Item 79 to Item 94, further comprising an immunogenic protein or peptide fragment selected from a protein or peptide fragment, which is not derived from HIV-1 envelope.

Item 96. The vaccine composition according to any of Item 79 to Item 95, wherein the vaccine composition comprises antigen presenting biological entity according to any of Item 62 to Item 78.

Item 97. The vaccine composition according to any of Item 79 to Item 96, comprising a vector according to any of Item 30 to Item 61.

Item 98. A kit-of-parts comprising the vaccine composition as defined in any of Item 79 to Item 97, and a second active ingredient.

Item 99. The kit-of-parts according to Item 98, wherein the second active ingredient is an immunostimulating composition.

Item 100. The kit-of-parts according to any of Item 98 or Item 99, wherein the further immunostimulating composition comprises one or more interleukins.

Item 101. The kit-of-parts according to any of Item 98 to Item 100, wherein the interleukins are selected from IL-2 and or IL-21.

Item 102. The kit-of-parts according to Item 98, wherein the second active ingredient is an antibiotic.

Item 103. The kit-of-parts according to Item 102, wherein the antibiotic is selected from: amoxicillin, penicillin, acyclovir and/or vidarabine.

Item 104. The kits-of-parts according to any of Item 98 to Item 103, where the provided compositions are to be administered simultaneously or sequentially.

Item 105. A method of treating, preventing or ameliorating a clinical condition, said method comprising administering to an individual suffering from said clinical condition an effective amount of an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104.

Item 106. The method according to Item 105, wherein said clinical condition is an infection.

Item 107. The method according to Item 105, wherein said clinical condition is HIV infection and/or AIDS.

Item 108. The method according to Item 107, wherein said individual suffering from HIV infection and/or AIDS is a human being.

Item 109. The method according to Item 108, wherein said human being is HIV seronegative.

Item 110. The method according to Item 108, wherein said human being is HIV seropositive.

Item 111. The method according to any of Item 105 to Item 110, wherein said an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 is administered to said organism two or more times.

Item 112. The method of to any of Item 105 to Item 111, which is combined with a further treatment.

Item 113. The method of Item 112, wherein the further treatment is selected from the group consisting of chemotherapy, treatment with immunostimulating substances, gene therapy, treatment with antibodies and treatment using dendritic cells.

Item 114. Use of an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 for the manufacture of a medicament for the treatment, amelioration or prevention of a clinical condition.

Item 115. The use according to Item 114, wherein said clinical condition is an infection.

Item 116. The use according to Item 114 to Item 115, wherein said clinical condition is HIV infection and/or acquired immunodeficiency syndrome (AIDS).

Item 117. The use according to any of Item 114 to Item 116, which is combined with a further treatment.

Item 118. The use according to any of Item 114 to Item 117, wherein said further treatment is selected from the group consisting of treatment with immunostimulating substances, gene therapy, treatment with antibodies, treatment using dendritic cells and/or treatments against infections.

Item 119. An HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 for treating, ameliorating or preventing a clinical condition.

Item 120. The polypeptide, antigen, vector, biological entity, vaccine composition, or kit-of-parts according to Item 119, wherein said clinical condition is an infection.

Item 121. The peptide, antigen, vector, biological entity, vaccine composition, or kit-of-parts according to any of Item 119 and Item 120, wherein said clinical condition is HIV infection and/or acquired immunodeficiency syndrome (AIDS).

Item 122. A pharmaceutical composition comprising an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 for treating, ameliorating or preventing a clinical condition.

Item 123. The pharmaceutical composition according to Item 119, for treating, ameliorating or preventing an infection.

Item 124. The pharmaceutical composition according to any of Item 119 and Item 120, for treating, ameliorating or preventing HIV infection and/or acquired immunodeficiency syndrome (AIDS).

Item 125. A method of reducing the risk of an individual encountering a clinical condition, said method comprising administration of an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 to said individual in an amount sufficient to generate a protective immune response.

Item 126. The method according to Item 125, wherein the clinical condition is infection with HIV.

Item 127. A method of producing the vaccine composition of Item 79, comprising combining

a) an HIV-1 envelope polypeptide as defined in any of Item 1 to Item 26, or a functional homologue thereof having at least 70% identity to said peptide or an immunogenically active peptide fragment comprising a consecutive sequence of at least 10 residues of said peptide or said functional homologue thereof, or a nucleic acid encoding said peptide or said peptide fragment or said functional homologies thereof and/or
b) an antigen as defined in Item 27, and/or
c) a nucleic acid as defined in any of Item 28 to Item 29, and/or
d) a vector as defined in any of Item 30 to Item 61, and/or
e) a biological entity as defined in any of Item 62 to Item 78, and
f) an adjuvant.

Item 128. An antibody, antigen binding fragment or recombinant protein thereof, which is specific for an HIV-1 envelope polypeptide or part thereof as defined in any of Item 1 to Item 26, and/or a nucleic acid as defined in any of Item 28 to Item 29, and/or an antigen as defined in Item 27, and/or a biological entity as defined in any of Item 62 to Item 78.

Item 129. The antibody, antigen binding fragment or recombinant protein thereof according to Item 128, which is capable of initiating an immune response against HIV-1 retroviral particles.

Item 130. An antibody obtainable by immunizing a host with a peptide according to any of Item 1 to Item 14, an antigen according to Item 27, a vaccine composition according to any of Item 79 to Item 97, and/or a kit-of-parts according to any of Item 98 to Item 104.

Item 131. A method of producing an antibody as defined in any of Item 128 to Item 130, said method comprising the steps of

a) administering an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 to an animal
b) obtaining said antibody from said animal

Item 132. A method of monitoring immunization, said method comprising the steps of

a) providing a blood sample from an individual
b) providing an HIV-1 envelope polypeptide or part thereof as defined in any of Item 1 to Item 26, an antigen as defined in Item 27, a nucleic acid as defined in any of Item 28 to Item 29, a vector as defined in any of the preceding Item 30 to Item 61, a biological entity as defined in any of Item 62 to Item 78, a vaccine composition as defined in any of Item 79 to Item 97, or a kit-of-parts as defined in any of Item 98 to Item 104 to an animal, and
c) determining whether said blood sample comprises antibodies or T-cells comprising T-cell receptors specifically binding an HIV-1 envelope polypeptide or part thereof
d) thereby determining whether an immune response to said protein or peptide has been raised in said individual.

1-81. (canceled)

82. A method of treating, preventing or ameliorating an HIV infection and/or acquired immunodeficiency syndrome (AIDS), said method comprising administering to an individual in need thereof an effective amount of an isolated or recombinant HIV-1 envelope polypeptide, which comprises a mutated immunosuppressive region which represses immunosuppression of the envelope polypeptide but which retains the fusogenic properties of a viral particle comprising the envelope polypeptide.

83. The method according to claim 82 wherein the mutated immunosuppressive region comprises a mutation at amino acid position 10 and/or 19 and/or 24 and/or 34 and/or 40 of the polypeptide sequence of the immunosuppressive region.

84. The method according to claim 82 wherein the mutated immunosuppressive region represses immunosuppression of the envelope polypeptide relative to the wild type envelope polypeptide.

85. The method according to claim 82 wherein the mutated immunosuppressive region comprises a mutation at amino acid position 10 of the polypeptide sequence of the immunosuppressive region.

86. The method according to claim 85 wherein the amino acid residue at position 10 of the polypeptide sequence of the immunosuppressive region has been mutated to a residue selected from the group consisting of Arginine; Serine; Threonine; Lysine; Glycine; Alanine; Asparagine; Glutamine; and Isoleucine.

87. The method according to claim 86 wherein the amino acid residue at position 19 of the polypeptide sequence of the immunosuppressive region has been mutated to a Threonine residue.

88. The method according to claim 82 wherein the mutated immunosuppressive region comprises a mutation at amino acid position 19 of the polypeptide sequence of the immunosuppressive region.

89. The method according to claim 88 wherein the amino acid residue at position 19 of the polypeptide sequence of the immunosuppressive region has been mutated to a residue selected from the group consisting of: Arginine; Glycine; Asparagine; Serine; Glutamic acid; Threonine; Aspartic acid; Valine; Cysteine; Alanine.

90. The method according to claim 89 wherein the amino acid residue at position 19 of the polypeptide sequence of the immunosuppressive region has been mutated to an Arginine residue.

91. The method according to claim 82 wherein the mutated immunosuppressive region comprises a mutation at amino acid position 24 of the polypeptide sequence of the immunosuppressive region.

92. The method according to claim 91 wherein the amino acid residue at position 24 of the polypeptide sequence of the immunosuppressive region has been mutated to a residue selected from the group consisting of Serine; Lysine; Threonine; Arginine; Alanine; Proline; Tyrosine; Phenylalanine; Glycine; Glutamine; Isoleucine; and Histidine.

93. The method according to claim 92 wherein the amino acid residue at position 19 of the polypeptide sequence of the immunosuppressive region has been mutated to a Lysine residue.

94. The method according to claim 82 wherein the mutated immunosuppressive region comprises a mutation at amino acid position 34 of the polypeptide sequence of the immunosuppressive region.

95. The method according to claim 94 wherein the amino acid residue at position 34 of the polypeptide sequence of the immunosuppressive region has been mutated to a residue selected from the group consisting of Proline; Lysine; Arginine; Serine; Alanine; Leucine; Glutamine; Glutamic acid; Histidine; Threonine; Isoleucine; Valine; and Phenylalanine.

96. The method according to claim 95 wherein the amino acid residue at position 34 of the polypeptide sequence of the immunosuppressive region has been mutated to a Lysine residue.

97. The method according to claim 82 wherein the mutated immunosuppressive region comprises a mutation at amino acid position 40 of the polypeptide sequence of the immunosuppressive region.

98. The method according to claim 97 wherein the amino acid residue at position 40 of the polypeptide sequence of the immunosuppressive region has been mutated to a residue selected from the group consisting of Serine; Asparagine; Glycine; Threonine; Arginine; Isoleucine; Valine; Lysine; Tryptophan; Alanine; Proline; Tyrosine; Aspartic acid; Glutamine; Histidine; Glutamic acid; and Cysteine.

99. The method according to claim 98 wherein the amino acid residue at position 40 of the polypeptide sequence of the immunosuppressive region has been mutated to a Serine residue.

100. The method according to claim 82 wherein the mutated immunosuppressive region comprises the polypeptide sequence selected from the group consisting of: SEQ ID NO:126 to SEQ ID NO:323.

101. The method according to claim 82 wherein the mutated immunosuppressive region comprises the polypeptide sequence LQARILAVETLIQNQQRLNLWGCKGKLICYTSVKWNTSWSNKSLEQIWNH (SEQ ID NO: 69), or is a functional homologue with at least 70% or at least 90% identity to said polypeptide.

102. The method according to claim 82 wherein the mutated immunosuppressive region comprises the polypeptide sequence LQARVLAVERYLKDQQLLRIWGC (SEQ ID NO: 132), or is a functional homologue with at least 70% or at least 90% identity to said polypeptide.

103. The method according to claim 82 wherein the mutated immunosuppressive region comprises the polypeptide sequence LQARILAVELYLKDQQLLLIWGCEGKLICTTAVPWNASWENKSLEQIWNH (SEQ ID NO: 72), or is a functional homologue with at least 70% or at least 90% identity to said polypeptide.

104. The method according to claim 82 wherein the mutated immunosuppressive region comprises the polypeptide sequence LRARLLAALETFIQNQQRLRLWGCFGNLITYTSVKWNDTWKGNSDTSLENIWDN (SEQ ID NO: 180), or is a functional homologue with at least 70% or at least 90% identity to said polypeptide.

105. The method according to claim 82 wherein the mutated immunosuppressive region comprises the polypeptide sequence LQTRVQAVETFIRDQQFMGIWGCSGKLICTTAVPWNASWSNKSLEQIWNH (SEQ ID NO: 233), or is a functional homologue with at least 70% or at least 90% identity to said polypeptide.

106. The method according to claim 82 wherein the envelope polypeptide in which the immunosuppressive region is mutated comprises a polypeptide sequence selected from the group consisting of: SEQ ID NO: 74-119.

107. The method according to claim 82 which comprises the polypeptide sequence selected from the group consisting of: SEQ ID NO: 324 and 332.

108. A biological entity comprising at least one HIV-1 envelope polypeptide as defined in claim 82.

109. The biological entity according to claim 108 wherein the HIV envelope polypeptide is expressed on the surface of the biological entity.

110. The biological entity according to claim 108 which is capable of inducing an immunogenic response in a host animal, such as a human.

111. The biological entity according to claim 108 which is selected from the group consisting of: a viral particle; a retroviral particle; a eukaryotic cell; a prokaryotic cell; a liposome; a nano-particle; a provirus.

112. A vaccine composition comprising an HIV-1 envelope polypeptide as defined in claim 82 and an adjuvant, for use in the treatment, amelioration or prevention of an HIV infection and/or acquired immunodeficiency syndrome (AIDS).

113. The vaccine composition of claim 112 comprising a biological entity comprising said HIV-1 envelope polypeptide.

114. A vaccine composition according to claim 112 wherein the adjuvant is selected from the group consisting of: AlK(SO₄)₂, AlNa(SO₄)₂, AlNH₄(SO₄), silica, alum, Al(OH)₃, Ca₃(PO₄)₂, kaolin, carbon, aluminium hydroxide, muramyl dipeptides, N-acetyl-muramyl-L-threonyl-D-isoglutamine (thr-DMP), N-acetyl-nornuramyl-L-alanyl-D-isoglutamine (CGP 11687, nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, MTP-PE), RIBI (MPL+TDM+CWS) in a 2% squalene/Tween 80 emulsion, lipopolysaccharides and its various derivatives, lipid A, Freund's Complete Adjuvant (FCA), Freund's Incomplete Adjuvants, Merck Adjuvant 65, polynucleotides, poly IC, poly AU, poly GC, wax D from Mycobacterium, tuberculosis, substances found in Corynebacterium parvum, Bordetella pertussis, and members of the genus Brucella, liposomes or other lipid emulsions, Titermax, ISCOMS, Nil A, ALUN, Lipid A derivatives, choleratoxin derivatives, HSP derivatives, LPS derivatives, synthetic peptide matrixes or GMDP, interleukin 1, interleukin 2, Montanide ISA-51 and QS-21.

115. The vaccine composition according to claim 112, which is capable of eliciting a clinical response in a subject, wherein the clinical response is characterised by a reduced susceptibility, resistance, stabilisation, remission or curing/recovery of an HIV infection and/or AIDS.

116. An antibody, antigen binding fragment or recombinant polypeptide thereof, which is specific for an HIV-1 envelope polypeptide as defined in claim 82.

117. The antibody, antigen binding fragment or recombinant polypeptide thereof according to claim 116, which is capable of initiating an immune response against HIV-1 retroviral particles.

118. A method of producing an antibody as defined in claim 116, said method comprising the steps of:
a) administering an HIV-1 envelope polypeptide to an animal, wherein said HIV-1 envelope polypeptide comprises a mutated immunosuppressive region which represses immunosuppression of the envelope polypeptide but which retains the fusogenic properties of a viral particle comprising the envelope polypeptide; and
b) obtaining said antibody from said animal.

119. An isolated or recombinant HIV-1 envelope polypeptide as defined in claim 82.

120. An isolated or recombinant HIV-1 envelope polypeptide according to claim 119 which comprises a mutated immunosuppressive region, which represses immunosuppression of the envelope polypeptide but which retains the fusogenic properties of a viral particle comprising the envelope polypeptide;
wherein the amino acid residue at position 19 of the polypeptide sequence encoding the immunosuppressive region has been mutated to an Arginine residue;
and wherein the envelope polypeptide in which the immunosuppressive region is mutated comprises a polypeptide sequence selected from the group consisting of: SEQ ID NO: 74-119;
or a functional homologue with at least 70% or at least 90% identity to said polypeptide.

121. A pharmaceutical composition comprising the HIV-1 envelope polypeptide of claim 119.


SEQUENCE LISTING (337 sequences)

SEQ ID NO: 1 - protein, 50 aa, HIV
  misc_feature (15)..(15): Xaa can be any naturally occurring amino acid
WGIKQLQARILAVEXLIQNQQRLXLWGCXGKLICYTSVXWNTSWXNKSLE

SEQ ID NO: 2 - protein, 50 aa, HIV; identical to SEQ ID NO: 1
  misc_feature (15)..(15): Xaa can be any naturally occurring amino acid

SEQ ID NO: 3 - protein, 50 aa, HIV
  misc_feature (15)..(15): Xaa can be any naturally occurring amino acid
WGIKQLQARVLAVEXLIQNQQLLXLWGCXGKLICYTSVXWNTSWXNKSLE

SEQ ID NO: 4 - protein, 53 aa, HIV
LRARLLALETFIQNQQLLNLWGCKGNLICYTSVKWNDTWKGNSDTSLENIWDN

SEQ ID NO: 5 - protein, 50 aa, HIV
LQARILAVETYLKDQQLLNIWGCKGKLICTTAVKWNASWSNKSLEQIWNH

SEQ ID NO: 6 - protein, 50 aa, HIV
LQARILAVETYLKDQQLLGIWGCSGKLICTTAVPWNASWSNKSLEQIWNH

SEQ ID NO: 7 - protein, 50 aa, HIV
LQARILAVERYLKDQQLLNIWGCSGKLICTTAVPWNASWSNKSLEQIWNH

SEQ ID NO: 8 - protein, 50 aa, HIV
LQARILAVERYLKDQQLLGIWGCKGKLICTTAVPWNASWSNKSLEQIWNH

SEQ ID NO: 9 - protein, 50 aa, HIV
LQARILAVERYLKDQQLLGIWGCSGKLICTTAVKWNASWSNKSLEQIWNH

SEQ ID NO: 10 - protein, 50 aa, HIV
LQARILAVETYLKDQQLLNIWGCSGKLICTTAVPWNASWSNKSLEQIWNH

SEQ ID NO: 11 - protein, 50 aa, HIV
LQARILAVETYLKDQQLLGIWGCKGKLICTTAVPWNASWSNKSLEQIWNH

SEQ ID NO: 12 - protein, 50 aa, HIV
LQARILAVETYLKDQQLLGIWGCSGKLICTTAVKWNASWSNKSLEQIWNH

SEQ ID NO: 13 - protein, 50 aa, HIV
LQARILAVETYLKDQQLLNIWGCKGKLICTTAVPWNASWSNKSLEQIWNH

SEQ ID NO: 14 - protein, 50 aa, HIV
LQARILAVETYLKDQQLLNIWGCSGKLICTTAVKWNASWSNKSLEQIWNH

SEQ ID NO: 15 - protein, 50 aa, HIV
LQARILAVETYLKDQQLLGIWGCKGKLICTTAVKWNASWSNKSLEQIWNH

SEQ ID NO: 16 - protein, 50 aa, HIV
LQARILAVERYLKDQQLLNIWGCKGKLICTTAVPWNASWSNKSLEQIWNH

SEQ ID NO: 17 - protein, 50 aa, HIV
LQARILAVERYLKDQQLLNIWGCSGKLICTTAVKWNASWSNKSLEQIWNH

SEQ ID NO: 18 - protein, 50 aa, HIV
LQARILAVERYLKDQQLLNIWGCKGKLICTTAVKWNASWSNKSLEQIWNH

SEQ ID NO: 19 - protein, 50 aa, HIV
LQARILAVERYLKDQQLLGIWGCKGKLICTTAVKWNASWSNKSLEQIWNH

SEQ ID NO: 20 - protein, 50 aa, HIV
LQARIMAVERYLKDQQLLGIWGCSGKLICTTAVPWNASWSNKSLEQIWNH

SEQ ID NO: 21 - protein, 50 aa, HIV
LQARILAMERYLKDQQLLGIWGCSGKLICTTAVPWNASWSNKSLEQIWNH

SEQ ID NO: 22 - protein, 50 aa, HIV
LQARILAVERYMKDQQLLGIWGCSGKLICTTAVPWNASWSNKSLEQIWNH

SEQ ID NO: 23 - protein, 50 aa, HIV
LQARILAVERYLKDQQLMGIWGCSGKLICTTAVPWNASWSNKSLEQIWNH

SEQ ID NO: 24 - protein, 50 aa, HIV
LQARILAVERYLKDQQLLGMWGCSGKLICTTAVPWNASWSNKSLEQIWNH

SEQ ID NO: 25 - protein, 50 aa, HIV
LQARILAVERYLKDQQLLGIWGCSGKLICTTAMPWNASWSNKSLEQIWNH

SEQ ID NO: 26 - protein, 50 aa, HIV
LQARILAVERYLKDQQLLGIWGCSGKLICTTAVPWNASWSNKSLEQMWNH

SEQ ID NO: 27 - protein, 50 aa, HIV
LRARLLALERYLKDQQLLGIWGCSGKLICTTAVPWNASWSNKSLEQIWNH

SEQ ID NO: 28 - protein, 50 aa, HIV
LRARLLALETFIQNQQLLNLWGCSGKLICTTAVPWNASWSNKSLEQIWNH

SEQ ID NO: 29 - protein, 50 aa, HIV
RQTEVLAIERYLKDQQLLGIWGCSGKLICTTAVPWNASWSNKSLEQIWNH

SEQ ID NO: 30 - protein, 50 aa, HIV
LRTRVLAIERYLKDQQLLGIWGCSGKLICTTAVPWNASWSNKSLEQIWNH

SEQ ID NO: 31 - protein, 50 aa, HIV
LRTRVQAIERYLKDQQLLGIWGCSGKLICTTAVPWNASWSNKSLEQIWNH

SEQ ID NO: 32 - protein, 50 aa, HIV
LRTKVQTLETLIRNRKFMNLWGCSGKLICTTAVPWNASWSNKSLEQIWNH

SEQ ID NO: 33 - protein, 50 aa, HIV
LRTRVLALETLIQNQQLLGIWGCSGKLICTTAVPWNASWSNKSLEQIWNH

SEQ ID NO: 34 - protein, 50 aa, HIV
LQTRIQAMETYIRDQQFMGIWGCSGKLICTTAVPWNASWSNKSLEQIWNH

SEQ ID NO: 35 - protein, 50 aa, HIV
LQTRIQAVETFIRDQQFMGIWGCSGKLICTTAVPWNASWSNKSLEQIWNH

SEQ ID NO: 36 - protein, 50 aa, HIV
SQARIQAVETFIRDQQFMGIWGCSGKLICTTAVPWNASWSNKSLEQIWNH

SEQ ID NO: 37 - protein, 50 aa, HIV
LQTRIQAVETFIRDQQLLGMWGCSGKLICTTAVPWNASWSNKSLEQIWNH

SEQ ID NO: 38 - protein, 50 aa, HIV
LQARILAMERYMKDQQLMGMWGCSGKLICTTAVPWNASWSNKSLEQIWNH

SEQ ID NO: 39 - protein, 50 aa, HIV
LRARILAMERYMKDQQLMGMWGCSGKLICTTAVPWNASWSNKSLEQIWNH

SEQ ID NO: 40 - protein, 50 aa, HIV
LRARILAMERYLKDQQLLGIWGCSGKLICTTAVPWNASWSNKSLEQIWNH

SEQ ID NO: 41 - protein, 50 aa, HIV
LRARILAMETYLKDQQLLGIWGCSGKLICTTAVPWNASWSNKSLEQIWNH

SEQ ID NO: 42 - protein, 50 aa, HIV
LRARILAMETYMKDQQLMGMWGCSGKLICTTAVPWNASWSNKSLEQIWNH

SEQ ID NO: 43 - protein, 50 aa, HIV
LRTRILAMETYLKDQQLLGIWGCSGKLICTTAVPWNASWSNKSLEQIWNH

SEQ ID NO: 44 - protein, 50 aa, HIV
LRTRVLALETLIQNQQLLNIWGCSGKLICTTAVPWNASWSNKSLEQIWNH

SEQ ID NO: 45 - protein, 50 aa, HIV
LQTRIQAVETFIRDQQLLNMWGCSGKLICTTAVPWNASWSNKSLEQIWNH

SEQ ID NO: 46 - protein, 50 aa, HIV
LQARILAVERYLKDQQLLGIWGCKGNLICTTAVPWNASWSNKSLEQIWNH

SEQ ID NO: 47 - protein, 50 aa, HIV
LQARILAVERYLKDQQLLGIWGCFGKLICTTAVPWNASWSNKSLEQIWNH

SEQ ID NO: 48 - protein, 50 aa, HIV
LQARILAVERYLKDQQLLRIWGCSGKLICTTAVPWNASWSNKSLEQIWNH

SEQ ID NO: 49 - protein, 53 aa, HIV
LRARLLALETFIQNQQLLRLWGCKGNLICYTSVKWNDTWKGNSDTSLENIWDN

SEQ ID NO: 50 - protein, 53 aa, HIV
LRARLLALETFIQNQQLLRLWGCFGNLICYTSVKWNDTWKGNSDTSLENIWDN

SEQ ID NO: 51 - protein, 53 aa, HIV
LRARLLALETFIQNQQLLNLWGCFGNLICYTSVKWNDTWKGNSDTSLENIWDN

SEQ ID NO: 52 - protein, 50 aa, HIV
LQTRIQAVETFIRDQQLLGMWGCKGNLICTTAVPWNASWSNKSLEQIWNH

SEQ ID NO: 53 - protein, 50 aa, HIV
LQTRIQAVETFIRDQQFMGIWGCKGNLICTTAVPWNASWSNKSLEQIWNH

SEQ ID NO: 54 - protein, 50 aa, HIV
LQTRIQAVETFIRDQQLLNMWGCKGNLICTTAVPWNASWSNKSLEQIWNH

SEQ ID NO: 55 - protein, 50 aa, HIV
LQTRIQAVETFIRDQQFMNIWGCKGNLICTTAVPWNASWSNKSLEQIWNH

SEQ ID NO: 56 - protein, 50 aa, HIV
LQTRIQAVETFIRDQQFMRIWGCFGNLICTTAVPWNASWSNKSLEQIWNH

SEQ ID NO: 57 - protein, 50 aa, HIV
LRTRVLALETLIQNQQLLRIWGCFGKLICTTAVPWNASWSNKSLEQIWNH

SEQ ID NO: 58 - protein, 50 aa, HIV
LQTRIQAMETYIRDQQFMRIWGCFGKLICTTAVPWNASWSNKSLEQIWNH

SEQ ID NO: 59 - protein, 50 aa, HIV
LQTRIQAVETFIRDQQFMRIWGCFGKLICTTAVPWNASWSNKSLEQIWNH

SEQ ID NO: 60 - protein, 50 aa, HIV
SQARIQAVETFIRDQQFMRIWGCFGKLICTTAVPWNASWSNKSLEQIWNH

SEQ ID NO: 61 - protein, 50 aa, HIV
LQTRIQAVETFIRDQQLLRMWGCFGKLICTTAVPWNASWSNKSLEQIWNH

SEQ ID NO: 62 - protein, 50 aa, HIV
LQARILAMERYMKDQQLMRMWGCFGKLICTTAVPWNASWSNKSLEQIWNH

SEQ ID NO: 63 - protein, 50 aa, HIV
LRARILAMERYMKDQQLMRMWGCFGKLICTTAVPWNASWSNKSLEQIWNH

SEQ ID NO: 64 - protein, 50 aa, HIV
LQARILAVERYLKDQQLLRIWGCFGKLICTTAVPWNASWSNKSLEQIWNH

SEQ ID NO: 65 - protein, 50 aa, HIV
LQARILAVETLIQNQQRLNLWGCKGKLICYTSVKWNTSWSNKSLEQIWNH

SEQ ID NO: 66 - protein, 50 aa, HIV; identical to SEQ ID NO: 5

SEQ ID NO: 67 - protein, 50 aa, HIV; identical to SEQ ID NO: 6

SEQ ID NO: 68 - protein, 50 aa, HIV
LQARILAVKTYLKDQQLLNIWGCKGKLICTTAVKWNASWSNKSLEQIWNH

SEQ ID NO: 69 - protein, 50 aa, HIV; identical to SEQ ID NO: 65

SEQ ID NO: 70 - protein, 50 aa, HIV; identical to SEQ ID NO: 5

SEQ ID NO: 71 - protein, 50 aa, HIV; identical to SEQ ID NO: 6

SEQ ID NO: 72 - protein, 50 aa, HIV; identical to SEQ ID NO: 64

SEQ ID NO: 73 - protein, 50 aa, HIV
LQARILAVEKTYLKDQQLLNIWGCKGKLICTTAVKWNASWSNKSLEQIWN

SEQ ID NO: 74 - protein, 870 aa, HIV
  misc_feature (86)..(86): Xaa can be any naturally occurring amino acid
MRAKGIQMNLHCLLKWGTMILGMILICSAAEQRWVTVYYGVPVWKDAETTLFCASDAKAYDTEVHNVWATHACVPTDPNPQEINLXNVTEEFNMWKNNMVEQMQEDIISLWDQSLKPCVKLTPLCVTLNCSHEVIFNSTLNNSTHSNKTLNNNTIEMKEEVRNCSYNVTTVLRDKKQKIYSLFYRLDVVPIGNNSDSEYILINCNTSTITQACPKVSFEPIPIHYCTPAGYAILKCNDKDFNGTGPCKNVSTVQCTHGIKPVVTTQLLLNGSLAENRTMIRSKNITDNKENIIVQLTEPVNITCIRPNNNTRKSVRIGPGQTFYATGEIIGDIRKAHCVVNKTEWNKNLKKVVVQLRTYFKNKTISFTNHSGGDPEVTTHSFNCGGEFFYCNTSELFNRTWNATDQLNSTEDSTALNETIILPCRIKQVINMWQTPGQAMYAPPIRGAIRCESNITGLILTRDGGNDNTSTNETFRPGGGDMRDNWRSELYKYKVVRIEPLGIAPTTAKRRVVQREKRAVGIGAVFIGFLGAAGSTMGAASITLTVQARQLLSGIVQQQSNLLRAIEAQQHMLKLTVWGIKQLQARVLALERYLKDQQLLGIWGCSGKLICTTNVPWNNTWSNKNKSEIWDKMTWLQWDKEISNYTQIIYNLIEESQTQQEINEQELLALDKWANLWNWFDISQWLWYIKIFIMIVGGLIGLRIVFAVLSIISRVRQGYSPLSFQTHTPNPEGLDRPGRTEEEGGEQGRDRSIRLVSGFLALAWDDLRSLCLFSYHRLRDLLSIVTRTVELLGHSSLKGLRLGWEGLKYLWNLLVYWSQELKISAVNLYDTIAIAVAGWTDRVIEIGQGICRAILNIPRRIRQGLERALL

SEQ ID NO: 75 - protein, 853 aa, HIV
MRVMGIQRNCQHLLTWGIMILGTIIFCSAVENLWVTVYYGVPVWRDADTTLFCASDAKAYETEKHNVWATHACVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRAKRRVVEREKRAVGIGAVFLGFLGAAGSTMGATSITLTVQARQLLSGIVQQQNNLLRAIEAQQHLLKLTVWGIKQLQARVLAVERYLRDQQLLGIWGCSGKLICTTNVPWNSSWSNKSLDEIWNNMTWLQWDKEINNYTQLIYRLIEESQNQQEKNEKELLELDKWANLWSWFDISNWLWYIKIFIIIVGGLIGLRIVFAVLSVINRVRQGYSPLSFQTHTPNPRGLDRPERIEEEDGEQGRGRSIRLVSGFLALAWDDLRSLCLFSYHRLRDFILIAARTVELLGHSSLKGLRLGWEGIKYLWNLLSYWGRELKISAINLVDTIAIAVAGWTDRVIEIAQRIGRAILHIPVRIRQGLERALL

SEQ ID NO: 76 - protein, 853 aa, HIV
MRVKGIQRNCQCLLTWGTMILGILIICRATENLWVTVYYGVPVWKDAKTTLFCASDAKAYETEKHNVWATHACVPTDPNPQEIHLENVTEDFNMWKNNMVEQMHTDIISLWDQSLKPCVKLTPLCVTLHCSNVTGANSTGTGGEEIKNCSYNITTELRDKRKKVYSLFYRLDIVQLSSNNSNSNEYRLINCNTSAITQACPKVSFEPIPIHYCAPAGFAILKCKDEEFNGTGPCKNVSTVQCTHGIKPVVSTQLLLNGSLAKEKVIIRSENITNNVKTIIVQLVKPVKINCTRPNNNTRTSIRIGPGQSFHATGDIIGDIRQAHCNVSRSEWNEALRQVVEQLRGHFGNKTIIFTNSSGGDIEITTHSFNCGGEFFYCDSSGLFNSTWDNTNITQPNSTGSNDTITLQCRIKQIINMWQRAGQAMYAPPIPGVISCVSNITGLLLTRDGGITSANETFRPGGGDMRDNWRSELYKYKVVKLEPLGVAPTRARRRVVEREKRAVGIGAVFIGFLGAAGSTMGAASMTLTVQARQLLSGIVQQQSNLLRAIEAQQHLLKLTVWGIKQLQARVLAVESYLRDQQLLGIWGCSGKLICTTTVPWNASWSNKSYSEIWENMTWLQWDKEISNYTNLIYGLIEESQNQQEKNEQDLLALDKWANLWSWFEISNWLWYIKIFIMIVGGLIGLRIVFAVLSIINRVRQGYSPLSFQTHTPNPGGPDRPGRIEEEDGELGRGRSIRLVNGFLALAWDDLRSLCLFSYHRLRDFILIAARTVELLGHSSLKGLRLGWEGLKYLWNLLVYWSRELRISATSLVDTIAIVVAGWTDRVIEIVQGIGRAILHIPRRIRQGLERALL

SEQ ID NO: 77 - protein, 857 aa, HIV
MRVMGIERNYPCWWTWGIMILGMIIICNTAENLWVTVYYGVPIWKDANTTLFCASDAKAYDTEVHNVWATHACVPTDPSPQELKMENVTEEFNMWKNNMVEQMHTDIISLWDQSLKPCVQLTPLCVTLDCSYNITNNITNSITNSSVNMREEIKNCSFNMTTELRDKNRKVYSLFYKLDVVQINNGNNSSNLYRLINCNTSALTQACPKVTFEPIPIHYCAPAGYAILKCNDKEFNGTGLCKNVSTVQCTHGIRPVVSTQLLLNGSLAEGKVMIRSENITNNVKNIIVQLNESVTINCTRPNNNTRRSVRIGPGQTFYATGDIIGDIRQAHCNVSGSQWNKTLHQVVEQLRKYWNNNTIIFNSSSGGDLEITTHSFNCAGEFFYCNTSGLFNSTWVNGTTSSMSNGTITLPCRIKQIINMWQRVGQAMYAPPIQGVIKCESNITGLILTRDGGVNSSDSETFRPGGGDMRDNWRSELYKYKVVKIEPLGVAPTKARRRVVEREKRAVTLGAVFIGFLGTAGSTMGAASITLTVQARKLLSGIVQQQSNLLRAIEAQQHLLKLTVWGIKQLQARVLAVERYLRDQQLLGIWGCSGKLICPTNVPWNSSWSNKSLDEIWENMTWLQWDKEISNYTIKIYELIEESQIQQERNEKDLLELDKWASLWNWFDISKWLWYIKIFIMIVGGLIGLRIVFAVLSVINRVRQGYSPLSFQTHTPNPRGLDRPGRIEEEGGEQDRGRSIRLVSGFLALAWDDLRNLCLFSYHRLRDFILIAARTVELLGHSSLKGLRLGWEGLKYLGNLLLYWGRELKISAINLLDTIAIAVAGWTDRVIETVQRLGRAILNIPRRIRQGFERALL

SEQ ID NO: 78 - protein, 861 aa, HIV
MRVMGTQTSYQHLWRWGILILGMLIICKATDWWVTVYYGVPVWKDAETTLFCASDDKAYETEAHNVWATHACVPTDPNPQEVNLKNVTEDFNMWKNNMVEQMHEDIISLWDQSLKPCVKLTPLCVTLNCSNANTNSTNSTSAPSMGPGEIKNCSFNVTTEVRDKEKKVYALFYKLDVVQINESDSNSTKDSTQYRLINCNTSAITQACPKVSFEPIPIHYCAPAGFAILKCEDPRFNGTGPCNNVSSVQCTHGIMPVASTQLLLNGSLAEKEVMIRSENITNNAKNIIVQFNESVPITCIRPNNNTRKGIPIGPGQVFYTSDIIGDIRQAYCSINKTKWDASLQKVAEQLRKHFPNKTINFTKPSGGDLEITTHSFNCGGEFFYCNTTSLFNSTWKNGATIQENSTETNGIMTLPCRIKQIVDMWQEVGQAMYAPPIAGVIYCTSNITGIILTRDGGSSNTNSEIFRPGGGDMRDNWRSELYKYKVVKIEPLGVAPSRAKRRVVEREKRAVGIGAVFLGFLGAAGSTMGAASITLTVQARQLLSGIVQQQSNLLKAIEAQQHLLKLTVWGIKQLQARVLALERYLQDQQLLGIWGCSGKLICTTTVPWNSSWSNKTYEEIWNNMTWLQWDREIDNYTNIIYNLLEESQNQQEKNEQDLLALDKWASLWNWFSITNWLWYIRIFIMIVGGLIGLRIVMAIISVVNRVRQGYSPLSFQIPTPNPEGLDRHGRIEEGGGEQDRTRSIRLVSGFLGLAWDDLRSLCLFSYHRLRDCILIVARTVELLGHSSLKGLRLGWEGLKYLGNLLLYWGRELKNSAISLLNSTAIAVAEWTDRVIEIGQRACRAILNIPRRIRQGFERALL

SEQ ID NO: 79 - protein, 850 aa, HIV
TRVMGTQRNCQKWWEWGILVFGMIMMCKAADLWVTVYYGVPVWRDADTTLFCASDAKAYATEKHNVWATHACVPTDPNPQEVNLANVTEDFNMWKNNMVEQMHADIISLWDQSLKPCVKLTPLCVTLNCSNANTTNTNSTEEIKNCSYNMPTELKDKTQKVYSLFYELDVVLLNRSKNSSYSTYRLISCNTSVITQACPKVSFEPIPIHYCAPAGYAILKCKDKEFNGKGSCSNVSSVQCAHGIRPVASTQLLLNGSLAEGKVMIRSENITDNAKNIIVQFNKPVPINCTRPNNNTRKSIRFGPGQAFYTNNNIIGDIRQAHCNISITEWNATLKKVVEQLREHFPNKTIIFNSSSGGDLEITTHSFNCGGEFFYCNTTGLFNSTWENGTNKQNYTESNDTITLQCRIKQIINMWQRVGRAMYAPPIAGVIKCTSNITGMILTRDGGKNSINETFRPGGGDMRDNWRSELYKYKVVKIEPLGIAPTEARRRVVQREKRAVGLGAVFLGFLGAAGSTMGAASITLTVQARQLLTGIVQQQSNLLKAIEAQQQMLRLTVWGIKQLQARVLALERYLQDQQLLGIWGCSGKLICATDVRWNSSWSNKTQEQIWKNMTWLQWDKEISTYTDIIYMLLEESQNQQEKNEQDLLALDKWANLWNWFDITRWLWYIKIFIMIVGGLIGLRIVIAIISVVKRVRQGYSPLSFQIPTPNPEGLDRPGRIEEEGGEQGRDRSIRLVSGFLALAWDDLRSLCLFSYHRLRDCILIAARIVELVGHSSLKGLRLGWEGLKHLWNLLVYWGQELKTSAIRLLDTIAVAVAEWTDRVIEIGQRACRAIRNIPRRIRQGLERALL

SEQ ID NO: 80 - protein, 865 aa, HIV
MRVMGTQRNYQHLWRGGILILGMLIMCKATDLWVTVYYGVPVWKDADTILFCASDAKAYDTEVHNVWATHACVPTDPNPQEINLENVTENFNMWKNNMVEQMQEDIISLWDQSLKPCVKLTPLCVILNCSNANTSTHSNSSSTQSPINEEIKNCSYNTTTILRDKTQKVYSLFYRLDVVQLDESENKNTSGSNTLYRLINCNTSTITQACPKVTFEPIPIHYCAPAGFAILKCKDPRFNGTGSCKNVSSVQCTHGIKPVASTQLLLNGSLAEGGKIMIRSENITNNAKNIIVQFTKPVLITCIRPNNNTRKSIRFGPGQAFYTNEIIGDIRQAHCNINKTLWNDTLQKVAEQLREKFPKKTIIFTNSSGGDPEITTLSFNCAGEFFYCNTTGLFNGTWWNNGTWNGPYTPNNTNGSIILPCRIKQIINMWQRVGRAMYAPPIAGIIKCTSNITGIILTRDGGNNGTNETFRPGGGDMRDNWRSELYKYKVVKLEPLGVAPTRAKRRVVEREKRAVGLGAVFLGFLGAAGSTMGAASLTLTVQARQLLSGIVQQQSNLLQAIEAQQHLLKLTVWGIKQLQARVLAVERYLKDQQLLGIWGCSGKLICATTVPWNTSWSNKSQDEIWDNMTWLQWDKEISNYTNIIYRLLEESQNQQEKNEQDLLALDKWADLWSWFNISHWLWYIRIFIMIVGGLIGLRIVFAIITVVNRVRQGYSPVSFQIPTPSPEGPDRPRGTEEGGGEQGRDRSIRLVNGFFALAWDDLRSLCLFSYHRLRDCILIAARTVELLGHCSLKGLRLGWEGLKNLWNLLLYWGRELKNSAISLFDTIAVAVAEWTDRVIEIGQRAFRAILNIPRRIRQGLERALL

SEQ ID NO: 81 - protein, 856 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 82 - protein, 855 aa, HIV
MKVKGIRKNYQLLWRWGIMLLGTLMICSATENLWVTVYYGVPVWKEATTTLFCASDAKAYETEVHNVWATHACVPTDPNPQELVLENVTENFNMWKNNMVEQMHEDIISLWDESLKPCVKLTPLCVTLNCTDANITSSNNITGSNNNSNLEQMAREISNCSFNITTTIKNKRQREFALLSKLDIVPIDNDSYSYMLINCNTSVITQACPKVSFQPIPIHYCTPAGFAILKCNDKKFNGTGPCKNVSTVQCTHGIRPVVSTQLLLNGSLAEEDVVIRSKNFTDNTKTIIVQLKESVEINCTRPNNNTRKSIHIGPGRAFYATGEIIGDIRQAHCNLSRAKWNDTLNQIVGKLRELYKNKTIVFNSSSGGDPEIVMHSFNCRGEFFYCNTTQLFNSTWDVNATGNGTTEPNSTITLPCRIKQIINRWQEVGKAMYAPPIAGQISCSSNITGLLLTRDGGGGENNSTEIFRPGGGDMRDNWRSELYKYKVVKIEPLGVAPTKAKRRVVQREKRAITLGAMFLGFLGAAGSTMGAASMALTVQARQLLSGIVQQQNNLLRAIEAQQHLLQLTVWGIKQLQARVLAIERYLQDQQLLGIWGCSGKLICTTTVPWNASWSNKSLDQIWENMTWMQWEREIDNYTSLIYTLIEDSQKQQEKNEQELLALDTWASLWNWFSITNWLWYIKIFIMIVGGLVGLRIVFIVLSIVNRVRKGYSPLSFQTHLPAPRGPDRPEGIEEEGGERDRDGSGPLVNGFLAIIWVDLRSLCLFSYHRLRDLLLIVVRIVELLGRRGWEALKYWWNLLQYWIQELRGSAVSLFNAIAIAVAEGTDRVIETIQRAFRAILHIPRRIRQGLERILL

SEQ ID NO: 83 - protein, 849 aa, HIV
  misc_feature (93)..(93): Xaa can be any naturally occurring amino acid
MRVKEIRKNCQHLWRWGILLLGILMISSAAENLWVTVYYGVPVWKEATTTLFCASDAKAYDTEVHNVWATHACVPTDPNPQEVVLVNVTENFXMWTNNMAEQMHEDIISLWDQSLKPCVKLTPLCVTLNCTDLRNTTNTNSTAEEMEAKGEMKNCSFNITTSIRNKLQKEYALFYKLDIVPINNDNTSYRLISCNTSVITQACPKVSFEPIPIHYCAPAGFAILKCNDKKFSGNGPCKNVSTVQCTHGIKPVVSTQLLLNGSLAEEEVVIRSENFTDNAKTIIVQLKEPVEINCTRPNNYTRKRITMGPGRVYYTTGEIIGDIRRAHCNISSTKWNNTLGQIVKKLKEQFNNNTIVFKKSSGGDPEIVMHSFICGGEFFFCNSTKLFNSTWNSTEGNDDGEERNITLPCRIKQIVNMWQEVGKAMYAPPIGGQIRCTSNITGLLLTRDGGNQNGTNETEIFRPGGGNMRDNWRSELYKYKVVKIEPLGVAPTKAKRRVVQREKRAVGIGAVFLGFLGAAGSTMGAASVTLTVQARLLLSGIVQQQNNLLRAIEAQQHLLQLTVWGIKQLQARVLAVERYLKDQQLLGIWGCSGKLICTTAVPWNASWSNKSLDEIWNNMTWMQWEREINNYTGLIYTLIEESQNQQEKNELDLLQLDKWASLWNWFDITNWLWYIKIFIMIVGGLVGLRIIFTVLSIVNRVRQGYSPLSFQTHLPAPRGPDRPGGIEEEGGERDRDTSGRLVDGFLAIFWVDLRNLCLFSYHRLRDLLLIVTRIVELLGRRGWEALKYWWNLLQYWSQELKNSAVSLLNATAIAVAEGTDRVIEVLQRVYRAILNIPTRIRQGLERALL

SEQ ID NO: 84 - protein, 856 aa, HIV
MRVKGIRRNCQHSWRWGTTLTMLLGILMICRAAEQLWVTVYYGVPVWREAKTTLFCASDAKAYDTEVHNVWATHACVPTDPNPQELVLVNVTENFNAWENNMVEQMHEDIISLWDQSLKPCVKLTPLCVTLNCNDLNTTTSNTTGTEGLTMDKGEMKNCSFNITTDISNKKQKQYALFYKLDVVQMNNNNNSYRLISCNTSVITQACPKVSFEPIPIYYCAPAGFAILKCNDKSFSGKGECKNVSTVQCTHGIRPVVSTQLLLNGSLAEEDVIIRSDNFTDNAKTIIVQLNETVDIHCIRPNNNTRKRITMGPGKVYYTTGQIIGDIRQAHCNLSEAKWNNTLRRVVRKLREKFNKTIVFNQSSGGDPEIVMHTFNCGGEFFYCNSTKLFNSIWDNNKDSTKTNEPNDGKNITLPCRIKQIINMWQGVGKAMYAPPIRGQIRCTSNITGLLLTRDGGKNNGTNGTEVFRPGGGNMKDNWRSELYKYKVVKIEPLGVAPTTAKRRVVQREKRAVTLGALFLGFLGAAGSTMGAASMTLTVQARLLLSGIVQQQNNLLRAIEAQQHLLQLTVWGIKQLQARVLAVERYLEDQQLLGIWGCSGKLICTTAVPWNASWSNKSRSEIWNNMTWMQWDKEIHNYTNLIYTLIGESQIQQEKNEQELLGLDKWASLWNWFDITKWLWYIKIFIMIVGGLIGLRIVFTVLSIMNRVRQGYSPLSFQTRLPTQRGPDRPEGTEEEGGERDRDRSGPLVDGFLAIIWVDLRSLCLFLYHRLRDLLLIVTRTLELLGRRGWEILKYWWNLLQYWSQELKNSAVSLLNATAIAVAEGTDRVIEIVQRTFRAILHIPVRIRQGLERALL

SEQ ID NO: 85 - protein, 870 aa, HIV
MKVKETRRNYQHLWRWGTMLLGMLMICRAAENLWVTVYYGVPVWKEATTTLFCASDAKAYETEVHNVWATHACVPTDPNPQEIVLENVTENFNMWKNNMVEQMHEDIISLWDQSLKPCVKLTPLCVTLNCTDYLGNTTKTNTTSAPTTSTTTTNTTNNKGELKNCSFQVTTGIGDRTKKEYALFYKHDVVPIDNDNNKTNNSNFILIHCNSSVITQACPKVSFEPIPIHYCAPAGFAILKCKDKKFNGTGPCKNVSTVQCTHGIRPVVSTQLLLNGSLAEEEIVIRSQNFTDNVKSIIVQLNETVKINCTRPNNNTRKAIRIGRGRAIYATDRIIGDIRQAYCNISRTKWNDTLGQIATKLREQFGNKTIVFNSSSGGDPEIVMHSFNCGGEFFYCNTTQLFNGMWHANGTWNSTWNDTGGSNDTIRLPCRIKQIVNMWQEVGKAMYAPPIKGQIQCSSNITGLILTRDGGNSTNETGEVFRPGGGNMKDNWRSELYKYKVVEIEPLGVAPTKAKRRVVQREKRAVGLIGAVFLGFLGAAGSTMGAASIALTVQTRHLLSGIVQQQNNLLRAIEAQQHLLQLTVWGIKQLQARILAVERYLRDQQLLGIWGCSGKLICPTAVPWNASWSNKSLEEIWENMTWREWEREIDNYTGKIYDLLAKSQNQREMNEQELLKLDKWADLWNWFDITQWLWYIKIFIMIVGGLIGLRIIFAVISIVNRVRQGYSPLSLQTLLPTQRGPDRPEGIEEEGGERDRDRSIRLVEGFSALIWDDLRSLFLFSYHRLRDLLLIVTRIVELLGRRGWEALKYWWNLLQYWIQELKNSAINLLNTTAIVVAEGTDRVIEVVLRAYRAILHIPRRIRQGLERLLL

SEQ ID NO: 86 - protein, 856 aa, HIV
MRVEGIQRNWKQWWIWGILGFWMVMIYNVRGNLWVTVYYGVPVWKEAKTTLFCASDAKAYDAEVHNVWATHACVPTDPNPQEMVLENVTENFNMWENDMVEQMHQDIISLWDQSLKPCVKLTPLCVTLHCSNRTIDYNNRTDNMGGEIKNCSFNMTTEVRDKREKVHALFYRLDIVPLKNESSNTSGDYRLINCNTSAITQACPKVSFDPIPIHYCAPAGYAILKCNNKTFNGTGPCNNVSTIQCTHGTKPVVSTQLLLNGSLAEEEIIIRSKNLTDNVKTIIVHLNESVEINCTRPNNNTRKSIRIGPGQAFYATGEIIGDIRQAHCNISRTAWNKTLQEVGKKLAEHFPNKAIKFAKHSGGDLEITTHSFNCRGEFFYCNTSSLFNSTYTPNSTENITGTENSIITIPCRIKQIINMWQGVGRAMYAPPIEGILTCRSNITGLLLTRDGGTGMHDTEIFRPEGGDMRDNWRSELYKYKVVEIKPLGIAPTKAKRRVVEREKRAVGIGAVFLGFLGAAGSTMGAASITLTVQVRQLLSGIVQQQSNLLRAIEAQQHMLQLTVWGIKQLQTRVLAIERYLRDQQLLGIWGCSGKLICTTAVPWNSSWSNRSQEDIWNNMTWMQWDREISNYTNTIYRLLEDSQNQQEKNEQDLLALDKWQNLWTWFGITNWLWYIKIFIKIVGGLIGLRIIFAVLSIVNRVRQGYSPLSFQTLTPNPRGPDRLGGIEEEGGEQDRDRSIRLVSGFLALAWDDLRSLCLFSYHRLRDLILIAARAVELLGRSSLRGIQRGWEILKYLGGLVQYWSLELKKSAISLFDTIAIAVAEGTDRIIEVIQGIWRAICNIPRRIRQGFEAALQ

SEQ ID NO: 87 - protein, 851 aa, HIV
MKVMGIQRNCQQWWIWGILGFWMLMICNGMGNLWVTVYYGVPVWKDASPTLFCASDAKAYDTEVHNVWGTFACVPTDPSPQELGLENVTENFNMWKNDMVEQMHQDIISLWDQGLKPCVKLTPLCVTLNCNAIKNNTKVTNNSINSANDEMKNCSFNITTELRDKKRKAYALFYKLDIVPLNNGSTDYRLINCNTSTITQACPKVSLDPIPIHYCAPAGYAILKCRDKTFTGTGPCHNVSTVQCTHGIKPVVSTQLLLNGSIAEGETIIRFENLTNNAKIIIVQLNESVEITCTRPSNNTRESIRIGPGQTFYATGDIIGDIRQAHCNISEEKWNKTLQKVKEKLQKHFPNKTIEFKPSSGGDLEITTHSFNCGGEFFYCNTSNLFNSTKLELFNSSTNLNITLQCRIKQIINMWQGVGRAMYAPPIEGIIMCRSNITGLLLTRDGAKEPHSTKEIFRPEGGDMRDNWRSELYKYKVVEIKPLGVAPTKPKRRVVEREKRAALGALFLGFLGAAGSTMGAASITLTVQARQLLSGIVQQQSNLLKAIEAQQHMLQLTVWGIKQLQTRVLAIERHLRDQQLLGIWGCSGKLICTTAVPWNSSWSNKSQEEIWDNMTWMQWDREISNYTDIIYNLLEVSQNQQDKNEKDLLALDKWENLWNWFNITNWLWYIKIFIMIVGGVIGLRIIFAVLSIVNRVRQGYSPLSFQTLIPHPRGPDRLGGIEEEGGEQGRDRSIRLVNGFLAIFWDDLRSLCLFSYHRLRDLILIAARTVELLGRSSLKGLQRGWETLKYLGSLVQYWGLELKKSAINLLNTTAIVVGEGTDRFIELIQRIWRAFCNIPRRIRQGLEAALQ

SEQ ID NO: 88 - protein, 870 aa, HIV
MRVRGILRNYQQWWIWGVLGFWMLMICNVVGNLWVTVYYGVPVWKEANTTLFCASDAKAYEKEVHNVWATHACVPTDPNPQEIVMENVTENFNMWKNDMVNQMHEDVISLWDQSLKPCVKLTPLCVTLECRNVNSTGNGTHSKTYNESMKEIKNCSFNATTVIKDKKQTVYALFYKLDIVPLDNEEQENDSNSSGYYRLINCNTSALTQACPKVTFDPIPIHYCAPAGYAILKCNNKTFNGTGPCHNVSTVQCTHGIKPVVSTQLLLNGSLAEGGIIIRSENLTNNVKTIIVHLNQPVEIMCTRPDNNTRKSIRIGPGQTFYATGDIIGDIRQAHCNISEDKWNETLQNVSKKLAEHFPNKTIIFNSSSGGDLEITTHSFNCRGEFFYCNTSGLFNRTYMPNDTKSNSSSNPNANITIPCRIKQIINMWQEVGRAMYAPPIEGKITCRSNITGLLLVRDGGEDKNNTETNKTETFRPGGGDMRDNWRSELYKYKVVEVKPLGVAPTTAKRRVVEREKRAVGIGAVFLGFLGAAGSTMGAASITLTVQARQLLSGIVQQQSNLLRAIEAQQHLLQLTVWGIKQLQTRVLAIERYLKDQQLLGIWGCSGKLICTTAVPWNSSWSNRTQKEIWDNMTWMQWDREINNYTNTIYRLLEESQNQQEENEKDLLALDSWKNLWNWFDITKWLWYIKIFIIIVGGLIGLRIIFAVISIVNRVRQGYSPLSFQTLTPNPGGPDRLGRIEEEGGEQDKDRSIRLVSGFLALFWDDLRNLCLFSYHRLRDFILVAARVLELLGRRSLRGLQRGWEALKYLGSLVQYWGLELKKSAINLLDRIAIAVAEGTDRILELVQRICRAIRNIPRRIRQGFEAALQ

SEQ ID NO: 89 - protein, 846 aa, HIV
MRVRGILRNWPQWWIWGILGFWMIIICRGEENSWVTVYYGVPVWTEAKTTLFCASDAKAYEKEVHNVWATHACVPTDPSPQELVLENVTESFNMWENDMVDQMHEDIIGLWDESLKPCVKLTPLCVTLNCNTTSHNNSSPSPMTNCSFNATTELRDKTQKVNALFYRSDIVPLEKNSSEYILINCNTSTITQACPKVSFDPIPIHYCAPAGYAILKCNNKTFNGTGPCSNVSTVQCTHGIKPVVSTQLLLNGSLAEGEIIIRSENLTDNAKTIIVHLNKSVAIVCTRPNNNTRKSIRIGPGQVFYTNEIIGNIRQAHCNISRELWNNTLEQVKKKLKEHFQNKTIEFQPPAGGDLEVTTHSFNCRGEFFYCNTSNLFNITASNASDANNNTITLPCKIKQIINMWQEVGRAMYAPPIAGNITCNSSITGLLLTRDGGNNNDTGNNNDTEIFRPGGGNMKDNWRSELYKYKVVEIKPLGIAPTKAKRRVVEREKRAVGLGAVLLGFLGTAGSTMGAASITLTVQARQLLSGIVQQQSNLLRAIEAQQHMLQLTVWGIKQLQARVLAIERYLKDQQLLGLWGCSGKLICTTAVHWNSSWSNKSQDYIWGNMTWMQWDREINNYTDIIYTLLEESQSQQEKNEKDLLALDSWNNLWNWFSITKWLWYIKIFIMIVGGLIGLRIILGVLSIVKRVRQGYSPLSFQTLPPNPRGPDRLRGIEEEGGEQDKDRSIRLVSGFLALVWEDLRSLCLFSYHRLRDFILIAGRAAELLGRSSLRGLQTGWQALKYLGSLVQYWGLELKKSAINLFDTTAIVVAEGTDRLIEGLQGIGRAIYNIPRRIRQGFEAALL

SEQ ID NO: 90 - protein, 853 aa, HIV
MRARGIERNCQNWWKWGIMLLGILMTCSAADNLWVTVYYGVPVWKEATTTLFCASDAKSYETEAHNIWATHACVPTDPNPQEIALENVTENFNMWKNNMVEQMHEDIISLWDQSLKPCVKLTPLCVTLNCSDELRNNGTMGNNVTTEEKGMKNCSFNVTTVLKDKKQQVYALFYRLDIVPIDNDSSTNSTNYRLINCNTSAITQACPKVSFEPIPIHYCAPAGFAILKCRDKKFNGTGPCTNVSTVQCTHGIRPVVSTQLLLNGSLAEEEVIIRSENLTNNAKNIIAHLNESVKITCARPYQNTRQRTPIGLGQSLYTTRSRSIIGQAHCNISRAQWSKTLQQVARKLGTLLNKTIIKFKPSSGGDPEITTHSFNCGGEFFYCNTSGLFNSTWNISAWNNITESNNSTNTNITLQCRIKQIIKMVAGRKAIYAPPIERNILCSSNITGLLLTRDGGINNSTNETFRPGGGDMRDNWRSELYKYKVVQIEPLGVAPTRAKRRVVEREKRAIGLGAMFLGFLGAAGSTMGARSVTLTVQARQLMSGIVQQQNNLLRAIEAQQHLLQLTVWGIKQLQARILAVERYLKDQQLLGIWGCSGKHICTTNVPWNSSWSNRSLNEIWQNMTWMEWEREIDNYTGLIYSLIEESQTQQEKNEKELLELDKWASLWNWFSITQWLWYIKIFIMIIGGLIGLRIVFAVLSLVNRVRQGYSPLSFQTLLPAPRGPDRPEGTEEEGGERGRDRSVRLLNGFSALIWDDLRSLCLFSYHRLRDLILIAVRIVELLGRRGWDILKYLWNLLQYWSQELRNSASSLFDAIAIAVAEGTDRVIEIIQRACRAVLNIPRRIRQGLERSLL

SEQ ID NO: 91 - protein, 848 aa, HIV
MRVMGIERNYQHSWKWGTMLLGMLLMTYSAAGNLWVTVYYGVPVWKEAKTTLFCASDAKSYKTEAHNIWATHACVPTDPDPQELVLDNVTENFNMWKNNMVEQMHEDIISLWDQSLKPCVKLTPLCVTLNCSNPNITNNSSANNISIVKKMKNCSFNTTTILKDKQKQEYALFYILDIVGIDNSSNRLINCTTSVITQACPKITSEPIPIHYCAPAGFAILKCNDKLFNGTGPCRNVSAVQCTHGIKPVVSTQLLLNGSLAEEVMVRSENLTDNAKNIIVQLNNTINITCVRPNSNTRKSINLGPGQAFYATYATNIIGNIRQAHCNLSATQWNKTLHQVAQKLGKLLNKTKINFNSSSGGDPEITTHSFVCGGEFFYCNTSGLFNGTWDNGTWTWNTSAVPNETITIPCRIKQIINMWQGVGRAMYAPPIEGLIKCSSNITGLLLTRDGGNTSDSAETFRPGGGDMRDNWRSELYKYKVVKIEPLGVAPTRAKRRVVEREKRAIGLGAMFLGFLGAAGSTMGAASVTLTVQARQLLSGIVQQQNNLLRAIEAQQHLLQLTVWGIKQLQARVLAVERYLKDQQLLGIWGCSGKHICTTNVPWNSSWSNRSLDDIWQNMTWMQWEREIENYTGVIYSLIEESQIQQEKNEKELLELDKWASLWNWFSISNWLWYIRIFIMIVGGLIGLRIVFAVLSMVRRVRQGYSPLSFQTLLPAPRGPDRPEGIEEEGGEQDRGRSIRLVNGFSALIWDDLRNLCLFSYHRLRDLILIAARIVDLLGRRGWEALKYLWNLLRYWSQELKNSAINLLNTTAIAVAEGTDRVIEIIQRAGRAVLHIPRRIRQGFERALL

SEQ ID NO: 92 - protein, 852 aa, HIV
MRVMETQRNYQHLWRWGIMLLGMWMTYSVAEQLWVTIYYGVPVWREANTTLFCASDAKSFDTEAHNIWATHACVPTDPNPQEMDLVNVSENFNMWKNNMVEQMHEDIISLWEESLKPCVKLTPLCVTLHCSDANTTNSGNGTNTTDPRLIEKGEMKNCSFNITTEIRDKRKQVQALFYKLDVVPIDKKNNNSYTLMHCNTSAIKQACPKVSFEPIPIHYCAPAGFAILKCKDKKFNGTGPCKKVSTVQCTHGIRPVVSTQLLLNGSLAGEEIIIRSENLTNNVKTIIVQLNETVKINCTRPNNNTRKGIRIGPGQTFFTAEVTGDIRKAYCNISGAEWDKTLQQVATKLGDLLNKTIINFSPSSGGDPEITTHSFNCGGEFFYCNTSLLFNTTWIKGTQNNTETNNSTITIPCRIKQIINMWQGVGKAMYAPPIAGLIRCTSNITGLLLTRDGGNVNNSREEIFRPGGGDMRDNWRSELYKYKVVRIEPIGVAPTRAKRRVVEREKRAIGLGAMFLGFLGAAGSTMGAASLTLTVQARQLLSGIVQQQNNLLRAIEAQQHLLQLTVWGIKQLQARVLAVESYLKDQQLLGIWGCSGKHICTTAVPWNSSWSNKSLDDIWNNMTWMEWEKEIDNYTGVIYSLIEESQVQQEKNEKELLELDKWASLWNWFSITKWLWYIKLFIMIVGGLIGLKIVFTVFSLVNRVRQGYSPLSLQTLLPASRGPDRPEGIEEEGGEQGRGRSIRLVNGFSALIWDDLRNLCLFSYRHLRDLILIATRIVGLLGHRGWEAIKYLWNLLQYWIQELKNSAISLLNVTAIAVAEGTDRIIEIIQRAFRAVLHIPRRVRQGLERALL

SEQ ID NO: 93 - protein, 844 aa, HIV
MRVRETKRNYQHLWKWGTMLLGMLMICSVTGKSWVTVYYGVPVWKEATTTLFCASDAKAYKAEAHNIWATHACVPTDPNPQEIKLENVTENFNMWKNNMVEQMHEDIISLWDQSLKPCVKLTPLCVTLNCTNWVTDTTNTTGMANCSFNITTEIRDKKKQVQALFYKLDVVKINDNDSDNTSYRLINCNTSAITQACPKMTFEPIPIHYCAPAGFAILKCNEKKFNGTGPCKNVSTVQCTHGIKPVVSTQLLLNGSLAEEEIIIRSENLTNNAKIIIVQLNESVPINCIRPYNNTRQSTRIGPGQALFTTKVIGDIRQAHCNISGAGWNKTLQQVAEKLGNLLNQTTIIFKPSSGGDPEITTHSFNCGGEFFYCNTTRLFNSTWKRNNSEWRSDNTPDETITLQCRIKQIINMWQEVGKAMYAPPIEGFINCSSNITGLLLTRDGGAINSSQNETFRPGGGDMRNNWRSELYKYKVVKLEPIGLAPTAAKRRVVEREKRAIGLGALFLGFLGTAGSTMGAVSLTLTVQARQVLSGIVQQQNNLLRAIEAQQHLLQLTVWGIKQLQARILAVESYLKDQQLLGIWGCSGKHICTTNVPWNSSWSNRSVDEIWNNMTWMEWEREIDNYTELVYSLLEVSQIQQEKNEQELLKLDTWASLWNWFSITQWLWYIKIFIMIVGGLIGLRIVFAVLSVVNRVRQGYSPLSFQTLLPAPREPDRPEGIEEEGGERDRGRSIRLVNGLSALIWDDLRNLCLFSYHRLRDLILIAARIVELLGRRGWEAIKYLWNLLQYWIQELKNSAVSLFNTIAIAVAEGTDRAIELVQRAVRAILNIPVRIRQGLERALL

SEQ ID NO: 94 - protein, 832 aa, HIV
MRVRGMQRNWQHLGKWGLLFLGILIICNAADNLWVTVYYGVPVWKEATTTLFCASDAKAYEREAHNVWATHACVPTDPNPQEVFLKNVTENFDMWKNNMVEQMHTDIISLWDQSLKPCVKLTPLCVTLNCTNATNNSQEKPGAMQNCSFNMTTEVRDKKLKLSALFYRLDIVPIGNNNSSEYRLINCNTSTITQACPKVSWDPIPIHYCAPAGYAILKCNDKRFNGTGPCKNVSTVQCTHGIKPVVSTQLLLNGSLAEEGIVIRSQNISNNAKTIIVHLNESVQINCTRPNNNTRKGIHLGPGQTFYATGAIIGDIRKAHCNISGTQWNNTLEYVKAELKSHFPNNTAIKFNQSSGGDLEITMHSFNCRGEFFYCDTSGLFNDTGSNNGTITLPCRIKQIVNMWQGVGRAMYTSPIAGNITCNSNITGLLLTRDGGNESNIETFRPEGGNMKDNWRSELYKYKVVEIEPLGVAPTKAKRQVVQREKRAAGLGALFLGFLGDSREHMGAASITLTVQARQLLSGIVQQQNNLLRAIEAQQHLLQLTVWGIKQLQARVLAVERYLKDQQLLGIWGCSGKLICTTNVPWNSSWSNKSQEEIWNNMTWMEWEKEISNYSNIIYKLIEESQNQQEKNEQELLALDKWASLWNWFDISNWLWYIKIFIMIVGGLIGLRIVFAVLSIVNRVRKGYSPLSLQTLIPSPRGPDRPEGIEEGGGEQGKDRSVRLVTGFLALAWDDLRNLCLFSYRHLRDFILIAARIVDRGLRRGWEALKYLGNLTRYWSQELKNSAISLFNTTAIVVAEGTDRIIEVLQRAGRAVLNIPRRIRQGAERALL

SEQ ID NO: 95 - protein, 846 aa, HIV
MRVRGMQRNWQHLGKWGLLFLGTLIICNAAENLWVTVYYGVPVWKEATTTLFCASDAKSYEKEAHNVWATHACVPTDPNPQEVVLENVTERFNMWENNMVEQMHTDIISLWDQSLKPCVKLTPLCVTLDCRNIATNGTNDTIAINDTLKEDPEAIQNCSFNTTTEIRDKQLKVHALFYKLDIVQINKDDNRTYRLINCDASTITQACPKVSWDPIPIHYCAPAGYAILKCNEKNFTGTGSCKNVSTVQCTHGIKPVVSTQLLLNGSLAEGEIVIRSQNISDNAKTIIVHLNESVQINCTRPNNNTRKRISLGPGRVFYTTGEIIGDIRKAHCNVSGTQWRNTLAKVKAKLGSYFPNATIKFNSSSGGDLEITRHNFNCMGEFFYCNTDELFNDTKFNDTGFNGTITLPCRIKQIVNMWQEVGRAMYANPIAGNITCNSNITGLLLTRDGGLNSTNETFRPGGGNMKDNWRSELYKYKVVEIEPLGVAPTKAKRQVVKRERRAVGLGALFLGFLGAAGSTMGAASITLTVQARQLLSGIVQQQSNLLRAIEAQQHLLQLTVWGIKQLQARVLAVERYLKDQQLLGLWGCSGKLICTTNVPWNSSWSNKSLEEIWGNMTWMEWEKEVSNYSKEIYRLIEDSQNQQEKNEQELLALDKWASLWNWFDITQWLWYIKIFIMIVGGLIGLRIVFTVLSIVNRVRKGYSPLSFQTHIPSPREPDRPEGIEEGGGEQGKDRSVRLVTGFLALAWDDLRNLCLFSYRHLRDFILIAARIVDRGLKRGWEALKYLGNLTQYWGQELKNSAISLLNATAIAVAEWTDRVIEALQRAGRAILNIPRRIRQGLERALL

SEQ ID NO: 96 - protein, 842 aa, HIV
MRVRGMQRNWQHLGKWGLLFLGMLIICKAADDLWVTIYYGVPVWKEANTTLFCASDAKSYEKEVHNVWATHACVPTDPNPQEVALNVTENFNMWENDMVEQMHKDIISLWDQSLKPCVKLTPLCVTLNCTNATTTNDTLSDQSSTLKEEPGAIQNCSFNMTTEVEDKKQKVHALFYRLDIEPISNNNSREEYRLITCNTSTITQACPKVSWDPIPIHYCAPAGYAILKCKDKRFNGTGPCRNVSTVQCTHGIRPVVSTQLLLNGSLSEGGIIIRSQNLSDNAKTIIVHLNESVQINCTRPNNNTRKSIRIGPGQSFYATGEIIGDIRKAHCNISGEQWNKTLDRVKAELKLHFNKTIQFNSSSGGDLEITMHSFNCRGEFFYCNTSLLFNNTVPNNGTITLPCRIKQFVNMWQEVGRAMYAAPIAGNITCNSNITGLLLTRDGGQSNNSDSETFRPGGGDMKDNWRSELYKYKVVEIEPLGVAPTRPKRPVVRRERRAVAIGAVFLGFLSAAGSTMGAASLTLTVQARQLLSGIVQQQNNLLQAIEAQQHMLQLTVWGIKQLQARVLAVERYLKDQQLLGLWGCSGKLICTTNVPWNSSWSNKSQDEIWNNMTWMQWEKEISNYSKTIYMLIEKSQSQQERNEQELLELDKWDSLWSWFDITNWLWYIKIFIMIVGGLIGLRIVFAVLSIVNRVRKGYSPLSLQTLIPAPTEPDRPEGIEEGGGEQGKDRSVRLVNGFLALVWDDLRNLCLFSYRHLRDFILIAARIVDRGLRRGWEALKYLGNIIQYWSQELKNSAISLFNTTAIVVAEGTDRVIEALQRAVRAVLNIPRRIRQRVERALI

SEQ ID NO: 97 - protein, 843 aa, HIV
MRVRVMQRNWQHLGKWGLLFLGILIICSAADNLWVTVYYGVPVWKEATTTLFCASDAKGYEREVHNVWATHACVPTDPNPQEIWLKNVTENFDMWKNNMVEQMHEDIISLWDQSLKPCVKLTPLCVTLHCSDVNITSNATTTNDTSTPEESGAIQNCSFNMTTEVKDKKLRVNALFYKLDIIPINNSSSSDYRLINCNTSTIKQACPKVSWDPIPIHYCAPAGYAILKCRDPRFNGTGPCKNVSTVQCTHGIRPVVSTQLLLNGSLAEEDIIIRSQNISDNAKTIIVHLNESVQINCTRPNNNTRKSIHLGPGQAFYATGDIIGDIKKAYCEINGTQWSKTKTQVQEKLRALFNKTIKFNQSSGGDLEITMHSFNCRGEFFYCDTSGLFNESEKYNGTIILPCKIKQIINMWQGVGQAMYSAPIAGRINCNSTITGLLLTRDGGQSNDTNRTETFRPEGGNMKDNWRNELYKYKVVEIEPLGVAPTKARRRVVQRERRAVGIGALFLRFLGAAGSNIGAASITLTVQARQLLSGIVQQQNNLLRAIEAQQHLLQLTVWGIKQLQARVLAVERYLKDQQLLGIWGCSGKLICTTNVPWNTSWSNKSHDEIWNNMTWMQWEKEINNYSNTIYRLIEESQNQQEKNEQELLALDKWASLWSWFDISNWLWYIKIFIMIVGGMIGLRIVFAVLSIVNRVRKGYSPLSLQTLIPSPRGPDRPEGIEEGGGEQDRNRSVRLVNGFLSLVWDDLRNLCLFSYRHLRDFILIAARTVDRGLTRGWETLKYLWNLAQYWSQELKNSAISLLNTTAIVVAEGTDRVIEVLQRAGRAVLNVPRRIRQGLERSLL

SEQ ID NO: 98 - protein, 845 aa, HIV
MRVRGMQRNWQHLGKWGFLFLGILIICNAADNLWVTVYYGVPVWKEATTTLFCASDAKAYEKEAHNVWATHACVPTDPDPQEIFLDNVTENFNMWKNNMVDQMHEDIISLWDQSLKPCVKITPLCVTLHCSDVNITANNTNQPNNITLEEQGEIKNCSFNITTEIKDKRKNEFATFYKHDIVPIEKNTTSYRLTSCNTSTVTQACPKVSFDPIPIYYCAPAGYAILKCNDKRFNGKGLCTNVSTVQCTHGIKPVVSTQLLLNGSLAEKNIIIRSENITDNAKTIIVQFNESVKINCTRPNNNTRKSIRIGPGQVFYATGEIIGDIRKAHCTINGTLWNATLNRVAAEVKNLTNITIKFEPSSGGDLEVTTHSFSCGGEFFYCDTTALFNTTLLNTTMDNNGTIIIPCRIKQIVNVWQRVGRAMYAPPIAGKIQCNSNITGLLLTRDGGSKANNTDILRPIGGEMRDNWRSELYKYKVVQIQPLGIAPSRAKRQVVKRERRAVGIGAVFLGFLGAAGSTMGAASITLTVQARQLLSGIVQQQNNLLKAIEAQQHLLQLTVWGIKQLQARILAVERYLKDQQLLGIWGCSGKLICTTNVPWNSSWSNKSQEEIWGNMTWMQWEKEIDNYTDTIYRLIEEAQNQQEKNEQDLLALDKWDSLWSWFTITNWLWYIRIFIMVVGGLIGLRIVFAVLSIINRVRQGYSPLSLQTLIPSPRGPDRPGGIEEEGGEQDKDRSVRLVSGFLALAWDDLRSLCLFSYRHLRDFILIAARTVDRGLKGGWEVLKYLWNLAQYWGRELKISAISLLNTTAIVVAEGTDRVIEILQRAGRAILHIPRRIRQGFERALL

SEQ ID NO: 99 - protein, 843 aa, HIV
MRVREMQRNWQHLGKWGLLFLGILIICNANATDDLWVTVYYGVPVWKEPTTTLFCASDAKAYDPEVHNVWATYACVPTDPNPQELVLGNVTENFNMWENNMVDQMHLDIISLWDQSLKPCVKSTPLCVTLNCTDVNITMSDINGTSLKEDQGEIKNCSFNVTTELKDKKRKQQALFYRLDVEPIKNSSNIYKLISCNMSTVTQACPKVSFDPIPIHYCAPAGYAILKCNDKRFNGTGPCEKVSTVQCTHGIRPVVSTQLLLNGSLAQEDIIIRSKNITDNTKNIIVQFNRSVIIDCRRPNNNTRKGIRIGPGQTFFATGEIIGDIRKAYCNINRTLWNETLKNVSGEFKKHFNFSVAFNSSSGGDVEITTHSFNCRGEFFYYNTSGLFNETEVANNTNENITLPCRIRQFVNMWQRIGRAMYAPPIEGEIQCTSNITGLLLTRDGSKDIDGKEILRPIGGDMRDNWRSELYKYKVVRIEPVGVAPTKAKRRVVQRAKRAVGMGAVLFGFLGAAGSTMGAAAITLTAQARQLLSGIVQQQSNLLKAIEAQQHLLQLTVWGIKQLQARILAVERYLKDQQLLGIWGCSGKLICTTNVRWNSSWSNKSYDDIWDNMTWMQWEKEIDNYTKTIYSLIEDAQNQQERNEQELLALDKWDSLWSWFSITNWLWYIKIFIMIVGGLIGLRIVFAVLSVVNRVRQGYSPLSLQTLIPNPRGPDRPGGIEEEGGEPDRDRSMRLVSGFLPLTWDDLRSLCSFSYRHLRDLLLIAARTVDRGVKGGWEALKYLWNLTQHWGRELKNSAISLFDTIAIAVAEGTDRIIEVLQRAGRAVLHIPRRIRQGAERFLL

SEQ ID NO: 100 - protein, 850 aa, HIV
MRVREMQRNWQHLGRWGLLFLGILIICSAADKLWVTVYYGVPVWKEATTTLFCASDAKAYEREVHNVWATYACVPTDPSPQELVLGNVSEKFNMWKNNMVDQMHEDIISLWDESLKPCVKLTPLCVTLNCTKAIINVTSSNNTTLAPNVTISEEMKNCSFNITTEIRDKQKKEYALFYKLDVVQINNSNTSYRLINCNTSTLTQACPKVSFDPIPIHYCAPAGFAILKCNNKTFNGTGLCRNVSTVQCTHGIKPVVSTQLLLNGSLAEEKMIIRSENISDNTKTIIVQFKNPVKINCTRPNNNTRRSIHIGPGRAFYATGEIIGDTRKAHCNISEKQWYDTLIKIATEFKDQYNKTVGFQPSAGGDLEITTHSFNCRGEFFYCNTTILFNHTRVNDILSNNHTRENDTITLPCRIKQIVNMWQRVGQAMYAPPIAGKIQCNSNITGLLLTIDGGEGNESETLRPGGGDMRDNWRSELYKYKVVKIEPLGVAPTKAKRQVVQREKRAVGMGAMFLGFLGAAGSTMGAASITLTVQARNLLSGIVQQQSNLLKAIEAQQHLLQLTVWGIKQLQARILAVERYLKDQQLLGIWGCSGKLICPTTVPWNLSWSNKSQDEIWGNMTWMEWEKEIGNYTDTIYRLIESAQNQQEKNEQDLLALDKWDNLWNWFSITRWLWYIEIFIMIIGSLIGLRIVFTVLSIINRVRQGYSPLSLQTLIPNSRGPERPGGIEEEGGEQDKDRSIRLVSGFLALAWDDFRSLCVFSYHCLRNFILIAARTVDKGLKRGWEVLKYLWNLAQYWGQELKNSAISLLDRTAIAVAEGTDRIIEILQRAGRAVLNIPRRIRQGLERALL

SEQ ID NO: 101 - protein, 849 aa, HIV
MRVREMQRNWQHLGKWGLLFLGILIICNAADNLWVTVYYGVPVWKEATTTLFCASDAKAYEREIHNVWATYACVPTDPNPQELVLGNVTENFNMWKNNMVDQMHEDIISLWDQSLKPCVQITPLCVTLNCTDVPVNITNGNSTLDNITLEEQGEIKNCSFNITTEINDIKKKESAIFYRLDVVPINNSTSEYRLLSCNTSTVTQACPKVSFDPIPIHYCAPAGFAILKCNDKEFNGTGLCRNVSTVQCTHGIKPVVSTQLLLNGSLAEGDIVIRSENISDNAKTIIVQFNRSVAINCTRPTNITRRSMRIGPGRVFYATGTVLGDIRKAYCTINGTLWNKTLEGVAKEVQSHLNKSITFAPSSGGDLEVTTHSFNCRGEFFYCNTVALFNATNMTNAMNRSNGIITLPCRIRQIVNMWQRVGRAMYAAPIAGQIQCNSSITGLILTRDGGKNNTNNDTLRPGGGDMRDNWRSELYKYKVVKIEPLGVAPTKAKRQVVKREREKRAVGIGAVLLGFLGAAGSTMGAASMTLTVQARQLLSGIVQQQNNLLKAIEAQQHLLQLTVWGIKQLQARILAVERYLKDQQLLGIWGCSGKLICTTNVPWNSSWSNKSQNEIWENMTWMQWEKEISNYTGTIYKLIENAQNQQEKNEQDLLALDKWDNLWSWFTITNWLWYIKLFIMIVGGLIGLRIVFAVLAVINRVRQGYSPLSLQTLTPSRREPERPGGIEEEGGEQDKTRSVRLVSGFLALAWDDLRSLCLFSYRHLRDFILIAARTVNKGLIRGWEILKYLGNLAQYWGREIKNSAIDLLNTTAIVVAEGTDRIIEVLQRAGRAILHIPRRIRQGAERALL

SEQ ID NO: 102 - protein, 854 aa, HIV
MRVKGIQRNWQHLWNWGILILGLVIICSAEKLWVTVYYGVPVWEDANAPLFCASDAKAHSTESHNIWATHACVPTDPSPQEINMRNVTENFNMWKNNMVEQMHEDIISLWDESLKPCVKLTPLCVTLNCTEINNNSTRNITEEYRMTNCSFNMTTELRDKKKAEYALFYRTDVVPINEMNNENNGTNSTWYRLTNCNVSTIKQACPKVTFEPIPIHYCAPAGFAILKCVDKKFNGTGTCNNVSTVQCTHGIKPVVSTQLLLNGSLAEKDIIISSENISDNAKVIIVHLNRSVEINCTRPNNNTRRSVAIGPGQAFYTTGEVIGDIRKAHCNVSWTKWNETLRDVQAKLQEYFINKSIEFNSSSGGDLEITTHSFNCGGEFFYCNTSGLFNNSILKSNISENNDTITLNCKIKQIVRMWQRVGQAMYAPPIAGNITCRSNITGLILTRDGGDNNSTSEIFRPGGGDMKNNWRSELYKYKTVKIKSLGIAPTRARRRVVEREKRAVGVGAIFLGFLGTAGSTMGAASITLTVQVRQLLSGIVQQQSNLLRAIEAQQHLLQLTVWGIKQLRARVLALERYLKDQQLLGIWGCSGKLICTTNVPWNTSWSNKSYNEIWENMTWIEWEREIDNYTYHIYSLIEQSQIQQEKNEQDLLALDQWASLWSWFSISNWLWYIRIFVMIVGGLIGLRIVFAVLSIVNRVRQGYSPLSFQTLLHHQREPDRPAGIEEGGGEQDRDRSIRLVSGFLALAWDDLRSLCLFSYHRLRDFILIAARTVELLGRNSLKGLRLGWEALKYLWNLLLYWARELKNSAINLLDTIAIAVANWTDRVIEVAQRAGRAVLNIPRRIRQGLERALL

SEQ ID NO: 103 - protein, 850 aa, HIV
  misc_feature (405)..(405): Xaa can be any naturally occurring amino acid
MRVKGIERNWQHLWKWGTLILGLVIICSASNNLWVTVYYGVPVWEDAKTTLFCASDAKAYSTERHNIWATHACVPTDPDPQEIPLGNVTENFNVWKNDMVEQMHEDIISLWDESLKPCVKLTPLCVTLNCTDANVTTVANESVSAQEIKNCSFNITTEIRDRKRKEYALFYRLDVIPINDDSSNSTGNYSNYRLINCNVSTIKQACPKVDFDPIPIHYCAPGGFAILKCKEKEFNGTGPCQNVSTVQCTHGIKPVVSTQLLLNGSLAEGEIIIKSENITDNTKVIIVQLNETVEITCVRPNNNTRKSIHLGPGQALYATGDIIGNIRQAHCDVSGRNWSNMIEKVKAQLRKIFNKTITFDSSAGGDLEITTHSFNCRGEFFYCNTSGLFNNETISNGTITLPCGXKQIVRLWQRVGQAMYSPPIARNITCKSNITGLLLTRDGGNANNASETETFRPAGGNMKDNWRNELYKYKVVKIKPLGVAPTKARRRVVGREKRAVGVGAVFLGFLGAAGSTMGAASITLTVQVRQLLSGIVQQQSNLLRAIEAQQHLLQLTVWGIKQLQARVLALERYLRDQQLLGIWGCSGKLICTTNVPWNASWSNKTYNDIWDNMTWIQWDREISNYTQQIYSLIEESQNQQEKNEQDLLALDNWASLWTWFDITKWLWYIKIFIMIVGGLISLKIIFAVLSIVNRVRKGYSPLSFQTLTHHQREPDRPERIEEEGGEQDKDRSIRLVSGFLALAWDDLRSLCLFSYHRLRDFILIAARTVELLGHNSLKGLRLGWEGLKYLWNLLLYWGRELKNSAINLLDTIAIAVANWTDRVIEIVQRAFRAFLNIPTRIRQGLERALL

SEQ ID NO: 104 - protein, 849 aa, HIV
MRVKGIQRNWQHLWKWGTLILGLVIICSASDNLWVTVYYGVPVWEDADTPLFCASDAKSYSSEKHNVWATHACVPTDPNPQEIAIENVTENFNMWKNNMVEQMQEDIISLWEESLKPCVKLTPLCITLNCTNVNSANHTEANNTVENKEEIKNCSFKITTERGGKKKEEYALFYKLDVVPISNGNKTSYRLIHCNVSTIKQACPKVNFDPIPIHYCAPAGFAILKCRDKEYNGTGPCKNVSTVQCTHGIKPVVSTQLLLNGSLAEEDIRIRSENFTDNTKVIIVQLNNSIEINCIRPNNNTRKSIPIGPGQAFYATGDIIGDIRQAHCNVSRIKWREMLKNVTAQLRKIYNNKNITFNSSAGGDLEITTHSFNCRGEFFYCNTSGLFNNNISNINNETITLPCKIKQIVRMWQKVGQAMYALPIAGNLVCKSNITGLILTRDGGNNNDSTEETFRPGGGDMRDNWRSELYKYKTVKIKSLGVAPTRARRRVVEREKRAVGLGAVFLGFLGAAGSTMGAASITLTAQVRQLLSGIVQQQSNLLRAIEAQQHLLQLTVWGIKQLQSRVLAIERYLKDQQLLGIWGCSGKLICTTNVPWNTSWSNKSYNEIWDNMTWLEWEREIHNYTQHIYSLIEESQNQQEKNEQDLLALDKWASLWNWFDISNWLWYIRIFIMIVGGLIGLRIVFAVLSIVNRVRQGYSPLSFQTLTHHQREPDRLGKTEEGGGEQDRDRSTRLVSGFLALAWDDLRSLCLFSYHRLRDLVLIAARTVELLGRSSLKGLRLGWEGLKYLWNLLLYWGRELKNSAINLLDTIAIATANGTDRVIEVAQRAYRAILNVPTRIRQGLERALL

SEQ ID NO: 105 - protein, 883 aa, HIV
MRVRGTQTNWQLLWKWGTLILGLVIICSASNNLWVTVYYGVPVWEDADTTLFCASDAKAYSTESHNVWATHACVPTDPNPQEIPMKNVTENFNMWKNNMVEQMHEDIISLWDESLKPCVKLTPLCVTLTCTNVTINVTSSSNSSVTSTSTVGSTTSTSTVGSTASTSTVGSTAGYREELKNCSFNITTEIKDRRKQEYALFYKLDIVPINDGRNSSANNYRLINCNVSTIKQACPKVSFDPIPIHYCAPAGFAILKCRDKEFNGTGLCKNVSTVQCTHGIKPVVSTQLLLNGSLAEGEIMIRSENITNNAKNIIVQLNETVPITCARPSNNTRKSIRFGPGQAFYATDAIIGDIRQAHCNISRIRWENMKQNVTAKLEKIFKKNITFNPPAGGDLEITTHSFICRGGFFYCNTSALFNSSSLSNSNSSNDTITLPCRIKQIVRMWQRVGQAMYAPPIEGNITCMSNITGLLLTRDGGENNGTNETEIFRPVGGDMRDNWRSELYKYKVVKIKPLGVTPTRARRRVVGREKRAVGLGAVLLGFLGTAGSTMGAASITLTVQVRQLLSGIVQQQSNLLRAIEAQQHLLQLTVWGIKQLQARVLAVERYLKDQQLLGIWGCSGRLICTTNVPWNASWSNKSYNQIWDNLTWVQWEREISNYTQQIYTLLEESQNQQEKNEQDLLALDKWADLWNWFDISRWLWYIKIFIMIVGGLIGLRIVFAVLSIINRVRKGYSPLSFQTLTHHQREPDRPGRIEEGDGEQDRDKSIRLVSGFLALAWDDLRSLCLFSYHHLRDFILIAARTVELLGRSSLKGLSLGWEGLKYLWNLLLYWGRELKNSAINLIDTVAIAVANWTDRAIEVVQRVGRAILNIPVRIRQGLERLLL

SEQ ID NO: 106 - protein, 862 aa, HIV
TRVMETQRNYPSLWRWGTLILGMLLICSVVGNLWVTVYYGVPVWKEAKTTLFCASDAKAYDTERHNVWATHACVPTDPNPQEMVLENVTETFNMWVNDMVEQMHTDIISLWDQSLKPCVKLTPLCVTLDCSSVNATNVTKSNNSTDINIGEIQEQRNCSFNVTTAIRDKNQKVHALFYRADIVQIDEGERNKSDNHYRLINCNTSVIKQACPKVSFEPIPIHYCAPAGFAILKCNGKKFNGTGPCTNVSTVQCTHGIRPVVSTQLLLNGSLAEVEEVIIRSKNITDNTKNIIVQLNEPVQINCTRTGNNTRKSIRIGPGQAFYATGDIIGDIRRAYCNISGKQWNETLHKVITKLGSYFDNKTIILQPPAGGDIEIITHSFNCGGEFFYCNTTKLFNSTWTNSSYTNDTYNSNSTEDITGNITLQCKIKQIVNMWQRVGQAMYAPPIRGNITCISNITGLILTFDRNNTNNVTFRPGGGDMRDNWRSELYKYKVVKIEPLGVAPTEARRRVVEREKRAVGMGAFFLGFLGAAGSTMGAASITLTVQARQLLSGIVQQQSNLLRAIQAQQHMLQLTVWGIKQLQARVLAVERYLKDQQLLGIWGCSGKLICTTNVPWNSSWSNKSLDEIWDNMTWMEWDKQINNYTDEIYRLLEVSQNQQEKNEQDLLALDKWANLWNWFSITNWLWYIRIFIMIVGGIIGLRIVFAVLSIVNRVRQGYSPLSLQTLIPNQRGPDRPREIEEEGGEQDRDRSIRLVNGFLPLVWEDLRNLCLFSYRRLRDLLSIVARTVELLGRRGWEALKLLGNLLLYWGQELKNSAISLLNTTAIAVAEGTDRIIELVQRAWRAILHIPRRIRQGFERALL

SEQ ID NO: 107 - protein, 855 aa, HIV
TRVMRNYPQWWRGGILLLGMLLIYSAAGNLWVTVYYGVPVWKEAKTTLFCASDAKAYEPEKHNVWATHACVPTDPSPQEMVLANVTENFNMWDNDMVEQMQTDIISLWDQSLKPCVKLTPLCVTLDCSNITRNDTNSSSTVNATSSPSANELTNCSFNVTTVIRDKQQRVHALFYRLDVVPIDETSNNNNSNSTKYRLINCNTSVITQACPKVSFDPIPIHYCAPAGFAILKCNNKTFNGTGPCTNVSTVQCTHGIKPVVSTQLLLNGSLAEGQVIIRSKNISDNTKNIIVQLDSPIEITCTRPNNNTRKGIHFGPGQAFYATGDIIGNIRQAHCNVSEEKWNKTLQQIATQLSKYFVNRTLIFKPHSGGDLEVTTHSFNCRGEFFYCNTSGLFNSSWTGDNINMPNDTGKNITLPCRIKQIVNMWQRVGQAMYAPPIKGSITCVSNITGLILTYDEDKGNNDNVTFRPGGGDMRDNWRSELYKYKVVKIEPLGVAPTEARRRVVEREKRAVGMGAFFLGFLGAAGSTMGAASITLTVQARQLLSGIVQQQSNLLRAIQAQQHMLQLTVWGVKQLQARVLAVERYLKDQQLLGIWGCSGKLICTTNVPWNSTWSNKSLAEIWDNMTWMEWDRQIDNYTEVIYRLLELSQTQQEQNEQDLLALDKWDSLWNWFSITNWLWYIKIFIIIVGALIGLRIIFAVLSIVGRVRQGYSPLSFQTLIPNPRGPDRPEGIEEEGGEQDRGRSVRLVNGFLPIVWDDLRSLCLFSYRLLRDSLLIVIRTVELLGRRGREALKYLWNLLQYWGQELKNSAINLLNTTAIVVAEGTDRIIEIVQRAWRAVLHIPRRIRQGLERILL

SEQ ID NO: 108 - protein, 848 aa, HIV
TRVMETQRNYPSLWRWGTLILGMLLICSAAQNLWVTVYYGVPVWKEAKTTLFCASDAKAYETEKHNVWATHACVPTDPNPQEMVMENVTESFNMWENNMVEQMHTDIISLWDQSLKPCVKLTPLCVTLNCTNVRNNTSNSTSSMEAGGELTNCSFNVTTVLRDKQQKVHALFYRLDVVPIDNNSTQYRLINCNTSVITQACPKVSFEPIPIHYCAPAGFAILKCNNKTFNGTGLCTNVSTVQCTHGIRPVVSTQLLLNGSLAEEQIIIRTKNISDNTKNIIVQLKTPVNITCTRPNNNTRTSIHLGPGRAFYATGDIIGDIRQAHCNISRTDWNKTLHQVVTQLGIHLNNRTISFKPNSGGDMEVRTHSFNCRGEFFYCNTSGLFNSSWEMHTNYTSNDTKGNENITLPCRIKQIVNMWQRVGRAMYAPPIQGNIMCVSNITGLILTIDEGNASAENYTFRPGGGDMRDNWRSELYKYKVVKIEPLGIAPTKTRRRVVEREKRAVGMGASFLGFLGAAGSTMGAASITLTVQARQLLSGIVQQQSNLLRAIQARQHMLQLTVWGIKQLQARVLAVERYLRDQQLLGIWGCSGKLICTTNVPWNSSWSNKSQSEIWDNMTWMEWDKQISNYTEEIYRLLEVSQTQQEKNEQDLLALDKWASLWTWFDISHWLWYIKIFIMIVGGLIGLRIIFAVLSIVNRVRQGYSPLSFQTLVPNPRGPDRPEGTEEGGGEQDRDRSVRLVNGFLPVVWDDLRSLSLFSYRLLRDLLLIVVRTVELLGRRGREALKYLWNLLQYWGQELKNSAIDLLNTTAIAVAEGTDGIIVIVQRAWRAILHIPRRIRQGFERSLL

SEQ ID NO: 109 - protein, 849 aa, HIV
TKVMETQMNWKNLWKWGLMIFGMLMICNGAEKLWVTVYYGVPVWKDAKTTLFCASDAKSYSTEKHNVWATHACVPTDPNPREITMENVTENFNIWKNDMVEQMHDDIISVWDESLKPCVKITPLCVTLNCTEATINNSTDNSNTTSPSPTITNPLGMKNCSFNITTEIGDRRKKEYALFYKQDVVSIDNSNSSYILINCNTSVIKQACPKVSFEPIPIHYCAPAGFAILKCNDNKFNGTGPCKNVSTVQCTHGIKPVVSTQLLLNGSIAEKEVIIKSKNISDNAKTIIVQLNQTVEINCTRPANNTRKGIPIGPGQVLYATGAVIGNIRQAHCNISGVKWNNTLHKVAEELREQEHFKNKTIVFAPANSGGDIEIMMHTFNCGGEFFYCNTSILFNSSWDENSTVTINETVTLPCKIRQIVRMWQRVGQAIYAPPIAGNITCTSNITGLLLTRDGGNTKESNSSETFRPTGGDMKDNWRNELYKYKIVKVEPLGVAPTRAKRRVVEREKRAIVGMGAVFLGFLGTAGSTMGAASIALTVQARQLLSGIVQQQSNLLKAIEAQQHLLRLTVWGIKQLQARILAVERYLKDQQLLGIWGCSGKLICTTNVPWNSSWSNKSHDEIWNNMTWVEWEREIDNYTRIIYNLIEVSQNQQEKNEQDLLALDKWTSLWSWFKISNWLWYIRIFIMIVGGLIGLRIIFAVLAIVNRVRQGYSPLSLQTLIPNPTGVDRPGEIEEGGGEQGRTRSIRLVSGFLALAWDDLRSLCLFSYHRLRDFVLIVARTVETLGRRGWEILKYLGNLVCYWGQELKNSAISLLNATAIAVAAGTDRIIEIVQGIFRAILHIPRRIRQ

SEQ ID NO: 110 - protein, 851 aa, HIV
  misc_feature (402)..(402): Xaa can be any naturally occurring amino acid
TRVMETQKNWQTLWRGGLMIFGMLMICKAKEDLWVTVYYGVPVWKDAKTTLFCASDAKAYSTEKHNVWATHACVPTDPSPQEMNLPNVTENFNMWKNDMVDQMQEDIISVWDESLKPCVKITPLCVTLNCSNITSNSNTTSNSSVSSPDIMTNCSFNITTEIRNKRKQEYALFYRQDVVPIDSNNKNYILINCNTSVIKQACPKVSFQPIPIHYCAPAGFAILKCNDKNFNGTGSCKNVSTVQCTHGIKPVVSTQLLLNGSIAEGDIIIRSENISDNAKNIIVQLNKTVEIVCYRPNNNTRKGIHMGPGQVLYATGEIIGNIRETHCNISERDWSNTLRRVATKLREHFNKTINFTSPSGGDIEIVTHSFNCGGEFLYCNTSKLFNSSWDKNSIEATNDTSXATITIPCKIKQIVRMWQRTGQAIYAPPIAGNITCTSNITGLLLTRDGGNRGNGSENGTETFRPTGGNMKDNWRSELYKYKVVEIEPLGVAPTKAKRRVVEREKRAVGIGAVFLGFLGTAGSTMGAASITLTVQVRQLLSGIVQQQSNLLKAIEAQQHLLKLTVWGIKQLQARVLAVERYLKDQQLLGIWGCSGKLICTTNVPWNASWSNKSYEDIWENMTWIQWEREINNYTGIIYSLIEEAQNQQENNEKDLLALDKWTNLWNWFNISNWLWYIKIFIMIIGGLIGLRIIFAVLAIVNRVRQGYSPLSFQTLIPNPTEADRPGGIEEGGGEQGRTRSIRLVNGFLALAWDDLRNLCLFSYHRLRDFVLIAARTVGTLGLRGWEILKYLVNLVWYWGQELKNSAISLLNTTAIAVAEGTDRIIEIAQRAFRAILHIPRRIRQGLERALL

SEQ ID NO: 111 - protein, 853 aa, HIV
  misc_feature (557)..(557): Xaa can be any naturally occurring amino acid
TRVMETQTSWLSLWRWGLMIFGMLMICSARENLWVTVYYGVPVWRDAKTTLFCASDAKAYSTEKHNVWATHACVPTDPNPQEMSLPNVTENFNMWKNDMVDQMQEDIISVWDESLKPCVKITPLCVTLNCSDVNSNNSTDSNSSASNNSPEIMKNCSFNVTTEIRNKRKQEYALFYRQDVVPINSDNKSYILINCNTSVIKQACPKVSFQPIPIHYCAPAGFAILKCNNKTFNGTGPCKNVSTVQCTHGIKPVVSTQLLLNGSVAEGDIIIRSENISDNAKNIIVQLNDTVEIVCTRPNNNTRKGIHMGPGQVLYATGEIIGDIRKAYCNISRKDWNNTLRRVAKKLREHFNKTIDFTSPSGGDIEITTHSFNCGGEFFYCNTSTLFNSSWDENNIKDTNSTNDNTTITIPCKIKQIVRMWQRTGQAIYAPPIAGNITCKSNITGLLLTRDGGNRNGSENGTETFRPTGGNMKDNWRSELYKYKVVELEPLGVAPTKAKRRVVEREKRAVGIGAVFLGFLGTAGSTMGAASITLTVQVRQLLSGIVQQQSNLLKAIXAQQHLLKLTVWGIKQLQARVLAVERYLKDQQLLGIWGCSGKLICTTNVPWNASWSNKSYEDIWENMTWIQWEREINNYTGIIYSLIEEAQNQQETNEKDLLALDKWTNLWNWFNISNWLWYIKIFIMIIGGLIGLRIIFAVLAIVNRVRQGYSPLSFQTLIPNPTEADRPGGIEEGGGEQGRTRSIRLVNGFLALAWDDLRSLCLFSYHRLRDFVLIAARTVGTLGLRGWEILKYLVNLVWYWGQELKNSAISLLNTTAIAVAEGTDRIIEIAQRAFRAILHIPRRIRQGLERALL

SEQ ID NO: 112 - protein, 851 aa, HIV
MRAREIQRNWQHLGKRGILFLGILIICSAANNLWVTVYYGVPVWKEATTTLFCASDAKAYETEVHNVWATHACVPTDPNPQEVVLENVTENFNMWKNNMVEQMHTDIISLWDESLKPCVKLTPLCVTLTCTNVTNNRTNANKNDTNINATVTSTDEIKNCSFNITTELKDKKKRVSALFYKLDIVQIKQSEINQSESEDRLINCNTSTVTQACPKVSFEPIPIHYCAPAGFAILKCNNNTCNGTGPCTNVSTVQCTHGIKPVVSTQLLLNGSLAEEEIIIRSEDITKNTKNIIVQLNEAVEINCTRPSNNTRKSIHIGPGRAFYATGDIIGDIRQAHCNISGGQWNKTVNQVKKELGKHFNKTIIFQPSSGGDPQVTRHIFNCRGEFSYCDTTDTVDDTEEEEDTTITIPCRIKQIINMWQKVGQAIYAPPTAGNITCRSNITGMILTRDGGNDNNTRTEETFRPGGGDMRDNWRSELYKYKVVQIEPLGIAPTRARRRVVQREKRAVGIGALFLGFLGAAGSTMGAASITLTVQARQLLSGIVQQQNNLLRAIEAQQQMLQLTVWGIKQLRARVLAVERYLRDQQLLGIWGCSGKLICTTNVPWNSSWSNKSQSEIWENMTWMQWEKEISNHTSTIYRLIEESQIQQEKNEQDLLALDKWASLWNWFDISNWLWYIKIFIMIVGGLIGLRIVFTVLSVVNRVRQGYSPLSFQTLTPSPRGPDRPEGIEEGGGEQDKDRSVRLVSGFLALAWDDLRNLCLFSYRHLRDLVLIATRILDRGLKGSWEALKYLWNLILYWGQEIKNSAINLLNTTAIAVAEGTDRIIEIVYRAFRALLHIPRRIRQGFERLLL

SEQ ID NO: 113 - protein, 842 aa, HIV
MRVRGMQRNWQTLGNWGILFLGILIICSNADKLWVTVYYGVPVWKEATPTLFCASDAKAYEKEVHNVWATHACVPTDPNPQEVEMENVTENFNMWKNNMVEQMHTDIISLWDESLKPCVELTPLCVTLNCTDYKGTNSTNNATSTVVSPAEIKNCSFNITTEIKDKKKKESALFYRLDVLPLNGEGNNSSTEYRLINCNTSTITQTCPKVTFEPIPIHYCAPAGFAILKCKDKRFNGTGPCKNVSTVQCTHGIKPVVSTQLLLNGSLAEEEIIIRSENITDNTKNIIVQLNETVQINCTRPNNNTRKSIHMGPGKAFYTTGDIIGDIRQAHCNISGEKWNMTLSRVKEKLKEHFKNGTITFKPPNPGGDPEILTHMFNCAGEFFYCNTTKLFNETGENGTITLPCRIKQIINMWQKVGKAIYAPPIAGSINCSSNITGMILTRDGGNNTHNETFRPGGGDMRDNWRSELYKYKVVQIEPLGIAPTRARRRVVQREKRAVGLGAVFFGFLGAAGSTMGAASITLTVQARQLLSGIVQQQSNLLRAIEAQQHLLQLTVWGIKQLRARILAVERYLKDQQLLGIWGCSGKLICTTNVPWNSSWSNKSWEEIWNNMTWMEWEKEIGNYSDTIYKLIEESQTQQEKNEQDLLALDKWASLWNWFDITKWLWYIKIFIMIIGGLIGLRIAFAVLSVVNRVRQGYSPLSFQTLIPTSRGADRPEGIEEEGGEQDKNRSVRLVSGFLALAWDDLRNLCLFSYRQLRNLILIVTRILERGLRGGWEALKYLWNLVQYWSQELKNSAISLLNTTAIAVAGGTDRIIEIGQRAFRALLHIPRRIRQGLERALL

SEQ ID NO: 114 - protein, 842 aa, HIV
  misc_feature (598)..(598): Xaa can be any naturally occurring amino acid
VRMMGMQIGWPFFCLMISLTIGSKKYWATVYYGVPVWRDVETVLFCASDAKAHSTEAHNIWATQACVPTDPNPQEVPLDNVTEPFNMWENKMAEQVQEDIISLWEQSLKPCVKLTPLCVTMNCSNSNGNRTTDEKEKPGNGTDLEARHMKNCSFNITTEIHDKKKQAYSLFYVEDVVPLNDGNNSTYRLINCNTTAVTQACPKTTFEPIPIHYCAPPGFAIMKCNEANFNGTGECKNVSTVQCTHGIKPVISTQLILNGSLDKDIVIRNNSGGNLLVQWNEIVTMNCTRPGNNTGGQVQIGPAMTFYNIEKIVGDIRQAHCNVSNEWRSMWNKTKEKIKSLLGNNITFKAQEKNGGDPEVTHLMFNCGGEFIYCNTSRLFNESMNTNGTNGTITLPCRIRQIVNLWTRVGKGIYAPPIRGNLTCNSTITGLILEHSGGSNGTVYPTGGNMVNLWRQELYKYKTVSIEPIGVAPGKAKRRTVSREKRAAFGLGALFLGFLGAAGSTMGAASITLTVQARTLLSGIVQQQNNLVRAIEAQQHLLQLSIWGIKQLRAKVLAIERYLRDQQILSLWGCSGKTICYTTVPWNETWSNNTSYDXIWGNLTWQQWDRKVRNYSGVIFELIXKAQEQQNTNEKSLLELDQWASLWNWFSITNWLWYIKIAIMVVAGIIGIRIISVIITIIARVRQGYSPLSLQTLIPTTTTRGPDRPEETEEGVGGQGRDRSVRLVSGFLTLVWEDFRNLLIFLYHRLTDSLLILQRTLELLGRSLIRGLQLLNELRTRLWGIIAYWGKELKDSAISLLNTIAIVVAEGTDRLIELAQRIGRGILHIPRRIRQGLERALL

SEQ ID NO: 115 - protein, 852 aa, HIV
MKVMGMQSGWMGMKSGWLLFYLLVSLIKVIGSEQHWVTVYYGVPVWREAETTLFCASDAKAHSTEAHNIWATQACVPTDPNPQEVLLPNVTEKFNMWENKMADQMQEDIISLWEQSLKPCVKLTPLCVTMLCNDSYGEERNNTNMTTREPDIGYKQMKNCSFNATTELTDKKKQVYSLFYVEDVVPINAYNKTYRLINCNTTAVTQACPKTSFEPIPIHYCAPPGFAIMKCNEGNFSGNGSCTNVSTVQCTHGIKPVISTQLILNGSLNTDGIVIRNDSHSNLLVQWNETVPINCTRPGNNTGGQVQIGPAMTFYNIEKIVGDIRQAYCNVSKELWEPMWNRTREEIKKILGKNNITFRARERNEGDLEVTHLMFNCRGEFFYCNTSKLFNEELLNETGEPITLPCRIRQIVNLWTRVGKGIYAPPIRGVLNCTSNITGLVLEYSGGPDTKETIVYPSGGNMVNLWRQELYKYKVVSIEPIGVAPGKAKRRTVSREKRAAFGLGALFLGFLGAAGSTMGAASITLTVQARTLLSGIVQQQNILLRAIEAQQHLLQLSIWGIKQLQAKVLAIERYLRDQQILSLWGCSGKTICYTTVPWNETWSNNTSYDTIWNNLTWQQWDEKVRNYSGVIFGLIEQAQEQQNTNEKSLLELDQWDSLWSWFGITKWLWYIKIAIMIVAGIVGIRIISIVITIIARVRQGYSPLSLQTLIPTARGPDRPEETEGGVGEQDRGRSVRLVSGFSALVWEDLRNLLIFLYHRLTDSLLILRRTLELLGQSLSRGLQLLNELRTHLWGILAYWGKELRDSAISLLNTTAIVVAEGTDRIIELAQRIGRGILHIPRRIRQGLERALI

SEQ ID NO: 116 - protein, 839 aa, HIV
  misc_feature (367)..(367): Xaa can be any naturally occurring amino acid
RRVMGMQSGWPFFCLLISLTIGSDPHWVTVYYGVPVWRDAETVLFCASDAKAHSTEAHNIWATQACVPTDPNPQEVLLTNVTEYFNMWENKMAEQMQEDIISLWEQSLKPCVKLTPLCVTMLCNNSNGNSAGNSTTNRTEDLEDRQMKNCSFNITTEIRDRKKQVYSLFYVEDVVPIKDGTDNNTYRLINCNTTAVTQACPKTTFEPIPIHYCAPPGFAIMKCNEGNFSGNGSCTNVSTVQCTHGIKPVISTQLILNGSLDTDDIVIRHHGGNLLVQWNETVSINCTRPGNNTGGQVQIGPAMTFYNIEKIVGDVRQAYCNVSEEWGSMWNKTKKKIKRLLGNNTTFKAQDKNGGDLEVTHLMFNCXGEFFYCNTSRLFNESENKTNKTIILPCRIKQIVXLWTRVXKGIYAPPIRGNLSCXSSITGLILEHSGENGNKTVYPSGGNMVNLWRQELYKYKVVSIEPIGVAPGKAKRRTVSREKRAAFGLGALFLGFLGAAGSTMGAASITLTVQARTLLSGIVQQQNNLLRAIEAQQHLLQLSIWGIKQLRAKVLAIERYLRDQQILSLWGCSGKTICYTTVPWNDXWSSNTSYDTIWXNLTWQQWDRKVRNYSGVIFDLIEQAQEQQNTNEKALLELDQWASLWNWFDITKWLWYIKIAIMVVAGIIGIRIISAIITIIARVRQGYSPLSLQTLIPTAARGPDRPEETEEGVGGQDRGRSVRLVSGFLALIWEDLRNLLIFLYHRLADSLLIIRRTLEILGQSLSRGLQLLNELRIRLWGIIAYWGKELKDSAISLLNTTAIVVAEGTDRFIELAQRIGRGILHIPRRIRQGLERALL

SEQ ID NO: 117 - protein, 859 aa, HIV
  misc_feature (568)..(568): Xaa can be any naturally occurring amino acid
MKAMEKRNKKLWTLYLAMALITPCLSLRQLYATVYAGVPVWEDATPVLFCASDANLTSTEKHNIWASQACVPTDPTPYEYPLHNVTDDFNIWKNYMVEQMQEDIISLWDQSLKPCVQMTFLCVQMECTNIAGTTNENLMKKCEFNVTTVIKDKKEKKQALFYVSDLMELNETSSTNKTNSKMYTLTNCNSTTITQACPKVSFEPIPIHYCAPAGYAIFKCNSTEFNGTGTCRNITVVTCTHGIRPTVSTQLILNGTLSKGKIRMMAKDILEGGKNIIVTLNSTLNMTCERPQIDIQEMRIGPMAWYSMGIGGTAGNSSRAAYCKYNATDWGKILKQTAERYLELVNNTGSINMTFNHSSGGDLEVTHLHFNCHGEFFYCNTAKMFNYTFSCNGTTCSVSNVSQGNNGTLPCKLRQVVRSWIRGQSGLYAPPIKGNLTCMSNITGMILQMDNTWNSSNNNVTFRPIGGDMKDIWRTELFNYKVVRVKPFSVAPTRIARPVISTRTHREKRAVGLGMLFLGVLSAAGSTMGAAATTLAVQTHTLLKGIVQQQDNLLRAIQAQQQLLRLSXWGIRQLRARLLALETLLQNQQLLSLWGCKGKLVCYTSVKWNRTWIGNESIWDTLTWQEWDRQISNISSTIYEEIQKAQVQQEQNEKKLLELDEWASIWNWLDITKWLWYIKIAIIIVGALVGVRVIMIVLNIVKNIRQGYQPLSLQIPNHHQEEAGTPGRTGGGGGEEGRPRWIPSPQGFLPLLYTDLRTIILWTYHLLSNLASGIQKVISYLRLGLWILGQKIINVCRICAAVTQYWLQELQNSATSLLDTLAVAVANWTDGIIAGIQRIGTGIRNIPRRIRQGLERSLL

SEQ ID NO: 118 - protein, 872 aa, HIV
MKVMKKNNRKSWSLYIAMALLIPCLSYSKQLYATVYSGVPVWEEAAPVLFCASDANLTSTEQHNIWASQACVPTDPNPHEFPLGNVTDNFDIWKNYMVDQMHEDIISLWEQSLKPCEKMTFLCVQMNCVDLQTNKTGLLNETINEMRNCSFNVTTVLTDKKEQKQALFYVSDLSKVNDSNAVNGTTYMLTNCNSTIIKQACPKVSFEPIPIHYCAPTGYAIFKCNDTDFNGTGLCHNISVVTCTHGIKPTVSTQLILNGTLSREKIRIMGKNITESAKNIIVTLNTPINMTCIREGIAEVQDIYTGPMRWRSMTLKRSNNTSPRSRVAYCTYNKTVWENALQQTAIRYLNLVNQTENVTIIFSRTSGGDAEVSHLHFNCHGEFFYCNTSGMFNYTFINCTKSGCQEIKGSNETNKNGTIPCKLRQLVRSWMKGESRIYAPPIPGNLTCHSNITGMILQLDQPWNSTGENTLRPVGGDMKDIWRTKLYNYKVVQIKPFSVAPTKMSRPIINIHTPHREKRAVGLGMLFLGVLSAAGSTMGAAATALTVRTHSVLKGIVQQQDNLLRAIQAQQHLLRLSVWGIRQLRARLQALETLIQNQQRLNLWGCKGKLICYTSVKWNTSWSGRYNDDSIWDNLTWQQWDQHINNVSSIIYDEIQAAQDQQEKNVKALLELDEWASLWNWFDITKWLWYIKIAIIIVGALIGIRVIMIILNLVKNIRQGYQPLSLQIPVPHRQEAETPGRTGEEGGEGDRPKWTALPPGFLQQLYTDLRTIILWTYHLLSNLISGIRRLIDYLGLGLWILGQKTIEACRLCGAVMQYWLQELKNSATNLLDTIAVSVANWTDGIILGLQRIGQGFLHIPRRIRQGAERILV

SEQ ID NO: 119 - protein, 868 aa, HIV
MKVMEKRNRKLGILCMVMALITPCLSHNQHYATVYAGVPVWEEATPVLFCASDVNLTSTEQHNIWASQACVPTDPSPYEYPLKNVTDNFNIWENYMVEQMQEDIISLWEQSLKPCVQMTFLCVQMNCTNVNDETNSSVKNDTSSSENLMKKCEFNVTTVLKDKKEKQQALFYVSDLMKVNENNDTMYTLINCNSTTIKQTCPKVSFEAIPIHYCAPAGYAIFKCNNTGFNGTGPCTNVTVVTCTHGIRPTVSTQLILNGTISEGKIRIMGKNISDTGKNIIVTINSTINMTCERPGNQTVQKILTGPVAWYSMGLKNNLTNSRAASCKYNSSVWEEALKQTAERYLELMNNTNTVNITFNHSTGGDPEVTHLHFNCHGEFFYCNTSQMFNYTFSCTRTNCIRQSNSSINGTISCRIKQVVRSWIQGGSGLYAPPRPGYLTCNSSITGMILQLDKTWNRTNNSESTFRPIGGDMKDIWRTELFKYKVVKIKPFSVAPTKIARPVIGTGTRREKRAVGLGMLFLGVLSAAGSTMGAAATTLAVQTHTLMKGIVQQQDNLLRAIQAQQQLLRLSVWGIRQLRARLLALETLIQNQQLLNLWGCKGRLVCYTSVKWNRTWTNNNTDLDTIWGNLTWQEWDQQISNISATIYDEIQKAQVQQEHNEKKLLELDEWASIWNWLDITKWLWYIKIAIIIVGALIGVRIVMIVLNLVRNIRHGYQPLSFQTPTHHQQPEAQAPGGTGEGGGERDRLRSIPSPQGFLPLLYTDLRTIILWSYHLLSNLASGIQTVISHLGLGLWILGQKIISACRICIAVIQYWLQELQNSATSLLDTLAVAVANWTDGIILGLQRIGRGILNIPRRIRQGLERALL

SEQ ID NO: 120 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVETLIQNQQRLNLWGCKGKLICYTSVKWNTSWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 121 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVETYLKDQQLLNIWGCKGKLICTTAVKWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 122 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVETYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 123 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLRIWGCFGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 124 - protein, 713 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVEKTYLKDQQLLNIWGCKGKLICTTAVKWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 125 - protein, 132 aa, HIV
WGIKQLQARILAVETLIQNQQRLNLWGCKGKLICYTSVKWNTSWSNKSLEGGGGGTGCAAAGGTAAGCTAATTTGTTATACTAGTGTTAAATGGAACACCTCATGGAGCAATAAATCTCTGGAACAGATTTG

SEQ ID NO: 126 - protein, 31 aa, HIV
TYLKDQQLLNIWGCKGKLICTTAVKWNASWS

SEQ ID NO: 127 - protein, 31 aa, HIV
TYLKDQQLLGIWGCSGKLICTTAVPWNASWS

SEQ ID NO: 128 - protein, 31 aa, HIV
RYLKDQQLLRIWGCFGKLICTTAVPWNASWS

SEQ ID NO: 129 - protein, 32 aa, HIV
KTYLKDQQLLNIWGCKGKLICTTAVKWNASWS

SEQ ID NO: 130 - protein, 18 aa, HIV
LQNRRGLDLLFLKEGGLC

SEQ ID NO: 131 - protein, 23 aa, HIV
LQARVLAVERYLKDQQLLGIWGC

SEQ ID NO: 132 - protein, 23 aa, HIV
LQARVLAVERYLKDQQLLRIWGC

SEQ ID NO: 133 - protein, 23 aa, HIV
LQARILAVETLIQNQQLLNLWGC

SEQ ID NO: 134 - protein, 53 aa, HIV
LRARLLALETFIQNQQRLNLWGCKGNLICYTSVKWNDTWKGNSDTSLENIWDN

SEQ ID NO: 135 - protein, 50 aa, HIV
LQARILAVETYLKDQQRLNIWGCKGKLICTTAVKWNASWSNKSLEQIWNH

SEQ ID NO: 136 - protein, 50 aa, HIV
LQARILAVETYLKDQQRLGIWGCSGKLICTTAVPWNASWSNKSLEQIWNH

SEQ ID NO: 137 - protein, 50 aa, HIV
LQARILAVERYLKDQQRLNIWGCSGKLICTTAVPWNASWSNKSLEQIWNH

SEQ ID NO: 138 - protein, 50 aa, HIV
LQARILAVERYLKDQQRLGIWGCKGKLICTTAVPWNASWSNKSLEQIWNH

SEQ ID NO: 139 - protein, 50 aa, HIV
LQARILAVERYLKDQQRLGIWGCSGKLICTTAVKWNASWSNKSLEQIWNH

SEQ ID NO: 140 - protein, 50 aa, HIV
LQARILAVETYLKDQQRLNIWGCSGKLICTTAVPWNASWSNKSLEQIWNH

SEQ ID NO: 141 - protein, 50 aa, HIV
LQARILAVETYLKDQQRLGIWGCKGKLICTTAVPWNASWSNKSLEQIWNH

SEQ ID NO: 142 - protein, 50 aa, HIV
LQARILAVETYLKDQQRLGIWGCSGKLICTTAVKWNASWSNKSLEQIWNH

SEQ ID NO: 143 - protein, 50 aa, HIV
LQARILAVETYLKDQQRLNIWGCKGKLICTTAVPWNASWSNKSLEQIWNH

SEQ ID NO: 144 - protein, 50 aa, HIV
LQARILAVETYLKDQQRLNIWGCSGKLICTTAVKWNASWSNKSLEQIWNH

SEQ ID NO: 145 - protein, 50 aa, HIV
LQARILAVETYLKDQQRLGIWGCKGKLICTTAVKWNASWSNKSLEQIWNH

SEQ ID NO: 146 - protein, 50 aa, HIV
LQARILAVERYLKDQQRLNIWGCKGKLICTTAVPWNASWSNKSLEQIWNH

SEQ ID NO: 147 - protein, 50 aa, HIV
LQARILAVERYLKDQQRLNIWGCSGKLICTTAVKWNASWSNKSLEQIWNH

SEQ ID NO: 148 - protein, 50 aa, HIV
LQARILAVERYLKDQQRLNIWGCKGKLICTTAVKWNASWSNKSLEQIWNH

SEQ ID NO: 149 - protein, 50 aa, HIV
LQARILAVERYLKDQQRLGIWGCKGKLICTTAVKWNASWSNKSLEQIWNH

SEQ ID NO: 150 - protein, 50 aa, HIV
LQARIMAVERYLKDQQRLGIWGCSGKLICTTAVPWNASWSNKSLEQIWNH

SEQ ID NO: 151 - protein, 50 aa, HIV
LQARILAMERYLKDQQRLGIWGCSGKLICTTAVPWNASWSNKSLEQIWNH

SEQ ID NO: 152 - protein, 50 aa, HIV
LQARILAVERYMKDQQRLGIWGCSGKLICTTAVPWNASWSNKSLEQIWNH

SEQ ID NO: 153 - protein, 50 aa, HIV
LQARILAVERYLKDQQRMGIWGCSGKLICTTAVPWNASWSNKSLEQIWNH

SEQ ID NO: 154 - protein, 50 aa, HIV
LQARILAVERYLKDQQRLGMWGCSGKLICTTAVPWNASWSNKSLEQIWNH

SEQ ID NO: 155 - protein, 50 aa, HIV
LQARILAVERYLKDQQRLGIWGCSGKLICTTAMPWNASWSNKSLEQIWNH

SEQ ID NO: 156 - protein, 50 aa, HIV
LQARILAVERYLKDQQRLGIWGCSGKLICTTAVPWNASWSNKSLEQMWNH

SEQ ID NO: 157 - protein, 50 aa, HIV
LRARLLALERYLKDQQRLGIWGCSGKLICTTAVPWNASWSNKSLEQIWNH

SEQ ID NO: 158 - protein, 50 aa, HIV
LRARLLALETFIQNQQRLNLWGCSGKLICTTAVPWNASWSNKSLEQIWNH

SEQ ID NO: 159 - protein, 50 aa, HIV
RQTEVLAIERYLKDQQRLGIWGCSGKLICTTAVPWNASWSNKSLEQIWNH

SEQ ID NO: 160 - protein, 50 aa, HIV
LRTRVLAIERYLKDQQRLGIWGCSGKLICTTAVPWNASWSNKSLEQIWNH

SEQ ID NO: 161 - protein, 50 aa, HIV
LRTRVQAIERYLKDQQRLGIWGCSGKLICTTAVPWNASWSNKSLEQIWNH

SEQ ID NO: 162 - protein, 50 aa, HIV; identical to SEQ ID NO: 32

SEQ ID NO: 163 - protein, 50 aa, HIV
LRTRVLALETLIQNQQRLGIWGCSGKLICTTAVPWNASWSNKSLEQIWNH

SEQ ID NO: 164 - protein, 50 aa, HIV; identical to SEQ ID NO: 34

SEQ ID NO: 165 - protein, 50 aa, HIV; identical to SEQ ID NO: 35

SEQ ID NO: 166 - protein, 50 aa, HIV; identical to SEQ ID NO: 36

SEQ ID NO: 167 - protein, 50 aa, HIV
LQTRIQAVETFIRDQQRLGMWGCSGKLICTTAVPWNASWSNKSLEQIWNH

SEQ ID NO: 168 - protein, 50 aa, HIV
LQARILAMERYMKDQQRMGMWGCSGKLICTTAVPWNASWSNKSLEQIWNH

SEQ ID NO: 169 - protein, 50 aa, HIV
LRARILAMERYMKDQQRMGMWGCSGKLICTTAVPWNASWSNKSLEQIWNH

SEQ ID NO: 170 - protein, 50 aa, HIV
LRARILAMERYLKDQQRLGIWGCSGKLICTTAVPWNASWSNKSLEQIWNH

SEQ ID NO: 171 - protein, 50 aa, HIV
LRARILAMETYLKDQQRLGIWGCSGKLICTTAVPWNASWSNKSLEQIWNH

SEQ ID NO: 172 - protein, 50 aa, HIV
LRARILAMETYMKDQQRMGMWGCSGKLICTTAVPWNASWSNKSLEQIWNH

SEQ ID NO: 173 - protein, 50 aa, HIV
LRTRILAMETYLKDQQRLGIWGCSGKLICTTAVPWNASWSNKSLEQIWNH

SEQ ID NO: 174 - protein, 50 aa, HIV
LRTRVLALETLIQNQQRLNIWGCSGKLICTTAVPWNASWSNKSLEQIWNH

SEQ ID NO: 175 - protein, 50 aa, HIV
LQTRIQAVETFIRDQQRLNMWGCSGKLICTTAVPWNASWSNKSLEQIWNH

SEQ ID NO: 176 - protein, 50 aa, HIV
LQARILAVERYLKDQQRLGIWGCKGNLICTTAVPWNASWSNKSLEQIWNH

SEQ ID NO: 177 - protein, 50 aa, HIV
LQARILAVERYLKDQQRLGIWGCFGKLICTTAVPWNASWSNKSLEQIWNH

SEQ ID NO: 178 - protein, 50 aa, HIV
LQARILAVERYLKDQQRLRIWGCSGKLICTTAVPWNASWSNKSLEQIWNH

SEQ ID NO: 179 - protein, 53 aa, HIV
LRARLLALETFIQNQQRLRLWGCKGNLICYTSVKWNDTWKGNSDTSLENIWDN

SEQ ID NO: 180 - protein, 53 aa, HIV
LRARLLALETFIQNQQRLRLWGCFGNLICYTSVKWNDTWKGNSDTSLENIWDN

SEQ ID NO: 181 - protein, 53 aa, HIV
LRARLLALETFIQNQQRLNLWGCFGNLICYTSVKWNDTWKGNSDTSLENIWDN

SEQ ID NO: 182 - protein, 50 aa, HIV
LQTRIQAVETFIRDQQRLGMWGCKGNLICTTAVPWNASWSNKSLEQIWNH

SEQ ID NO: 183 - protein, 50 aa, HIV; identical to SEQ ID NO: 53

SEQ ID NO: 184 - protein, 50 aa, HIV
LQTRIQAVETFIRDQQRLNMWGCKGNLICTTAVPWNASWSNKSLEQIWNH

SEQ ID NO: 185 - protein, 50 aa, HIV; identical to SEQ ID NO: 55

SEQ ID NO: 186 - protein, 50 aa, HIV; identical to SEQ ID NO: 56

SEQ ID NO: 187 - protein, 50 aa, HIV
LRTRVLALETLIQNQQRLRIWGCFGKLICTTAVPWNASWSNKSLEQIWNH

SEQ ID NO: 188 - protein, 50 aa, HIV; identical to SEQ ID NO: 58

SEQ ID NO: 189 - protein, 50 aa, HIV; identical to SEQ ID NO: 59

SEQ ID NO: 190 - protein, 50 aa, HIV; identical to SEQ ID NO: 60

SEQ ID NO: 191 - protein, 50 aa, HIV
LQTRIQAVETFIRDQQRLRMWGCFGKLICTTAVPWNASWSNKSLEQIWNH

SEQ ID NO: 192 - protein, 50 aa, HIV
LQARILAMERYMKDQQRMRMWGCFGKLICTTAVPWNASWSNKSLEQIWNH

SEQ ID NO: 193 - protein, 50 aa, HIV
LRARILAMERYMKDQQRMRMWGCFGKLICTTAVPWNASWSNKSLEQIWNH

SEQ ID NO: 194 - protein, 50 aa, HIV
LQARILAVERYLKDQQRLRIWGCFGKLICTTAVPWNASWSNKSLEQIWNH

SEQ ID NO: 195 - protein, 50 aa, HIV; identical to SEQ ID NO: 65

SEQ ID NO: 196 - protein, 50 aa, HIV; identical to SEQ ID NO: 135

SEQ ID NO: 197 - protein, 50 aa, HIV; identical to SEQ ID NO: 136

SEQ ID NO: 198 - protein, 50 aa, HIV
LQARILAVKTYLKDQQRLNIWGCKGKLICTTAVKWNASWSNKSLEQIWNH

SEQ ID NO: 199 - protein, 50 aa, HIV
LQARILAVETLIQNQQLLNLWGCKGKLICYTSVKWNTSWSNKSLEQIWNH

SEQ ID NO: 200 - protein, 50 aa, HIV
LQARILAVETLIQNQQRLSLWGCKGKLICYTSVKWNTSWSNKSLEQIWNH

SEQ ID NO: 201 - protein, 50 aa, HIV
LQARILAVETLIQNQQRLDLWGCKGKLICYTSVKWNTSWSNKSLEQIWNH

SEQ ID NO: 202 - protein, 50 aa, HIV
LQARILAVETLIQNQQLLSLWGCKGKLICYTSVKWNTSWSNKSLEQIWNH

SEQ ID NO: 203 - protein, 50 aa, HIV
LQARILAVETLIQNQQLLDLWGCKGKLICYTSVKWNTSWSNKSLEQIWNH

SEQ ID NO: 204 - protein, 50 aa, HIV
LQARILAVETLIQNQHLLNLWGCKGKLICYTSVKWNTSWSNKSLEQIWNH

SEQ ID NO: 205 - protein, 50 aa, HIV
LQARILAVETLIQNQHRLSLWGCKGKLICYTSVKWNTSWSNKSLEQIWNH

SEQ ID NO: 206 - protein, 50 aa, HIV
LQARILAVETLIQNQHRLDLWGCKGKLICYTSVKWNTSWSNKSLEQIWNH

SEQ ID NO: 207 - protein, 50 aa, HIV
LQARILAVETLIQNQHLLSLWGCKGKLICYTSVKWNTSWSNKSLEQIWNH

SEQ ID NO: 208 - protein, 50 aa, HIV
LQARILAVETLIQNQHLLDLWGCKGKLICYTSVKWNTSWSNKSLEQIWNH

SEQ ID NO: 209 - protein, 53 aa, HIV; identical to SEQ ID NO: 134

SEQ ID NO: 210 - protein, 50 aa, HIV
LQARVLAVETYLKDQQRLNIWGCKGKLICTTAVKWNASWSNKSLEQIWNH

SEQ ID NO: 211 - protein, 50 aa, HIV
LQARVLAVETYLKDQQRLGIWGCSGKLICTTAVPWNASWSNKSLEQIWNH

SEQ ID NO: 212 - protein, 50 aa, HIV
LQARVLAVERYLKDQQRLNIWGCSGKLICTTAVPWNASWSNKSLEQIWNH

SEQ ID NO: 213 - protein, 50 aa, HIV
LQARVLAVERYLKDQQRLGIWGCKGKLICTTAVPWNASWSNKSLEQIWNH

SEQ ID NO: 214 - protein, 50 aa, HIV
LQARVLAVERYLKDQQRLGIWGCSGKLICTTAVKWNASWSNKSLEQIWNH

SEQ ID NO: 215 - protein, 50 aa, HIV
LQARVLAVETYLKDQQRLNIWGCSGKLICTTAVPWNASWSNKSLEQIWNH

SEQ ID NO: 216 - protein, 50 aa, HIV
LQARVLAVETYLKDQQRLGIWGCKGKLICTTAVPWNASWSNKSLEQIWNH

SEQ ID NO: 217 - protein, 50 aa, HIV
LQARVLAVETYLKDQQRLGIWGCSGKLICTTAVKWNASWSNKSLEQIWNH

SEQ ID NO: 218 - protein, 50 aa, HIV
LQARVLAVETYLKDQQRLNIWGCKGKLICTTAVPWNASWSNKSLEQIWNH

SEQ ID NO: 219 - protein, 50 aa, HIV
LQARVLAVETYLKDQQRLNIWGCSGKLICTTAVKWNASWSNKSLEQIWNH

SEQ ID NO: 220 - protein, 50 aa, HIV
LQARVLAVETYLKDQQRLGIWGCKGKLICTTAVKWNASWSNKSLEQIWNH

SEQ ID NO: 221 - protein, 50 aa, HIV
LQARVLAVERYLKDQQRLNIWGCKGKLICTTAVPWNASWSNKSLEQIWNH

SEQ ID NO: 222 - protein, 50 aa, HIV
LQARVLAVERYLKDQQRLNIWGCSGKLICTTAVKWNASWSNKSLEQIWNH

SEQ ID NO: 223 - protein, 50 aa, HIV
LQARVLAVERYLKDQQRLNIWGCKGKLICTTAVKWNASWSNKSLEQIWNH

SEQ ID NO: 224 - protein, 50 aa, HIV
LQARVLAVERYLKDQQRLGIWGCKGKLICTTAVKWNASWSNKSLEQIWNH

SEQ ID NO: 225 - protein, 50 aa, HIV
LQARVMAVERYLKDQQRLGIWGCSGKLICTTAVPWNASWSNKSLEQIWNH

SEQ ID NO: 226 - protein, 50 aa, HIV
LQARVLAMERYLKDQQRLGIWGCSGKLICTTAVPWNASWSNKSLEQIWNH

SEQ ID NO: 227 - protein, 50 aa, HIV
LQARVLAVERYMKDQQRLGIWGCSGKLICTTAVPWNASWSNKSLEQIWNH

SEQ ID NO: 228 - protein, 50 aa, HIV
LQARVLAVERYLKDQQRMGIWGCSGKLICTTAVPWNASWSNKSLEQIWNH

SEQ ID NO: 229 - protein, 50 aa, HIV
LQARVLAVERYLKDQQRLGMWGCSGKLICTTAVPWNASWSNKSLEQIWNH

SEQ ID NO: 230 - protein, 50 aa, HIV
LQARVLAVERYLKDQQRLGIWGCSGKLICTTAMPWNASWSNKSLEQIWNH

SEQ ID NO: 231 - protein, 50 aa, HIV
LQARVLAVERYLKDQQRLGIWGCSGKLICTTAVPWNASWSNKSLEQMWNH

SEQ ID NO: 232 - protein, 50 aa, HIV
LQTRVQAMETYIRDQQFMGIWGCSGKLICTTAVPWNASWSNKSLEQIWNH

SEQ ID NO: 233 - protein, 50 aa, HIV
LQTRVQAVETFIRDQQFMGIWGCSGKLICTTAVPWNASWSNKSLEQIWNH

SEQ ID NO: 234 - protein, 50 aa, HIV
SQARVQAVETFIRDQQFMGIWGCSGKLICTTAVPWNASWSNKSLEQIWNH

SEQ ID NO: 235 - protein, 50 aa, HIV
LQTRVQAVETFIRDQQRLGMWGCSGKLICTTAVPWNASWSNKSLEQIWNH

SEQ ID NO: 236 - protein, 50 aa, HIV
LQARVLAMERYMKDQQRMGMWGCSGKLICTTAVPWNASWSNKSLEQIWNH

SEQ ID NO: 237 - protein, 50 aa, HIV
LRARVLAMERYMKDQQRMGMWGCSGKLICTTAVPWNASWSNKSLEQIWNH

SEQ ID NO: 238 - protein, 50 aa, HIV
LRARVLAMERYLKDQQRLGIWGCSGKLICTTAVPWNASWSNKSLEQIWNH

SEQ ID NO: 239 - protein, 50 aa, HIV
LRARVLAMETYLKDQQRLGIWGCSGKLICTTAVPWNASWSNKSLEQIWNH

SEQ ID NO: 240 - protein, 50 aa, HIV
LRARVLAMETYMKDQQRMGMWGCSGKLICTTAVPWNASWSNKSLEQIWNH

SEQ ID NO: 241 - protein, 50 aa, HIV
LRTRVLAMETYLKDQQRLGIWGCSGKLICTTAVPWNASWSNKSLEQIWNH

SEQ ID NO: 242 - protein, 50 aa, HIV
LQTRVQAVETFIRDQQRLNMWGCSGKLICTTAVPWNASWSNKSLEQIWNH

SEQ ID NO: 243 - protein, 50 aa, HIV
LQARVLAVERYLKDQQRLGIWGCKGNLICTTAVPWNASWSNKSLEQIWNH

SEQ ID NO: 244 - protein, 50 aa, HIV
LQARVLAVERYLKDQQRLGIWGCFGKLICTTAVPWNASWSNKSLEQIWNH

SEQ ID NO: 245 - protein, 50 aa, HIV
LQARVLAVERYLKDQQRLRIWGCSGKLICTTAVPWNASWSNKSLEQIWNH

SEQ ID NO: 246 - protein, 50 aa, HIV
LQTRVQAVETFIRDQQRLGMWGCKGNLICTTAVPWNASWSNKSLEQIWNH

SEQ ID NO: 247 - protein, 50 aa, HIV
LQTRVQAVETFIRDQQFMGIWGCKGNLICTTAVPWNASWSNKSLEQIWNH

SEQ ID NO: 248 - protein, 50 aa, HIV
LQTRVQAVETFIRDQQRLNMWGCKGNLICTTAVPWNASWSNKSLEQIWNH

SEQ ID NO: 249 - protein, 50 aa, HIV
LQTRVQAVETFIRDQQFMNIWGCKGNLICTTAVPWNASWSNKSLEQIWNH

SEQ ID NO: 250 - protein, 50 aa, HIV
LQTRVQAVETFIRDQQFMRIWGCFGNLICTTAVPWNASWSNKSLEQIWNH

SEQ ID NO: 251 - protein, 50 aa, HIV
LQTRVQAMETYIRDQQFMRIWGCFGKLICTTAVPWNASWSNKSLEQIWNH

SEQ ID NO: 252 - protein, 50 aa, HIV
LQTRVQAVETFIRDQQFMRIWGCFGKLICTTAVPWNASWSNKSLEQIWNH

SEQ ID NO: 253 - protein, 50 aa, HIV
SQARVQAVETFIRDQQFMRIWGCFGKLICTTAVPWNASWSNKSLEQIWNH

SEQ ID NO: 254 - protein, 50 aa, HIV
LQTRVQAVETFIRDQQRLRMWGCFGKLICTTAVPWNASWSNKSLEQIWNH

SEQ ID NO: 255 - protein, 50 aa, HIV
LQARVLAMERYMKDQQRMRMWGCFGKLICTTAVPWNASWSNKSLEQIWNH

SEQ ID NO: 256 - protein, 50 aa, HIV
LRARVLAMERYMKDQQRMRMWGCFGKLICTTAVPWNASWSNKSLEQIWNH

SEQ ID NO: 257 - protein, 50 aa, HIV
LQARVLAVERYLKDQQRLRIWGCFGKLICTTAVPWNASWSNKSLEQIWNH

SEQ ID NO: 258 - protein, 50 aa, HIV
LQARVLAVETLIQNQQRLNLWGCKGKLICYTSVKWNTSWSNKSLEQIWNH

SEQ ID NO: 259 - protein, 50 aa, HIV; identical to SEQ ID NO: 210

SEQ ID NO: 260 - protein, 50 aa, HIV; identical to SEQ ID NO: 211

SEQ ID NO: 261 - protein, 50 aa, HIV
LQARVLAVKTYLKDQQRLNIWGCKGKLICTTAVKWNASWSNKSLEQIWNH

SEQ ID NO: 262 - protein, 50 aa, HIV
LQARVLAVETLIQNQQLLNLWGCKGKLICYTSVKWNTSWSNKSLEQIWNH

SEQ ID NO: 263 - protein, 50 aa, HIV
LQARVLAVETLIQNQQRLSLWGCKGKLICYTSVKWNTSWSNKSLEQIWNH

SEQ ID NO: 264 - protein, 50 aa, HIV
LQARVLAVETLIQNQQRLDLWGCKGKLICYTSVKWNTSWSNKSLEQIWNH

SEQ ID NO: 265 - protein, 50 aa, HIV
LQARVLAVETLIQNQQLLSLWGCKGKLICYTSVKWNTSWSNKSLEQIWNH

SEQ ID NO: 266 - protein, 50 aa, HIV
LQARVLAVETLIQNQQLLDLWGCKGKLICYTSVKWNTSWSNKSLEQIWNH

SEQ ID NO: 267 - protein, 50 aa, HIV
LQARVLAVETLIQNQHLLNLWGCKGKLICYTSVKWNTSWSNKSLEQIWNH

SEQ ID NO: 268 - protein, 50 aa, HIV
LQARVLAVETLIQNQHRLSLWGCKGKLICYTSVKWNTSWSNKSLEQIWNH

SEQ ID NO: 269 - protein, 50 aa, HIV
LQARVLAVETLIQNQHRLDLWGCKGKLICYTSVKWNTSWSNKSLEQIWNH

SEQ ID NO: 270 - protein, 50 aa, HIV
LQARVLAVETLIQNQHLLSLWGCKGKLICYTSVKWNTSWSNKSLEQIWNH

SEQ ID NO: 271 - protein, 50 aa, HIV
LQARVLAVETLIQNQHLLDLWGCKGKLICYTSVKWNTSWSNKSLEQIWNH

SEQ ID NO: 272 - protein, 50 aa, HIV
LQARVLAVETYLKDQQLLNIWGCKGKLICTTAVKWNASWSNKSLEQIWNH

SEQ ID NO: 273 - protein, 50 aa, HIV
LQARVLAVETYLKDQQLLGIWGCSGKLICTTAVPWNASWSNKSLEQIWNH

SEQ ID NO: 274 - protein, 50 aa, HIV
LQARVLAVERYLKDQQLLNIWGCSGKLICTTAVPWNASWSNKSLEQIWNH

SEQ ID NO: 275 - protein, 50 aa, HIV
LQARVLAVERYLKDQQLLGIWGCKGKLICTTAVPWNASWSNKSLEQIWNH

SEQ ID NO: 276 - protein, 50 aa, HIV
LQARVLAVERYLKDQQLLGIWGCSGKLICTTAVKWNASWSNKSLEQIWNH

SEQ ID NO: 277 - protein, 50 aa, HIV
LQARVLAVETYLKDQQLLNIWGCSGKLICTTAVPWNASWSNKSLEQIWNH

SEQ ID NO: 278 - protein, 50 aa, HIV
LQARVLAVETYLKDQQLLGIWGCKGKLICTTAVPWNASWSNKSLEQIWNH

SEQ ID NO: 279 - protein, 50 aa, HIV
LQARVLAVETYLKDQQLLGIWGCSGKLICTTAVKWNASWSNKSLEQIWNH

SEQ ID NO: 280 - protein, 50 aa, HIV
LQARVLAVETYLKDQQLLNIWGCKGKLICTTAVPWNASWSNKSLEQIWNH

SEQ ID NO: 281 - protein, 50 aa, HIV
LQARVLAVETYLKDQQLLNIWGCSGKLICTTAVKWNASWSNKSLEQIWNH

SEQ ID NO: 282 - protein, 50 aa, HIV
LQARVLAVETYLKDQQLLGIWGCKGKLICTTAVKWNASWSNKSLEQIWNH

SEQ ID NO: 283 - protein, 50 aa, HIV
LQARVLAVERYLKDQQLLNIWGCKGKLICTTAVPWNASWSNKSLEQIWNH

SEQ ID NO: 284 - protein, 50 aa, HIV
LQARVLAVERYLKDQQLLNIWGCSGKLICTTAVKWNASWSNKSLEQIWNH

SEQ ID NO: 285 - protein, 50 aa, HIV
LQARVLAVERYLKDQQLLNIWGCKGKLICTTAVKWNASWSNKSLEQIWNH

SEQ ID NO: 286 - protein, 50 aa, HIV
LQARVLAVERYLKDQQLLGIWGCKGKLICTTAVKWNASWSNKSLEQIWNH

SEQ ID NO: 287 - protein, 50 aa, HIV
LQARVMAVERYLKDQQLLGIWGCSGKLICTTAVPWNASWSNKSLEQIWNH

SEQ ID NO: 288 - protein, 50 aa, HIV
LQARVLAMERYLKDQQLLGIWGCSGKLICTTAVPWNASWSNKSLEQIWNH

SEQ ID NO: 289 - protein, 50 aa, HIV
LQARVLAVERYMKDQQLLGIWGCSGKLICTTAVPWNASWSNKSLEQIWNH

SEQ ID NO: 290 - protein, 50 aa, HIV
LQARVLAVERYLKDQQLMGIWGCSGKLICTTAVPWNASWSNKSLEQIWNH

SEQ ID NO: 291 - protein, 50 aa, HIV
LQARVLAVERYLKDQQLLGMWGCSGKLICTTAVPWNASWSNKSLEQIWNH

SEQ ID NO: 292 - protein, 50 aa, HIV
LQARVLAVERYLKDQQLLGIWGCSGKLICTTAMPWNASWSNKSLEQIWNH

SEQ ID NO: 293 - protein, 50 aa, HIV
LQARVLAVERYLKDQQLLGIWGCSGKLICTTAVPWNASWSNKSLEQMWNH

SEQ ID NO: 294 - protein, 50 aa, HIV; identical to SEQ ID NO: 232

SEQ ID NO: 295 - protein, 50 aa, HIV; identical to SEQ ID NO: 233

SEQ ID NO: 296 - protein, 50 aa, HIV; identical to SEQ ID NO: 234

SEQ ID NO: 297 - protein, 50 aa, HIV
LQTRVQAVETFIRDQQLLGMWGCSGKLICTTAVPWNASWSNKSLEQIWNH

SEQ ID NO: 298 - protein, 50 aa, HIV
LQARVLAMERYMKDQQLMGMWGCSGKLICTTAVPWNASWSNKSLEQIWNH

SEQ ID NO: 299 - protein, 50 aa, HIV
LRARVLAMERYMKDQQLMGMWGCSGKLICTTAVPWNASWSNKSLEQIWNH

SEQ ID NO: 300 - protein, 50 aa, HIV
LRARVLAMERYLKDQQLLGIWGCSGKLICTTAVPWNASWSNKSLEQIWNH

SEQ ID NO: 301 - protein, 50 aa, HIV
LRARVLAMETYLKDQQLLGIWGCSGKLICTTAVPWNASWSNKSLEQIWNH

SEQ ID NO: 302 - protein, 50 aa, HIV
LRARVLAMETYMKDQQLMGMWGCSGKLICTTAVPWNASWSNKSLEQIWNH

SEQ ID NO: 303 - protein, 50 aa, HIV
LRTRVLAMETYLKDQQLLGIWGCSGKLICTTAVPWNASWSNKSLEQIWNH

SEQ ID NO: 304 - protein, 50 aa, HIV
LQTRVQAVETFIRDQQLLNMWGCSGKLICTTAVPWNASWSNKSLEQIWNH

SEQ ID NO: 305 - protein, 50 aa, HIV
LQARVLAVERYLKDQQLLGIWGCKGNLICTTAVPWNASWSNKSLEQIWNH

SEQ ID NO: 306 - protein, 50 aa, HIV
LQARVLAVERYLKDQQLLGIWGCFGKLICTTAVPWNASWSNKSLEQIWNH

SEQ ID NO: 307 - protein, 50 aa, HIV
LQARVLAVERYLKDQQLLRIWGCSGKLICTTAVPWNASWSNKSLEQIWNH

SEQ ID NO: 308 - protein, 50 aa, HIV
LQTRVQAVETFIRDQQLLGMWGCKGNLICTTAVPWNASWSNKSLEQIWNH

SEQ ID NO: 309 - protein, 50 aa, HIV; identical to SEQ ID NO: 247

SEQ ID NO: 310 - protein, 50 aa, HIV
LQTRVQAVETFIRDQQLLNMWGCKGNLICTTAVPWNASWSNKSLEQIWNH

SEQ ID NO: 311 - protein, 50 aa, HIV; identical to SEQ ID NO: 249

SEQ ID NO: 312 - protein, 50 aa, HIV; identical to SEQ ID NO: 250

SEQ ID NO: 313 - protein, 50 aa, HIV; identical to SEQ ID NO: 251

SEQ ID NO: 314 - protein, 50 aa, HIV; identical to SEQ ID NO: 252

SEQ ID NO: 315 - protein, 50 aa, HIV; identical to SEQ ID NO: 253

SEQ ID NO: 316 - protein, 50 aa, HIV
LQTRVQAVETFIRDQQLLRMWGCFGKLICTTAVPWNASWSNKSLEQIWNH

SEQ ID NO: 317 - protein, 50 aa, HIV
LQARVLAMERYMKDQQLMRMWGCFGKLICTTAVPWNASWSNKSLEQIWNH

SEQ ID NO: 318 - protein, 50 aa, HIV
LRARVLAMERYMKDQQLMRMWGCFGKLICTTAVPWNASWSNKSLEQIWNH

SEQ ID NO: 319 - protein, 50 aa, HIV
LQARVLAVERYLKDQQLLRIWGCFGKLICTTAVPWNASWSNKSLEQIWNH

SEQ ID NO: 320 - protein, 50 aa, HIV; identical to SEQ ID NO: 258

SEQ ID NO: 321 - protein, 50 aa, HIV; identical to SEQ ID NO: 272

SEQ ID NO: 322 - protein, 50 aa, HIV; identical to SEQ ID NO: 273

SEQ ID NO: 323 - protein, 50 aa, HIV
LQARVLAVKTYLKDQQLLNIWGCKGKLICTTAVKWNASWSNKSLEQIWNH

SEQ ID NO: 324 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARVLAVERYLKDQQLLRIWGCFGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 325 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVETLIQNQQLLNLWGCKGKLICYTSVKWNTSWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 326 - protein, 50 aa, HIV
WGIKQLQARILAVETLIQNQQLLNLWGCKGKLICYTSVKWNTSWSNKSLE

SEQ ID NO: 327 - protein, 50 aa, HIV
WGIKQLQARVLAVERYLKDQQLLGIWGCSGKLICTTAVPWNASWSNKSLE

SEQ ID NO: 328 - protein, 50 aa, HIV
WGIKQLQARVLAVERYLKDQQLLRIWGCFGKLICTTAVPWNASWSNKSLE

SEQ ID NO: 329 - protein, 50 aa, HIV
WGIKQLQARVLAVETLIQNQQRLNLWGCKGKLICYTSVKWNTSWSNKSLE

SEQ ID NO: 330 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARVLAVETYLKDQQLLNIWGCKGKLICTTAVKWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 331 - protein, 712 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARVLAVETYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 332 - protein, 712 aa, HIV; identical to SEQ ID NO: 324

SEQ ID NO: 333 - protein, 713 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARVLAVEKTYLKDQQLLNIWGCKGKLICTTAVKWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGY

SEQ ID NO: 334 - protein, 50 aa, HIV; identical to SEQ ID NO: 272

SEQ ID NO: 335 - protein, 49 aa, HIV
LQARVLAVETYLKDQQLLGIWGCSGKLICTTAVPWNASWSNKSLEQIWN

SEQ ID NO: 336 - protein, 49 aa, HIV
LQARVLAVERYLKDQQLLRIWGCFGKLICTTAVPWNASWSNKSLEQIWN

SEQ ID NO: 337 - protein, 51 aa, HIV
LQARVLAVEKTYLKDQQLLNIWGCKGKLICTTAVKWNASWSNKSLEQIWNH